(19) United States
(12) Patent Application Publication
(10) Pub. No.: US 2024/0176238 A1
(43) Pub. Date: May 30, 2024

(54) SULFONIUM SALT, RESIST COMPOSITION AND PATTERN FORMING PROCESS

(71) Applicant: Shin-Etsu Chemical Co., Ltd., Tokyo (JP)

(72) Inventors: Masaki Ohashi, Joetsu-shi (JP); Jun Hatakeyama, Joetsu-shi (JP); Tatsuya Yamahira, Joetsu-shi (JP); Yuki Suda, Joetsu-shi (JP)

(73) Assignee: Shin-Etsu Chemical Co., Ltd., Tokyo (JP)

(21) Appl. No.: 18/490,857

(22) Filed: Oct. 20, 2023

(30) Foreign Application Priority Data
Nov. 2, 2022 (JP) 2022-176346

(51) Int. Cl. G03F 7/004; C07C 69/74; C07C 69/78; C07C 69/96; C07D 333/76; C08F 212/14; C08F 220/18; C08F 220/22; G03F 7/038; G03F 7/039

(52) U.S. Cl. CPC G03F 7/0045; C07C 69/74; C07C 69/78; C07C 69/96; C07D 333/76; C08F 212/24; C08F 220/1806; C08F 220/1807; C08F 220/22; G03F 7/0382; G03F 7/0392; C07C 2601/12; C07C 2603/74

(57) ABSTRACT

A sulfonium salt of specific structure has a high decomposition efficiency and a high acid diffusion controlling ability upon exposure. A resist composition comprising the sulfonium salt offers a high sensitivity, reduced LWR and improved CDU independent of whether it is of positive or negative tone.

### CROSS-REFERENCE TO RELATED APPLICATIONS

This non-provisional application claims priority under 35 U.S.C. § 119(a) on Patent Application No. 2022-176346 filed in Japan on Nov. 2, 2022, the entire contents of which are hereby incorporated by reference.

### TECHNICAL FIELD

This invention relates to a sulfonium salt, a resist composition comprising the same, and a patterning process using the composition.

### BACKGROUND ART

To meet the demand for higher integration density and operating speed of LSIs, the effort to reduce the pattern rule is in rapid progress. As the use of 5G high-speed communications and artificial intelligence (AI) is widely spreading, high-performance devices are needed for their processing. As the advanced miniaturization technology, manufacturing of microelectronic devices at the 5-nm node by the lithography using EUV of wavelength 13.5 nm has been implemented in a mass scale. Studies are made on the application of EUV lithography to 3-nm node devices of the next generation and 2-nm node devices of the next-but-one generation. IMEC in Belgium announced its successful development of 1-nm and 0.7-nm node devices.

As the feature size reduces, image blurs due to acid diffusion become a problem. To insure resolution for fine patterns with a size of 45 nm et seq., not only an improvement in dissolution contrast is important as previously reported, but the control of acid diffusion is also important as reported in Non-Patent Document 1. Since chemically amplified resist compositions are designed such that sensitivity and contrast are enhanced by acid diffusion, an attempt to minimize acid diffusion by reducing the temperature and/or time of post-exposure bake (PEB) fails, resulting in drastic reductions of sensitivity and contrast.

A triangular tradeoff relationship among sensitivity, resolution, and edge roughness (LWR) has been pointed out. Specifically, a resolution improvement requires to suppress acid diffusion whereas a short acid diffusion distance leads to a decline of sensitivity.

The addition of an acid generator capable of generating a bulky acid is an effective means for suppressing acid diffusion. It was then proposed to incorporate repeat units derived from an onium salt having a polymerizable unsaturated bond in a polymer. Since this polymer functions as an acid generator, it is referred to as polymer-bound acid generator. Patent Document 1 discloses a sulfonium or iodonium salt having a polymerizable unsaturated bond, capable of generating a specific sulfonic acid. Patent Document 2 discloses a sulfonium salt having a sulfonic acid directly attached to the backbone.

Resist compositions adapted for the EUV and ArF lithography processes are typically based on (meth)acrylate polymers having acid labile groups. These acid labile groups undergo deprotection reaction when a photoacid generator capable of generating a sulfonic acid which is substituted at α-position with fluorine (referred to as “α-fluorinated sulfonic acid, hereinafter) is used, but not when a photoacid generator capable of generating a sulfonic acid which is not substituted at α-position with fluorine (referred to as “α-non-fluorinated sulfonic acid, hereinafter) or carboxylic acid is used. When a sulfonium or iodonium salt capable of generating α-fluorinated sulfonic acid is mixed with a sulfonium or iodonium salt capable of generating α-non-fluorinated sulfonic acid, the sulfonium or iodonium salt capable of generating α-non-fluorinated sulfonic acid undergoes ion exchange with the α-fluorinated sulfonic acid. Through the ion exchange, the α-fluorinated sulfonic acid once generated upon light exposure is converted back to the sulfonium or iodonium salt. Then the sulfonium or iodonium salt of α-non-fluorinated sulfonic acid or carboxylic acid functions as a quencher. Patent Document 3 discloses a resist composition comprising a sulfonium or iodonium salt capable of generating carboxylic acid as the quencher.

Sulfonium salt type quenchers capable of generating carboxylic acid are proposed. So far known are sulfonium salts of salicylic acid or β-hydroxycarboxylic acid (Patent Document 4), salicylic acid derivatives (Patent Documents 5 and 6), fluorosalicylic acid (Patent Document 7), hydroxynaphthoic acid (Patent Document 8), and thiolcarboxylic acid (Patent Document 9). In particular, salicylic acid is highly effective for suppressing acid diffusion due to the intramolecular hydrogen bond between carboxylic acid and hydroxy group.

Also, Patent Document 10 discloses a sulfonium salt consisting of a carboxylic acid anion and a phenyldibenzothiophenium cation substituted with an alkoxycarbonyl group. Exemplary carboxylic acid anions mentioned therein include adamantane carboxylic acid and analogues thereof, salicylic acid, and fluoroalkylcarboxylic acids.

### CITATION LIST

Patent Document 1: JP-A 2006-045311 (U.S. Pat. No. 7,482,108)
Patent Document 2: JP-A 2006-178317
Patent Document 3: JP-A 2007-114431
Patent Document 4: WO 2018/159560
Patent Document 5: JP-A 2020-203984
Patent Document 6: JP-A 2020-091404
Patent Document 7: JP-A 2020-091312
Patent Document 8: JP-A 2019-120760
Patent Document 9: JP-A 2019-074588
Patent Document 10: JP-A 2021-035937
Non-Patent Document 1: SPIE Vol. 6520 65203L-1 (2007)

### SUMMARY OF THE INVENTION

It is desired to have a quencher capable of reducing the roughness (LWR) of line patterns, improving the dimensional uniformity (CDU) of hole patterns, and increasing the sensitivity of a resist composition. To this end, image blurs due to acid diffusion must be significantly reduced.

An object of the invention is to provide a resist composition which exhibits a high sensitivity, reduced LWR, and improved CDU independent of whether it is of positive or negative tone, and a pattern forming process using the same.

The inventors have found that a sulfonium salt of specific structure has a high decomposition efficiency and a high acid diffusion controlling ability upon exposure. Owing to these properties, a resist composition using the sulfonium salt as the quencher exhibits reduced LWR, improved CDU, high resolution, and wide process margin.

In one aspect, the invention provides a sulfonium salt having the formula (1).

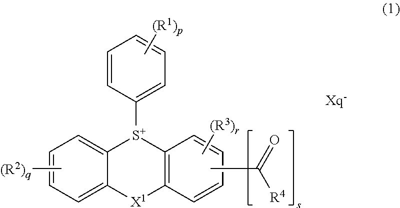

Herein p, q and r are each independently an integer of 0 to 3, s is 1 or 2, and r+s is from 1 to 3,
R¹ and R² are each independently halogen, trifluoromethyl, trifluoromethoxy, trifluoromethylthio, nitro, cyano, —C(═O)—R⁴, —O—C(═O)—R⁵ or —O—R⁵,
R³ is halogen, trifluoromethyl, trifluoromethoxy, trifluoromethylthio, nitro, cyano, —O—C(═O)—R⁵ or —O—R⁵,
R⁴ is a C₁-C₁₀ hydrocarbyl group, C₁-C₁₀ hydrocarbyloxy group, or —O—R^{4A}, the hydrocarbyl group and hydrocarbyloxy group may be substituted with fluorine or hydroxy, R^{4A} is an acid labile group,
R⁵ is a C₁-C₁₀ hydrocarbyl group,
X¹ is a single bond, ether bond, carbonyl group, —N(R)—, sulfide bond or sulfonyl group, R is hydrogen or a C₁-C₆ saturated hydrocarbyl group,

Xq⁻ is an anion having the formula (2):

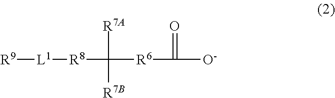

wherein R⁶ is a single bond or C₁-C₄ alkanediyl group,
R^{7A} and R^{7B} are each independently hydrogen, fluorine, or trifluoromethyl, at least one thereof being fluorine or trifluoromethyl,
R⁸ is a single bond or C₁-C₂₀ hydrocarbylene group which may contain a heteroatom,
R⁹ is a C₆-C₂₀ straight or branched hydrocarbyl group which may contain a heteroatom or C₃-C₂₀ cyclic hydrocarbyl group which may contain a heteroatom, and
L¹ is an ether bond, ester bond, carbonate bond or urethane bond.

In a preferred embodiment, R⁶ is a single bond, R^{7A} and R^{7B} are fluorine.

In another aspect, the invention provides a resist composition comprising a quencher containing the sulfonium salt defined herein.

In a preferred embodiment, the resist composition further comprises a base polymer.

The base polymer preferably comprises repeat units having the formula (a1) or repeat units having the formula (a2).

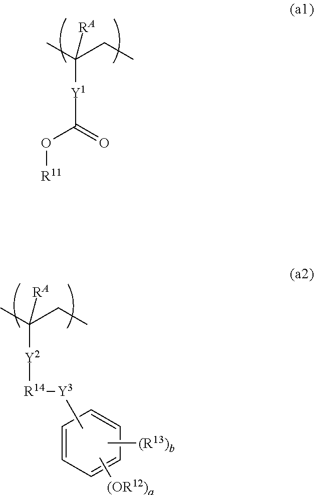

Herein R^{A} is each independently hydrogen or methyl,
Y¹ is a single bond, phenylene, naphthylene, or a C₁-C₁₂ linking group containing an ester bond and/or lactone ring,
Y² is a single bond or ester bond,
Y³ is a single bond, ether bond or ester bond,
R¹¹ and R¹² are each independently an acid labile group,
R¹³ is fluorine, trifluoromethyl, cyano or a C₁-C₆ saturated hydrocarbyl group,
R¹⁴ is a single bond or a C₁-C₆ alkanediyl group in which some carbon may be replaced by an ether bond or ester bond,
a is 1 or 2, b is an integer of 0 to 4, and a+b is from 1 to 5.

Typically, the resist composition is a chemically amplified positive resist composition.

In another embodiment, the base polymer is free of an acid labile group. Typically, the resist composition is a chemically amplified negative resist composition.

In a preferred embodiment, the base polymer further comprises repeat units of at least one type selected from repeat units having the formulae (f1) to (f3).

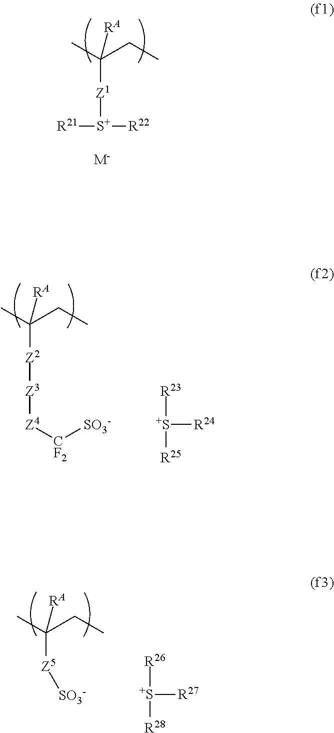

Herein R A is each independently hydrogen or methyl,
Z¹ is a single bond, a C₁-C₆ aliphatic hydrocarbylene group, phenylene group, naphthylene group, or C₇-Cis group obtained by combining the foregoing, or —O—Z¹¹—, —C(═O)—O—Z¹¹— or —C(═O)—NH—Z¹¹—, Z¹¹ is a C₁-C₆ aliphatic hydrocarbylene group, phenylene group, naphthylene group, or C₇-Cis group obtained by combining the foregoing, which may contain a carbonyl moiety, ester bond, ether bond or hydroxy moiety,
Z² is a single bond or ester bond,
Z³ is a single bond, —Z³¹—C(═O)—O—, —Z³¹—O— or —Z³¹—O—C(═O)—, Z³¹ is a C₁-C₁₂ aliphatic hydrocarbylene group, phenylene group, or C₇-Cis group obtained by combining the foregoing, which may contain a carbonyl moiety, ester bond, ether bond, urethane bond, nitro, cyano, fluorine, iodine or bromine,
Z⁴ is methylene, 2,2,2-trifluoro-1,1-ethanediyl or carbonyl,
Z⁵ is a single bond, methylene, ethylene, phenylene, fluorinated phenylene, trifluoromethyl-substituted phenylene group, —O—Z⁵¹—, —C(═O)—O—Z⁵¹—, or —C(═O)—NH—Z⁵¹—, Z⁵¹ is a C₁-C₆ aliphatic hydrocarbylene group, phenylene group, fluorinated phenylene group, or trifluoromethyl-substituted phenylene group, which may contain a carbonyl moiety, ester bond, ether bond, halogen or hydroxy moiety,
R²¹ to R²⁸ are each independently halogen or a C₁-C₂₀ hydrocarbyl group which may contain a heteroatom, a pair of R²³ and R²⁴ or R²⁶ and R²⁷ may bond together to form a ring with the sulfur atom to which they are attached, and
M⁻ is a non-nucleophilic counter ion.

The resist composition may further comprise an acid generator capable of generating a strong acid. Preferably, the acid generator generates a sulfonic acid, imide acid or methide acid.

The resist composition may further comprise an organic solvent and/or a surfactant.

In a further aspect, the invention provides a pattern forming process comprising the steps of applying the resist composition defined herein onto a substrate to form a resist film thereon, exposing the resist film to high-energy radiation, and developing the exposed resist film in a developer.

Typically, the high-energy radiation is KrF excimer laser, ArF excimer laser, EB or EUV of wavelength 3 to 15 nm.

### Advantageous Effects of Invention

The sulfonium salt of specific structure is able to fully control acid diffusion, improves the dissolution in developer of a resist film at exposed region, and serves as an effective quencher. A resist composition comprising the sulfonium salt exhibits a high sensitivity, reduced LWR and improved CDU.

### DETAILED DESCRIPTION OF THE INVENTION

As used herein, the singular forms “a,” “an” and “the” include plural referents unless the context clearly dictates otherwise. “Optional” or “optionally” means that the subsequently described event or circumstances may or may not occur, and that description includes instances where the event or circumstance occurs and instances where it does not. The notation (Cn-Cm) means a group containing from n to m carbon atoms per group. In chemical formulae, the broken line designates a valence bond. As used herein, the term “fluorinated” refers to a fluorine-substituted or fluorine-containing compound or group. The terms “group” and “moiety” are interchangeable.

The abbreviations and acronyms have the following meaning.
EB: electron beam
EUV: extreme ultraviolet
Mw: weight average molecular weight
Mn: number average molecular weight
Mw/Mn: molecular weight distribution or dispersity
GPC: gel permeation chromatography
PEB: post-exposure bake
PAG: photoacid generator
LWR: line width roughness
CDU: critical dimension uniformity

Sulfonium Salt

One embodiment of the invention is a sulfonium salt of specific structure, which is referred to as Sulfonium Salt A, hereinafter. Sulfonium Salt A has the formula (1).

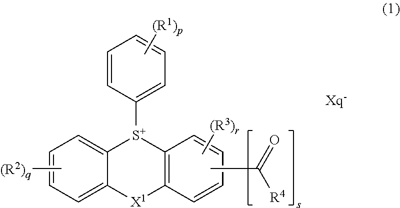

In formula (1), p, q and r are each independently an integer of 0 to 3, s is 1 or 2, and r+s is from 1 to 3.

In formula (1), R¹ and R² are each independently halogen, trifluoromethyl, trifluoromethoxy, trifluoromethylthio, nitro, cyano, —C(═O)—R⁴, —O—C(═O)—R⁵ or —O—R⁵.

In formula (1), R³ is halogen, trifluoromethyl, trifluoromethoxy, trifluoromethylthio, nitro, cyano, —O—C(═O)—R⁵ or —O—R⁵.

In formula (1), R⁴ is a C₁-C₁₀ hydrocarbyl group, C₁-C₁₀ hydrocarbyloxy group, or —O—R^{4A}. The hydrocarbyl group and hydrocarbyloxy group may be substituted with fluorine or hydroxy. R^{4A} is an acid labile group.

In formula (1), R⁵ is a C₁-C₁₀ hydrocarbyl group.

The hydrocarbyl group represented by R⁴ and R⁵ and the hydrocarbyl moiety in the hydrocarbyloxy group represented by R⁴ may be saturated or unsaturated and straight, branched or cyclic. Examples thereof include C₁-C₁₀ alkyl groups such as methyl, ethyl, n-propyl, isopropyl, n-butyl, isobutyl, sec-butyl, tert-butyl, n-pentyl, isopentyl, sec-pentyl, 3-pentyl, tert-pentyl, neopentyl, n-hexyl, n-octyl, n-nonyl, and n-decyl; C₃-C₁₀ cyclic saturated hydrocarbyl groups such as cyclopropyl, cyclobutyl, cyclopentyl, cyclohexyl, adamantyl, norbornyl, cyclopropylmethyl, cyclopropylethyl, cyclobutylmethyl, cyclobutylethyl, cyclopentylmethyl, cyclopentylethyl, cyclohexylmethyl, cyclohexylethyl, methylcyclobutyl, methylcyclopentyl, methylcyclohexyl, ethylcyclopropyl, ethylcyclobutyl, ethylcyclopentyl, and ethylcyclohexyl; C₂-C₁₀ alkenyl groups such as vinyl, 1-propenyl, 2-propenyl, butenyl, pentenyl, hexenyl, heptenyl, nonenyl, and decenyl; C₂-C₁₀ alkynyl groups such as ethynyl, propynyl, butynyl, pentynyl, hexynyl, heptynyl, octynyl, nonynyl, and decynyl; C₃-C₁₀ cyclic unsaturated aliphatic hydrocarbyl groups such as cyclopentenyl, cyclohexenyl, methylcyclopentenyl, methylcyclohexenyl, ethylcyclopentenyl, ethylcyclohexenyl, and norbornenyl; C₆-C₁₀ aryl groups such as phenyl, methylphenyl, ethylphenyl, n-propylphenyl, isopropylphenyl, n-butylphenyl, isobutylphenyl, sec-butylphenyl, tert-butylphenyl, and naphthyl; C₇-C₁₀ aralkyl groups such as benzyl, phenethyl, phenylpropyl, and phenylbutyl, and combinations thereof.

Typical of the acid labile group R^{4A} are acid labile groups having formula (AL-1) to (AL-3) to be described later.

In formula (1), X¹ is a single bond, ether bond, carbonyl group, —N(R)—, sulfide bond or sulfonyl group. R is hydrogen or a C₁-C₆ saturated hydrocarbyl group.

Examples of the cation in Sulfonium Salt A are shown below, but not limited thereto.

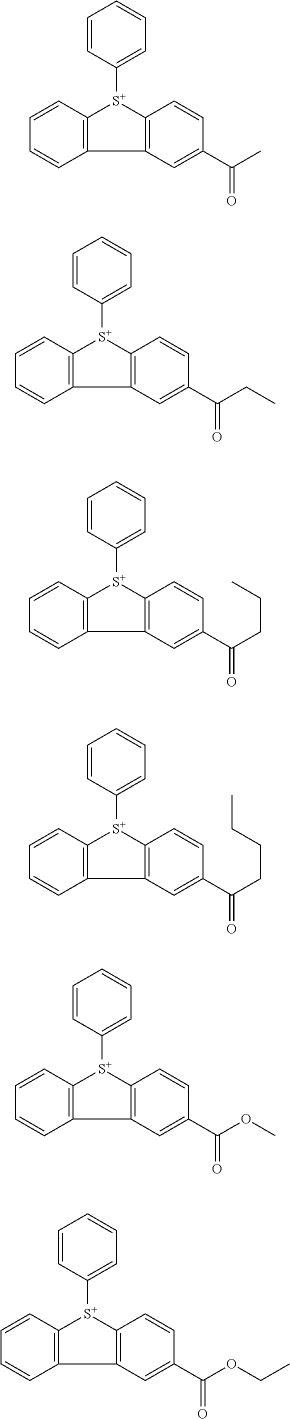 [SMILES: CC(=O)c1ccc2c(c1)c1ccccc1[s+]2-c1ccccc1 ; CCC(=O)c1ccc2c(c1)c1ccccc1[s+]2-c1ccccc1 ; CCCC(=O)c1ccc2c(c1)c1ccccc1[s+]2-c1ccccc1 ; CCCCC(=O)c1ccc2c(c1)c1ccccc1[s+]2-c1ccccc1 ; COC(=O)c1ccc2c(c1)c1ccccc1[s+]2-c1ccccc1 ; CCOC(=O)c1ccc2c(c1)c1ccccc1[s+]2-c1ccccc1]

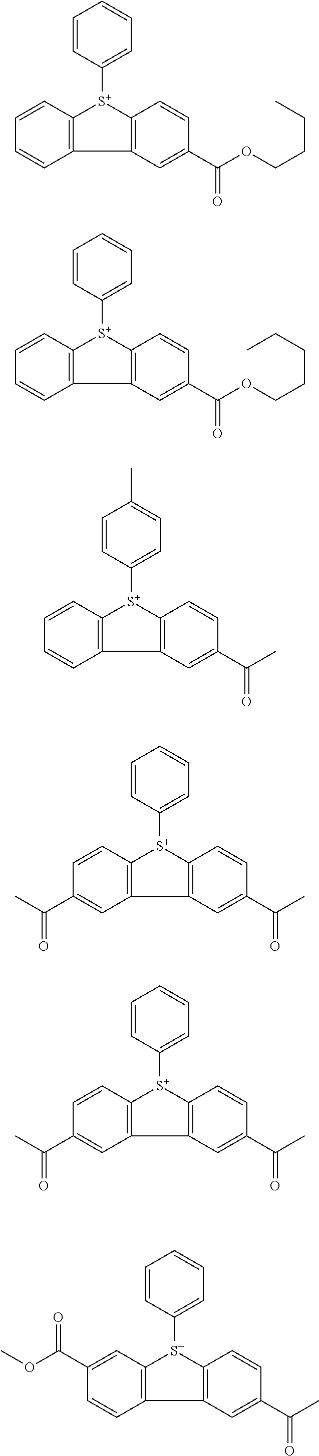

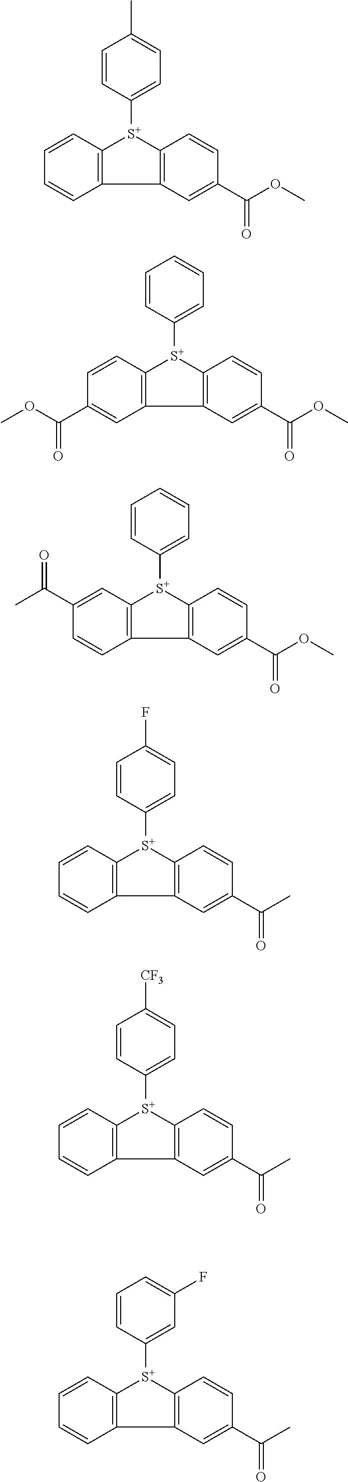

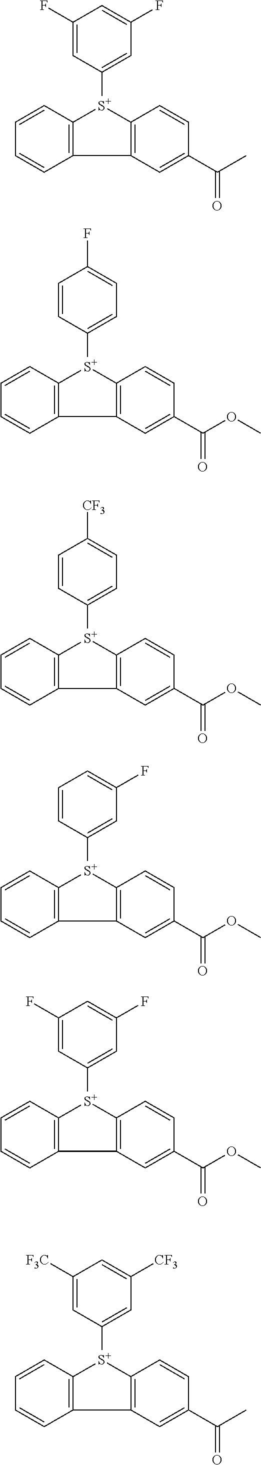 [SMILES: CC(=O)c1ccc2c(c1)c1ccccc1[s+]2-c1cc(F)cc(F)c1 ; COC(=O)c1ccc2c(c1)c1ccccc1[s+]2-c1ccc(F)cc1 ; COC(=O)c1ccc2c(c1)c1ccccc1[s+]2-c1ccc(C(F)(F)F)cc1 ; COC(=O)c1ccc2c(c1)c1ccccc1[s+]2-c1cccc(F)c1 ; COC(=O)c1ccc2c(c1)c1ccccc1[s+]2-c1cc(F)cc(F)c1 ; CC(=O)c1ccc2c(c1)c1ccccc1[s+]2-c1cc(C(F)(F)F)cc(C(F)(F)F)c1]

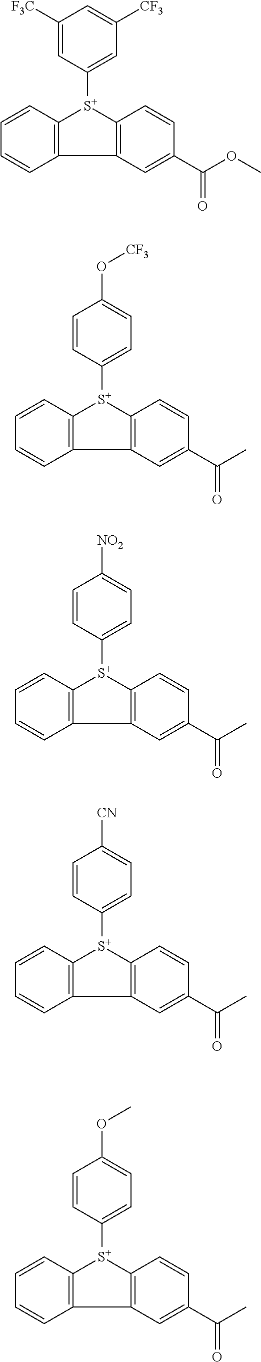 [SMILES: COC(=O)c1ccc2c(c1)c1ccccc1[s+]2-c1cc(C(F)(F)F)cc(C(F)(F)F)c1 ; CC(=O)c1ccc2c(c1)c1ccccc1[s+]2-c1ccc(OC(F)(F)F)cc1 ; CC(=O)c1ccc2c(c1)c1ccccc1[s+]2-c1ccc([N+](=O)[O-])cc1 ; CC(=O)c1ccc2c(c1)c1ccccc1[s+]2-c1ccc(C#N)cc1 ; COc1ccc(-[s+]2c3ccccc3c3cc(C(C)=O)ccc32)cc1]

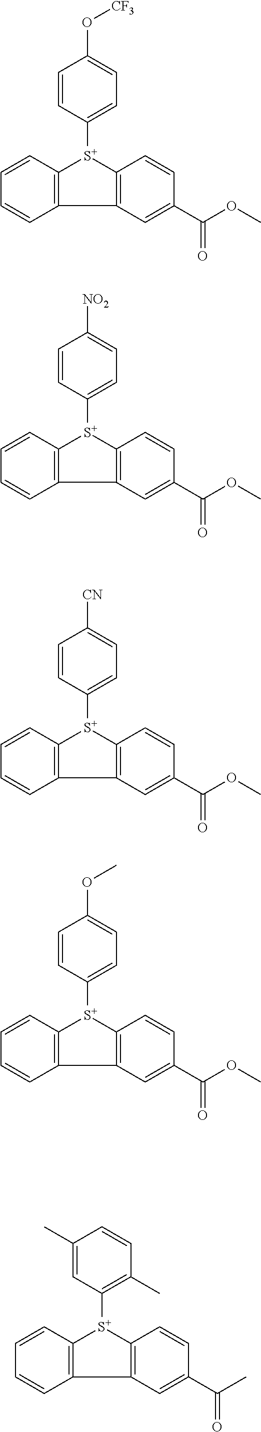 [SMILES: COC(=O)c1ccc2c(c1)c1ccccc1[s+]2-c1ccc(OC(F)(F)F)cc1 ; COC(=O)c1ccc2c(c1)c1ccccc1[s+]2-c1ccc([N+](=O)[O-])cc1 ; COC(=O)c1ccc2c(c1)c1ccccc1[s+]2-c1ccc(C#N)cc1 ; COC(=O)c1ccc2c(c1)c1ccccc1[s+]2-c1ccc(OC)cc1 ; CC(=O)c1ccc2c(c1)c1ccccc1[s+]2-c1cc(C)ccc1C]

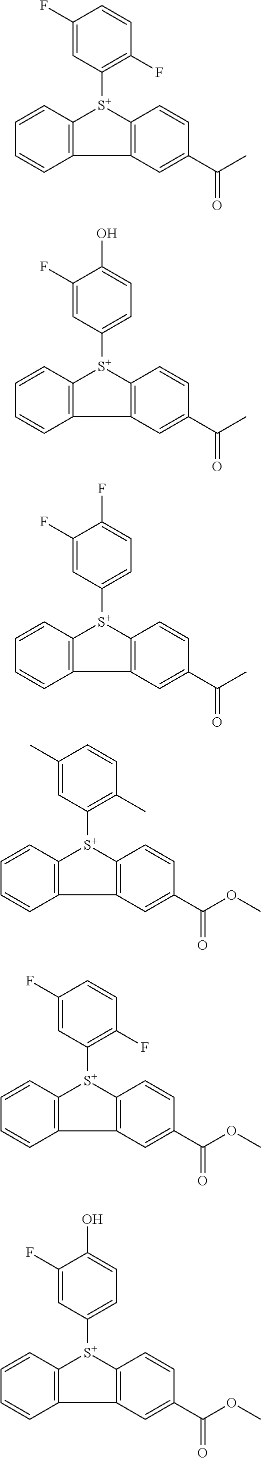 [SMILES: CC(=O)c1ccc2c(c1)c1ccccc1[s+]2-c1cc(F)ccc1F ; CC(=O)c1ccc2c(c1)c1ccccc1[s+]2-c1ccc(O)c(F)c1 ; CC(=O)c1ccc2c(c1)c1ccccc1[s+]2-c1ccc(F)c(F)c1 ; COC(=O)c1ccc2c(c1)c1ccccc1[s+]2-c1cc(C)ccc1C ; COC(=O)c1ccc2c(c1)c1ccccc1[s+]2-c1cc(F)ccc1F ; COC(=O)c1ccc2c(c1)c1ccccc1[s+]2-c1ccc(O)c(F)c1]

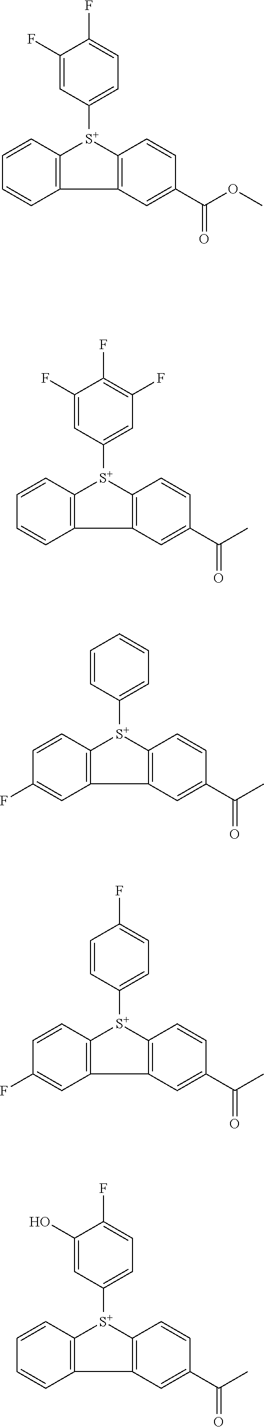 [SMILES: COC(=O)c1ccc2c(c1)c1ccccc1[s+]2-c1ccc(F)c(F)c1 ; CC(=O)c1ccc2c(c1)c1ccccc1[s+]2-c1cc(F)c(F)c(F)c1 ; CC(=O)c1ccc2c(c1)c1cc(F)ccc1[s+]2-c1ccccc1 ; CC(=O)c1ccc2c(c1)c1cc(F)ccc1[s+]2-c1ccc(F)cc1 ; CC(=O)c1ccc2c(c1)c1ccccc1[s+]2-c1ccc(F)c(O)c1]

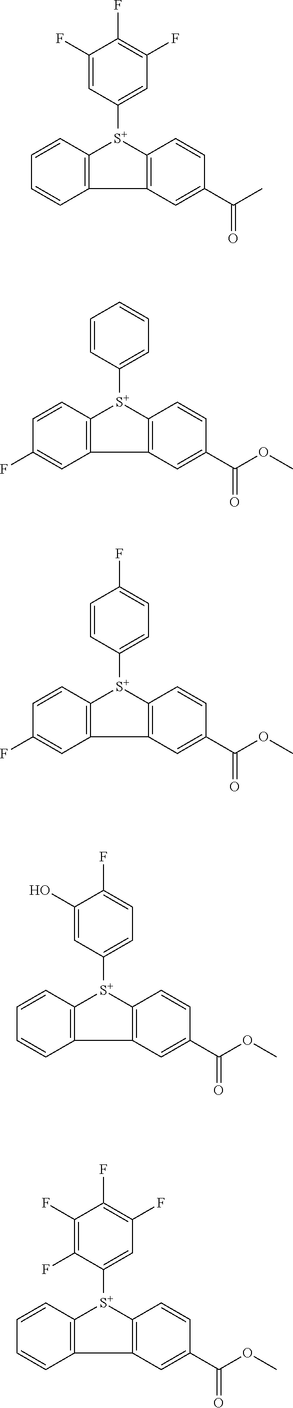 [SMILES: CC(=O)c1ccc2c(c1)c1ccccc1[s+]2-c1cc(F)c(F)c(F)c1 ; COC(=O)c1ccc2c(c1)c1cc(F)ccc1[s+]2-c1ccccc1 ; COC(=O)c1ccc2c(c1)c1cc(F)ccc1[s+]2-c1ccc(F)cc1 ; COC(=O)c1ccc2c(c1)c1ccccc1[s+]2-c1ccc(F)c(O)c1 ; COC(=O)c1ccc2c(c1)c1ccccc1[s+]2-c1cc(F)c(F)c(F)c1F]

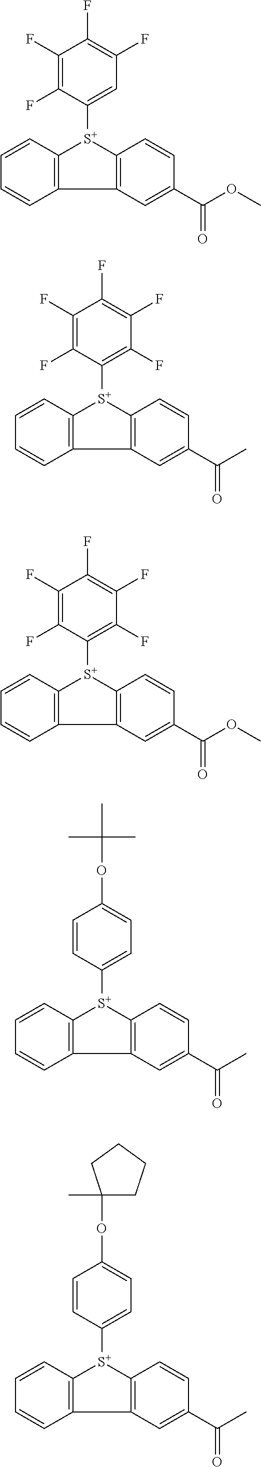 [SMILES: COC(=O)c1ccc2c(c1)c1ccccc1[s+]2-c1cc(F)c(F)c(F)c1F ; CC(=O)c1ccc2c(c1)c1ccccc1[s+]2-c1c(F)c(F)c(F)c(F)c1F ; COC(=O)c1ccc2c(c1)c1ccccc1[s+]2-c1c(F)c(F)c(F)c(F)c1F ; CC(=O)c1ccc2c(c1)c1ccccc1[s+]2-c1ccc(OC(C)(C)C)cc1 ; CC(=O)c1ccc2c(c1)c1ccccc1[s+]2-c1ccc(OC2(C)CCCC2)cc1]

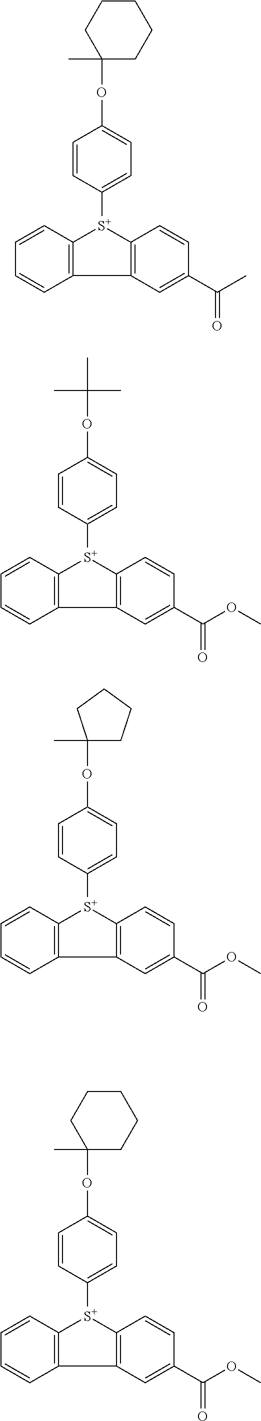 [SMILES: CC(=O)c1ccc2c(c1)c1ccccc1[s+]2-c1ccc(OC2(C)CCCCC2)cc1 ; COC(=O)c1ccc2c(c1)c1ccccc1[s+]2-c1ccc(OC(C)(C)C)cc1 ; COC(=O)c1ccc2c(c1)c1ccccc1[s+]2-c1ccc(OC2(C)CCCC2)cc1 ; COC(=O)c1ccc2c(c1)c1ccccc1[s+]2-c1ccc(OC2(C)CCCCC2)cc1]

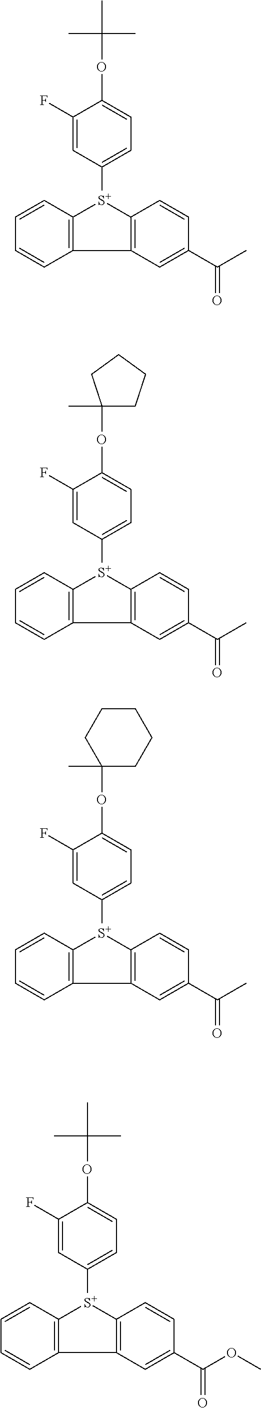 [SMILES: CC(=O)c1ccc2c(c1)c1ccccc1[s+]2-c1ccc(OC(C)(C)C)c(F)c1 ; CC(=O)c1ccc2c(c1)c1ccccc1[s+]2-c1ccc(OC2(C)CCCC2)c(F)c1 ; CC(=O)c1ccc2c(c1)c1ccccc1[s+]2-c1ccc(OC2(C)CCCCC2)c(F)c1 ; COC(=O)c1ccc2c(c1)c1ccccc1[s+]2-c1ccc(OC(C)(C)C)c(F)c1]

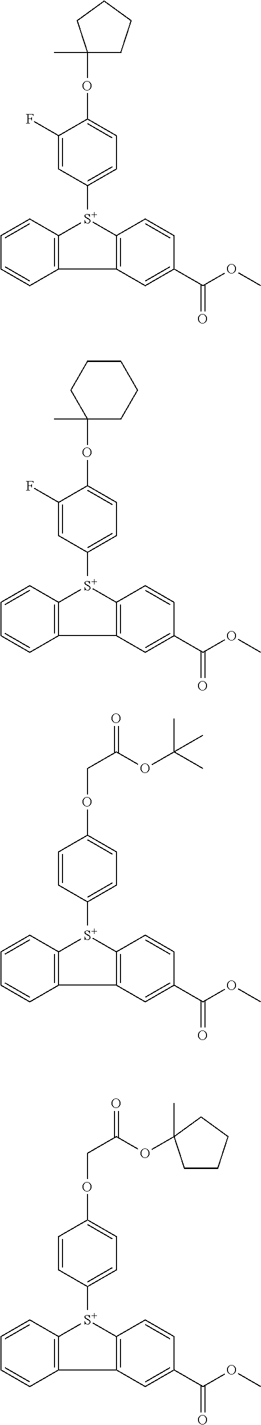 [SMILES: COC(=O)c1ccc2c(c1)c1ccccc1[s+]2-c1ccc(OC2(C)CCCC2)c(F)c1 ; COC(=O)c1ccc2c(c1)c1ccccc1[s+]2-c1ccc(OC2(C)CCCCC2)c(F)c1 ; COC(=O)c1ccc2c(c1)c1ccccc1[s+]2-c1ccc(OCC(=O)OC(C)(C)C)cc1 ; COC(=O)c1ccc2c(c1)c1ccccc1[s+]2-c1ccc(OCC(=O)OC2(C)CCCC2)cc1]

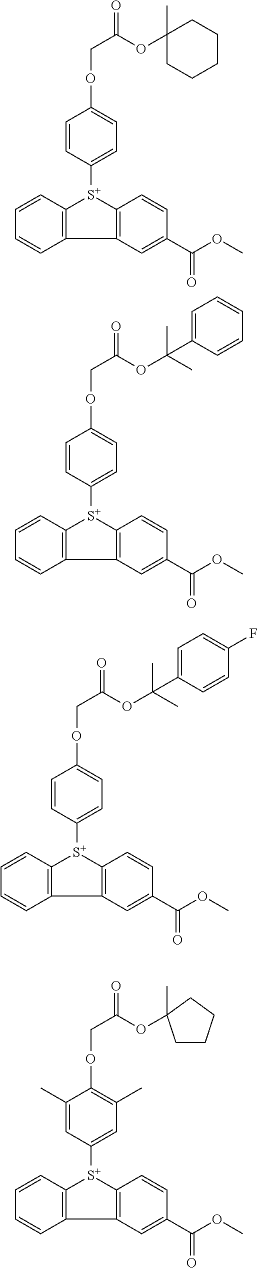 [SMILES: COC(=O)c1ccc2c(c1)c1ccccc1[s+]2-c1ccc(OCC(=O)OC2(C)CCCCC2)cc1 ; COC(=O)c1ccc2c(c1)c1ccccc1[s+]2-c1ccc(OCC(=O)OC(C)(C)c2ccccc2)cc1 ; COC(=O)c1ccc2c(c1)c1ccccc1[s+]2-c1ccc(OCC(=O)OC(C)(C)c2ccc(F)cc2)cc1 ; COC(=O)c1ccc2c(c1)c1ccccc1[s+]2-c1cc(C)c(OCC(=O)OC2(C)CCCC2)c(C)c1]

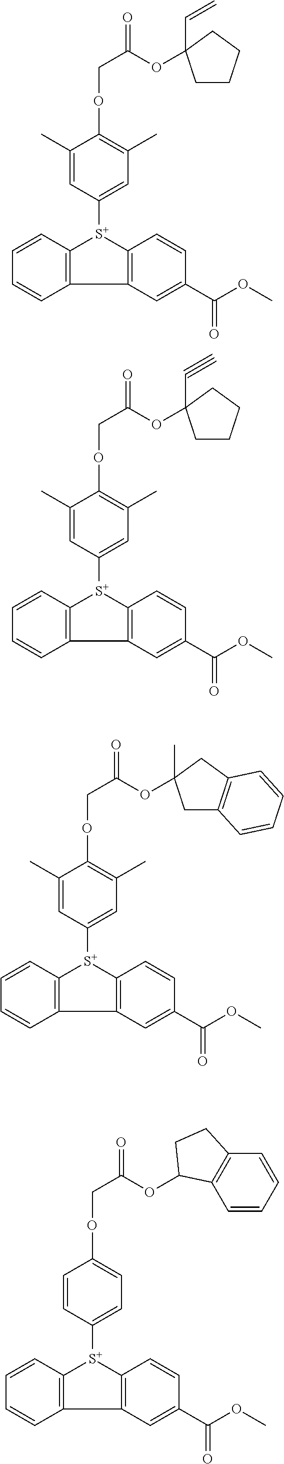 [SMILES: C=CC1(OC(=O)COc2c(C)cc(-[s+]3c4ccccc4c4cc(C(=O)OC)ccc43)cc2C)CCCC1 ; C#CC1(OC(=O)COc2c(C)cc(-[s+]3c4ccccc4c4cc(C(=O)OC)ccc43)cc2C)CCCC1 ; COC(=O)c1ccc2c(c1)c1ccccc1[s+]2-c1cc(C)c(OCC(=O)OC2(C)Cc3ccccc3C2)c(C)c1 ; COC(=O)c1ccc2c(c1)c1ccccc1[s+]2-c1ccc(OCC(=O)OC2CCc3ccccc32)cc1]

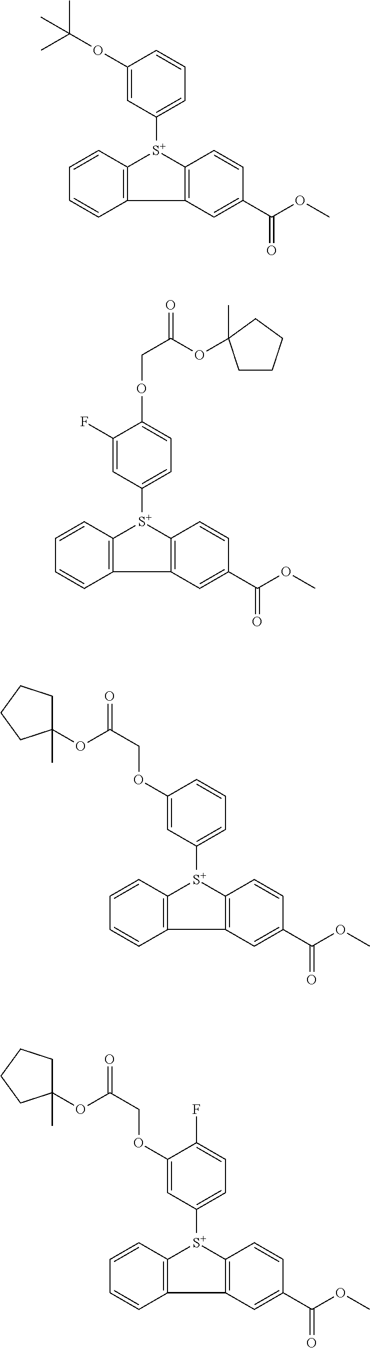 [SMILES: COC(=O)c1ccc2c(c1)c1ccccc1[s+]2-c1cccc(OC(C)(C)C)c1 ; COC(=O)c1ccc2c(c1)c1ccccc1[s+]2-c1ccc(OCC(=O)OC2(C)CCCC2)c(F)c1 ; COC(=O)c1ccc2c(c1)c1ccccc1[s+]2-c1cccc(OCC(=O)OC2(C)CCCC2)c1 ; COC(=O)c1ccc2c(c1)c1ccccc1[s+]2-c1ccc(F)c(OCC(=O)OC2(C)CCCC2)c1]

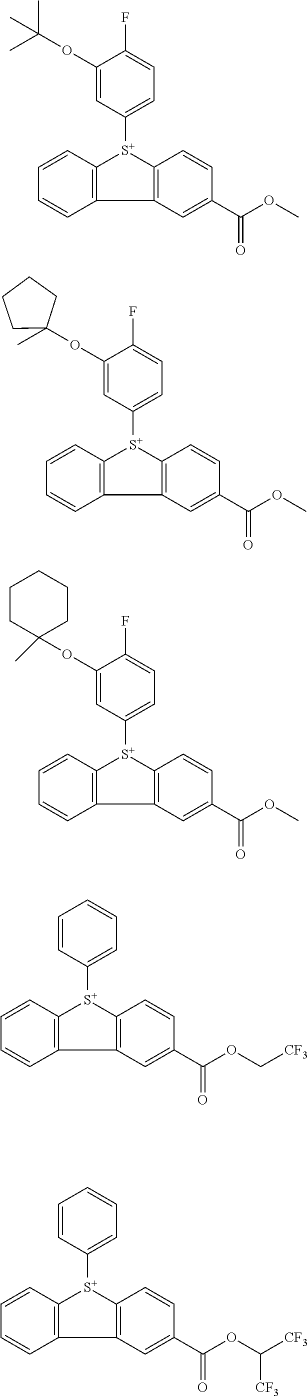 [SMILES: COC(=O)c1ccc2c(c1)c1ccccc1[s+]2-c1ccc(F)c(OC(C)(C)C)c1 ; COC(=O)c1ccc2c(c1)c1ccccc1[s+]2-c1ccc(F)c(OC2(C)CCCC2)c1 ; COC(=O)c1ccc2c(c1)c1ccccc1[s+]2-c1ccc(F)c(OC2(C)CCCCC2)c1 ; O=C(OCC(F)(F)F)c1ccc2c(c1)c1ccccc1[s+]2-c1ccccc1 ; O=C(OC(C(F)(F)F)C(F)(F)F)c1ccc2c(c1)c1ccccc1[s+]2-c1ccccc1]

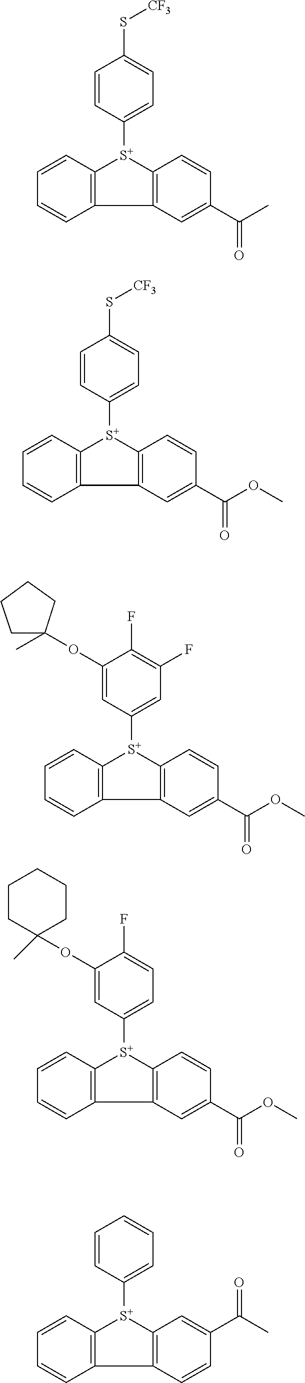 [SMILES: CC(=O)c1ccc2c(c1)c1ccccc1[s+]2-c1ccc(SC(F)(F)F)cc1 ; COC(=O)c1ccc2c(c1)c1ccccc1[s+]2-c1ccc(SC(F)(F)F)cc1 ; COC(=O)c1ccc2c(c1)c1ccccc1[s+]2-c1cc(F)c(F)c(OC2(C)CCCC2)c1 ; COC(=O)c1ccc2c(c1)c1ccccc1[s+]2-c1ccc(F)c(OC2(C)CCCCC2)c1 ; CC(=O)c1ccc2c3ccccc3[s+](-c3ccccc3)c2c1]

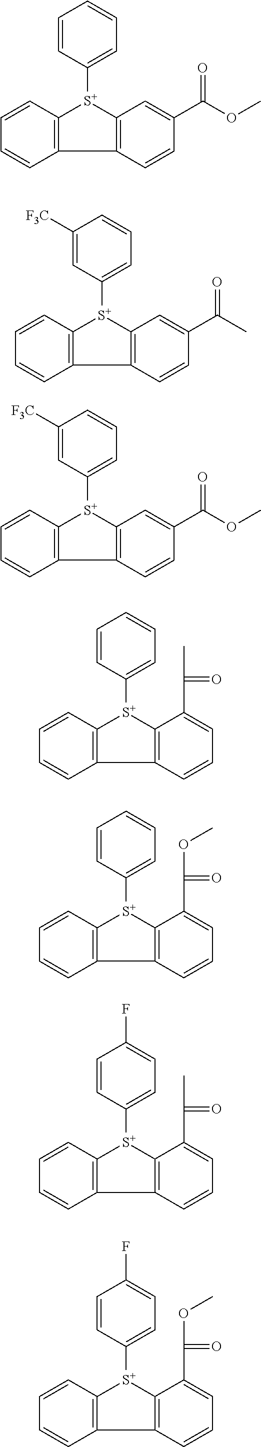

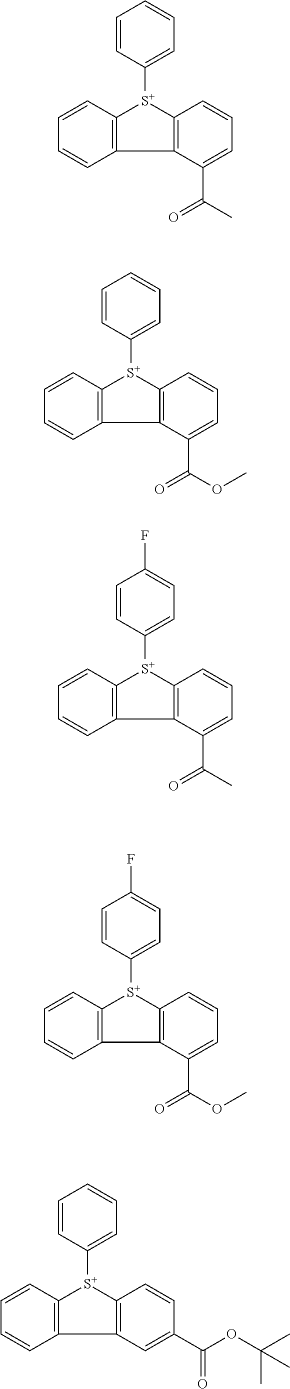 [SMILES: CC(=O)c1cccc2c1c1ccccc1[s+]2-c1ccccc1 ; COC(=O)c1cccc2c1c1ccccc1[s+]2-c1ccccc1 ; CC(=O)c1cccc2c1c1ccccc1[s+]2-c1ccc(F)cc1 ; COC(=O)c1cccc2c1c1ccccc1[s+]2-c1ccc(F)cc1 ; CC(C)(C)OC(=O)c1ccc2c(c1)c1ccccc1[s+]2-c1ccccc1]

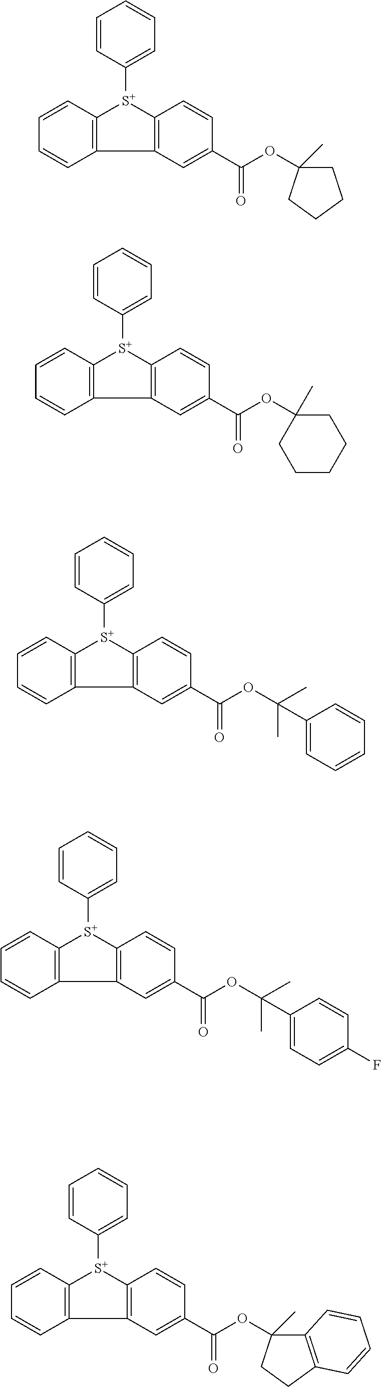 [SMILES: CC1(OC(=O)c2ccc3c(c2)c2ccccc2[s+]3-c2ccccc2)CCCC1 ; CC1(OC(=O)c2ccc3c(c2)c2ccccc2[s+]3-c2ccccc2)CCCCC1 ; CC(C)(OC(=O)c1ccc2c(c1)c1ccccc1[s+]2-c1ccccc1)c1ccccc1 ; CC(C)(OC(=O)c1ccc2c(c1)c1ccccc1[s+]2-c1ccccc1)c1ccc(F)cc1 ; CC1(OC(=O)c2ccc3c(c2)c2ccccc2[s+]3-c2ccccc2)CCc2ccccc21]

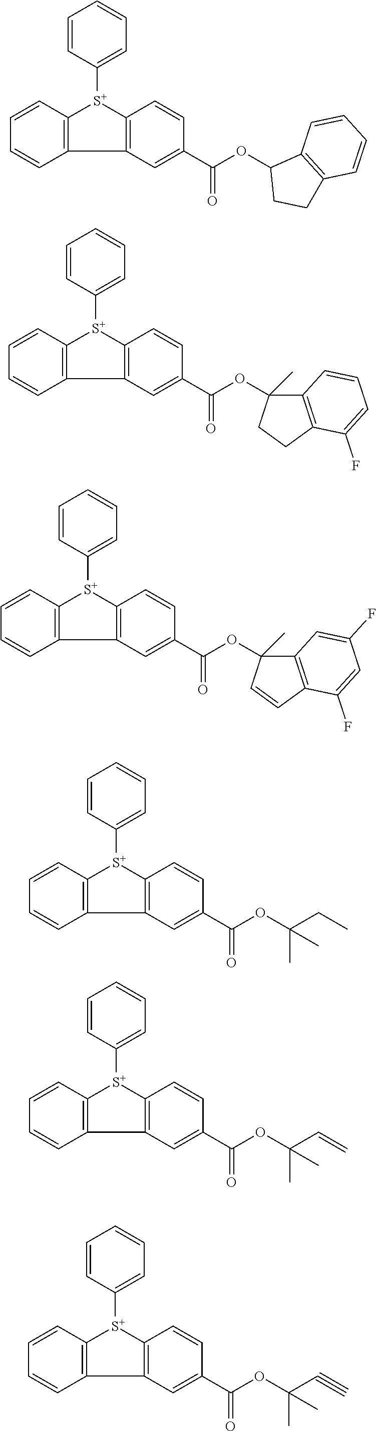 [SMILES: O=C(OC1CCc2ccccc21)c1ccc2c(c1)c1ccccc1[s+]2-c1ccccc1 ; CC1(OC(=O)c2ccc3c(c2)c2ccccc2[s+]3-c2ccccc2)CCc2c(F)cccc21 ; CC1(OC(=O)c2ccc3c(c2)c2ccccc2[s+]3-c2ccccc2)C=Cc2c(F)cc(F)cc21 ; CCC(C)(C)OC(=O)c1ccc2c(c1)c1ccccc1[s+]2-c1ccccc1 ; C=CC(C)(C)OC(=O)c1ccc2c(c1)c1ccccc1[s+]2-c1ccccc1 ; C#CC(C)(C)OC(=O)c1ccc2c(c1)c1ccccc1[s+]2-c1ccccc1]

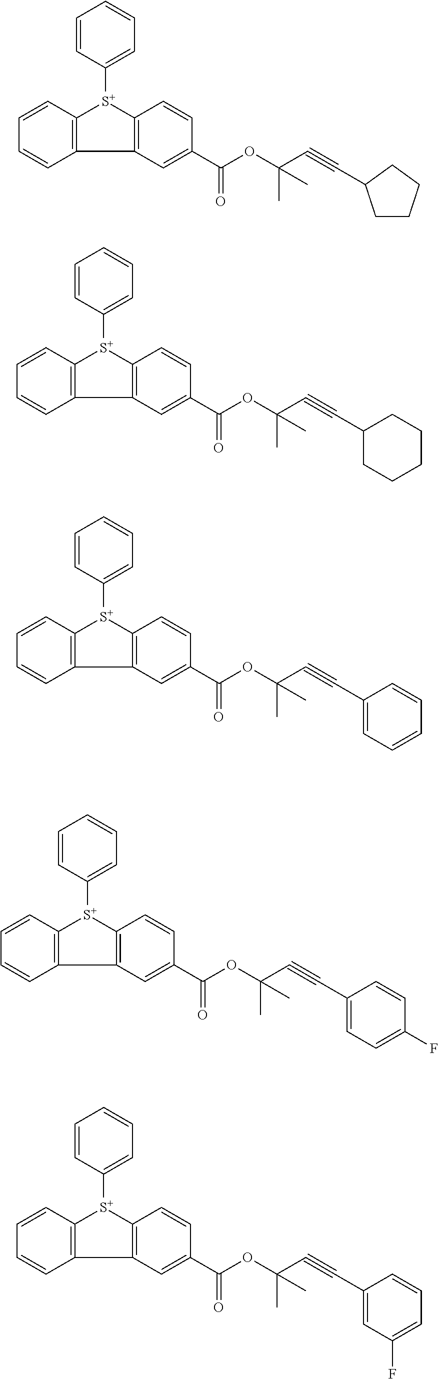 [SMILES: CC(C)(C#CC1CCCC1)OC(=O)c1ccc2c(c1)c1ccccc1[s+]2-c1ccccc1 ; CC(C)(C#CC1CCCCC1)OC(=O)c1ccc2c(c1)c1ccccc1[s+]2-c1ccccc1 ; CC(C)(C#Cc1ccccc1)OC(=O)c1ccc2c(c1)c1ccccc1[s+]2-c1ccccc1 ; CC(C)(C#Cc1ccc(F)cc1)OC(=O)c1ccc2c(c1)c1ccccc1[s+]2-c1ccccc1 ; CC(C)(C#Cc1cccc(F)c1)OC(=O)c1ccc2c(c1)c1ccccc1[s+]2-c1ccccc1]

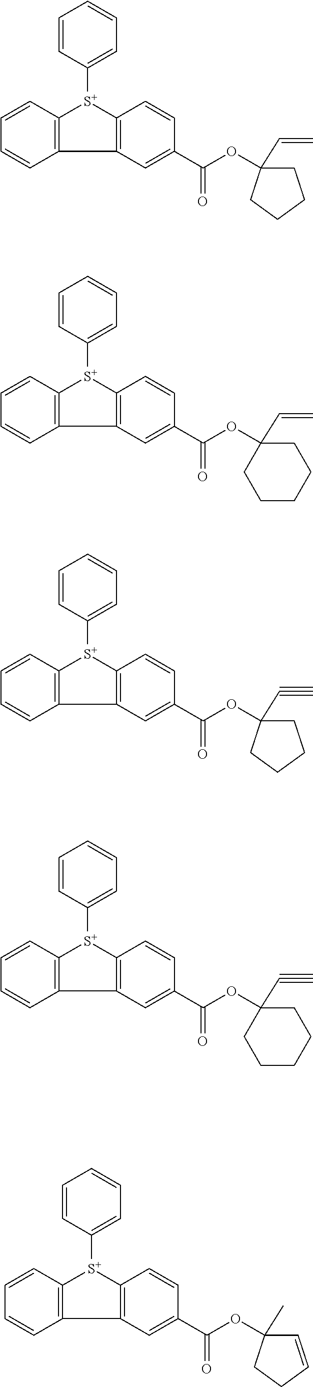 [SMILES: C=CC1(OC(=O)c2ccc3c(c2)c2ccccc2[s+]3-c2ccccc2)CCCC1 ; C=CC1(OC(=O)c2ccc3c(c2)c2ccccc2[s+]3-c2ccccc2)CCCCC1 ; C#CC1(OC(=O)c2ccc3c(c2)c2ccccc2[s+]3-c2ccccc2)CCCC1 ; C#CC1(OC(=O)c2ccc3c(c2)c2ccccc2[s+]3-c2ccccc2)CCCCC1 ; CC1(OC(=O)c2ccc3c(c2)c2ccccc2[s+]3-c2ccccc2)C=CCC1]

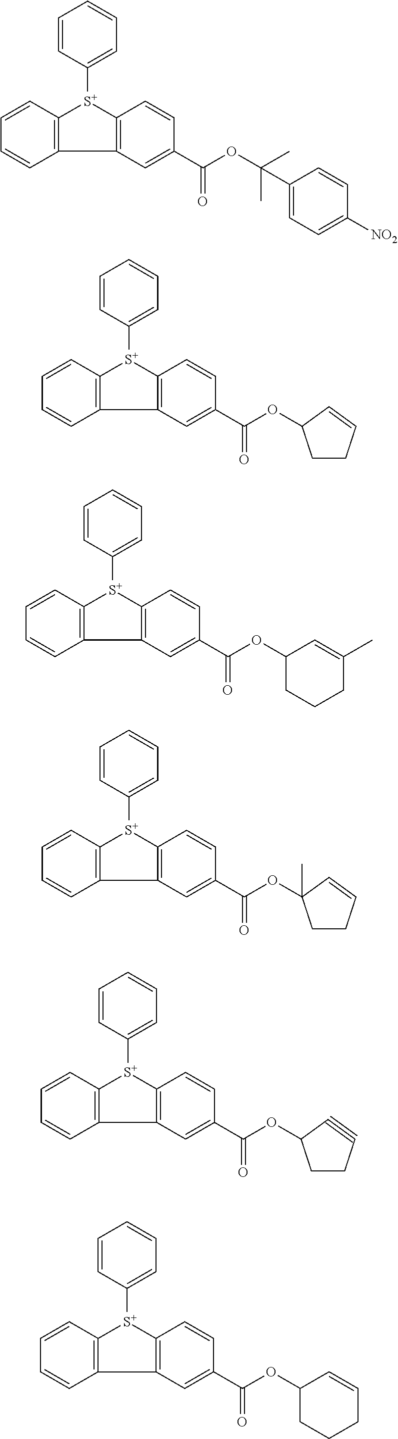 [SMILES: CC(C)(OC(=O)c1ccc2c(c1)c1ccccc1[s+]2-c1ccccc1)c1ccc([N+](=O)[O-])cc1 ; O=C(OC1C=CCC1)c1ccc2c(c1)c1ccccc1[s+]2-c1ccccc1 ; CC1=CC(OC(=O)c2ccc3c(c2)c2ccccc2[s+]3-c2ccccc2)CCC1 ; CC1(OC(=O)c2ccc3c(c2)c2ccccc2[s+]3-c2ccccc2)C=CCC1 ; O=C(OC1C#CCC1)c1ccc2c(c1)c1ccccc1[s+]2-c1ccccc1 ; O=C(OC1C=CCCC1)c1ccc2c(c1)c1ccccc1[s+]2-c1ccccc1]

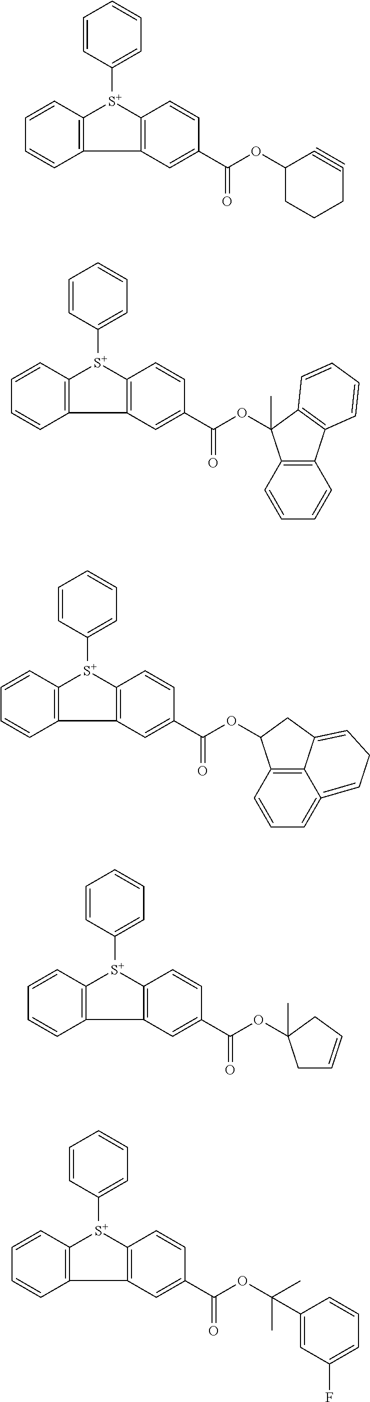

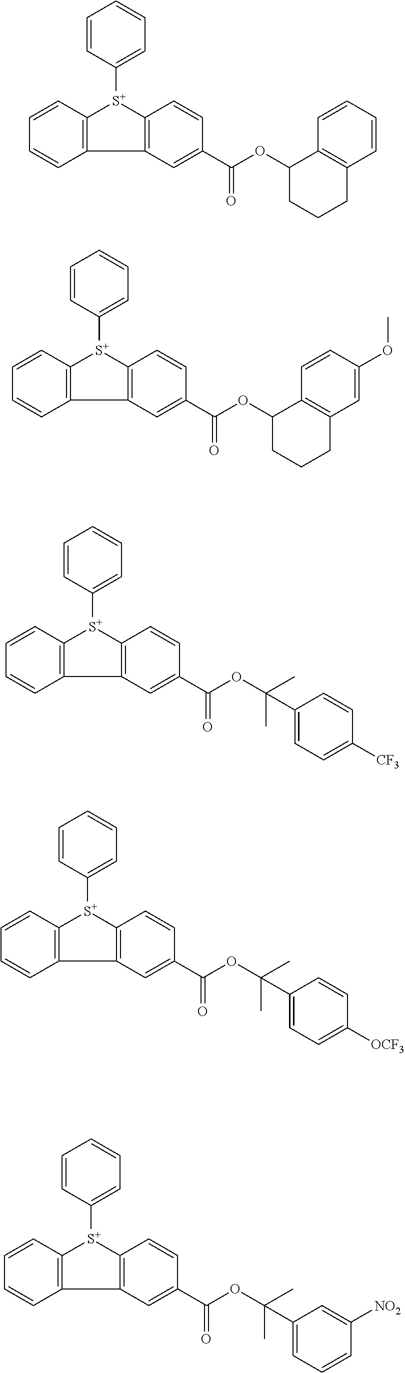 [SMILES: O=C(OC1CCCc2ccccc21)c1ccc2c(c1)c1ccccc1[s+]2-c1ccccc1 ; COc1ccc2c(c1)CCCC2OC(=O)c1ccc2c(c1)c1ccccc1[s+]2-c1ccccc1 ; CC(C)(OC(=O)c1ccc2c(c1)c1ccccc1[s+]2-c1ccccc1)c1ccc(C(F)(F)F)cc1 ; CC(C)(OC(=O)c1ccc2c(c1)c1ccccc1[s+]2-c1ccccc1)c1ccc(OC(F)(F)F)cc1 ; CC(C)(OC(=O)c1ccc2c(c1)c1ccccc1[s+]2-c1ccccc1)c1cccc([N+](=O)[O-])c1]

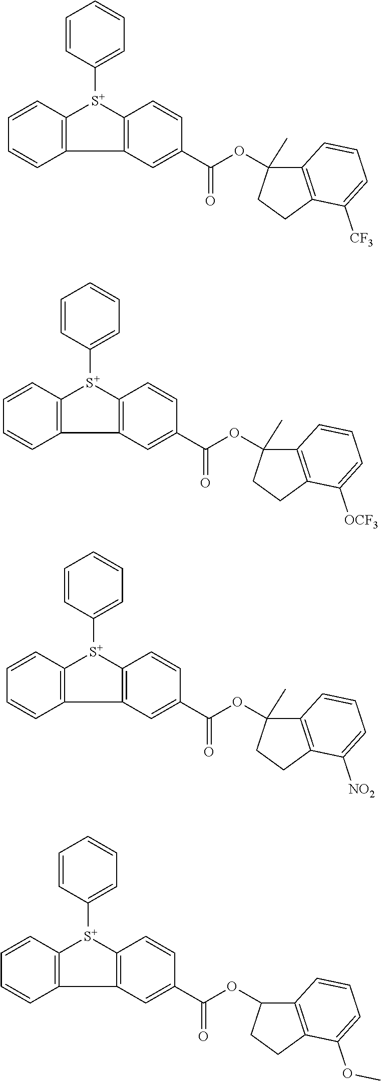 [SMILES: CC1(OC(=O)c2ccc3c(c2)c2ccccc2[s+]3-c2ccccc2)CCc2c(C(F)(F)F)cccc21 ; CC1(OC(=O)c2ccc3c(c2)c2ccccc2[s+]3-c2ccccc2)CCc2c(OC(F)(F)F)cccc21 ; CC1(OC(=O)c2ccc3c(c2)c2ccccc2[s+]3-c2ccccc2)CCc2c([N+](=O)[O-])cccc21 ; COc1cccc2c1CCC2OC(=O)c1ccc2c(c1)c1ccccc1[s+]2-c1ccccc1]

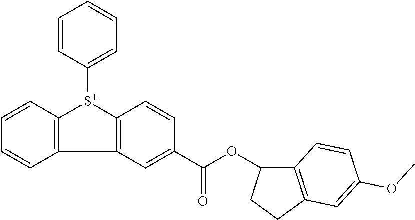 [SMILES: COc1ccc2c(c1)CCC2OC(=O)c1ccc2c(c1)c1ccccc1[s+]2-c1ccccc1]

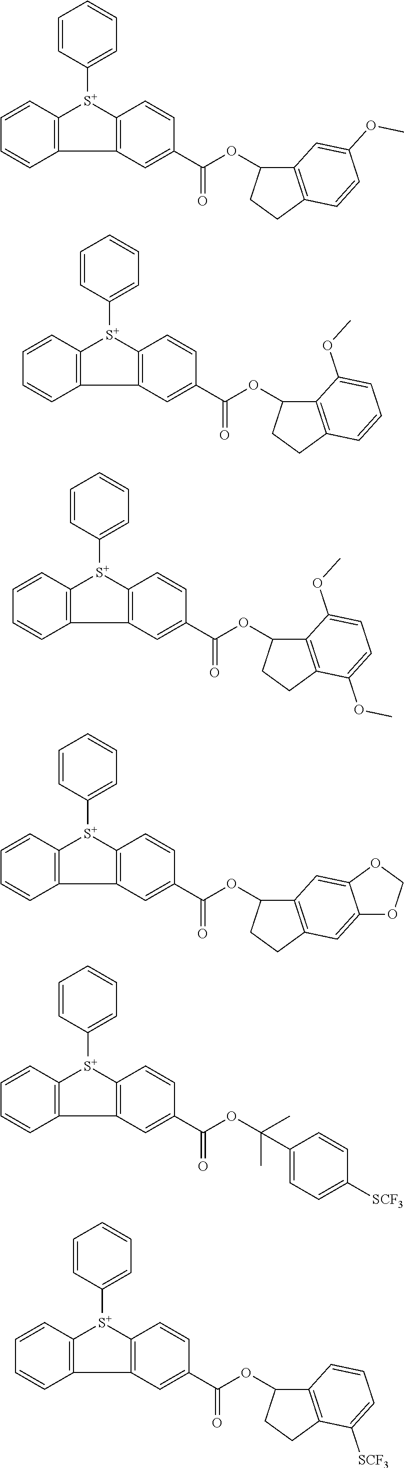

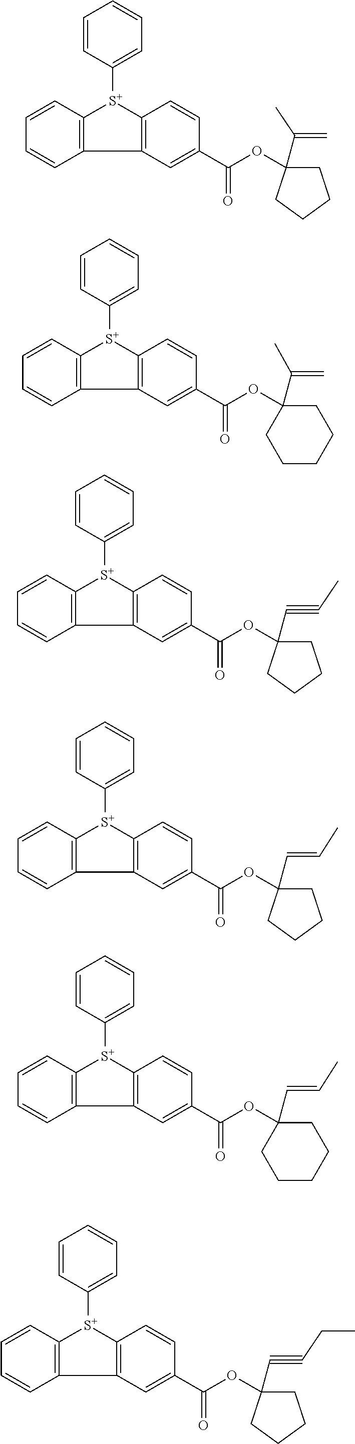 [SMILES: C=C(C)C1(OC(=O)c2ccc3c(c2)c2ccccc2[s+]3-c2ccccc2)CCCC1 ; C=C(C)C1(OC(=O)c2ccc3c(c2)c2ccccc2[s+]3-c2ccccc2)CCCCC1 ; CC#CC1(OC(=O)c2ccc3c(c2)c2ccccc2[s+]3-c2ccccc2)CCCC1 ; C/C=C/C1(OC(=O)c2ccc3c(c2)c2ccccc2[s+]3-c2ccccc2)CCCC1 ; C/C=C/C1(OC(=O)c2ccc3c(c2)c2ccccc2[s+]3-c2ccccc2)CCCCC1 ; CCC#CC1(OC(=O)c2ccc3c(c2)c2ccccc2[s+]3-c2ccccc2)CCCC1]

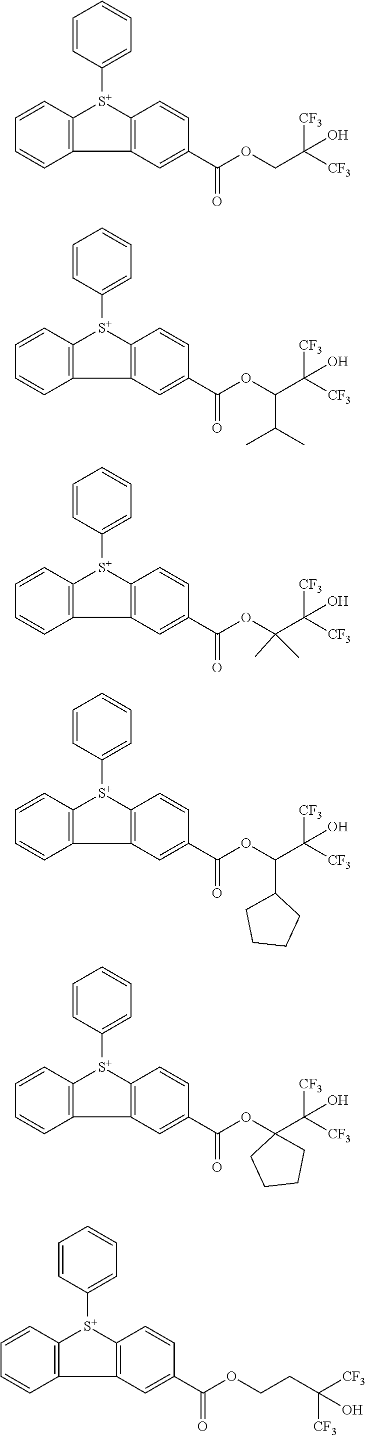 [SMILES: O=C(OCC(O)(C(F)(F)F)C(F)(F)F)c1ccc2c(c1)c1ccccc1[s+]2-c1ccccc1 ; CC(C)C(OC(=O)c1ccc2c(c1)c1ccccc1[s+]2-c1ccccc1)C(O)(C(F)(F)F)C(F)(F)F ; CC(C)(OC(=O)c1ccc2c(c1)c1ccccc1[s+]2-c1ccccc1)C(O)(C(F)(F)F)C(F)(F)F ; O=C(OC(C1CCCC1)C(O)(C(F)(F)F)C(F)(F)F)c1ccc2c(c1)c1ccccc1[s+]2-c1ccccc1 ; O=C(OC1(C(O)(C(F)(F)F)C(F)(F)F)CCCC1)c1ccc2c(c1)c1ccccc1[s+]2-c1ccccc1 ; O=C(OCCC(O)(C(F)(F)F)C(F)(F)F)c1ccc2c(c1)c1ccccc1[s+]2-c1ccccc1]

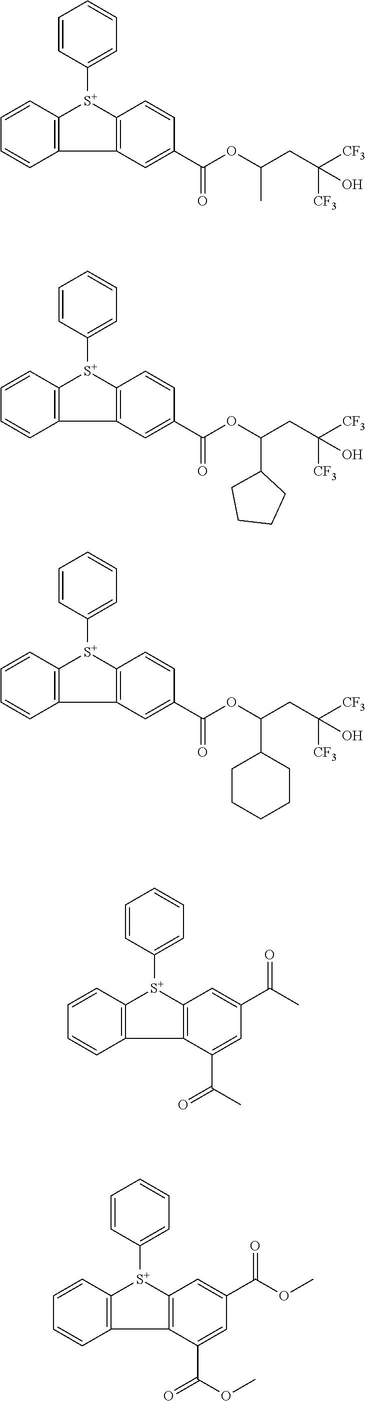 [SMILES: CC(CC(O)(C(F)(F)F)C(F)(F)F)OC(=O)c1ccc2c(c1)c1ccccc1[s+]2-c1ccccc1 ; O=C(OC(CC(O)(C(F)(F)F)C(F)(F)F)C1CCCC1)c1ccc2c(c1)c1ccccc1[s+]2-c1ccccc1 ; O=C(OC(CC(O)(C(F)(F)F)C(F)(F)F)C1CCCCC1)c1ccc2c(c1)c1ccccc1[s+]2-c1ccccc1 ; CC(=O)c1cc(C(C)=O)c2c3ccccc3[s+](-c3ccccc3)c2c1 ; COC(=O)c1cc(C(=O)OC)c2c3ccccc3[s+](-c3ccccc3)c2c1]

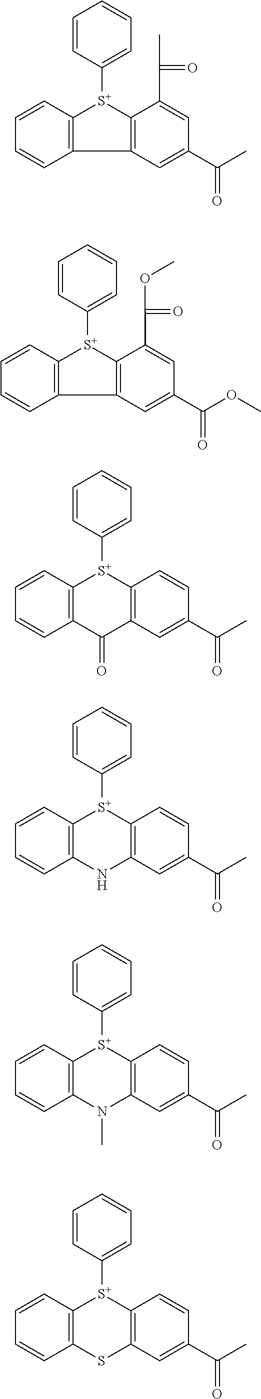 [SMILES: CC(=O)c1cc(C(C)=O)c2c(c1)c1ccccc1[s+]2-c1ccccc1 ; COC(=O)c1cc(C(=O)OC)c2c(c1)c1ccccc1[s+]2-c1ccccc1 ; CC(=O)c1ccc2c(c1)c(=O)c1ccccc1[s+]2-c1ccccc1 ; CC(=O)c1ccc2c(c1)Nc1ccccc1[S+]2c1ccccc1 ; CC(=O)c1ccc2c(c1)N(C)c1ccccc1[S+]2c1ccccc1 ; CC(=O)c1ccc2c(c1)Sc1ccccc1[S+]2c1ccccc1]

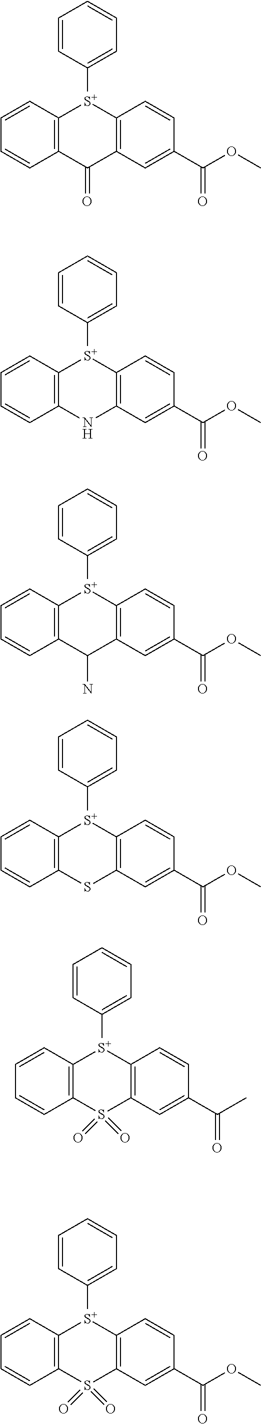 [SMILES: COC(=O)c1ccc2c(c1)c(=O)c1ccccc1[s+]2-c1ccccc1 ; COC(=O)c1ccc2c(c1)Nc1ccccc1[S+]2c1ccccc1 ; COC(=O)c1ccc2c(c1)C(N)c1ccccc1[S+]2c1ccccc1 ; COC(=O)c1ccc2c(c1)Sc1ccccc1[S+]2c1ccccc1 ; CC(=O)c1ccc2c(c1)S(=O)(=O)c1ccccc1[S+]2c1ccccc1 ; COC(=O)c1ccc2c(c1)S(=O)(=O)c1ccccc1[S+]2c1ccccc1]

The preferred sulfonium cations are dibenzothiophenium cations, that is, cations in formula (1) wherein X¹ is a single bond. These cations are preferred in the EUV lithography because of high photo-decomposition efficiency. More preferred are the cations whose substituent is an ester bond, that is, cations in formula (1) wherein R⁴ is an alkoxy group. Since the ester site is hydrolyzed during alkaline development, the dissolution of resist film in exposed region is improved and residue defects are minimized.

In formula (1), Xq⁻ is an anion having the formula (2).

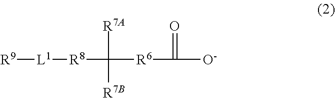

In formula (2), R⁶ is a single bond or C₁-C₄ alkanediyl group. Exemplary alkanediyl groups include methanediyl, ethane-1, 1-diyl, ethane-1,2-diyl, propane-1,2-diyl, propane-1,3-diyl, and butane-1,4-diyl.

In formula (2), R^{7A} and R^{7B} are each independently hydrogen, fluorine, or trifluoromethyl, at least one thereof being fluorine or trifluoromethyl.

In formula (2), R⁸ is a single bond or C₁-C₂₀ hydrocarbylene group which may contain a heteroatom. The hydrocarbylene group may be saturated or unsaturated and straight, branched or cyclic. Examples thereof include C₁-C₂₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, and dodecane-1,12-diyl; C₃-C₂₀ cyclic saturated hydrocarbylene groups such as cyclopentanediyl, cyclohexanediyl, norbornanediyl and adamantanediyl; C₆-C₂₀ arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene and tert-butylnaphthylene; and combinations thereof. In the hydrocarbylene group, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonic ester bond, carbonate bond, lactone ring, sultone ring, carboxylic anhydride (—C(═O)—O—C(═O)—) or haloalkyl moiety.

Also preferably, R⁸ is a group having the formula (2)-1.

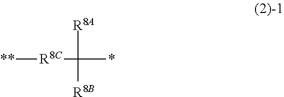

Herein * designates a point of attachment to the carbon atom to which R^{7A} and R^{7B} are attached, and ** designates a point of attachment to L¹.

In formula (2)-1, R^{8A} and R^{8B} are each independently hydrogen or C₁-C₁₀ hydrocarbyl group which may contain a heteroatom. R^{8A} and R^{8B} may bond together to form a ring with the carbon atom to which they are attached. The hydrocarbyl group may be saturated or unsaturated and straight, branched or cyclic. Examples thereof are as exemplified above for the hydrocarbyl group represented by R⁴ and R⁵ in formula (1). In the hydrocarbyl group, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonic ester bond, carbonate bond, lactone ring, sultone ring, carboxylic anhydride (—C(═O)—O—C(═O)—) or haloalkyl moiety.

In formula (2)-1, R^{8C} is a single bond or C₁-C₁₀ hydrocarbylene group which may contain a heteroatom. The hydrocarbylene group may be saturated or unsaturated and straight, branched or cyclic. Examples thereof are as exemplified above for the hydrocarbylene group represented by R⁸, but of 1 to 10 carbon atoms. In the hydrocarbylene group, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonic ester bond, carbonate bond, lactone ring, sultone ring, carboxylic anhydride (—C(═O)—O—C(═O)—) or haloalkyl moiety.

It is noted that the number of carbon atoms in the group having formula (2)-1 is 20 or less.

In formula (2), R⁹ is a C₆-C₂₀ straight or branched hydrocarbyl group which may contain a heteroatom or a C₃-C₂₀ cyclic hydrocarbyl group which may contain a heteroatom. Examples of the C₆-C₂₀ straight or branched hydrocarbyl group include C₆-C₂₀ alkyl groups such as hexyl, heptyl, octyl, nonyl, decyl, undecyl, dodecyl, tridecyl, tetradecyl, pentadecyl, heptadecyl, octadecyl, nonadecyl and icosyl. Examples of the C₃-C₂₀ cyclic hydrocarbyl group include C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₃-C₂₀ cyclic unsaturated aliphatic hydrocarbyl groups such as cyclohexenyl and norbornenyl; C₆-Cao aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, and tert-butylnaphthyl, and combinations thereof.

In formula (2), L¹ is an ether bond, ester bond, carbonate bond or urethane bond.

Those anions of formula (2) wherein R⁶ is a single bond and R^{7A} and R^{7B} are fluorine are preferred.

Examples of the anion having formula (2) are shown below, but not limited thereto.

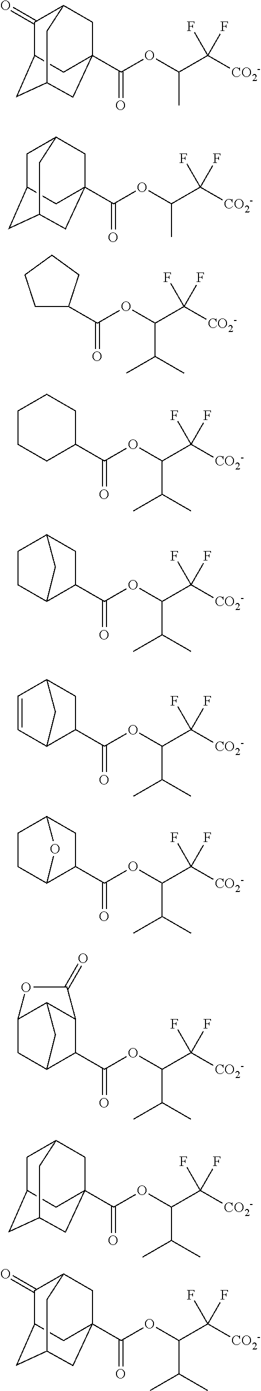

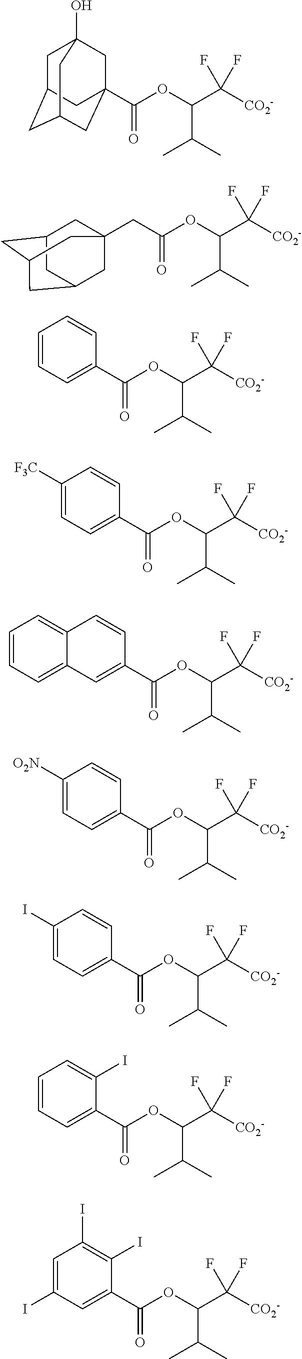

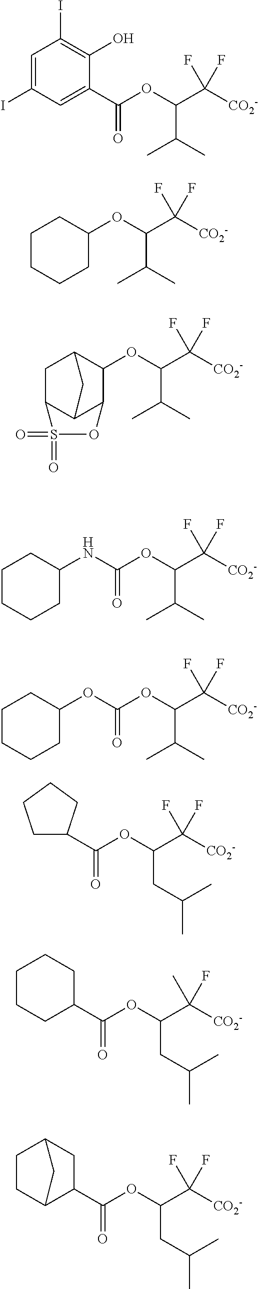

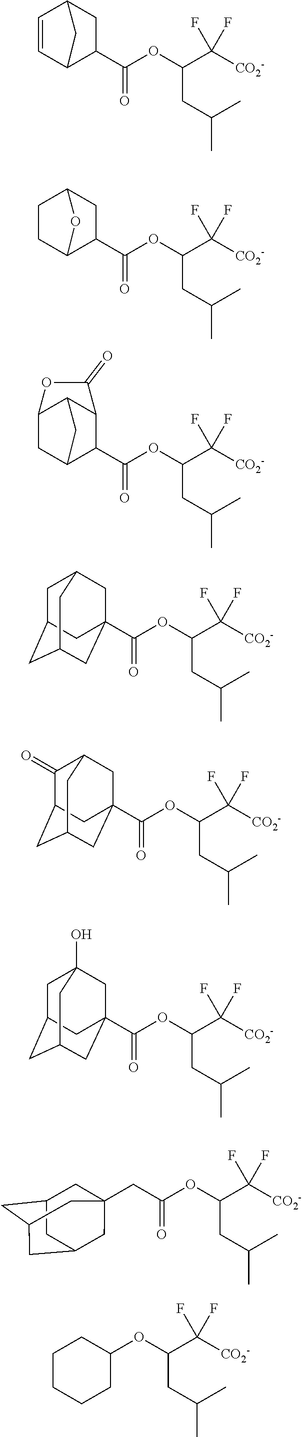

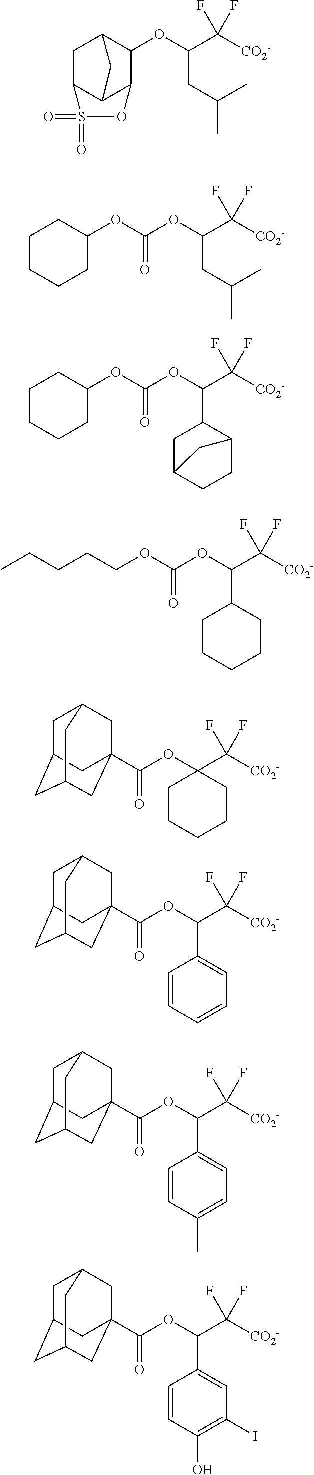

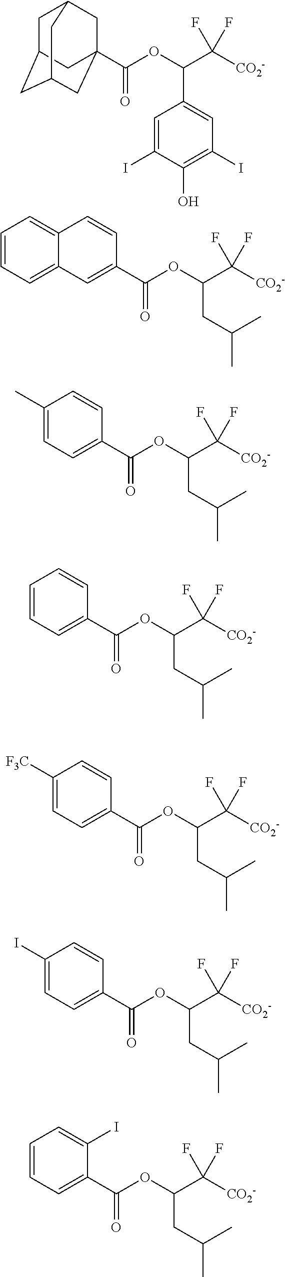

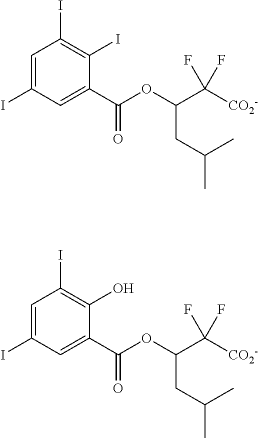

Examples of Sulfonium Salt A include arbitrary combinations of the cation with the anion, both as exemplified above.

Sulfonium Salt A may be synthesized, for example, by ion exchange between a sulfonium salt having a sulfonium cation as mentioned above and a ammonium salt having an anion as mentioned above or protic acid having an anion as mentioned above. The sulfonium cation can be obtained from reaction of a corresponding sulfide compound with a diphenyl iodonium salt.

Resist Composition

Another embodiment of the invention is a resist composition comprising a quencher containing Sulfonium Salt A.

In the resist composition, Sulfonium Salt A functions as a quencher. The resist composition comprising Sulfonium Salt A shows improved lithography properties including satisfactory acid diffusion control, reduced LWR and improved CDU. Despite low acid diffusion, high sensitivity and contrast are achievable. Although the reason is not well understood, the following mechanism is contemplated.

The cation in Sulfonium Salt A has a carbonyl group whose oxygen atom interacts with the acid generated from the PAG for thereby controlling acid diffusion. In addition, since Sulfonium Salt A has a condensed ring structure condensed via sulfur atom and hence, substantial distortion, it has a high efficiency of photo-decomposition, that is, high sensitivity. Although the sulfide structure of condensed ring form which is presumably formed after decomposition is generally low in dissolution in alkaline developer, Sulfonium Salt A has a high developer affinity due to appropriate polarity of its carbonyl group and is unlikely to form residue defects. Particularly when Sulfonium Salt A has an ester bond, which is hydrolyzed during development, Sulfonium Salt A becomes more soluble in alkaline developer (see Scheme 1 below). In Sulfonium Salt A, the positions of carbonyl group and ester bond are also important. These functional groups are positioned at the condensed ring site as shown in Scheme 1, which ensures the solubility of the sulfide compound as photo-decomposed. On the other hand, if the functional groups are positioned at the non-condensed ring site, the sulfide compound as photo-decomposed has a low dissolution in alkaline developer, causing defects.

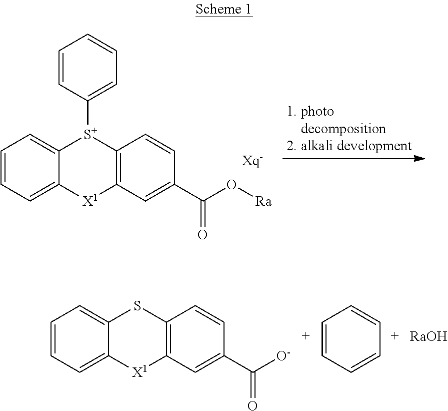

Herein Xq⁻ and X¹ are as defined above, and Ra is a hydrocarbyl group.

In the case of a dibenzothiophenium salt (i.e., X¹ is a single bond) as shown in Scheme 2, for example, dibenzothiophene which is presumably formed by photo-decomposition is substantially insoluble in alkali developer.

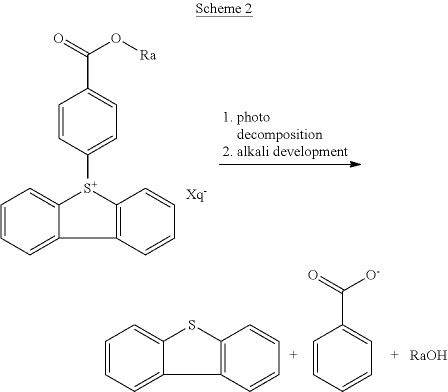

Herein Xq⁻ and Ra are as defined above.

It is believed that the anion structure of Sulfonium Salt A also contributes to the lithography performance of the inventive resist composition. Since the anion has a fluorinated carboxylate structure, it has an appropriate acidity. Since the anion structure also has a bulky hydrocarbyl group, it is expected to exert an acid diffusion suppressing effect. As a result, a resist composition having a good balance of sensitivity, roughness and resolution is established. Particularly in the EUV lithography, further improvements in the lithography performance are achievable by using the anion containing iodine which is highly absorptive to EUV radiation.

In the resist composition, Sulfonium Salt A is preferably used in an amount of 0.001 to 50 parts by weight, more preferably 0.01 to 40 parts by weight per 100 parts by weight of the base polymer to be described below. Sulfonium Salt A may be used alone or in admixture of two or more.

Base Polymer

In one embodiment, the resist composition contains a base polymer. In the case of positive resist compositions, the base polymer comprises repeat units containing an acid labile group. The preferred repeat units containing an acid labile group are repeat units having the formula (a1) or repeat units having the formula (a2), which are also referred to as repeat units (a1) or (a2).

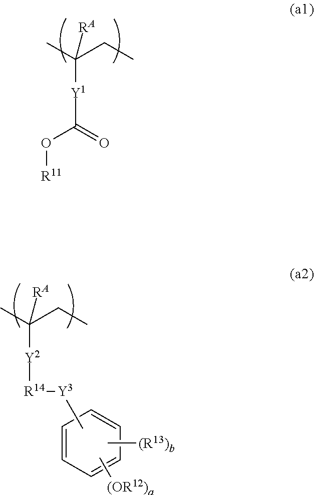

In formulae (a1) and (a2), R^{A} is each independently hydrogen or methyl. Y¹ is a single bond, phenylene group, naphthylene group, or a C₁-C₁₂ linking group containing an ester bond and/or lactone ring. Y 2 is a single bond or ester bond. Y 3 is a single bond, ether bond or ester bond. R¹¹ and R¹² are each independently an acid labile group. It is noted that when the base polymer contains both repeat units (a1) and (a2), R¹¹ and R¹² may be identical or different. R¹³ is fluorine, trifluoromethyl, cyano or a C₁-C₆ saturated hydrocarbyl group. R¹⁴ is a single bond or a C₁-C₆ alkanediyl group in which some carbon may be replaced by an ether bond or ester bond. The subscript “a” is 1 or 2, “b” is an integer of 0 to 4, and the sum of a+b is from 1 to 5.

Examples of the monomer from which repeat units (a1) are derived are shown below, but not limited thereto. Herein R A and R H are as defined above.

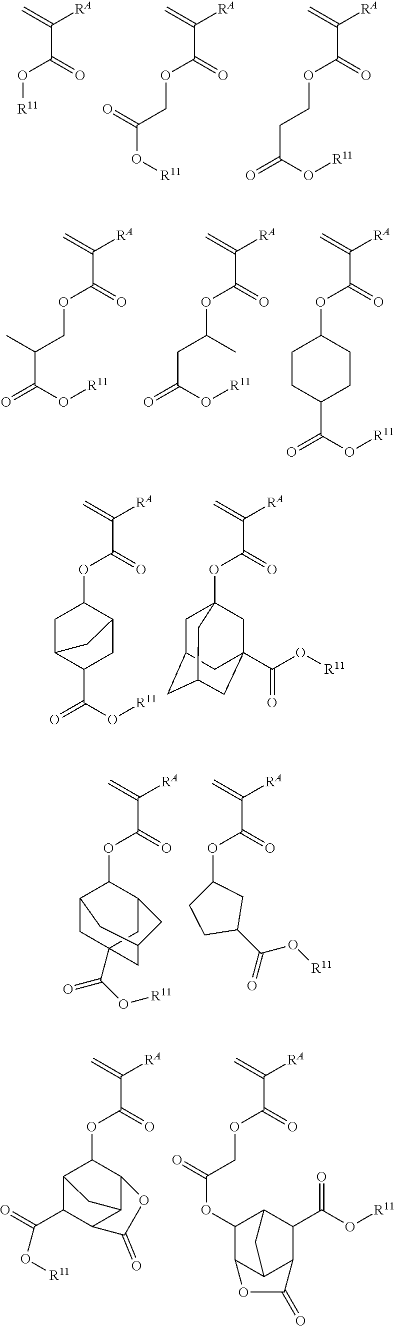

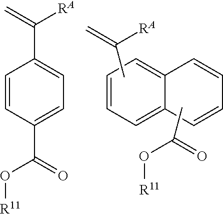

Examples of the monomer from which repeat units (a2) are derived are shown below, but not limited thereto. Herein R^{A} and R¹² are as defined above.

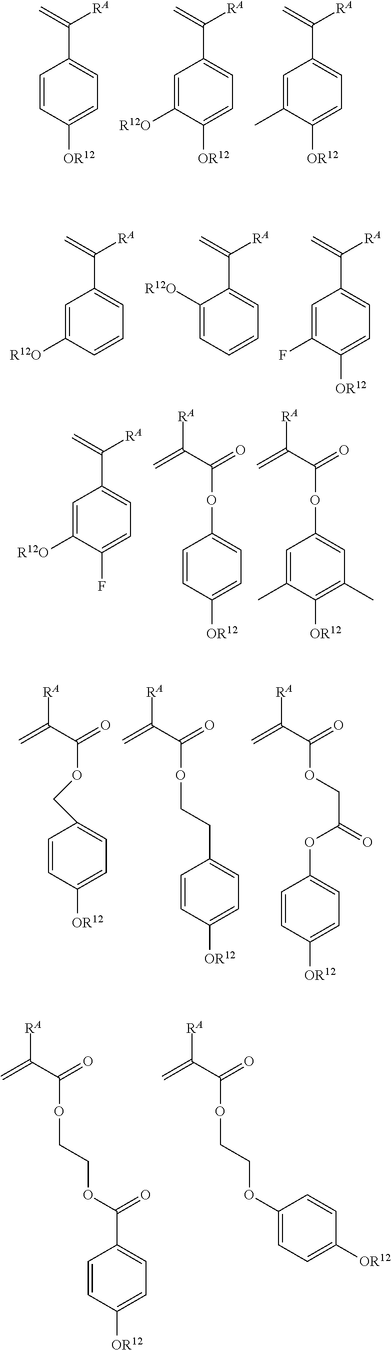

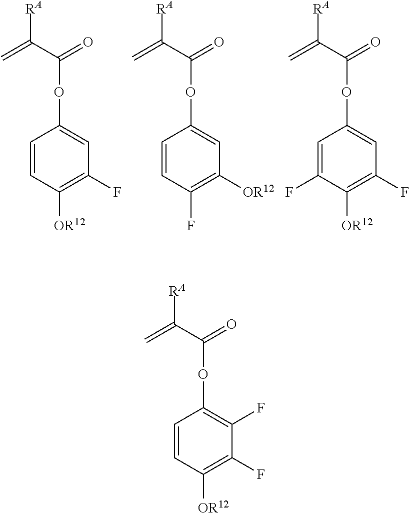

The acid labile groups represented by R^{4A}, R¹¹ and R^{L2} in formulae (1), (a1) and (a2) may be selected from a variety of such groups, for example, those groups described in JP-A 2013-080033 (U.S. Pat. No. 8,574,817) and JP-A 2013-083821 (U.S. Pat. No. 8,846,303).

Typical of the acid labile group are groups of the following formulae (AL-1) to (AL-3).

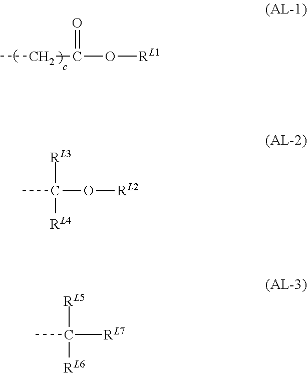

In formulae (AL-1) and (AL-2), R^{L1} and R^{L2} are each independently a C₁-C₄₀ hydrocarbyl group which may contain a heteroatom such as oxygen, sulfur, nitrogen or fluorine. The hydrocarbyl group may be saturated or unsaturated and straight, branched or cyclic. Inter alia, C₁-C₄₀ saturated hydrocarbyl groups are preferred, and C₁-C₂₀ saturated hydrocarbyl groups are more preferred.

In formula (AL-1), c is an integer of 0 to 10, preferably 1 to 5.

In formula (AL-2), R^{L3} and R^{L4} are each independently hydrogen or a C₁-C₂₀ hydrocarbyl group which may contain a heteroatom such as oxygen, sulfur, nitrogen or fluorine. The hydrocarbyl group may be saturated or unsaturated and straight, branched or cyclic. Inter alia, C₁-C₂₀ saturated hydrocarbyl groups are preferred. Any two of R^{L2}, R^{L3} and R^{L4} may bond together to form a C₃-C₂₀ ring with the carbon atom or carbon and oxygen atoms to which they are attached. The ring preferably contains 4 to 16 carbon atoms and is typically alicyclic.

In formula (AL-3), R^{L5}, R^{L6} and R^{L7} are each independently a C₁-C₂₀ hydrocarbyl group which may contain a heteroatom such as oxygen, sulfur, nitrogen or fluorine. The hydrocarbyl group may be saturated or unsaturated and straight, branched or cyclic. Inter alia, C₁-C₂₀ saturated hydrocarbyl groups are preferred. Any two of R^{L5}, R^{L6} and R^{L7} may bond together to form a C₃-C₂₀ ring with the carbon atom to which they are attached. The ring preferably contains 4 to 16 carbon atoms and is typically alicyclic.

The base polymer may further comprise repeat units (b) having a phenolic hydroxy group as an adhesive group. Examples of suitable monomers from which repeat units (b) are derived are given below, but not limited thereto. Herein R A is as defined above.

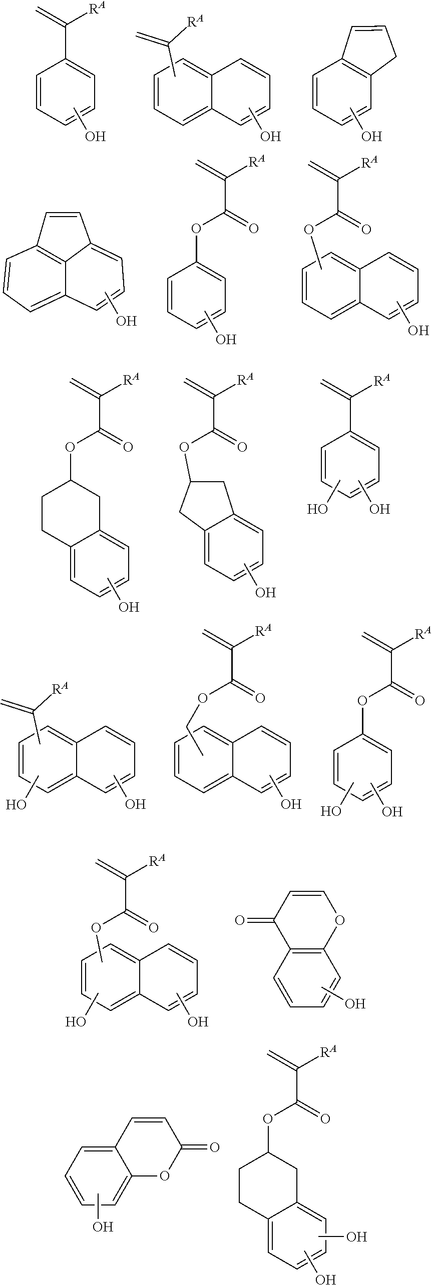

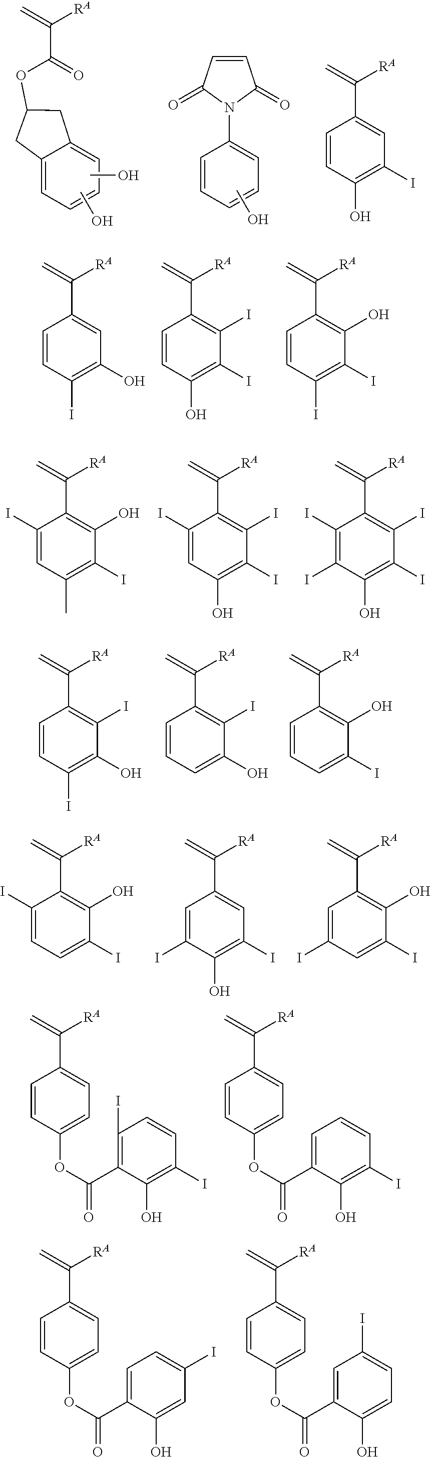

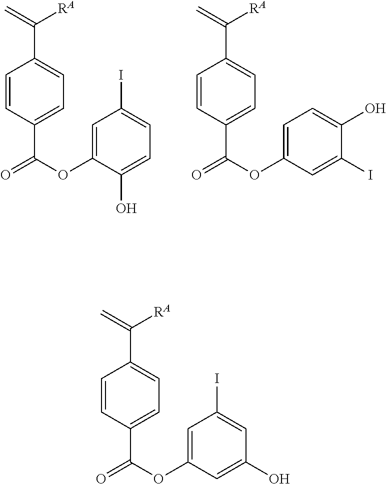

The base polymer may further comprise repeat units (c) having another adhesive group selected from hydroxy group (other than the foregoing phenolic hydroxy), lactone ring, sultone ring, ether bond, ester bond, sulfonic ester bond, carbonyl group, sulfonyl group, cyano group, and carboxy group. Examples of suitable monomers from which repeat units (c) are derived are given below, but not limited thereto. Herein R A is as defined above.

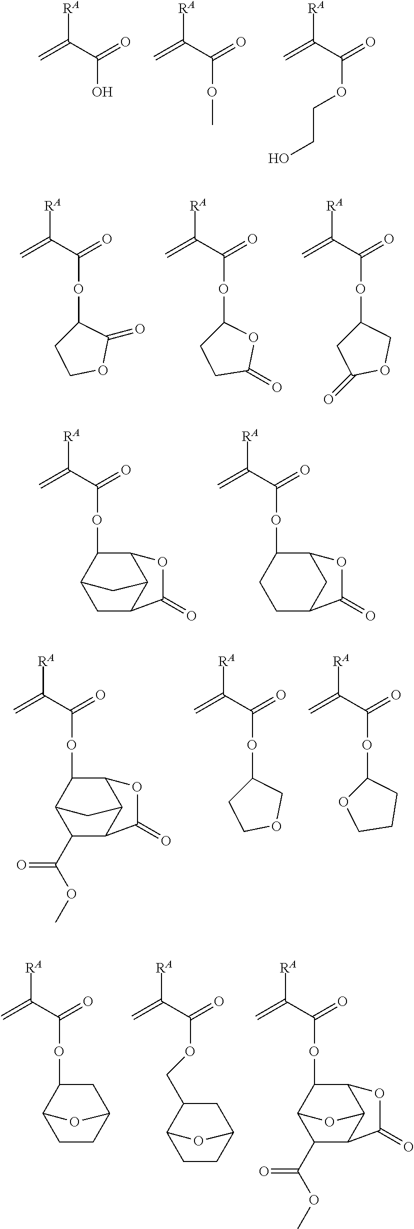

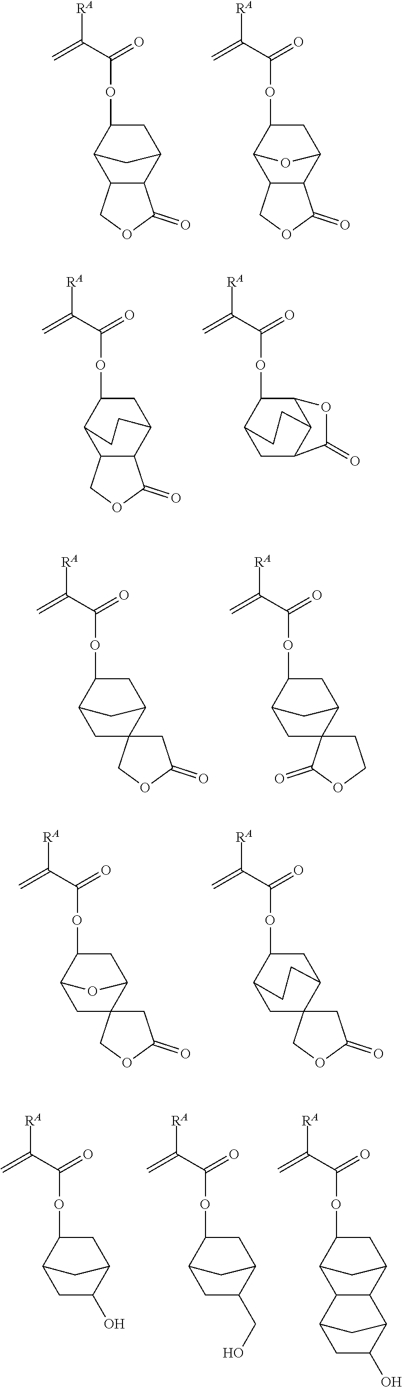

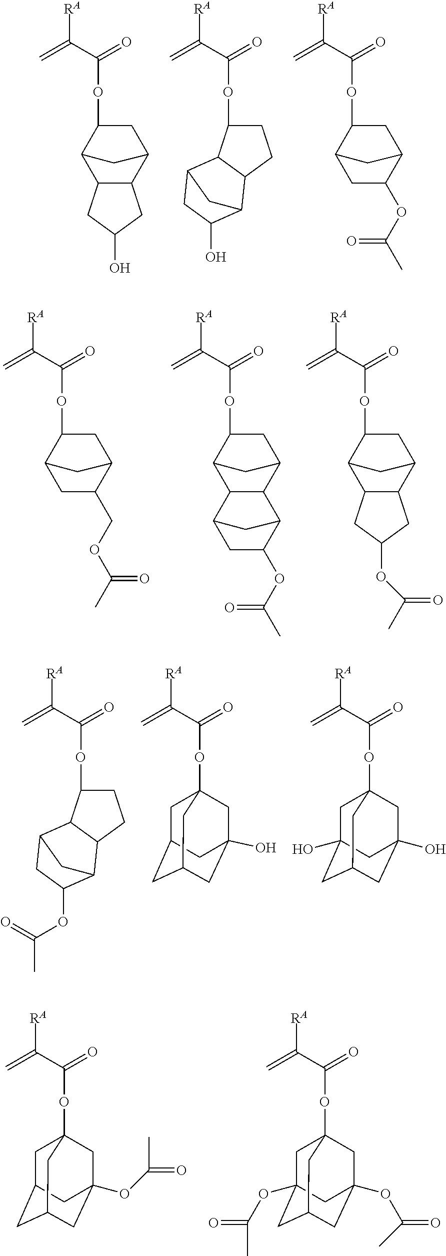

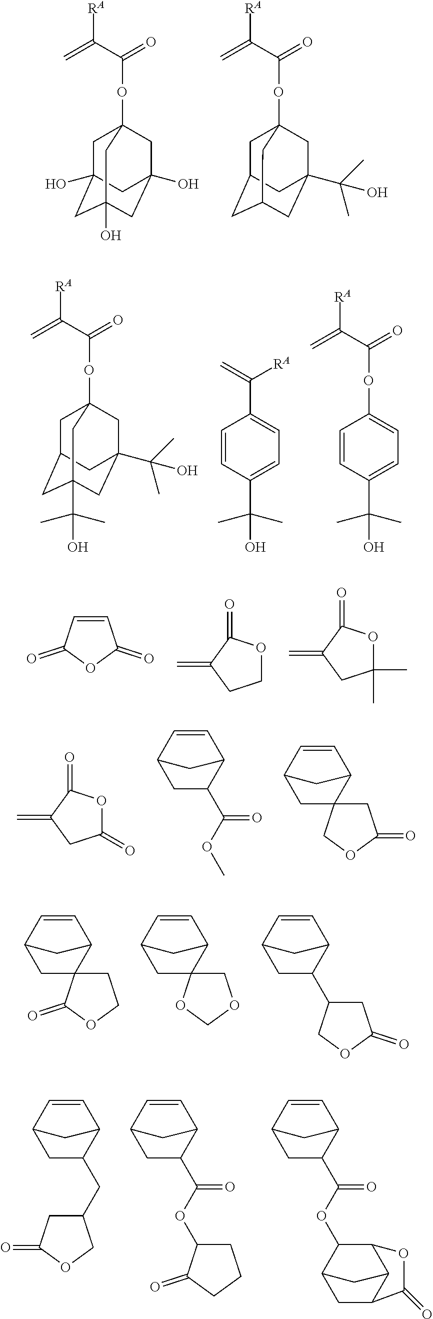

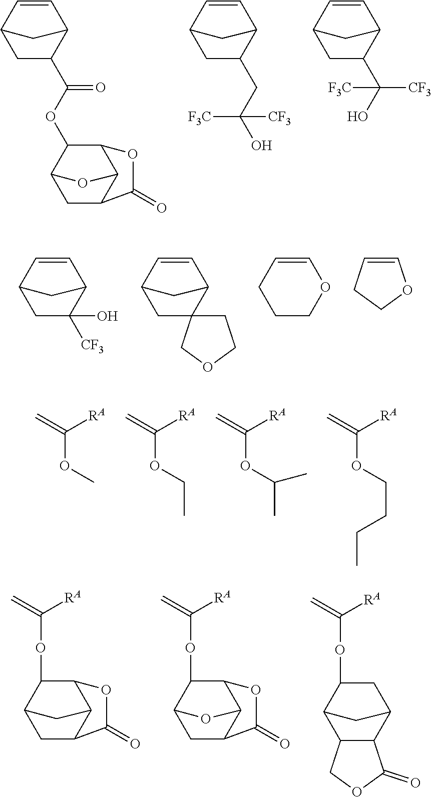

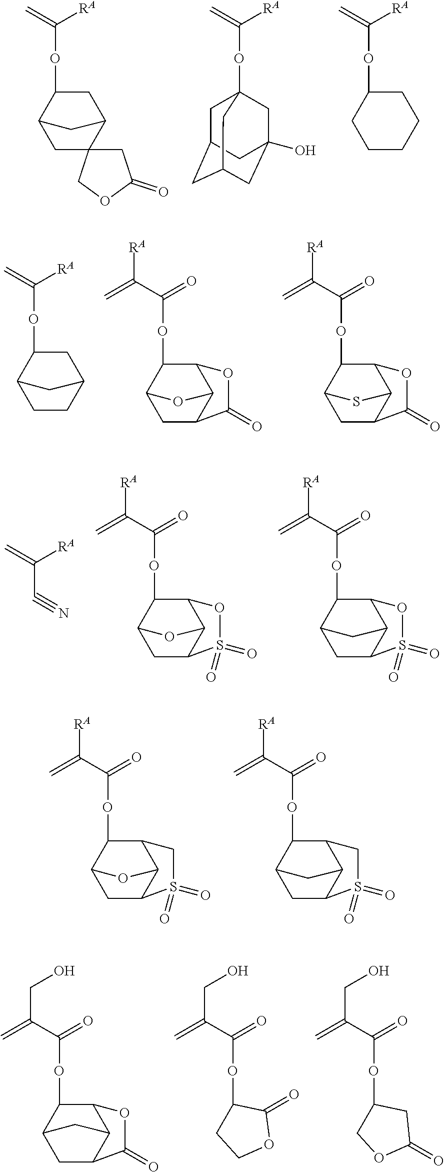

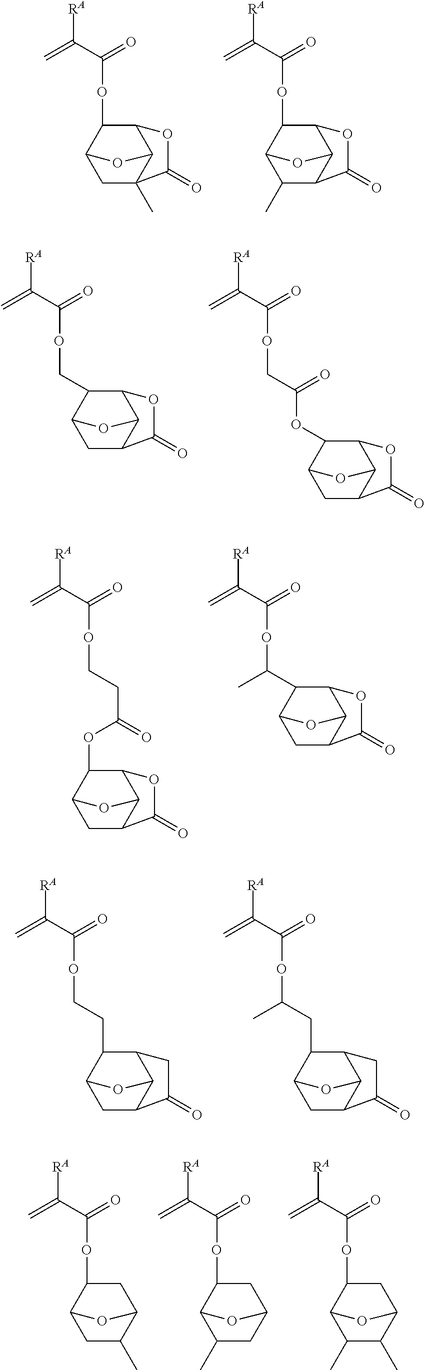

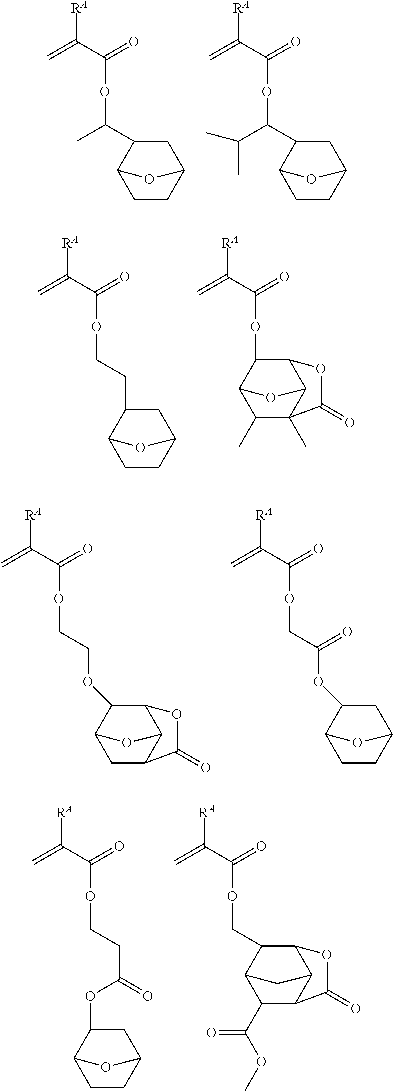

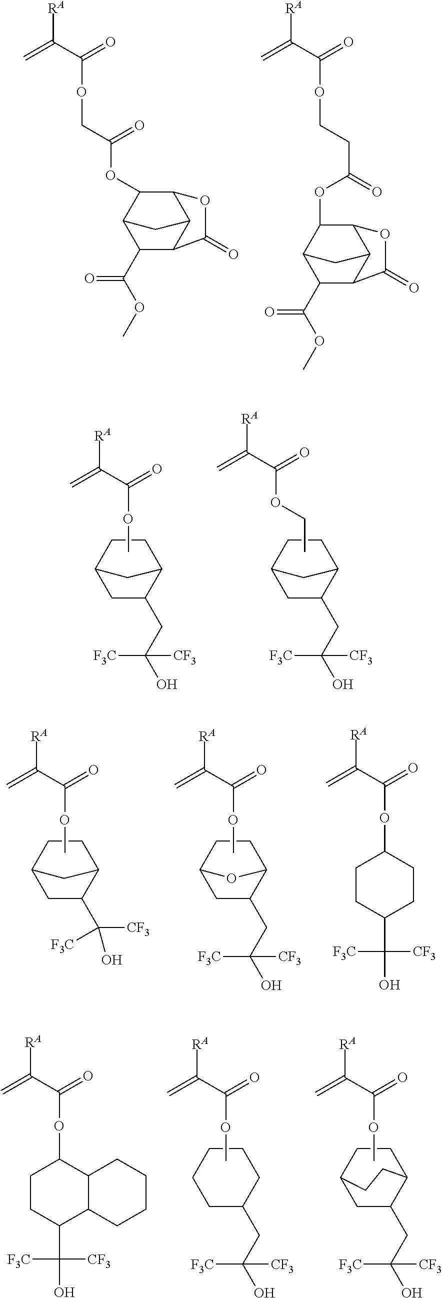

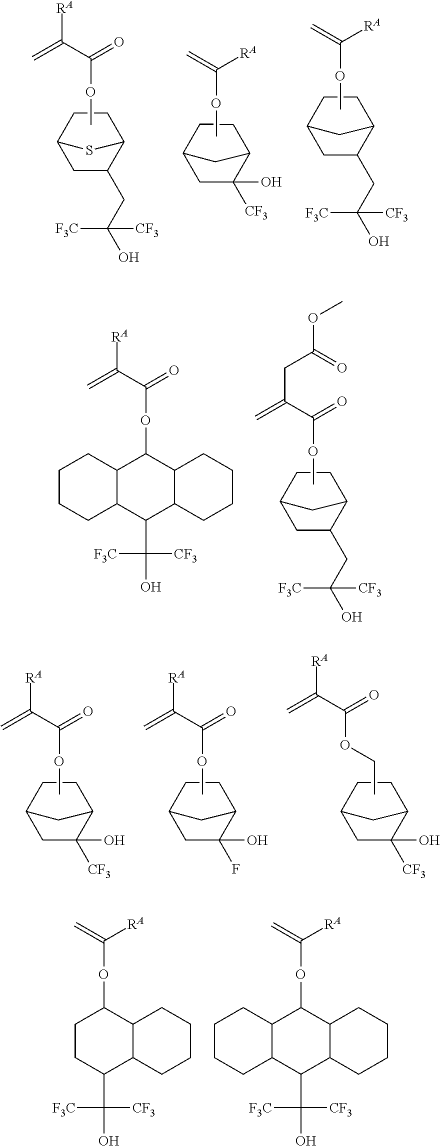

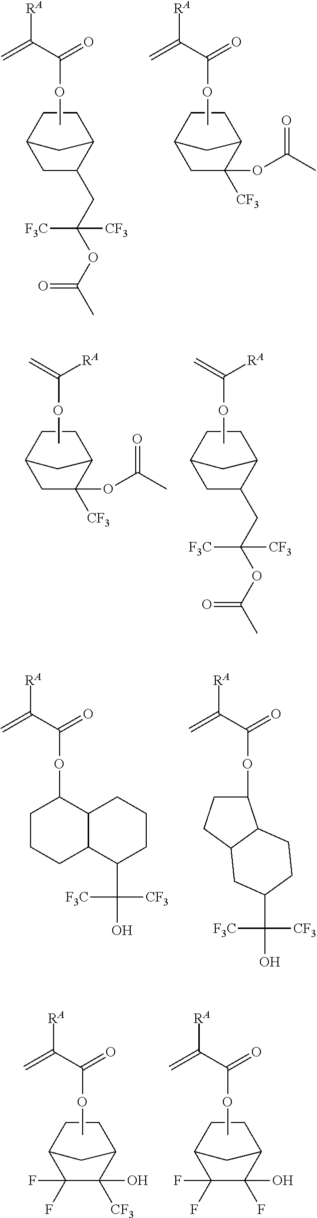

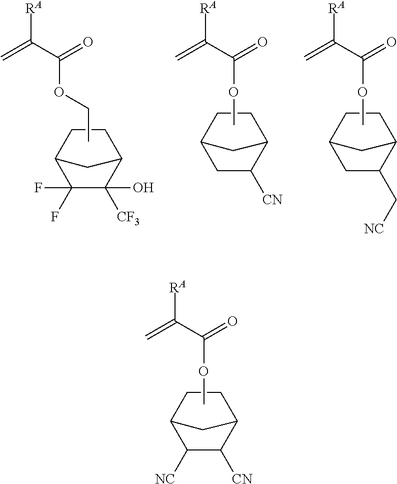

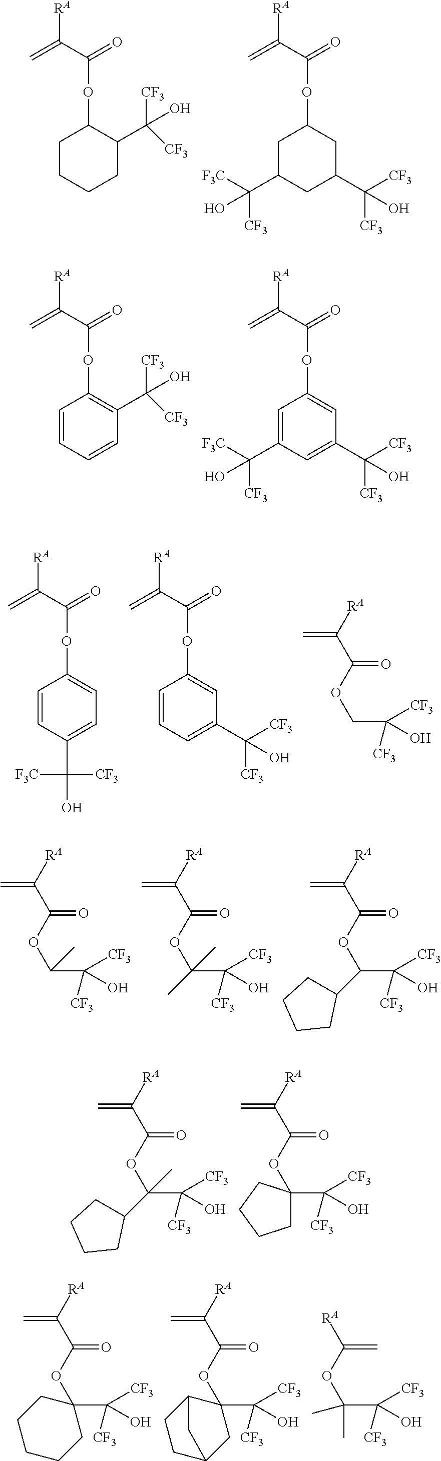

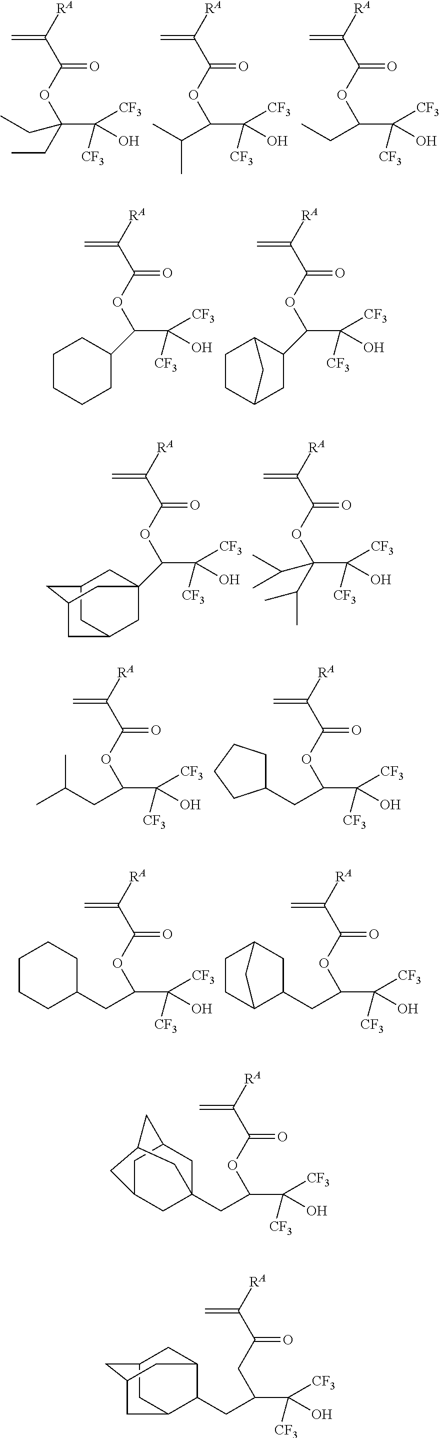

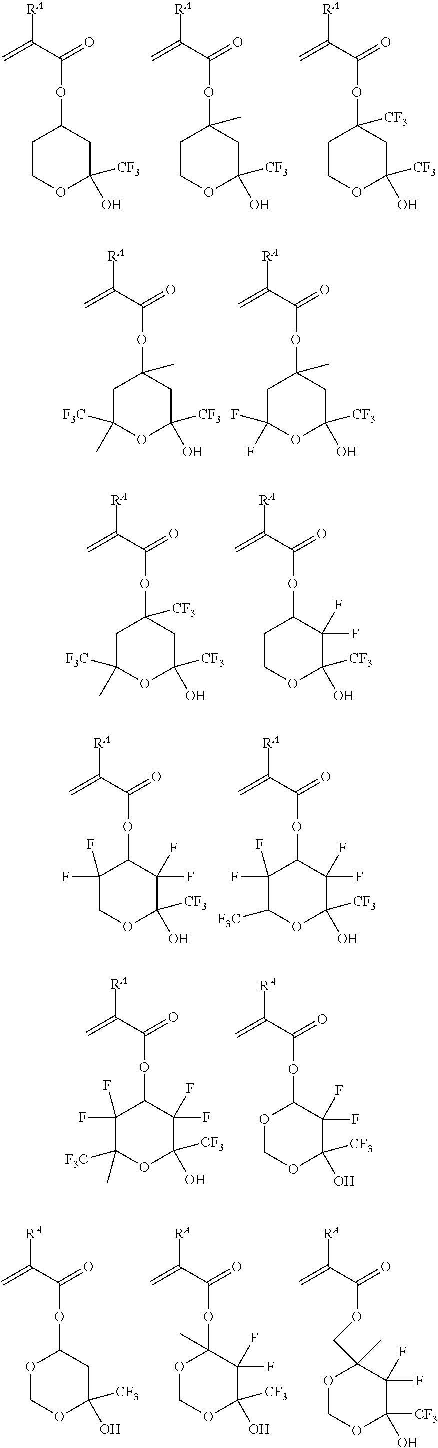

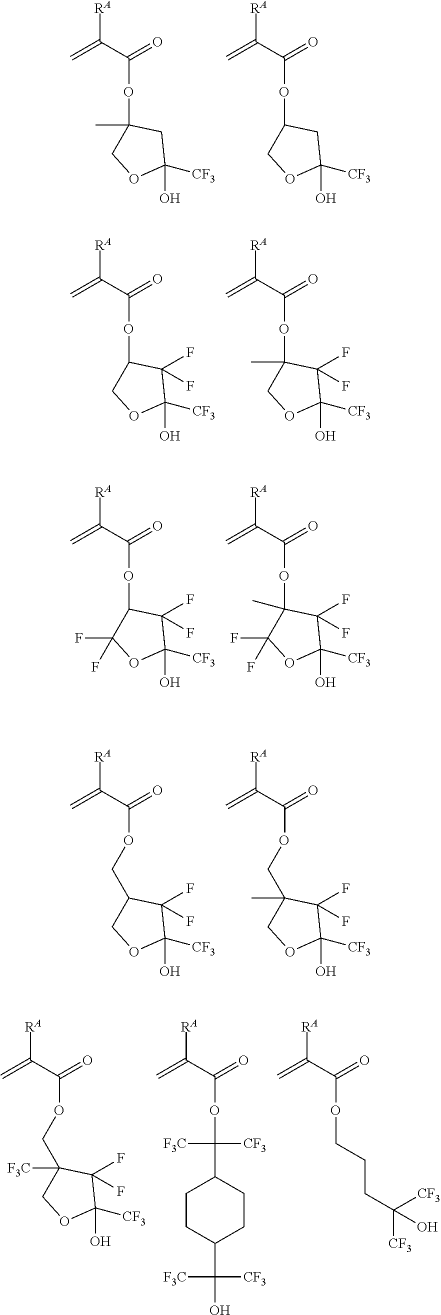

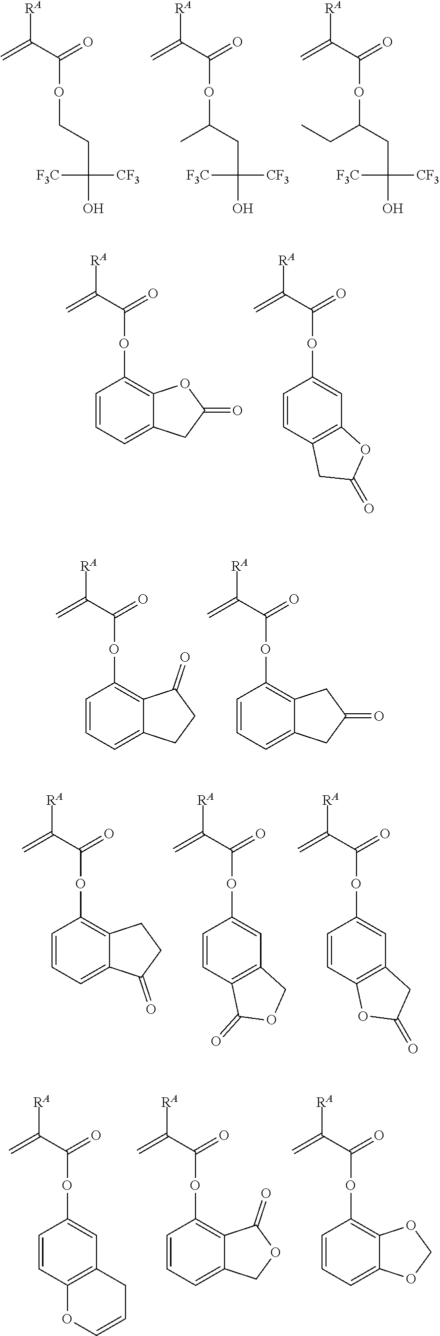

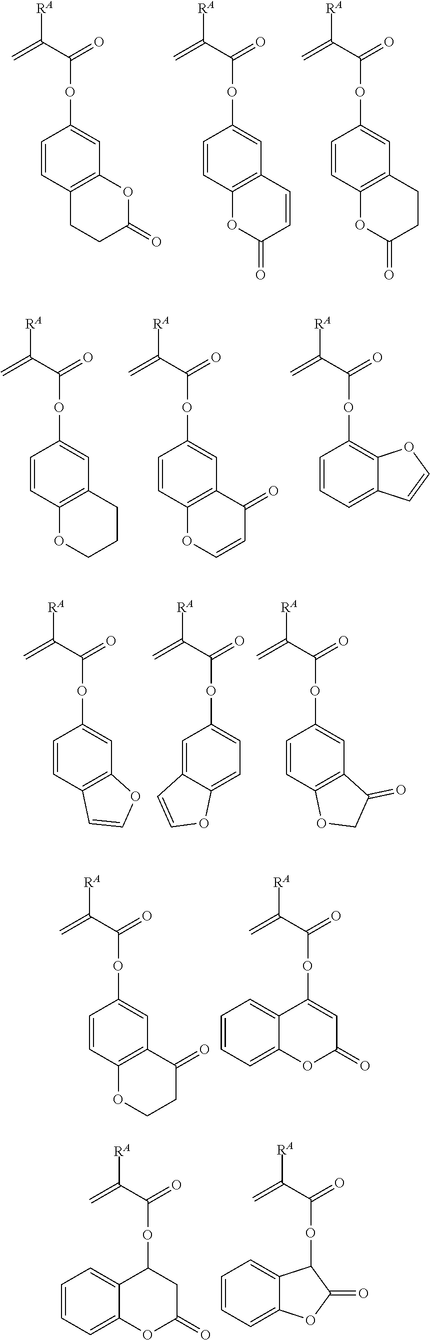

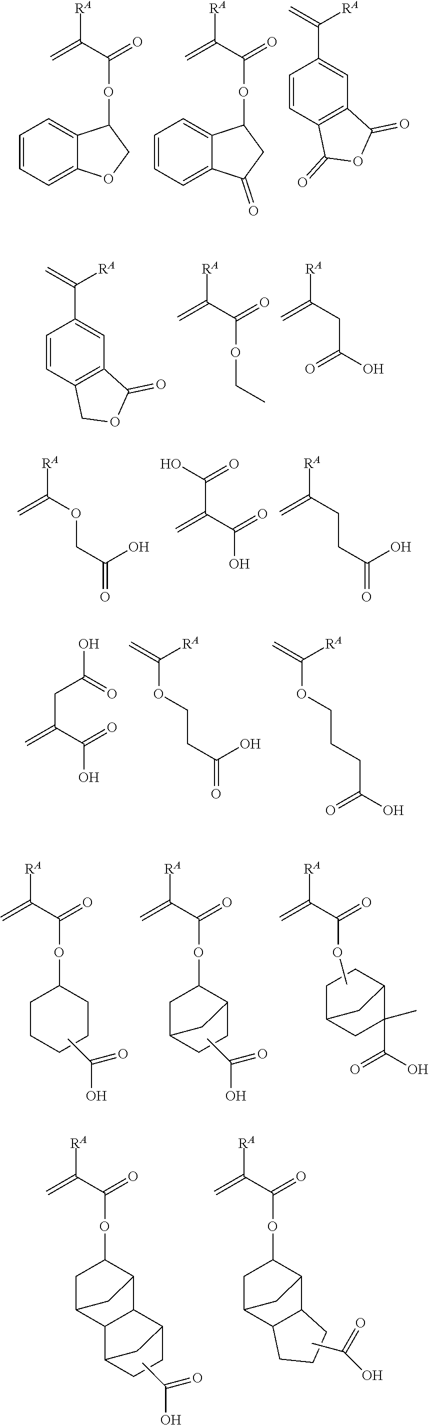

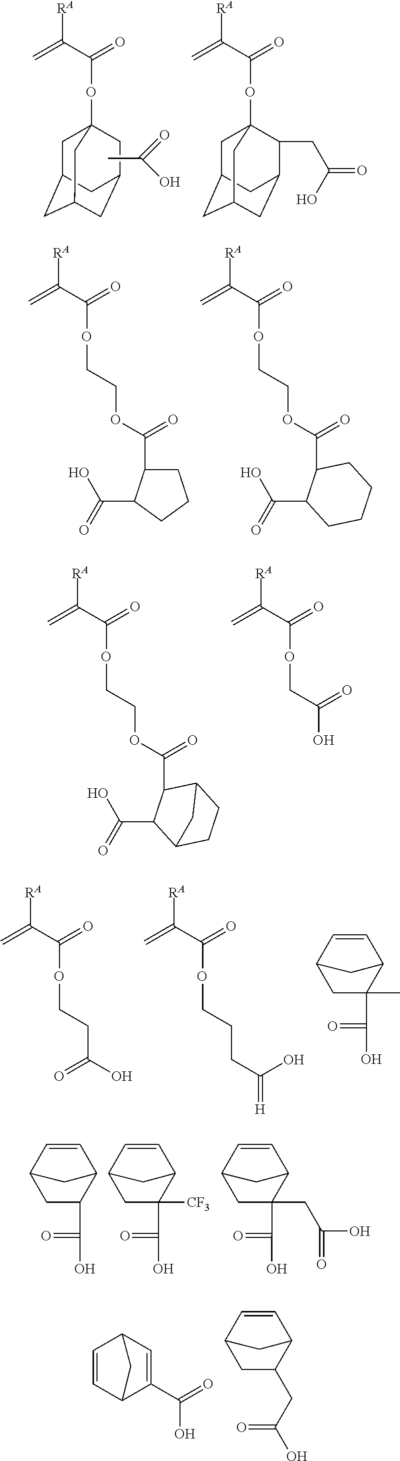

In another preferred embodiment, the base polymer may further comprise repeat units (d) derived from indene, benzofuran, benzothiophene, acenaphthylene, chromone, coumarin, and norbornadiene, or derivatives thereof. Suitable monomers are exemplified below.

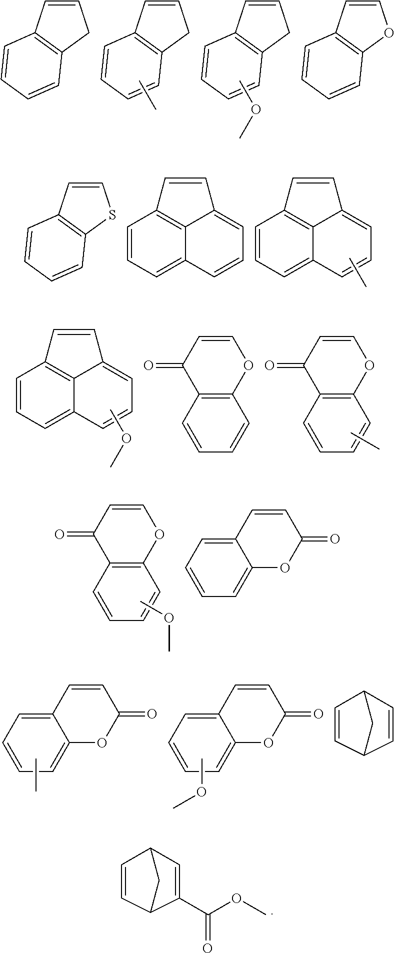

Furthermore, the base polymer may comprise repeat units (e) derived from styrene, vinylnaphthalene, vinylanthracene, vinylpyrene, methyleneindene, vinylpyridine, vinylcarbazole, or derivatives thereof.

In a further embodiment, the base polymer may comprise repeat units (f) derived from an onium salt having a polymerizable unsaturated bond. Specifically, the base polymer may comprise repeat units of at least one type selected from repeat units having formula (f1), repeat units having formula (f2), and repeat units having formula (f3), all shown below. These units are simply referred to as repeat units (f1), (f2) and (f3), which may be used alone or in combination of two or more types.

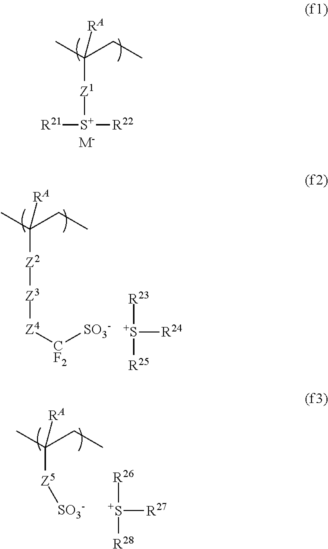

In formulae (f1) to (f3), R A is each independently hydrogen or methyl. Z¹ is a single bond, C₁-C₆ aliphatic hydrocarbylene group, phenylene group, naphthylene group, or C₇-Cis group obtained by combining the foregoing, —O—Z¹¹—, —C(═O)—O—Z¹¹—, or —C(═O)—NH—Z¹¹-Z¹¹ is a C₁-C₆ aliphatic hydrocarbylene group, phenylene group, naphthylene group, or C₇-C₁₈ group obtained by combining the foregoing, which may contain a carbonyl moiety, ester bond, ether bond or hydroxy moiety. Z² is a single bond or ester bond. Z³ is a single bond, —Z³¹—C(═O)—O—, —Z³¹—O— or —Z³¹—O—C(═O)—. Z³¹ is a C₁-C₁₂ aliphatic hydrocarbylene group, phenylene group, or C₇-Cis group obtained by combining the foregoing, which may contain a carbonyl moiety, ester bond, ether bond, iodine or bromine. Z⁴ is methylene, 2,2,2-trifluoro-1,1-ethanediyl or carbonyl group. Z⁵ is a single bond, methylene, ethylene, phenylene, fluorinated phenylene, trifluoromethyl-substituted phenylene, —O—Z⁵¹—, —C(═O)—O—Z⁵¹—, or —C(═O)—NH—Z⁵¹—. Z⁵¹ is a C₁-C₆ aliphatic hydrocarbylene group, phenylene group, fluorinated phenylene group, or trifluoromethyl-substituted phenylene group, which may contain a carbonyl moiety, ester bond, ether bond, halogen or hydroxy moiety.

In formulae (f1) to (f3), R²¹ to R²⁸ are each independently halogen or a C₁-C₂₀ hydrocarbyl group which may contain a heteroatom. The hydrocarbyl group may be saturated or unsaturated and straight, branched or cyclic. Examples of the halogen and hydrocarbyl group are as will be exemplified later for R¹⁰¹ to R¹⁰⁵ in formulae (3-1) and (3-2). In these hydrocarbyl groups, some or all of the hydrogen atoms may be substituted by a moiety containing a heteroatom such as oxygen, sulfur, nitrogen or halogen and some constituent —CH₂— may be replaced by a moiety containing a heteroatom such as oxygen, sulfur or nitrogen, so that the group may contain a hydroxy moiety, fluorine, chlorine, bromine, iodine, cyano moiety, nitro moiety, carbonyl moiety, ether bond, ester bond, sulfonic ester bond, carbonate bond, lactone ring, sultone ring, carboxylic anhydride (—C(═O)—O—C(═O)—), or haloalkyl moiety. A pair of R²³ and R²⁴, or R²⁶ and R²⁷ may bond together to form a ring with the sulfur atom to which they are attached. Examples of the ring are as will be exemplified later for the ring that R¹⁰¹ and R¹⁰² in formulae (3-1) and (3-2), taken together, form with the sulfur atom to which they are attached.

In formula (f1), M⁻ is a non-nucleophilic counter ion. Examples of the non-nucleophilic counter ion include halide ions such as chloride and bromide ions; fluoroalkylsulfonate ions such as triflate, 1,1,1-trifluoroethanesulfonate, and nonafluorobutanesulfonate; arylsulfonate ions such as tosylate, benzenesulfonate, 4-fluorobenzenesulfonate, and 1,2,3,4,5-pentafluorobenzenesulfonate; alkylsulfonate ions such as mesylate and butanesulfonate; imide ions such as bis(trifluoromethylsulfonyl)imide, bis(perfluoroethylsulfonyl)imide and bis(perfluorobutylsulfonyl)imide; methide ions such as tris(trifluoromethylsulfonyl)methide and tris(perfluoroethylsulfonyl)methide.

Also included are sulfonate ions having fluorine substituted at α-position as represented by the formula (f1-1) and sulfonate ions having fluorine substituted at α-position and trifluoromethyl at β-position as represented by the formula (f1-2).

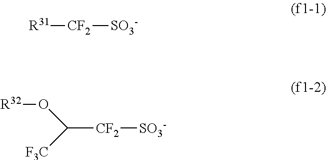

In formula (f1-1), R³¹ is hydrogen, or a C₁-C₂₀ hydrocarbyl group which may contain an ether bond, ester bond, carbonyl moiety, lactone ring, or fluorine atom. The hydrocarbyl group may be saturated or unsaturated and straight, branched or cyclic. Examples of the hydrocarbyl group are as will be exemplified later for R^{fa1} in formula (3A′).

In formula (f1-2), R³² is hydrogen, or a C₁-C₃₀ hydrocarbyl group or C₂-C₃₀ hydrocarbylcarbonyl group, which may contain an ether bond, ester bond, carbonyl moiety or lactone ring. The hydrocarbyl group and hydrocarbyl moiety in the hydrocarbylcarbonyl group may be saturated or unsaturated and straight, branched or cyclic. Examples of the hydrocarbyl group are as will be exemplified later for R^{fa1} in formula (3A′).

Examples of the cation in the monomer from which repeat unit (f1) is derived are shown below, but not limited thereto. R A is as defined above.

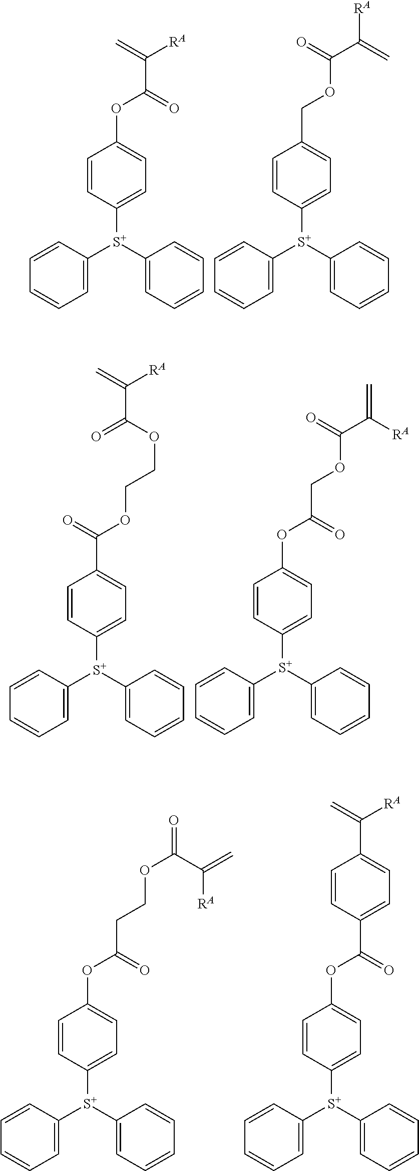

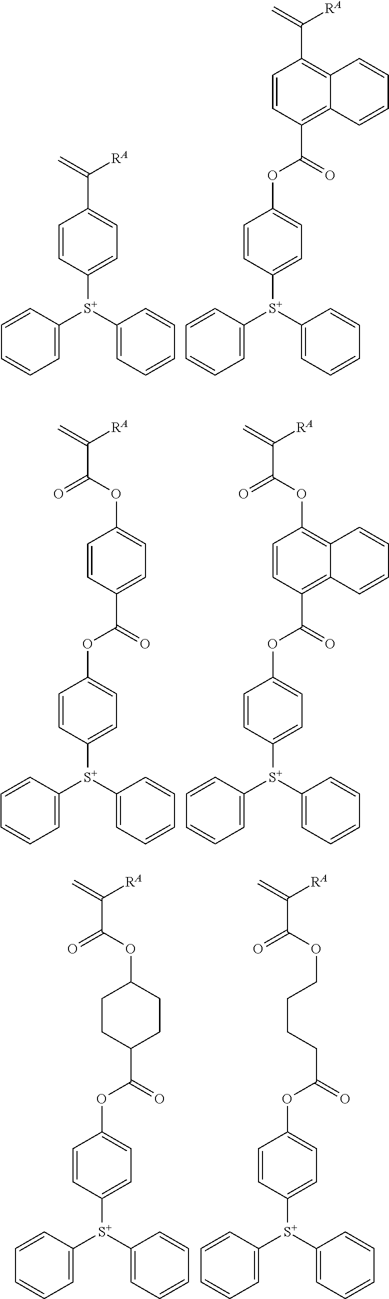

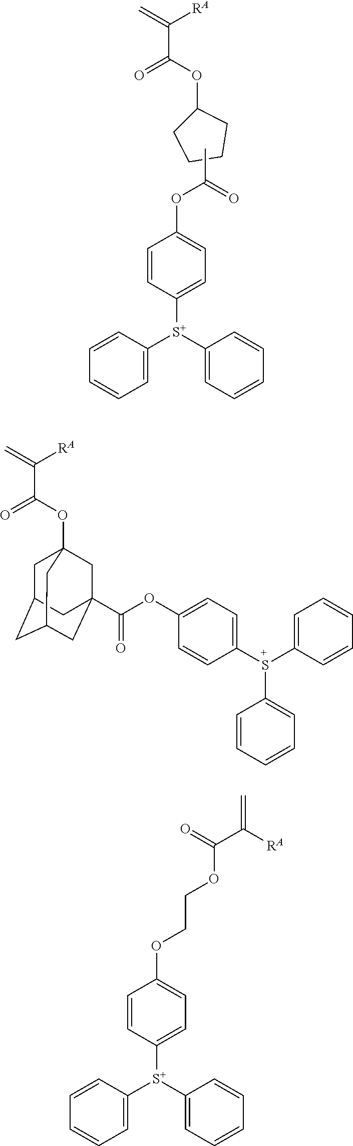

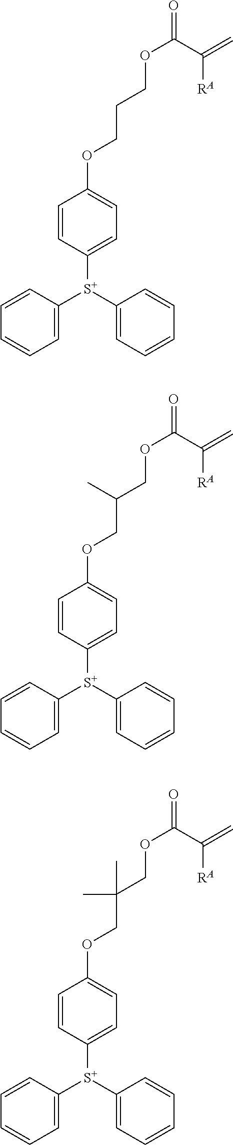

Examples of the cation in the monomer from which repeat unit (f2) or (f3) is derived are as exemplified above for the cation in the sulfonium salt having formula (1) or as will be exemplified later for the cation in the sulfonium salt having formula (3-1).

Examples of the anion in the monomer from which repeat unit (f2) is derived are shown below, but not limited thereto. R A is as defined above.

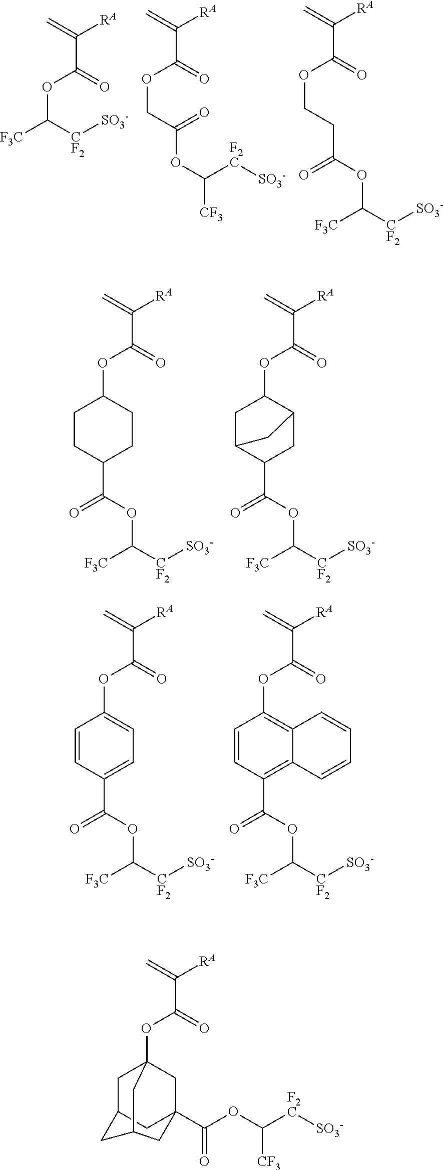

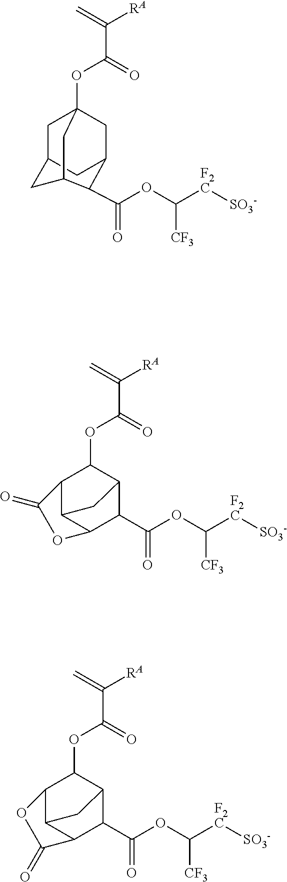

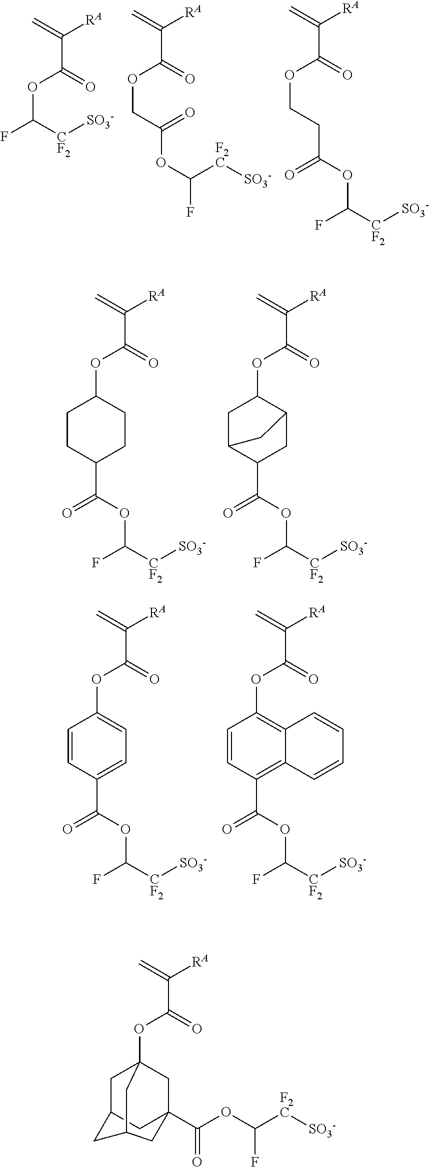

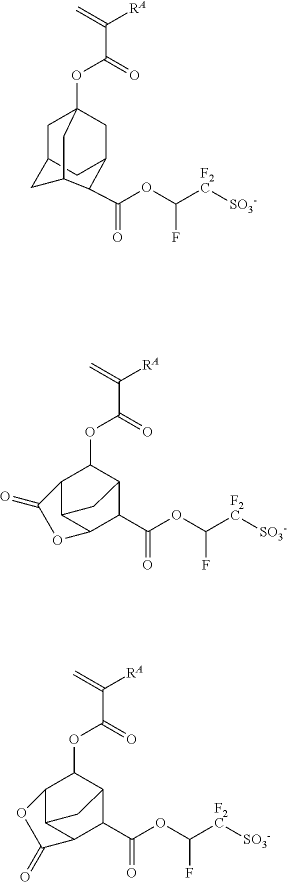

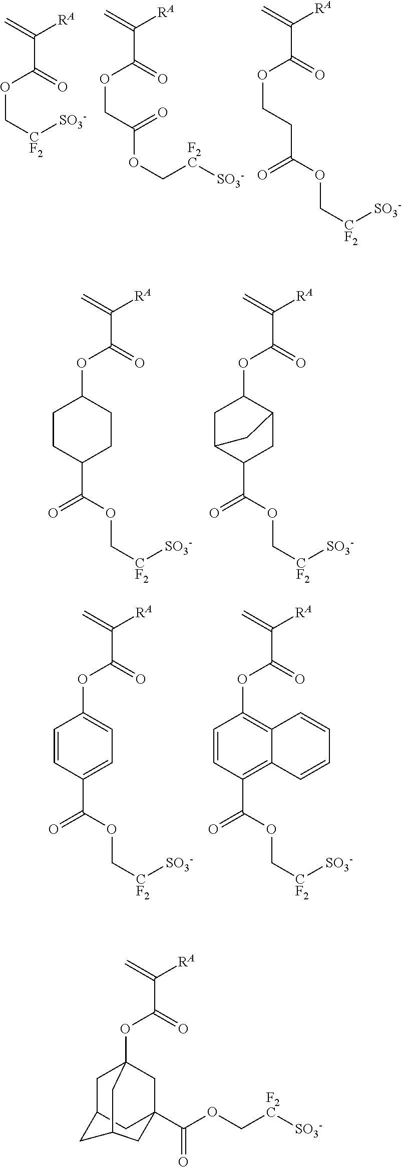

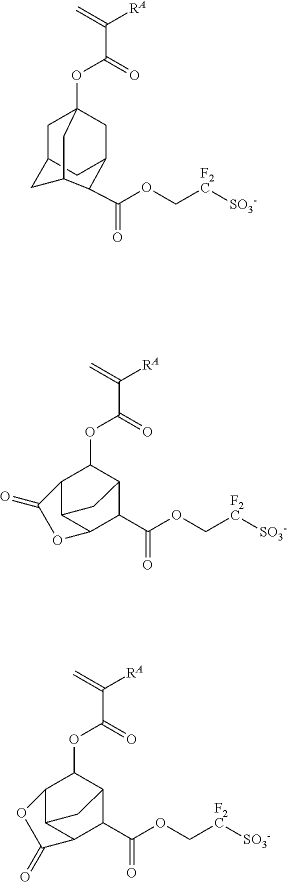

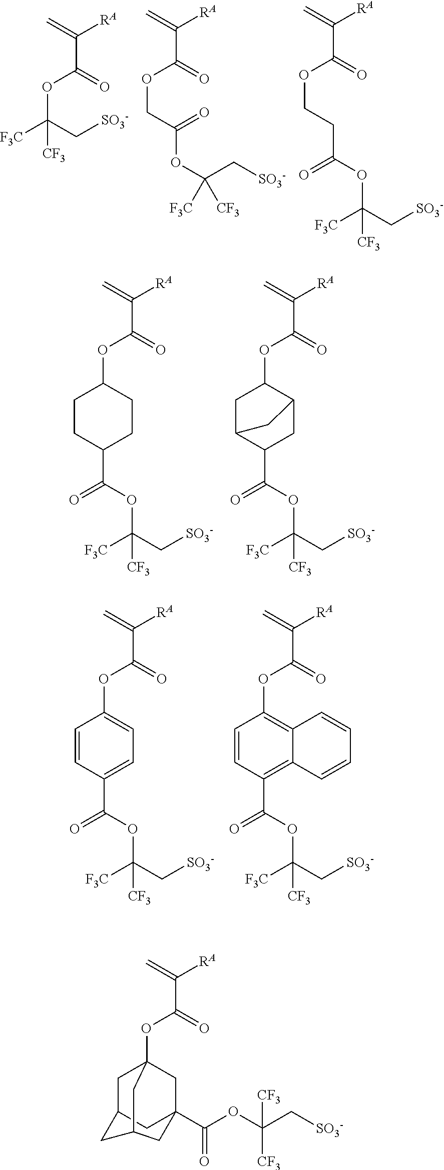

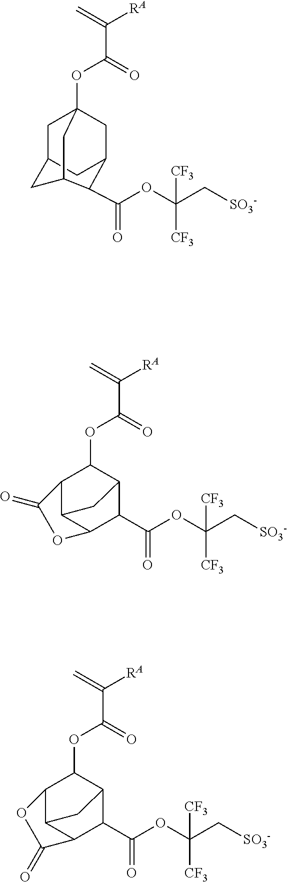

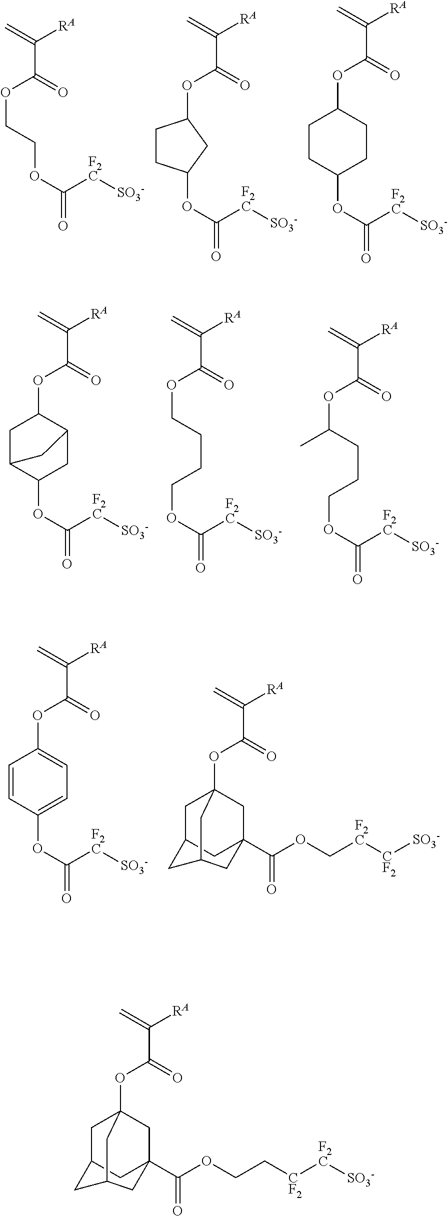

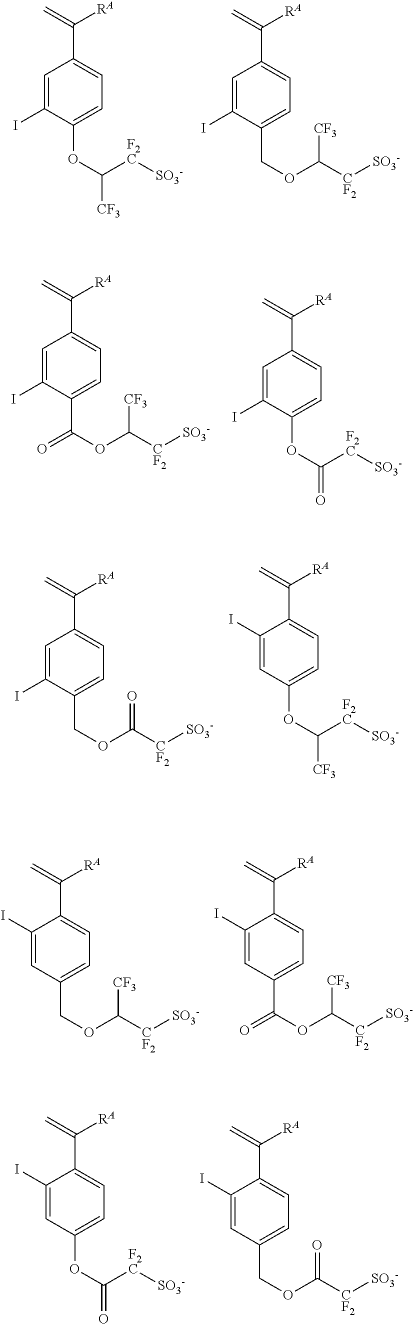

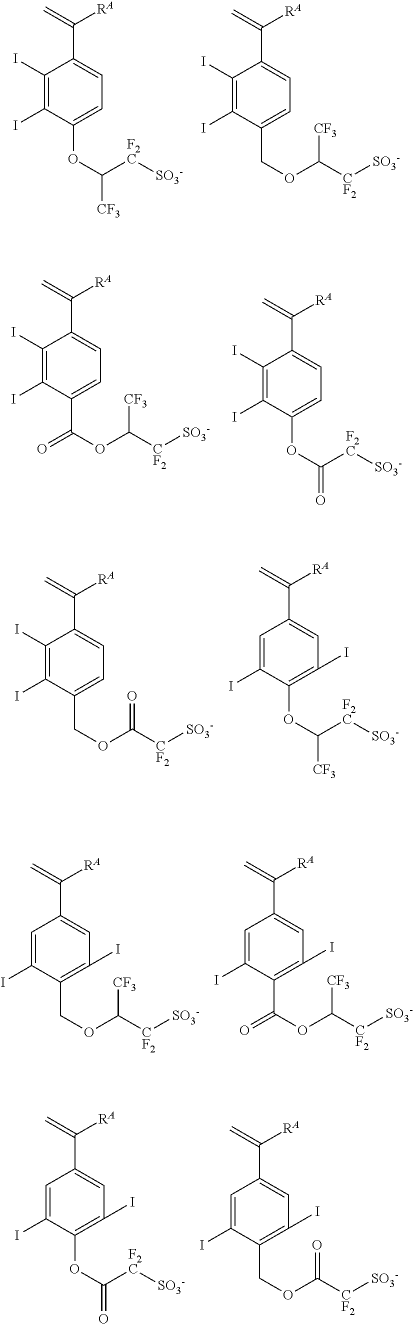

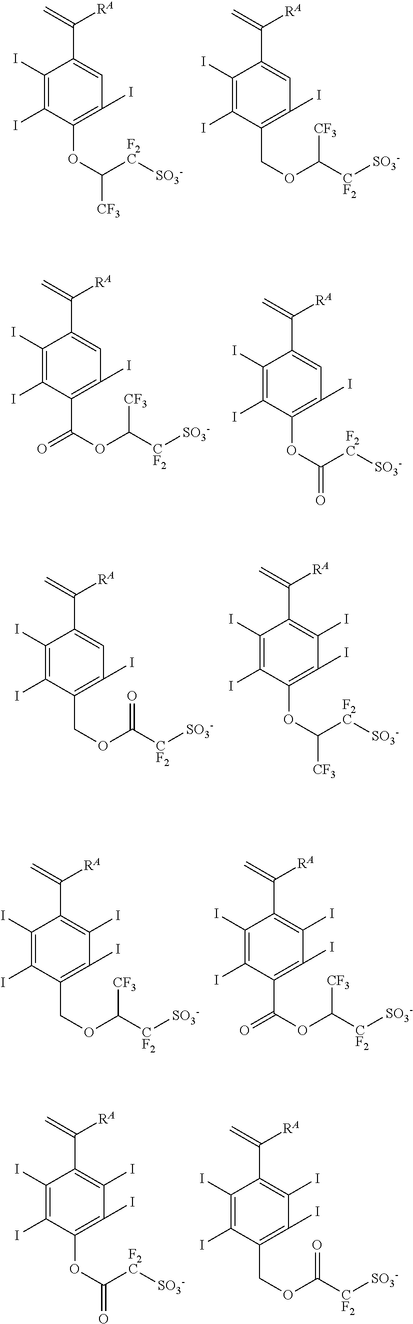

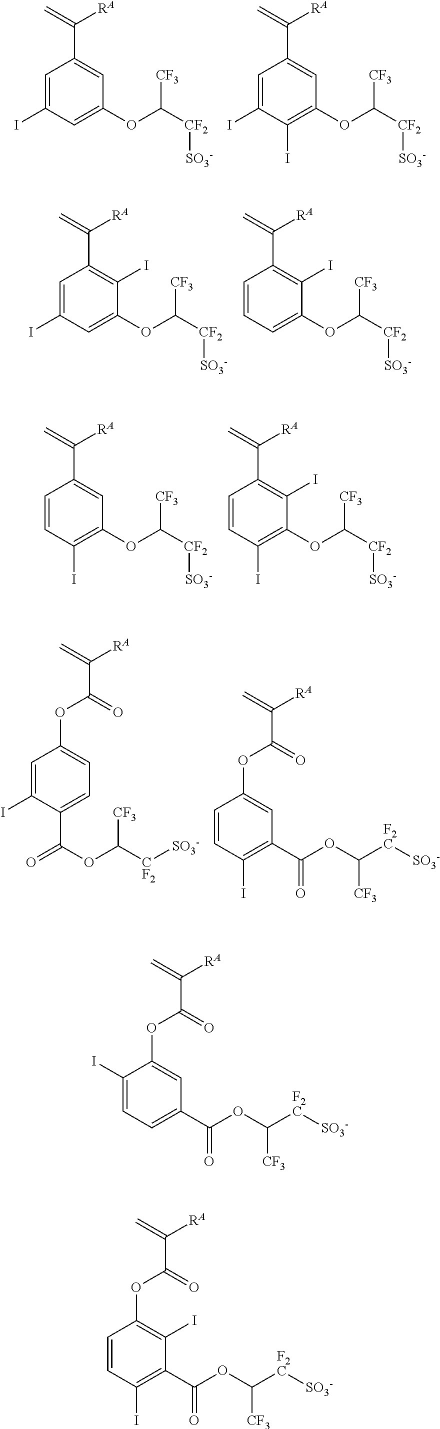

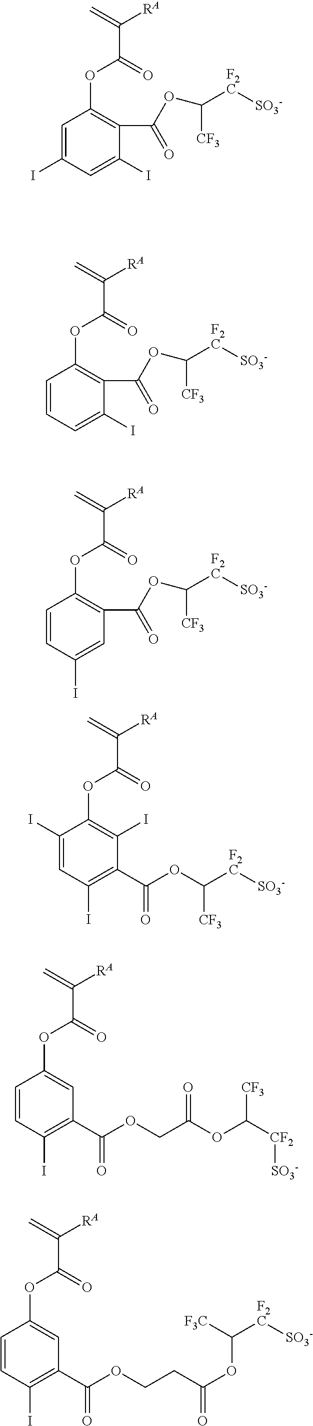

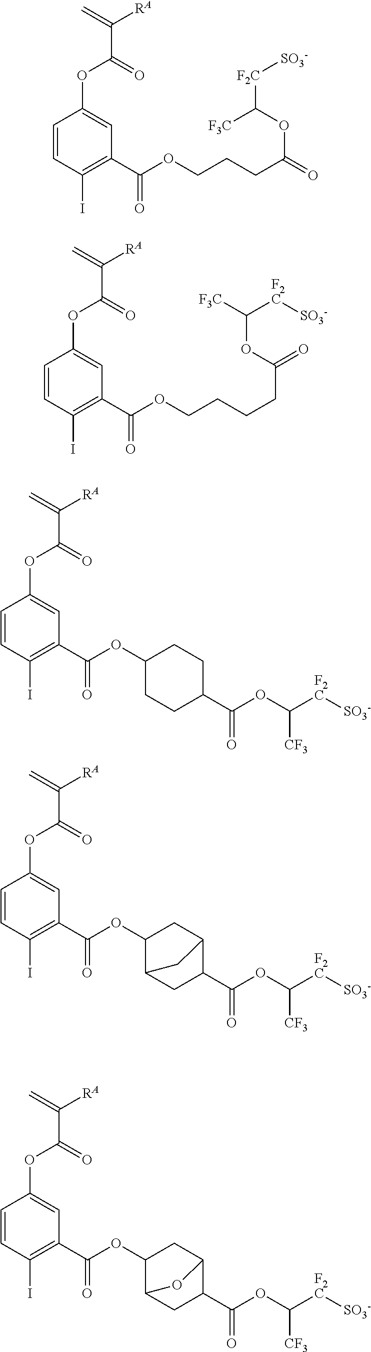

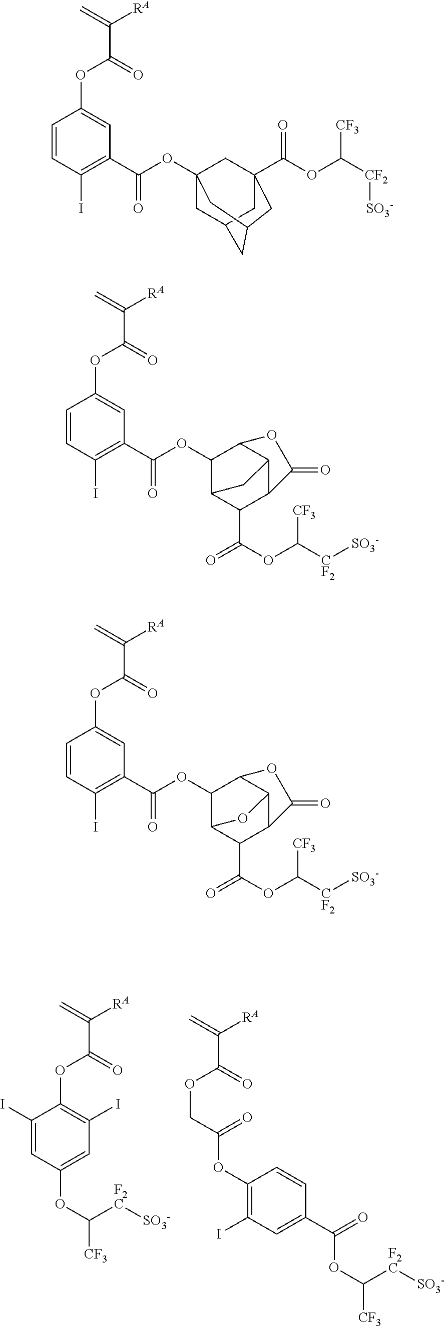

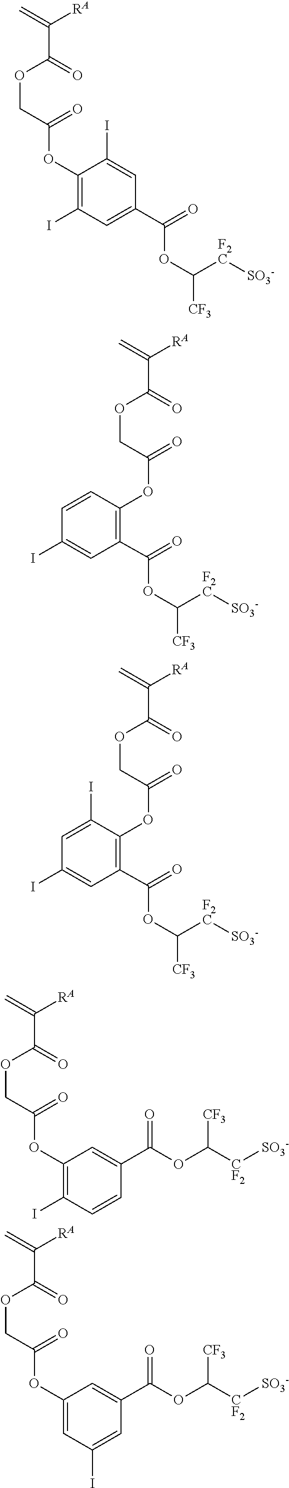

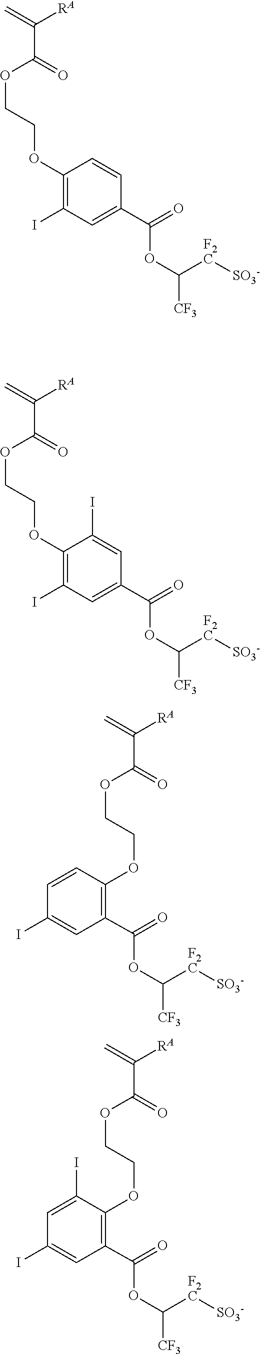

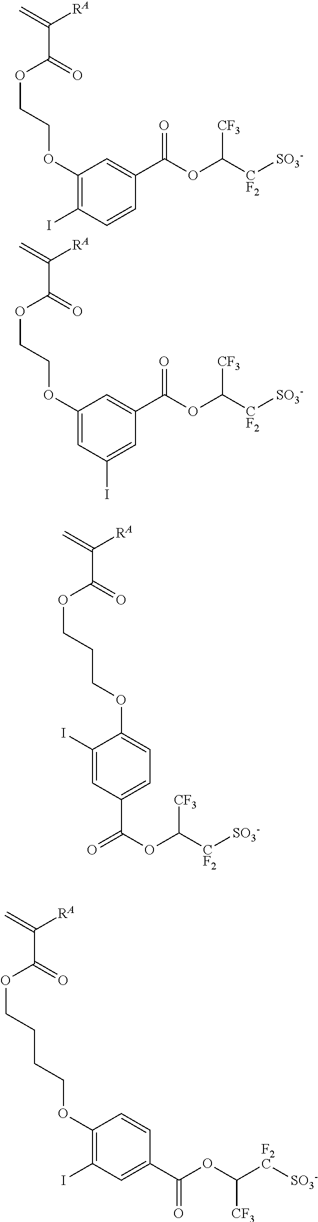

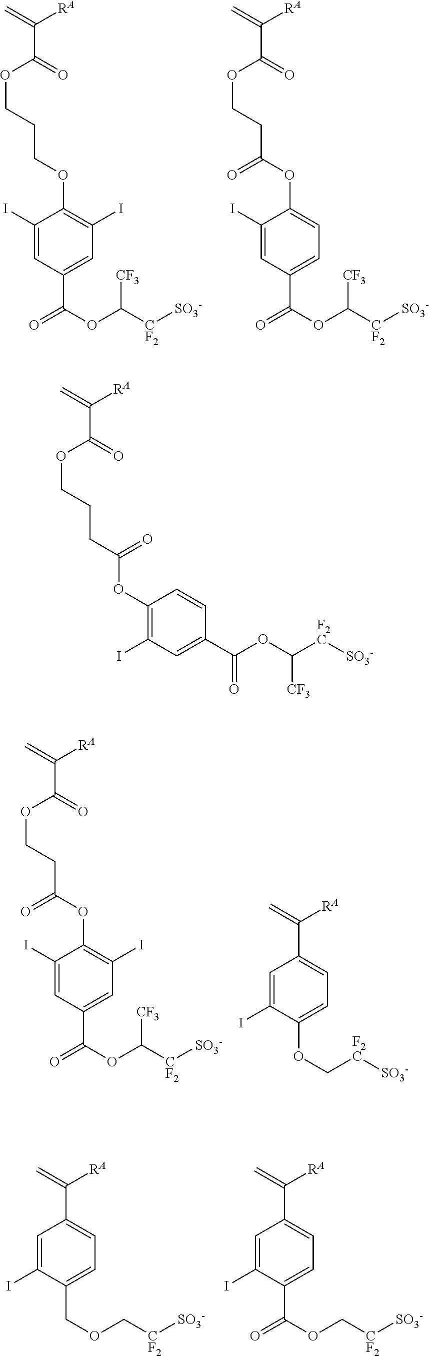

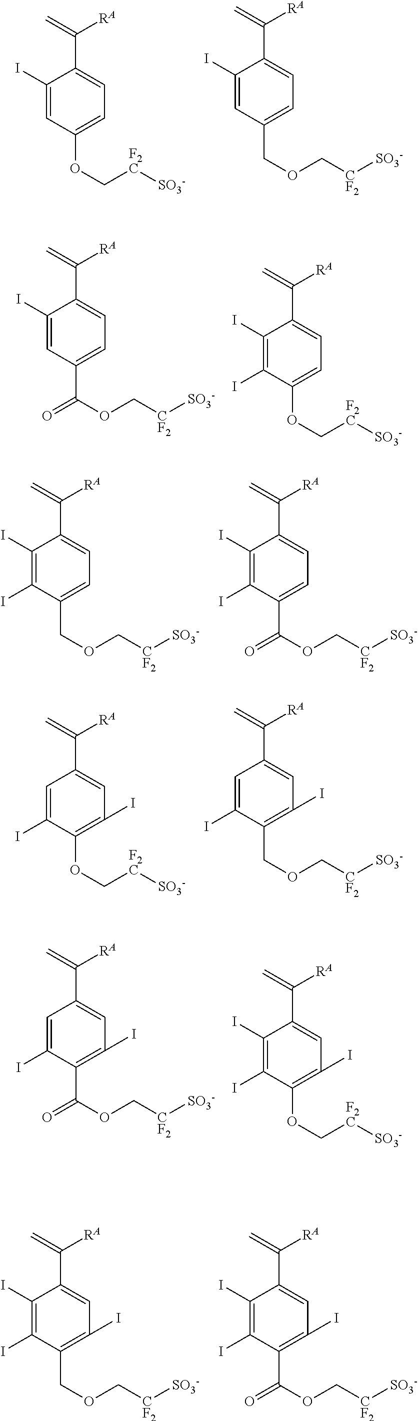

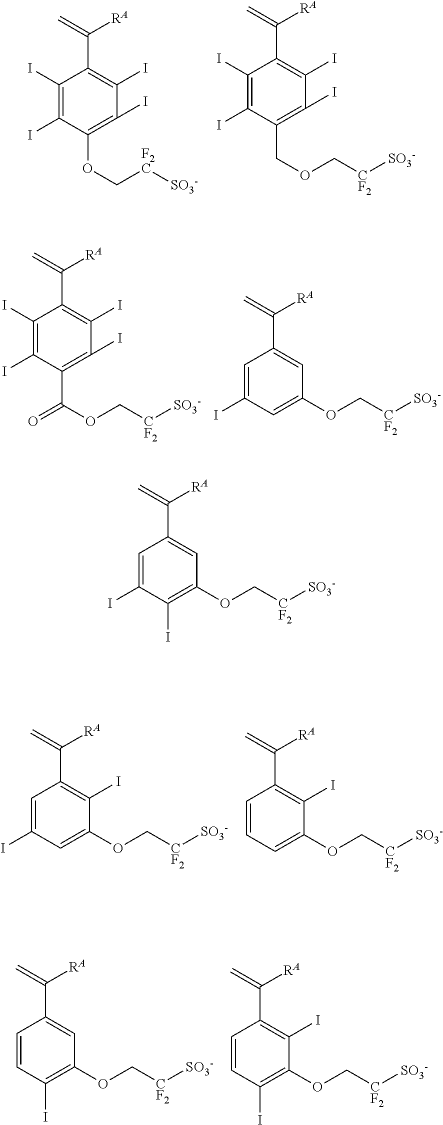

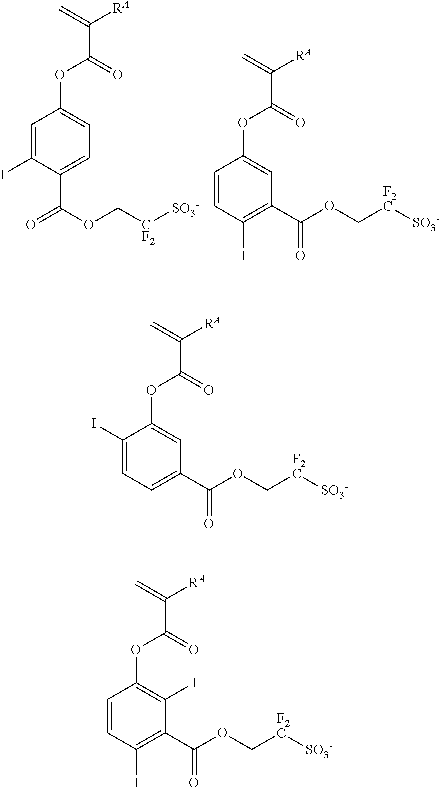

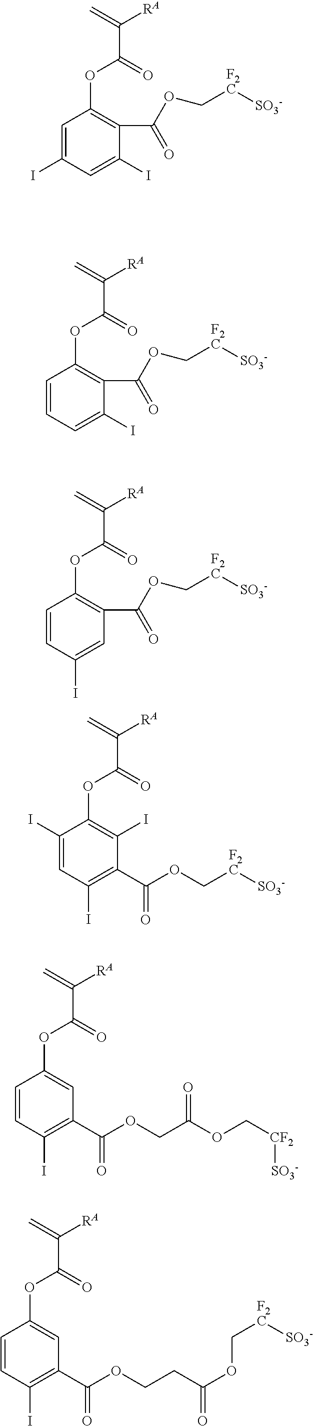

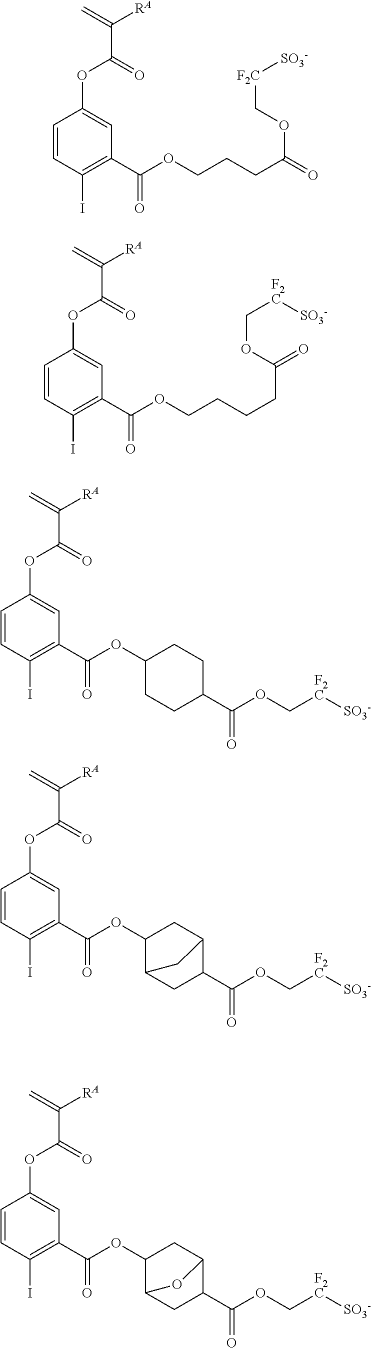

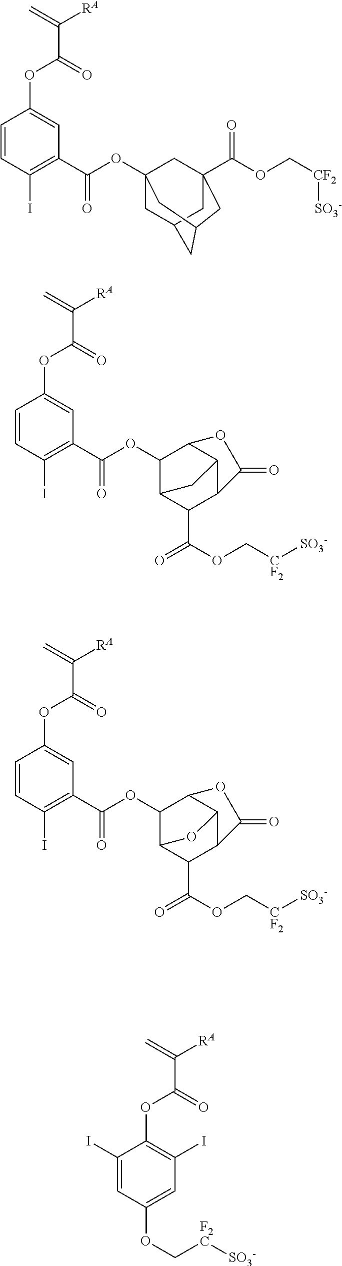

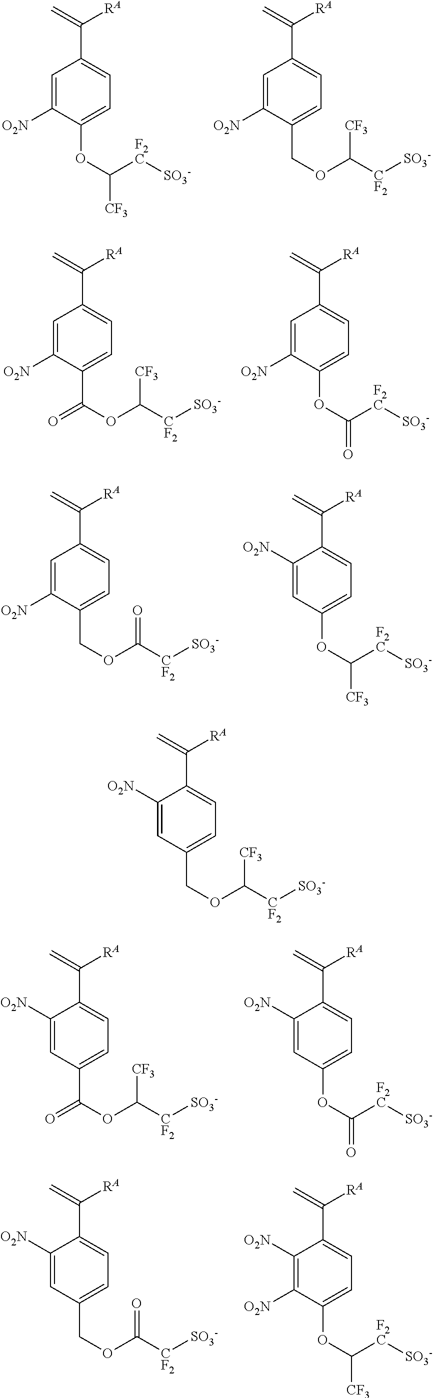

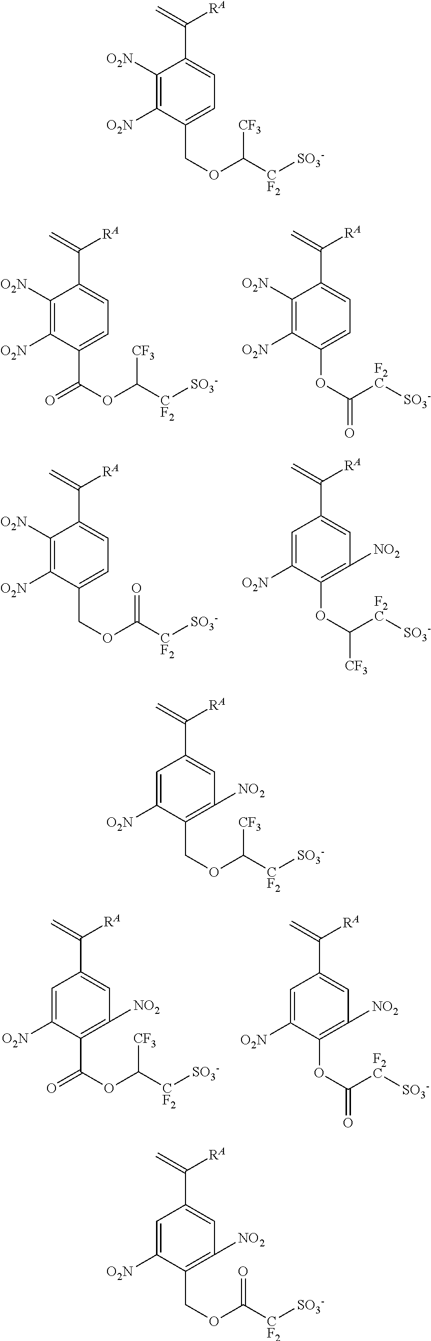

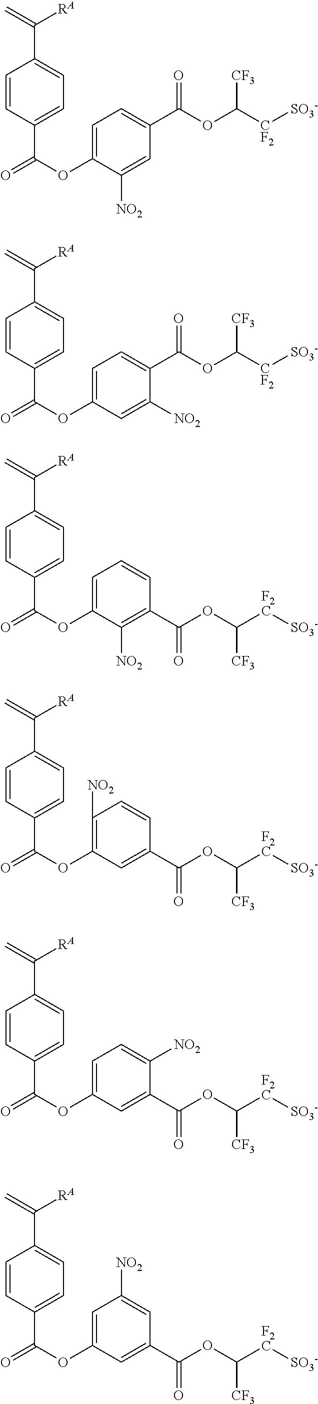

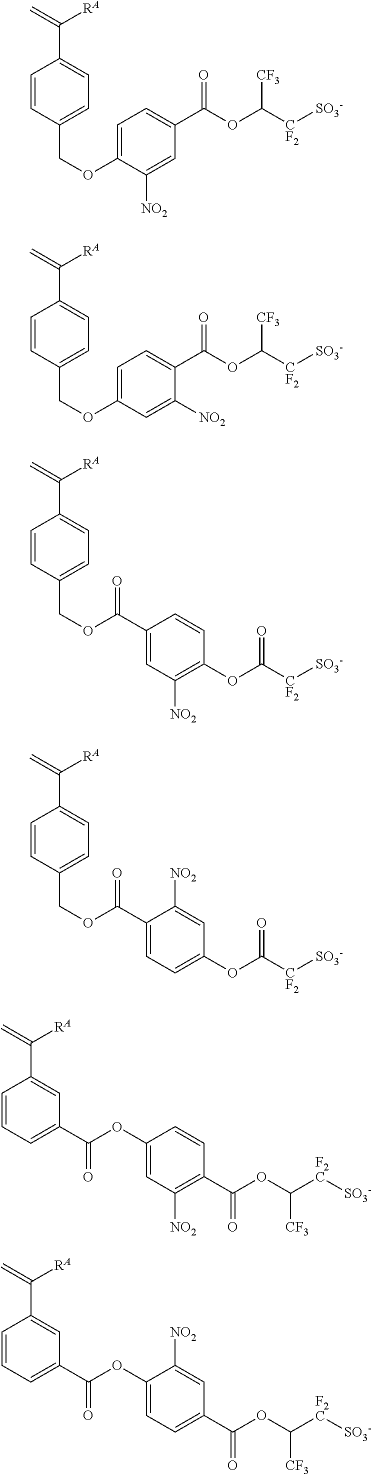

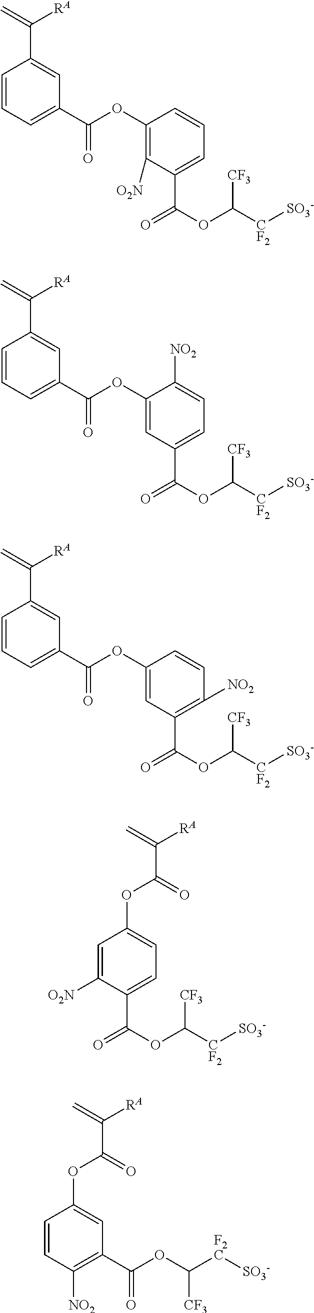

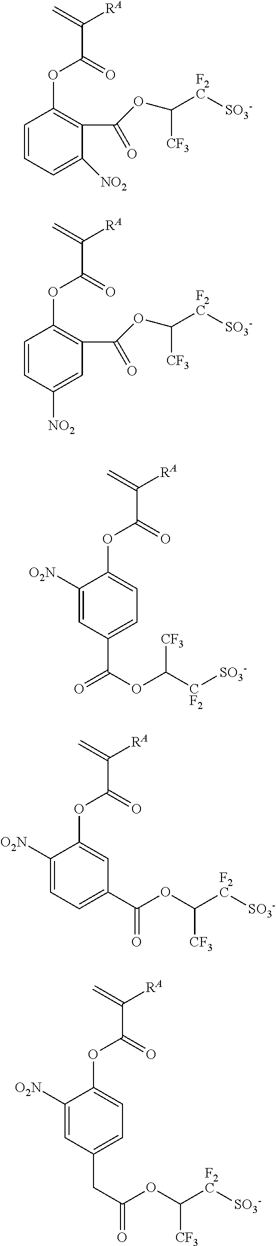

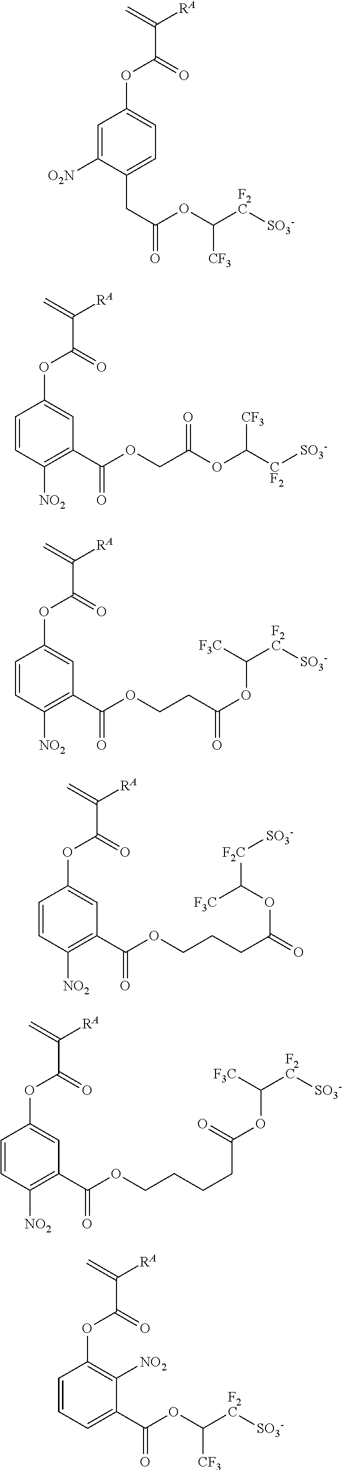

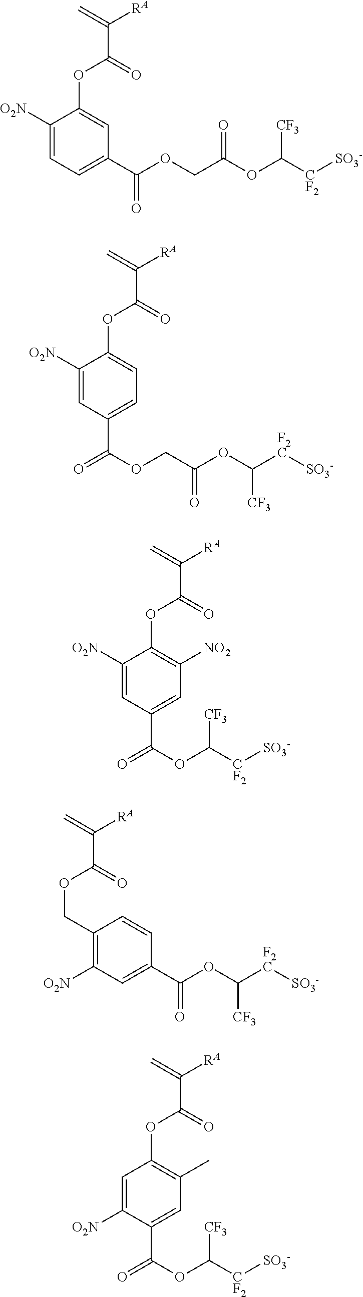

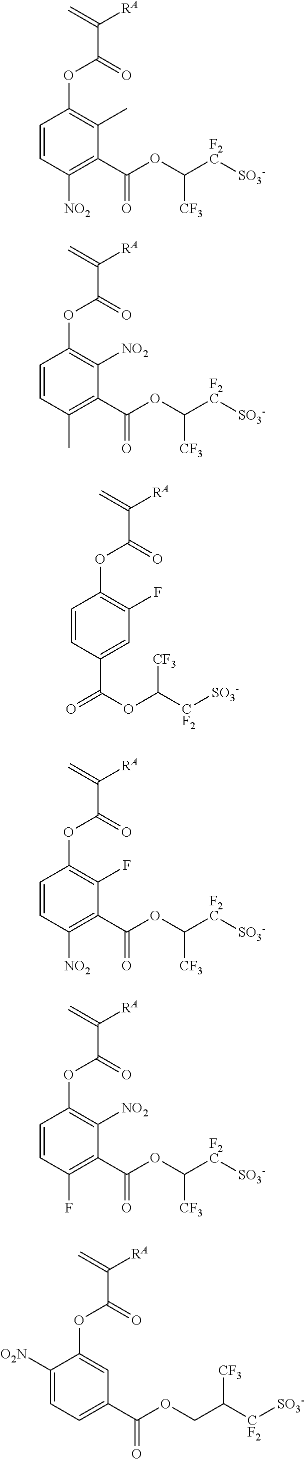

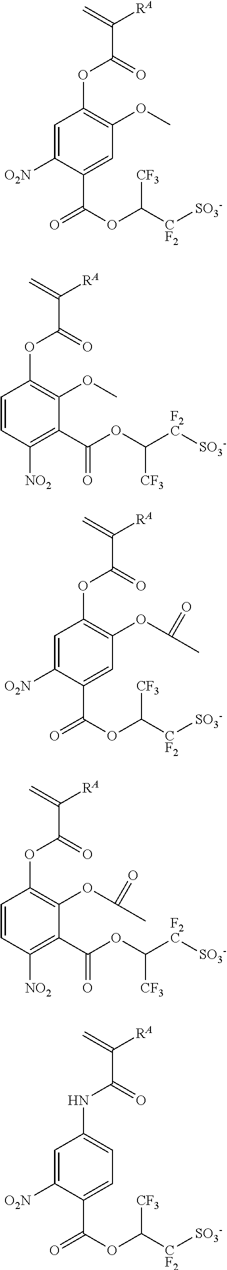

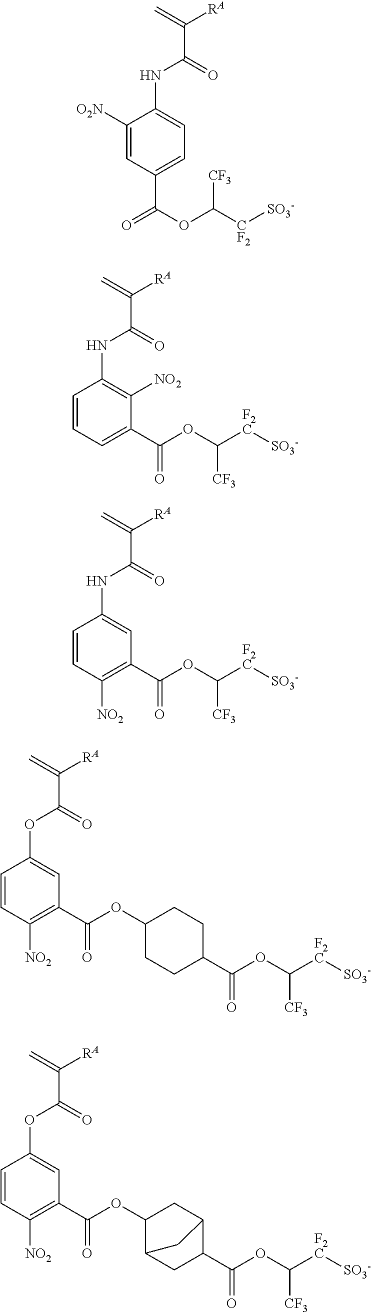

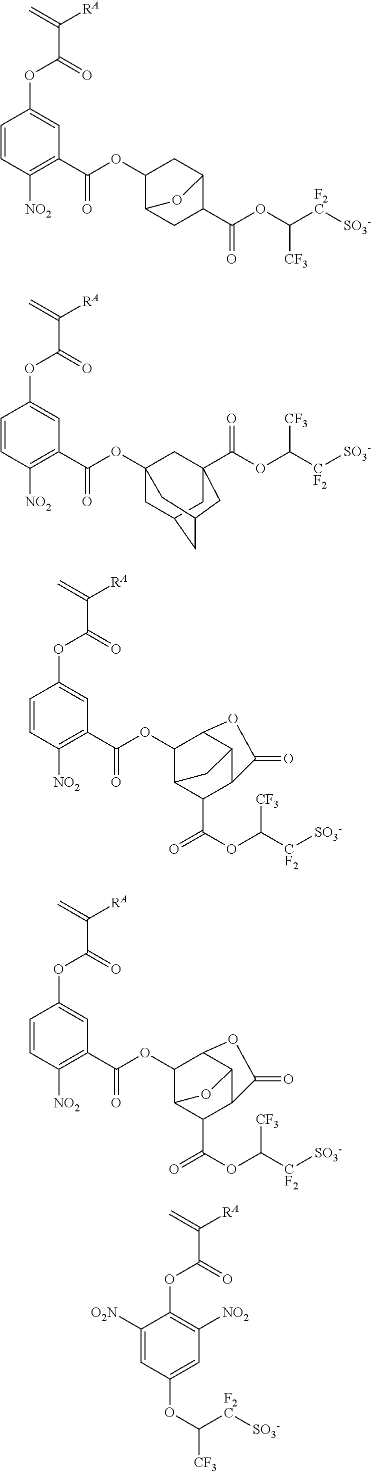

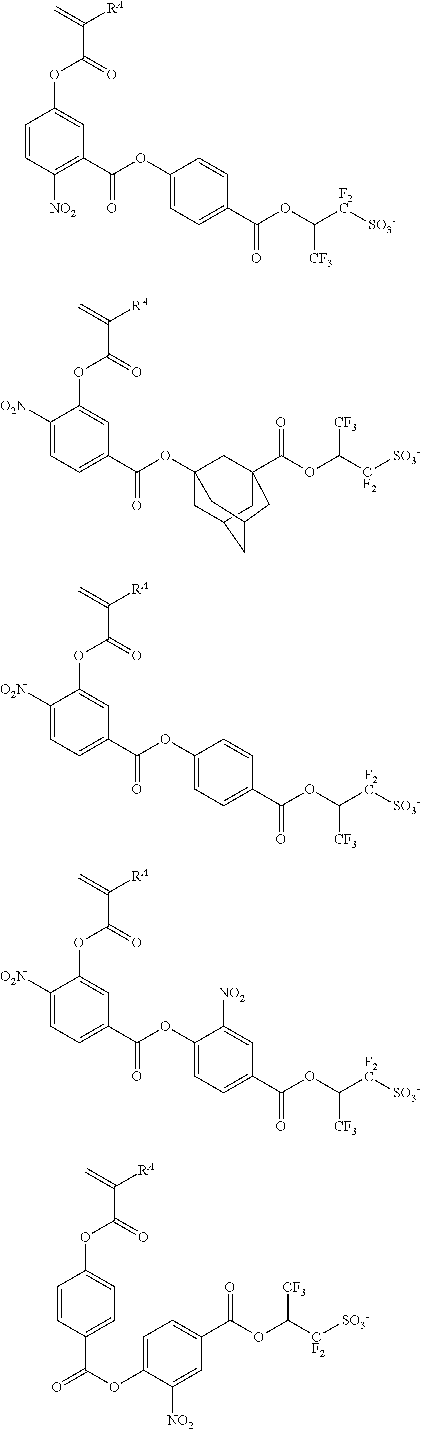

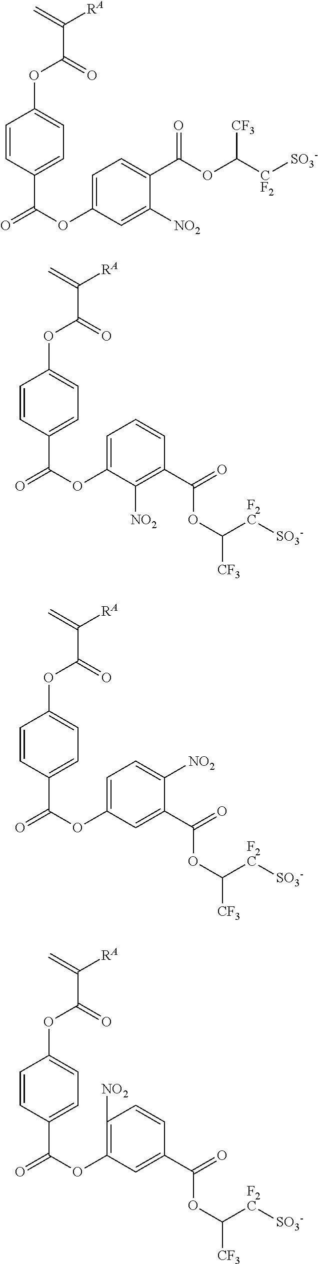

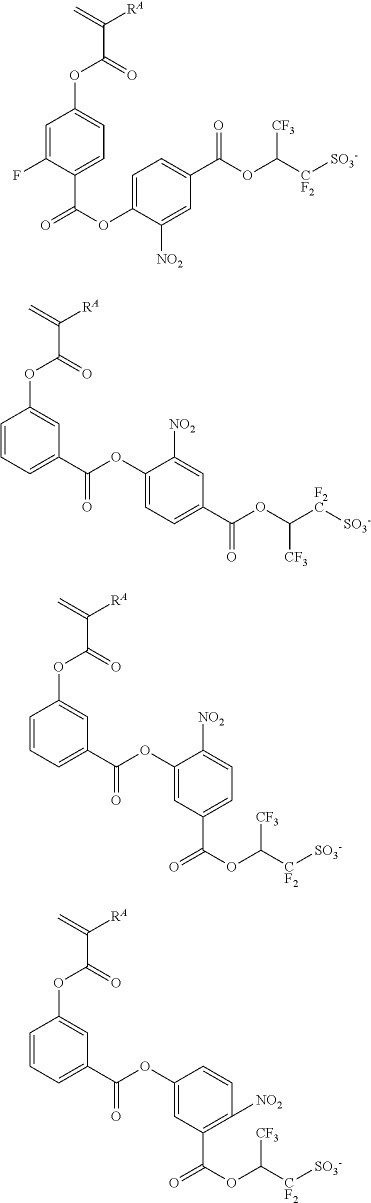

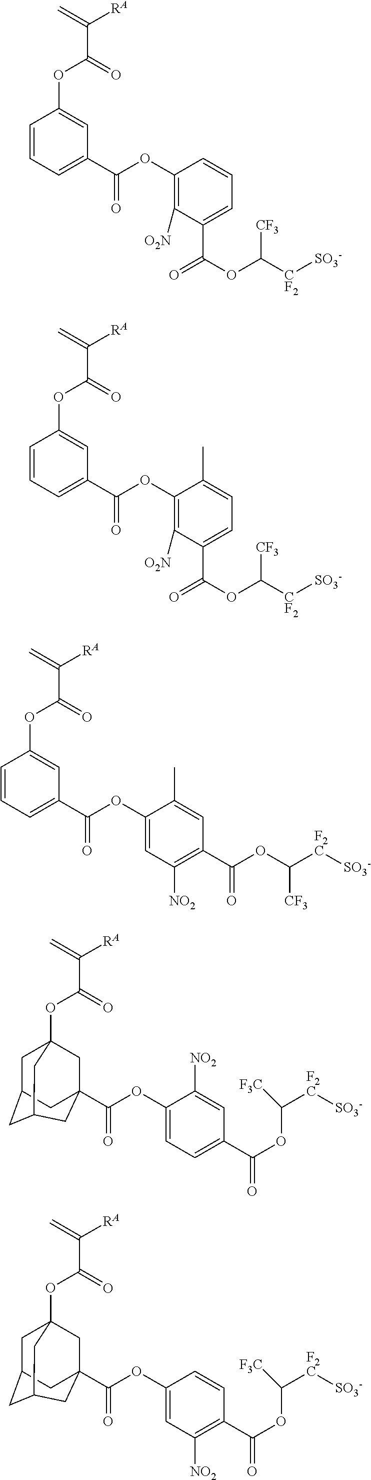

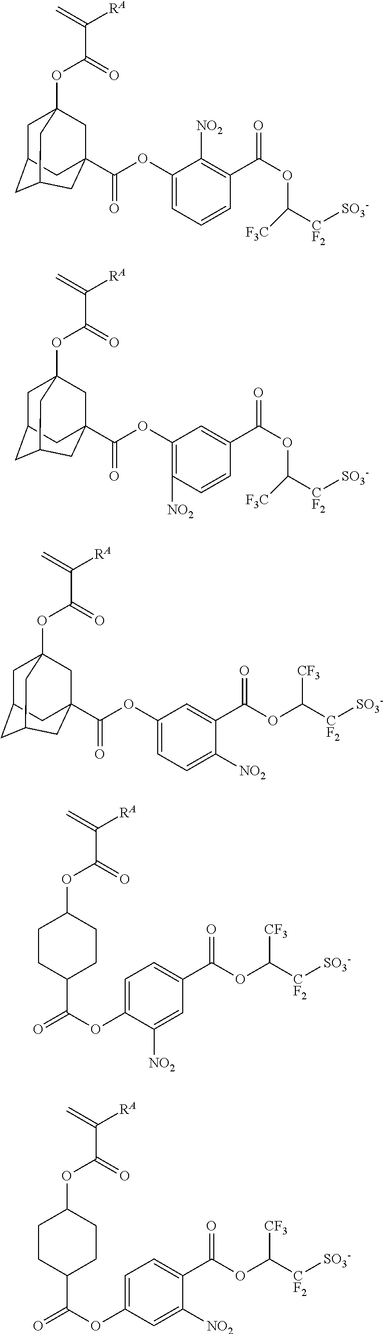

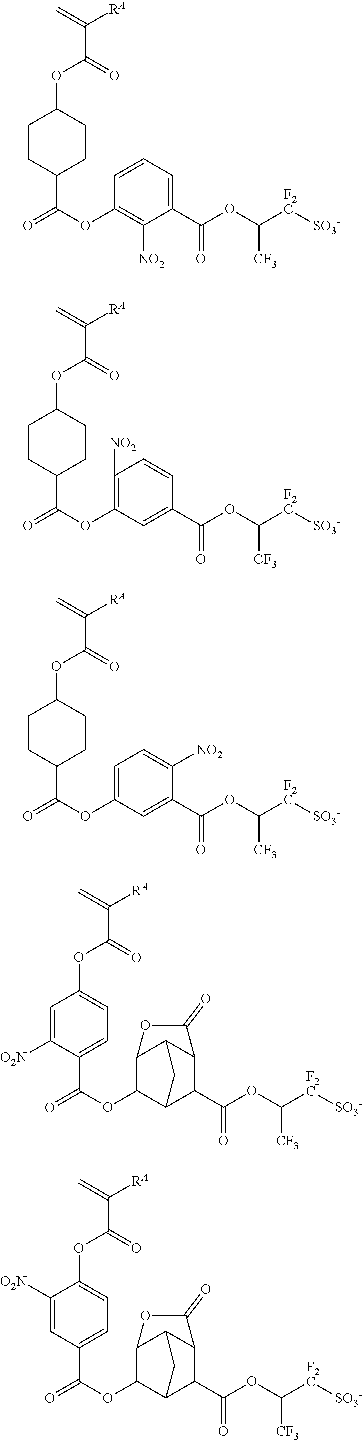

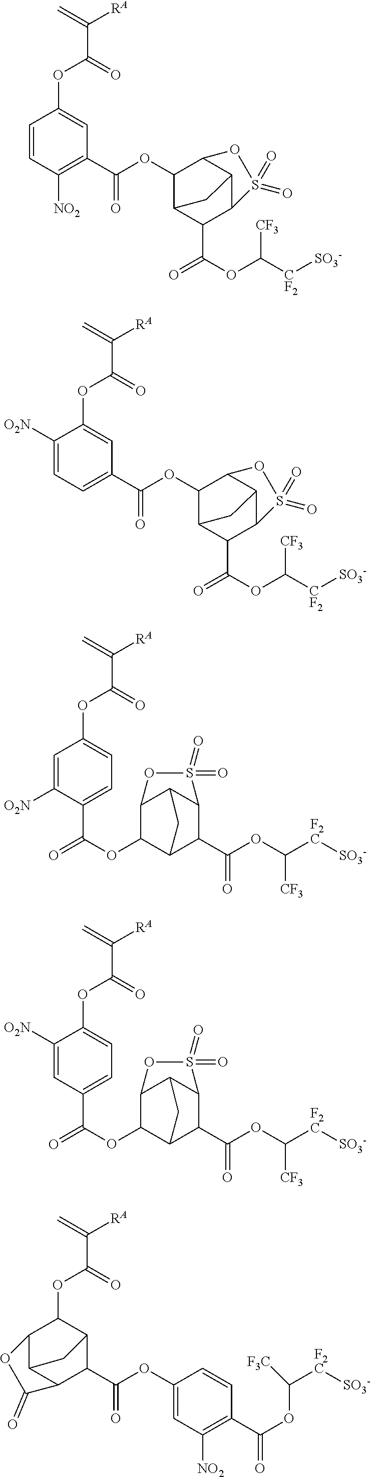

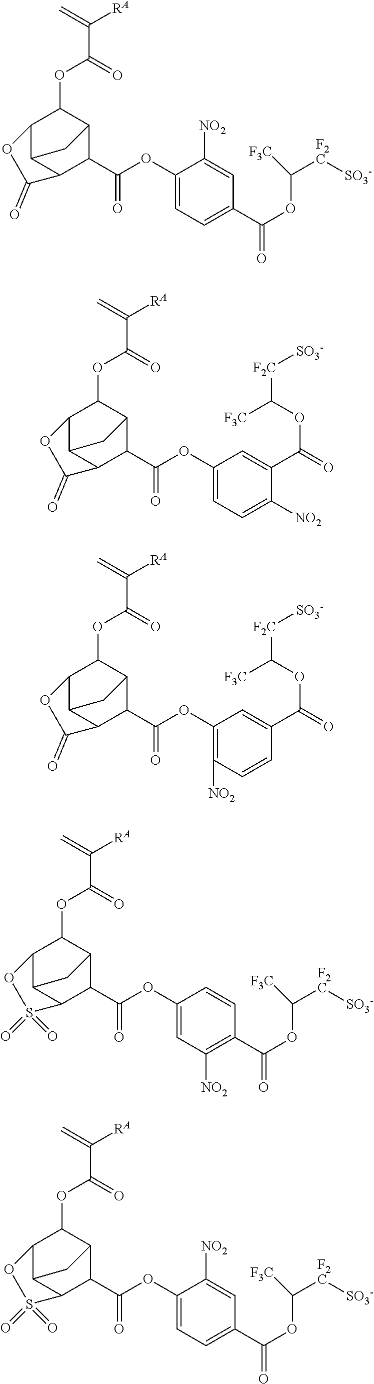

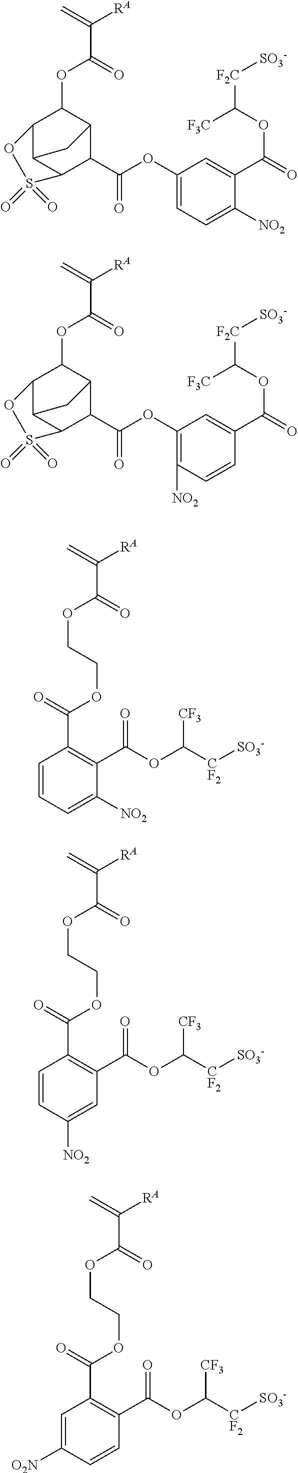

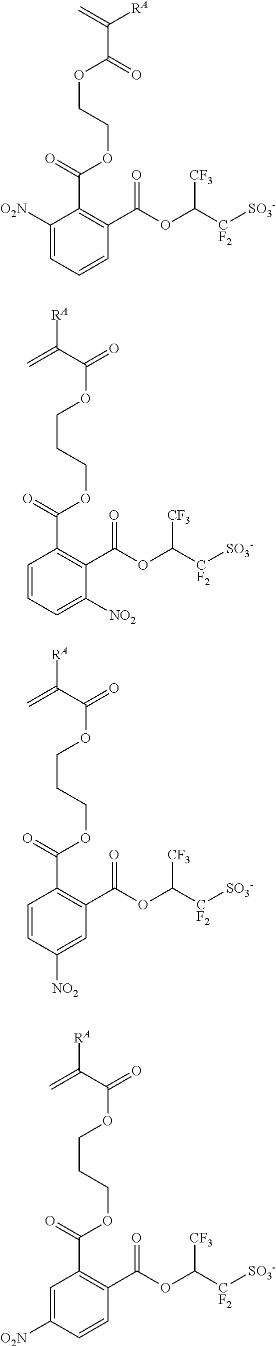

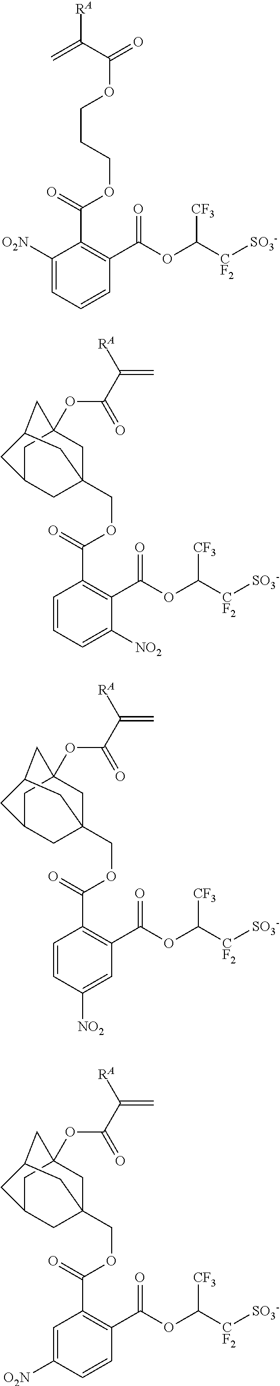

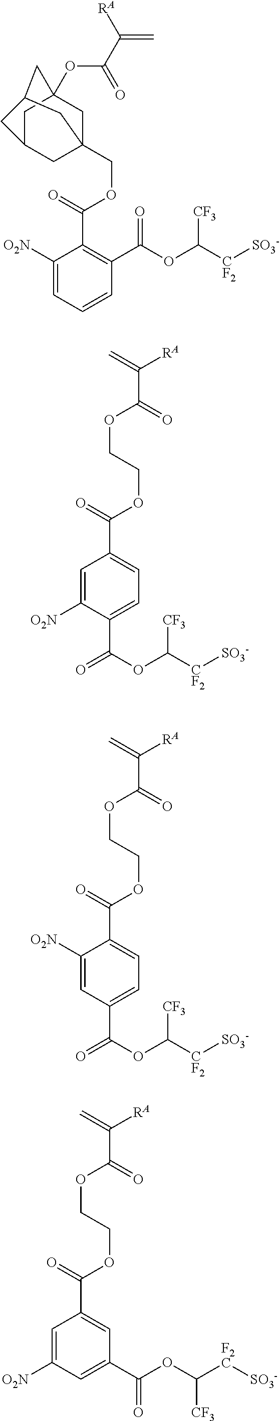

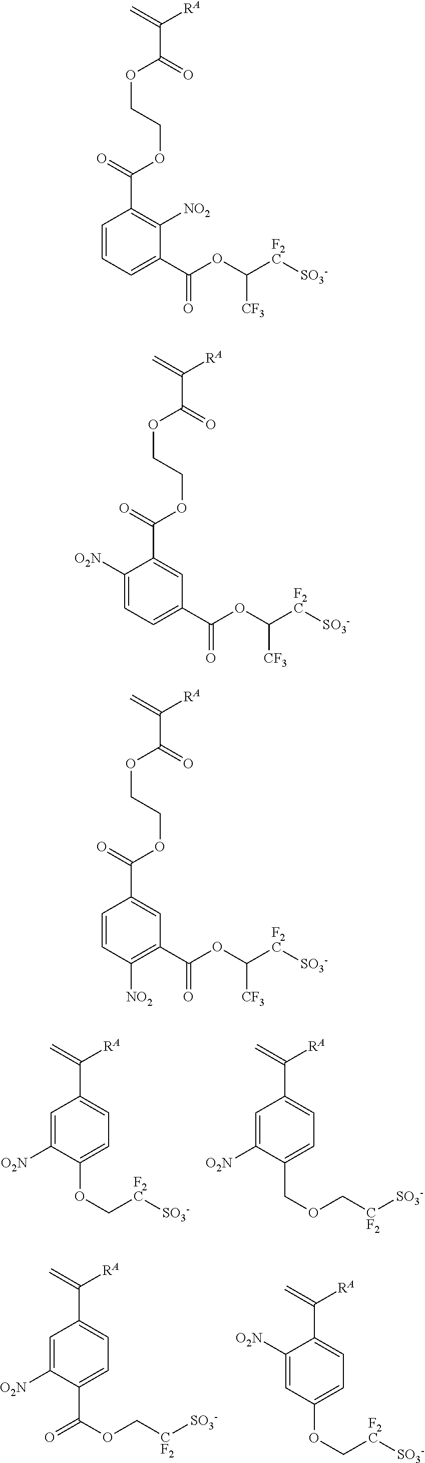

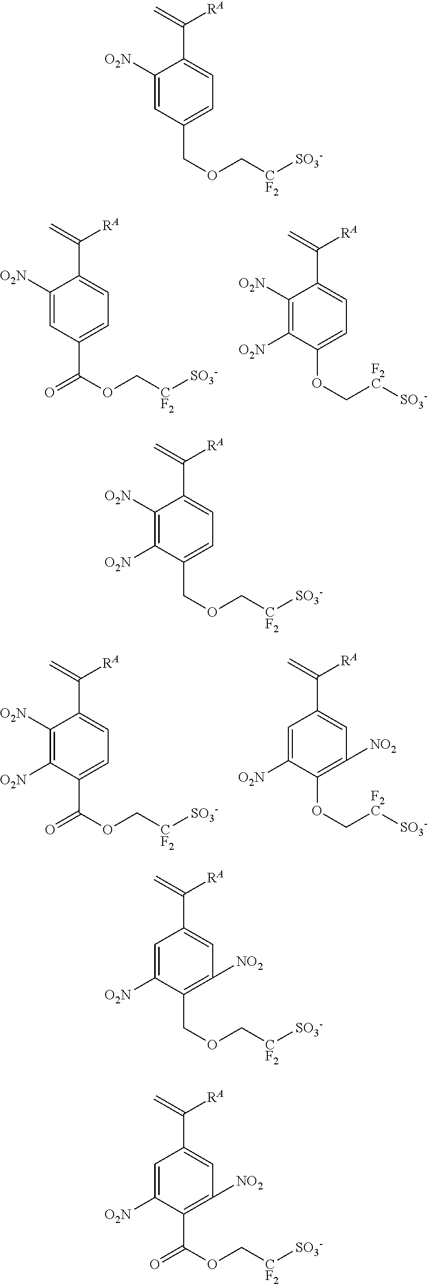

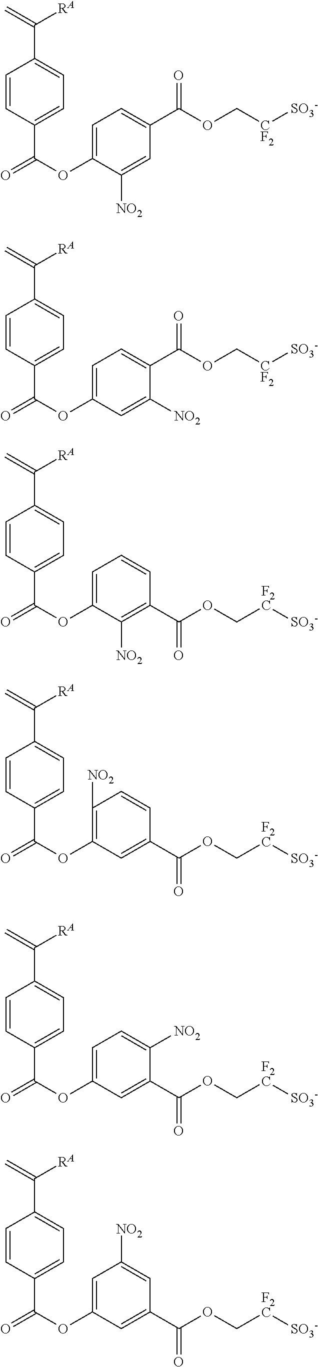

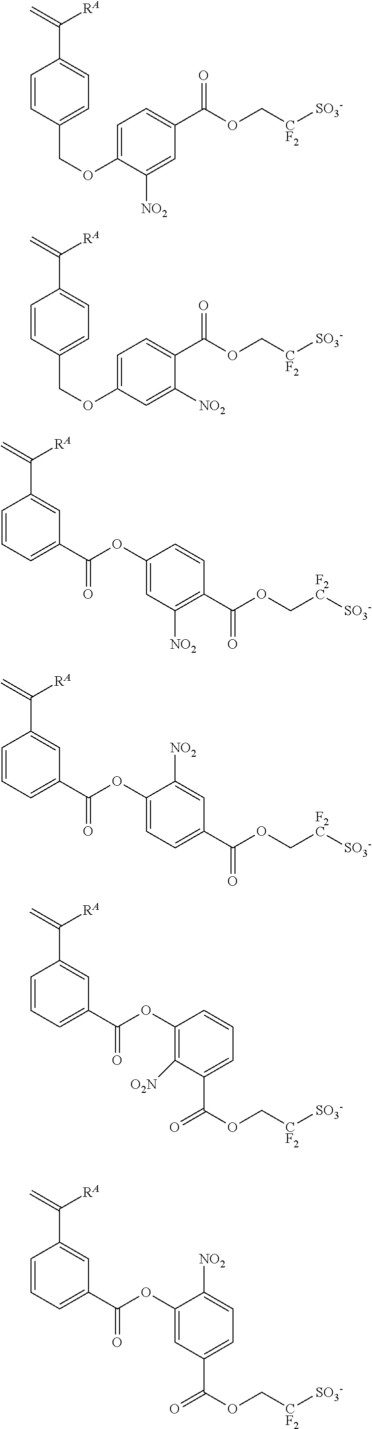

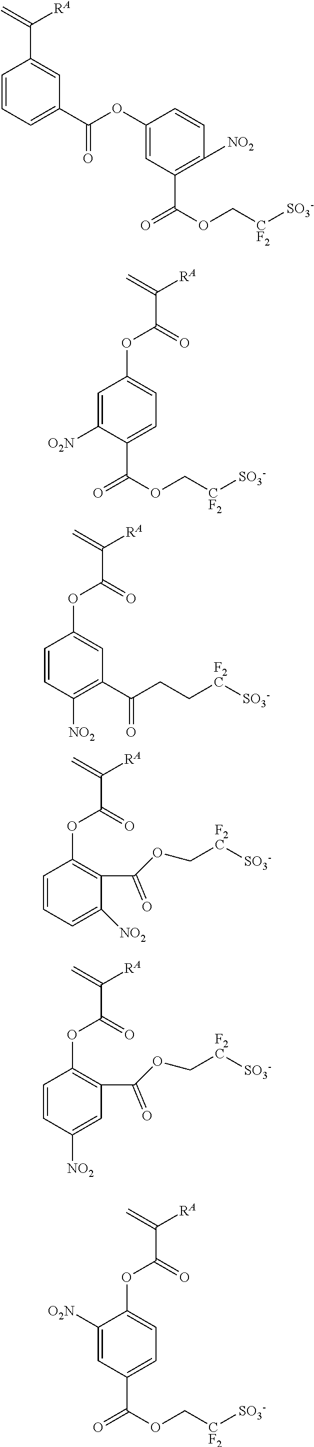

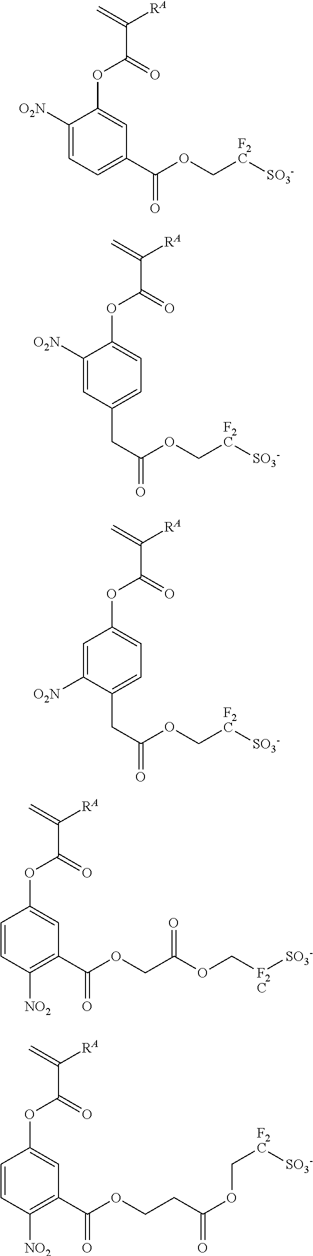

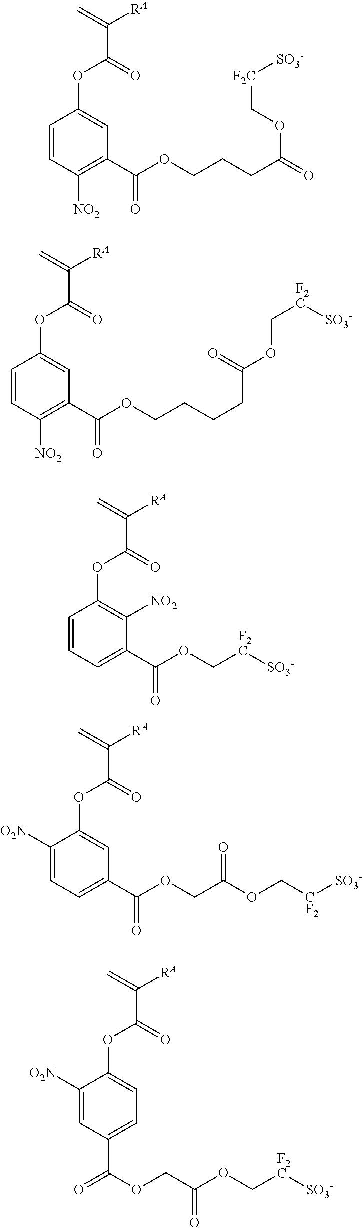

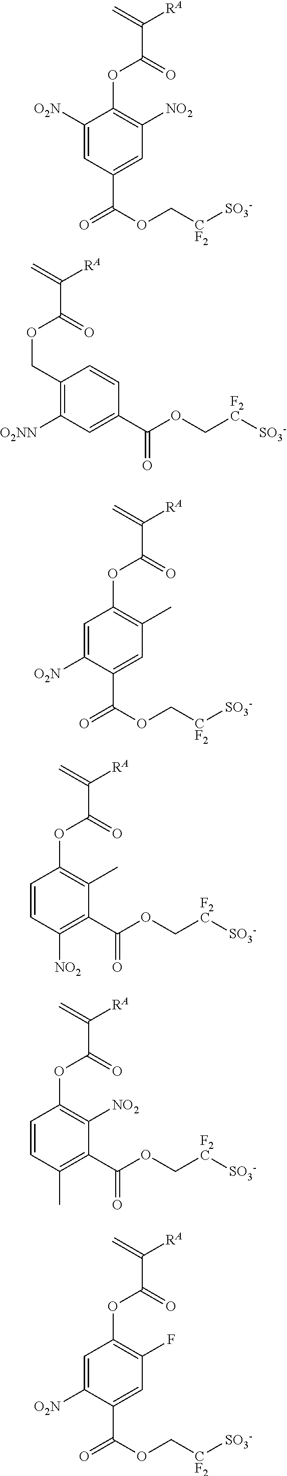

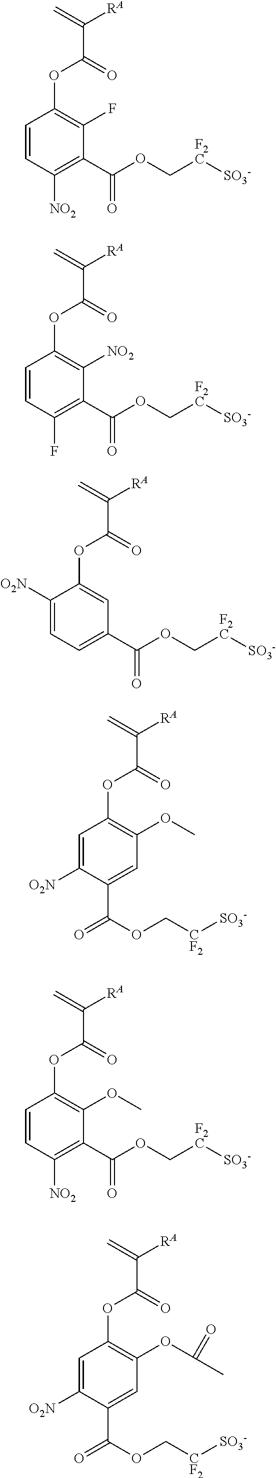

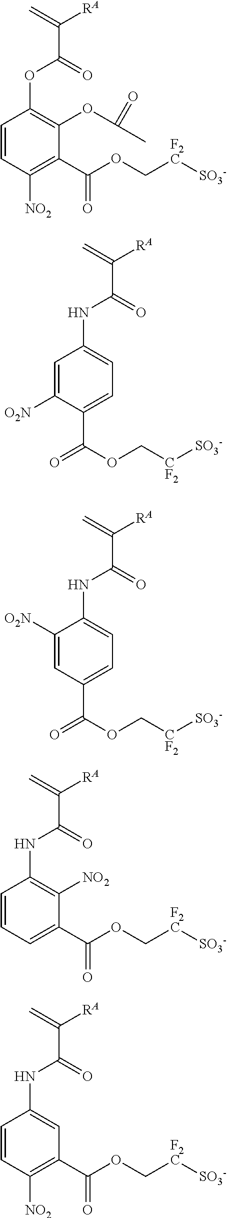

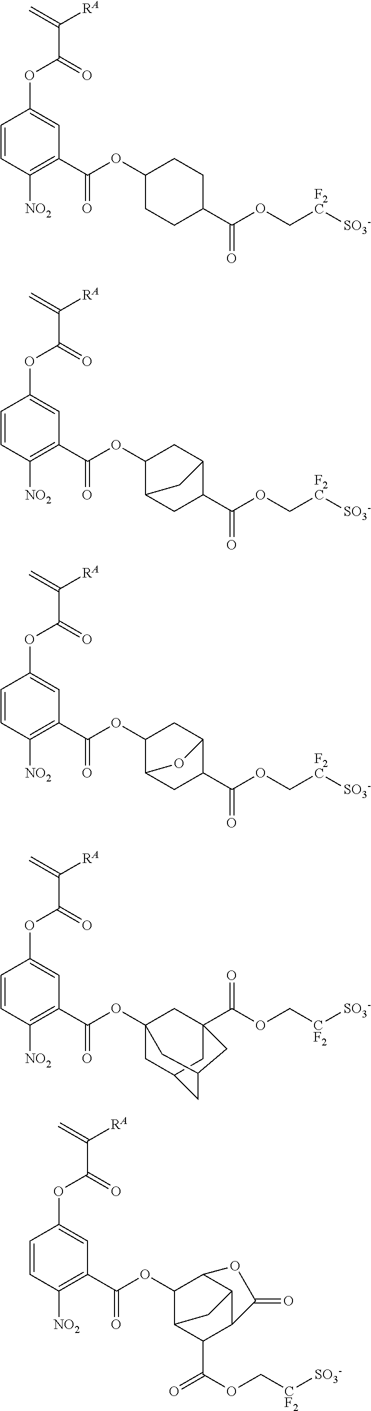

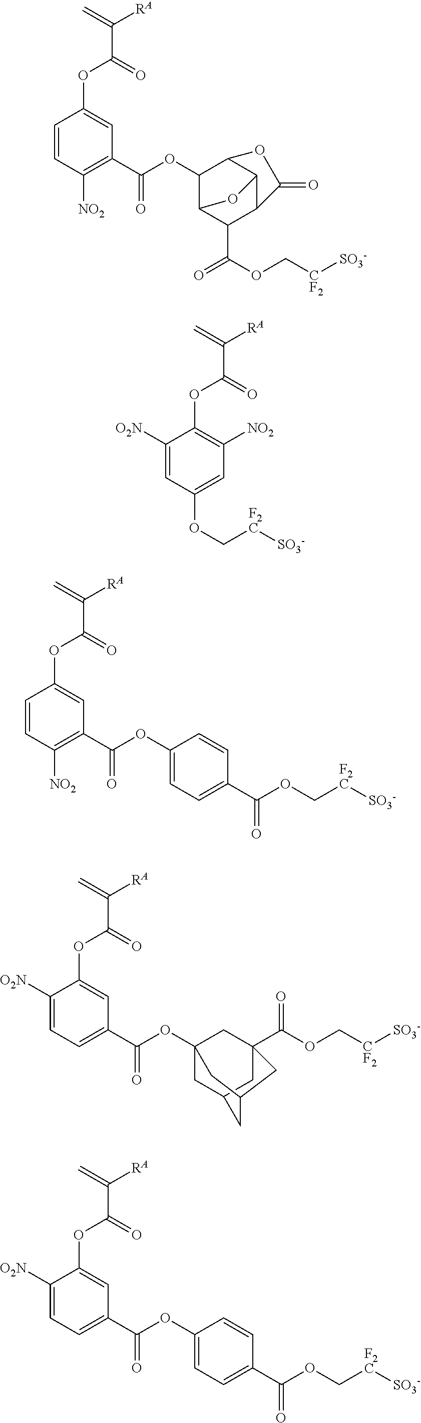

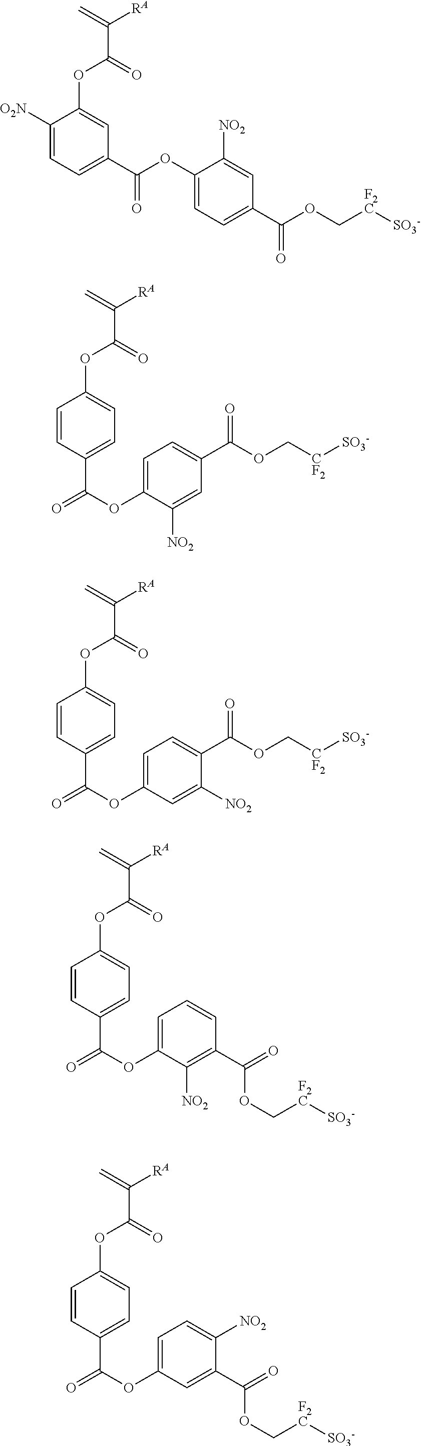

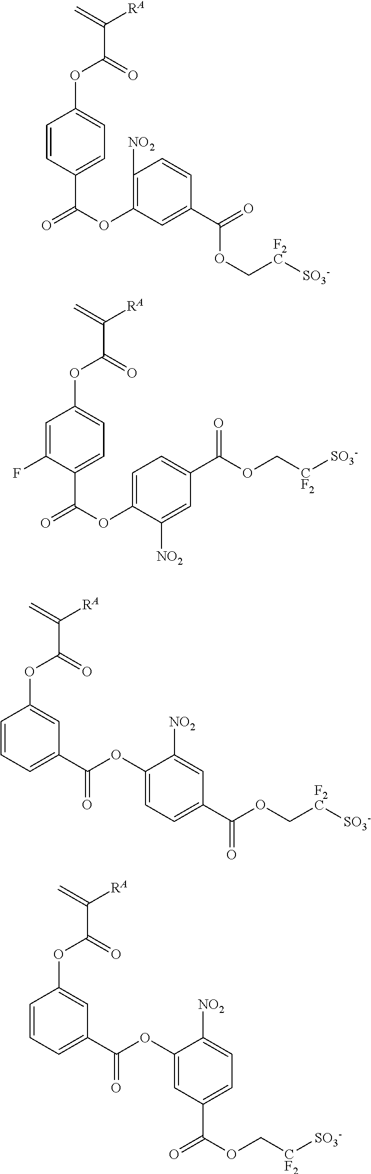

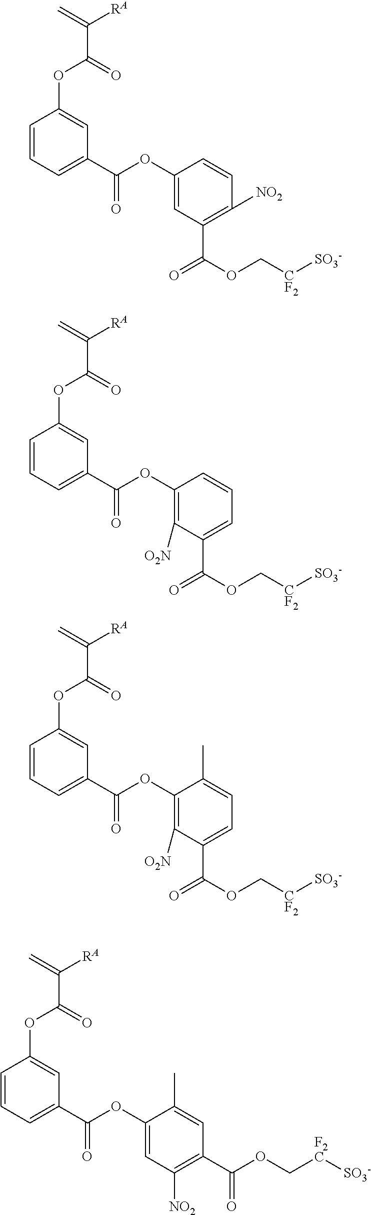

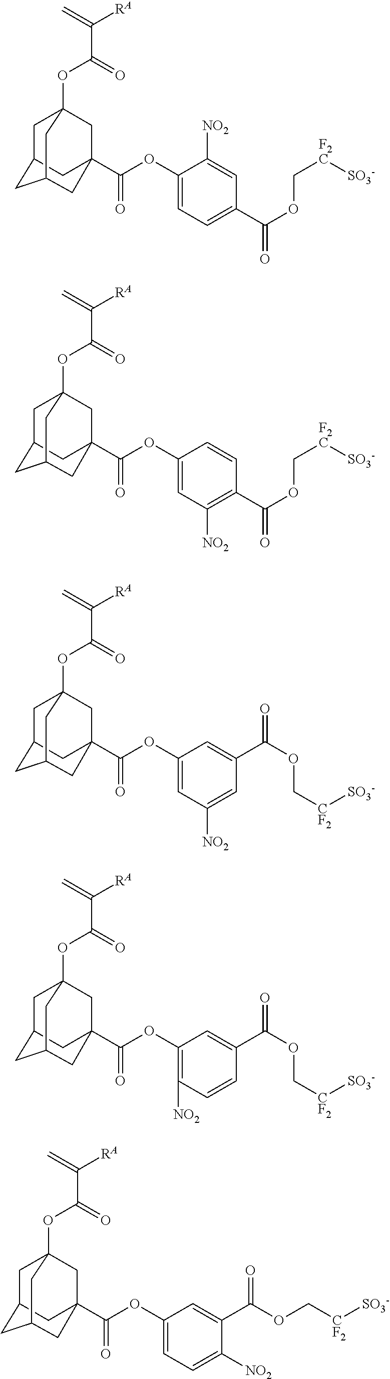

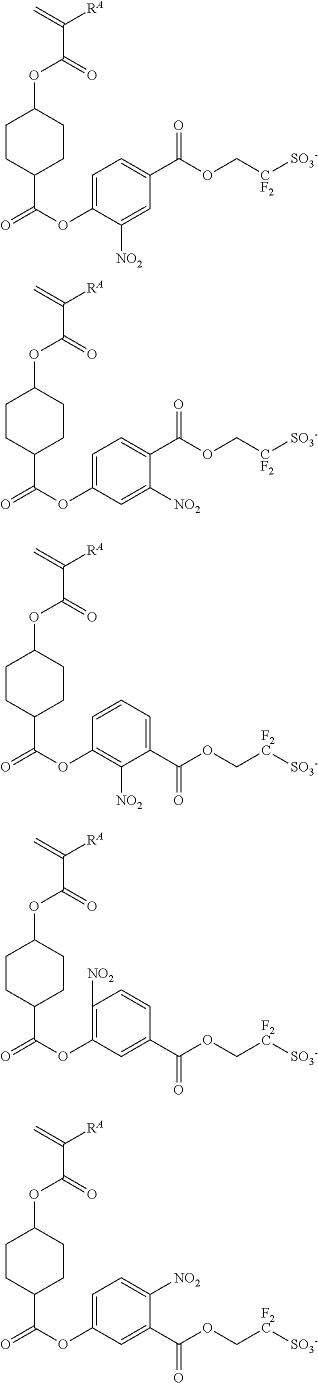

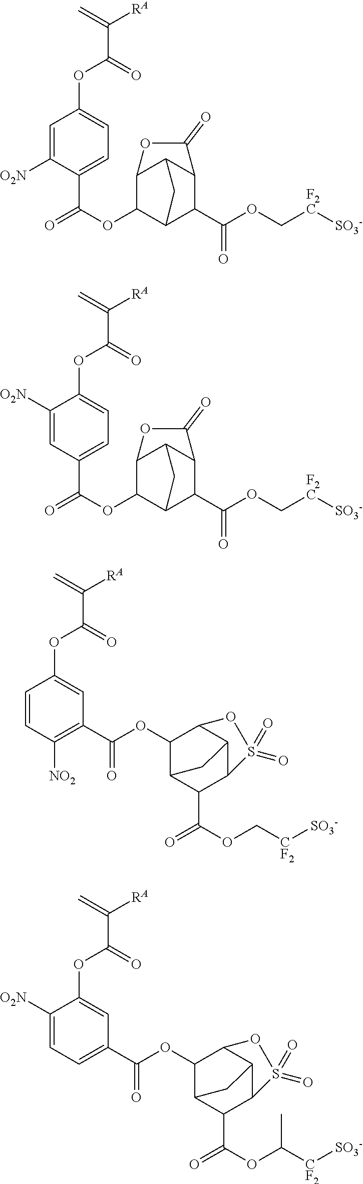

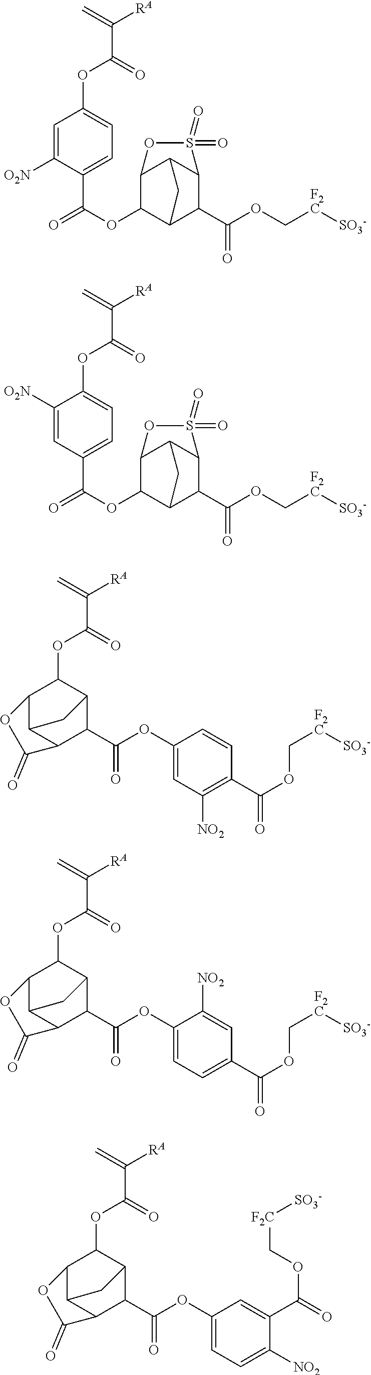

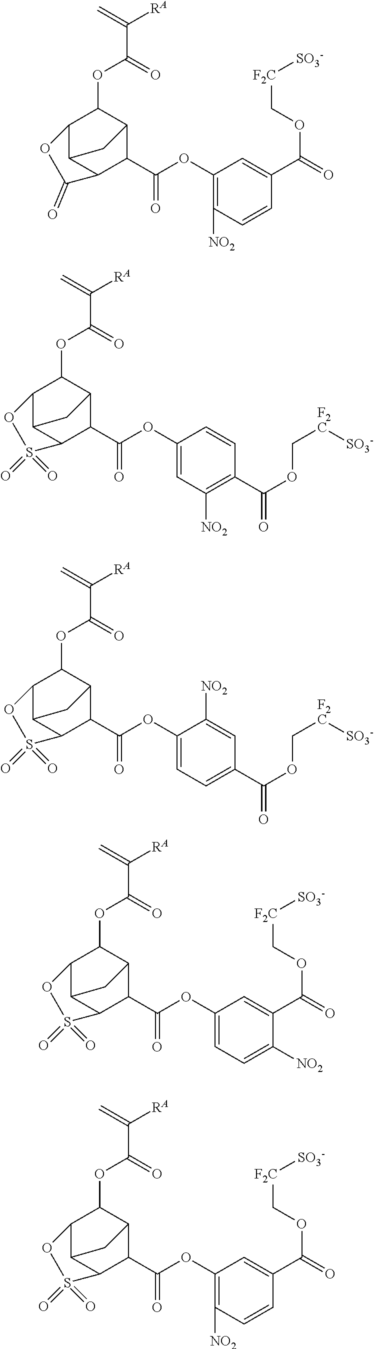

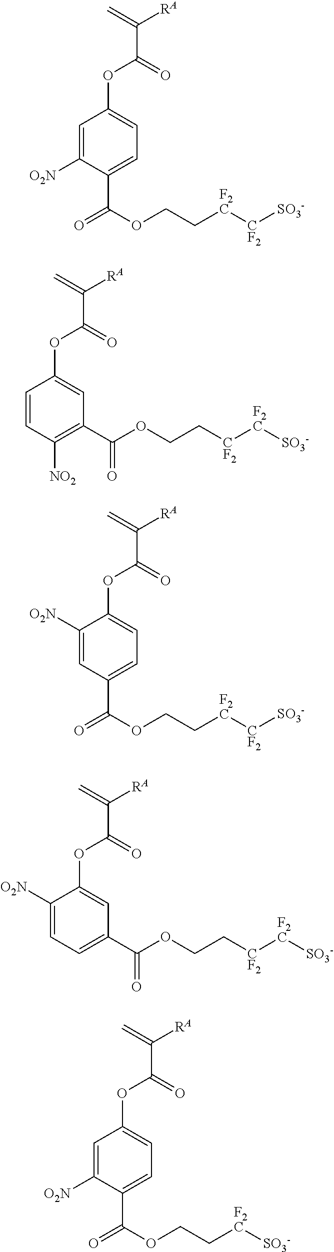

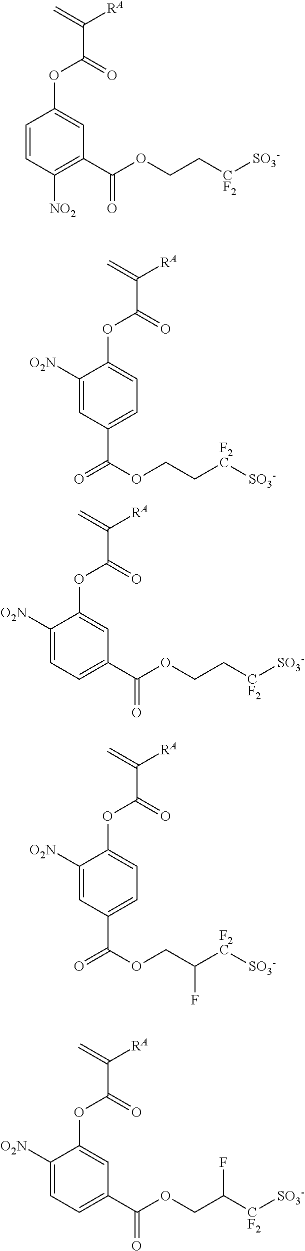

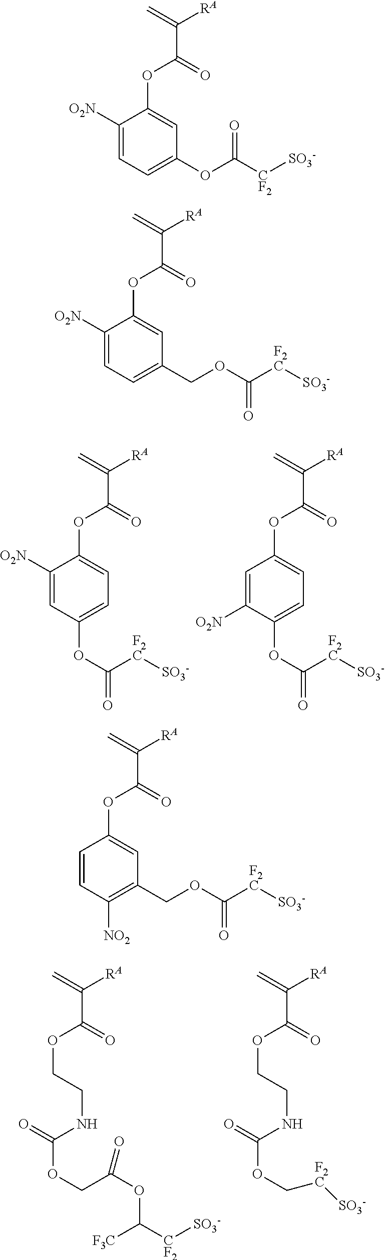

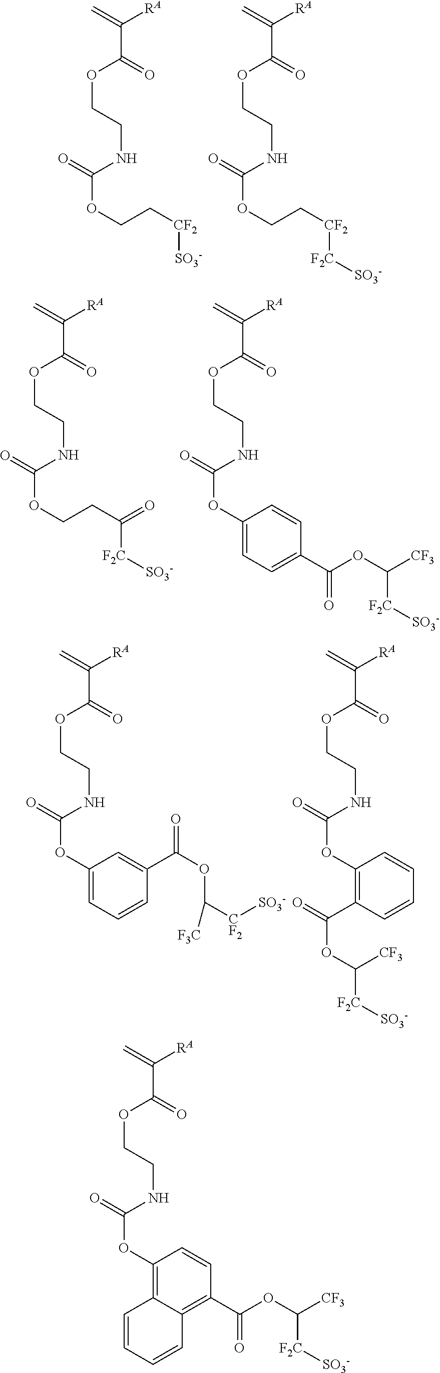

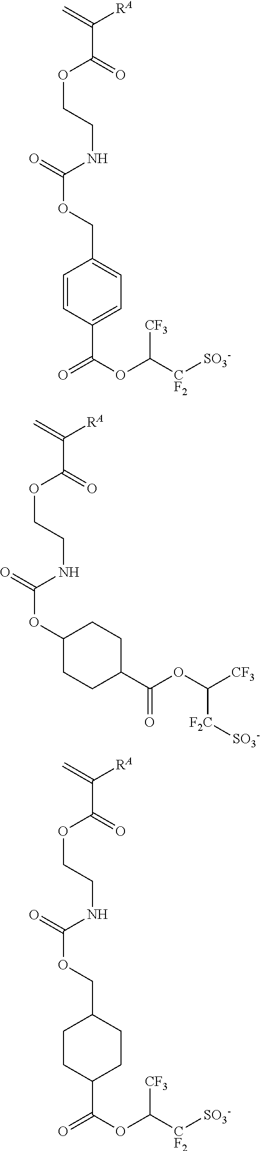

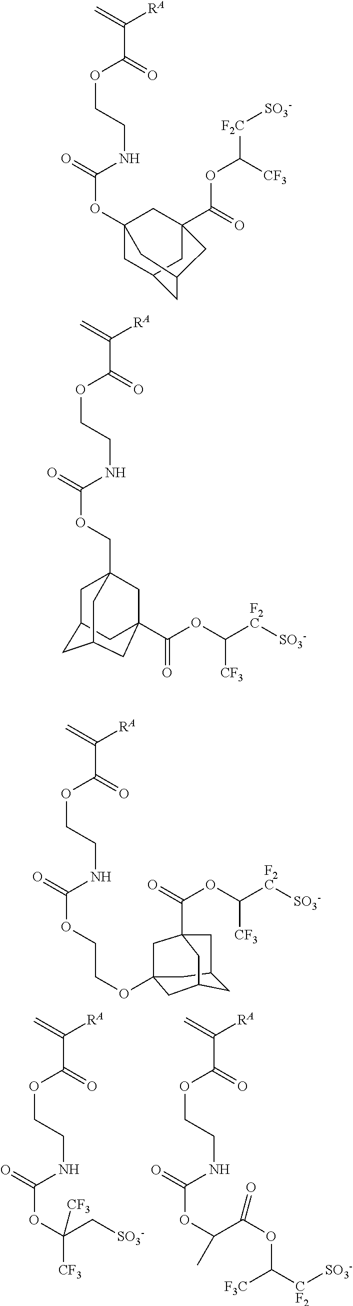

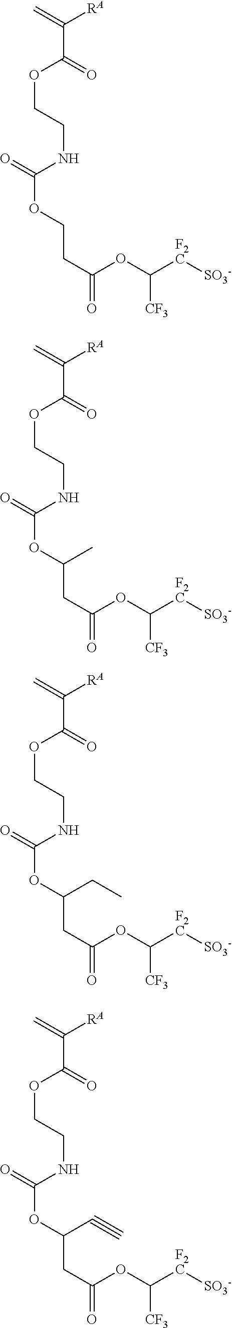

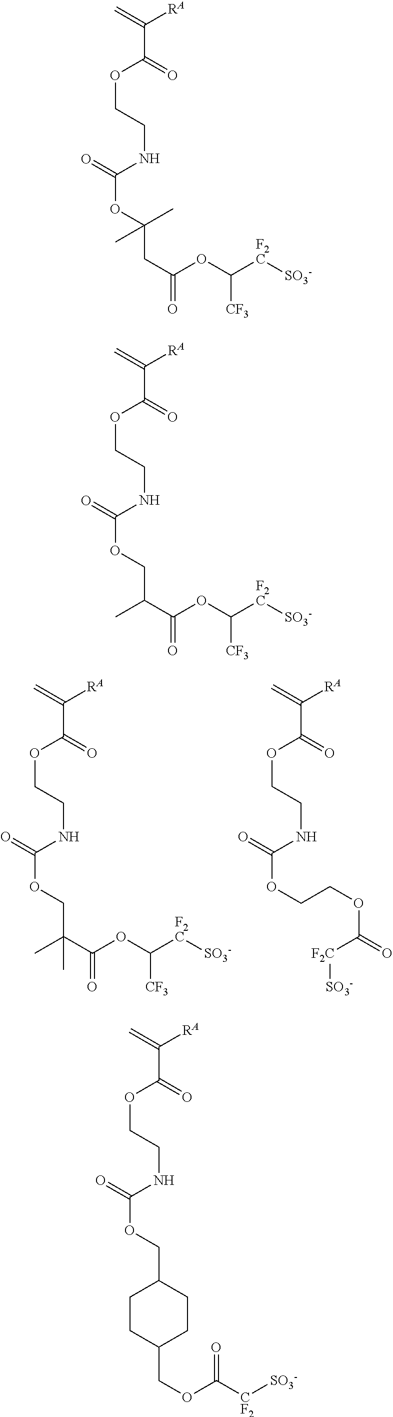

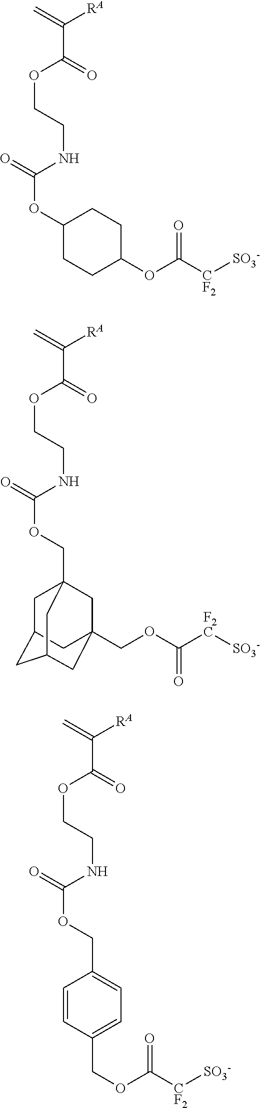

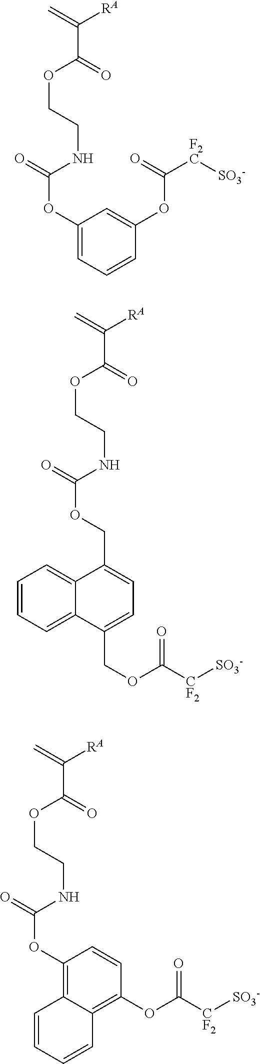

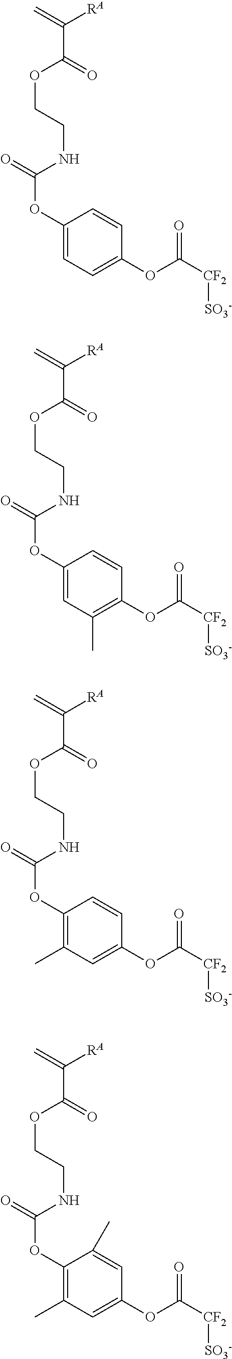

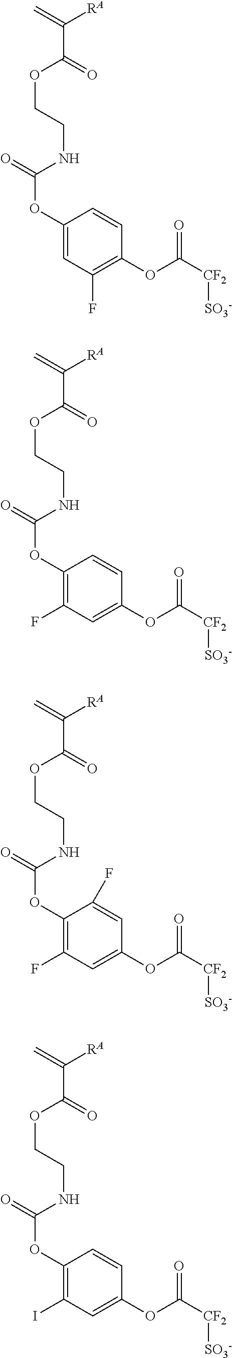

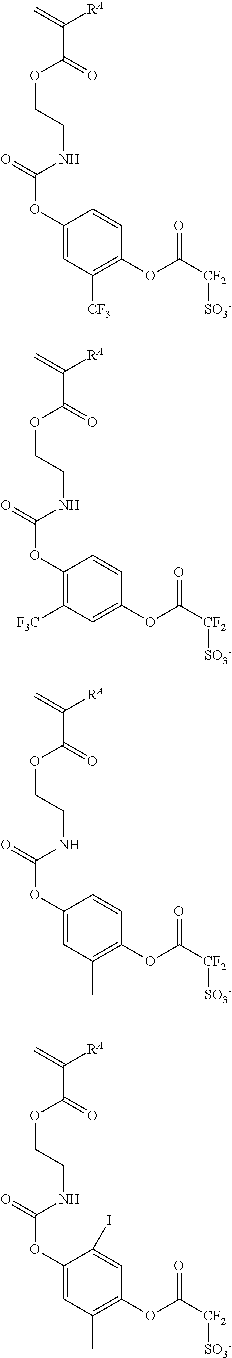

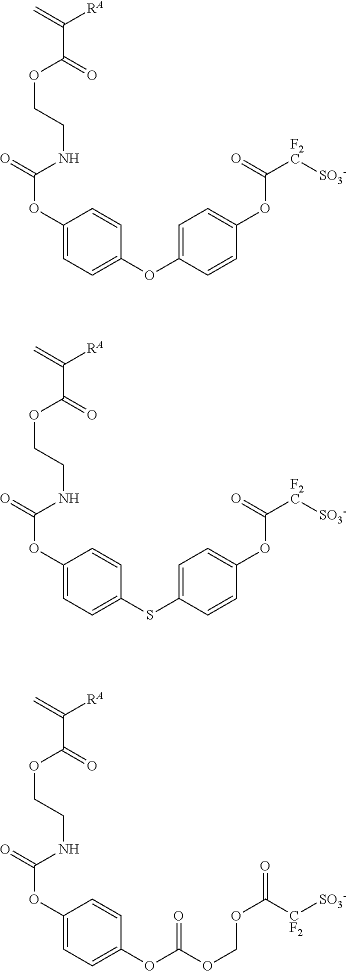

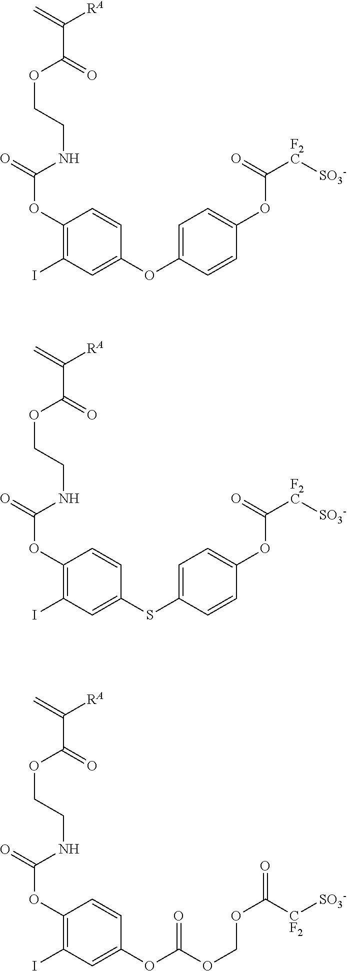

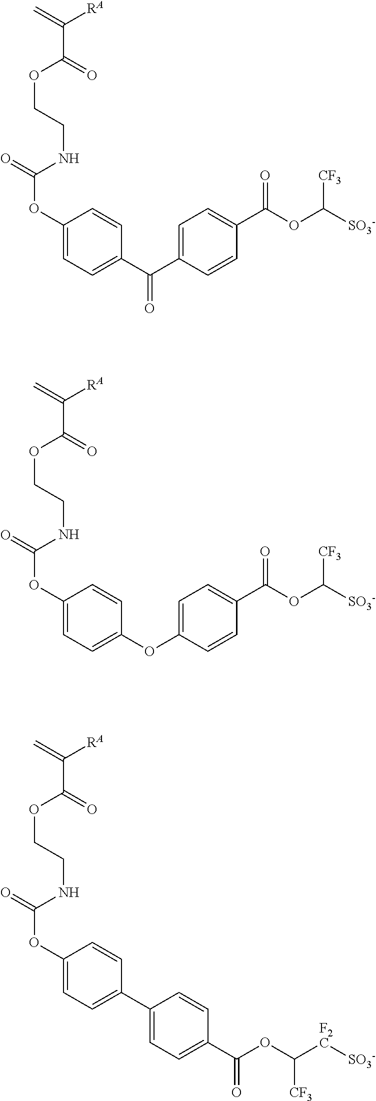

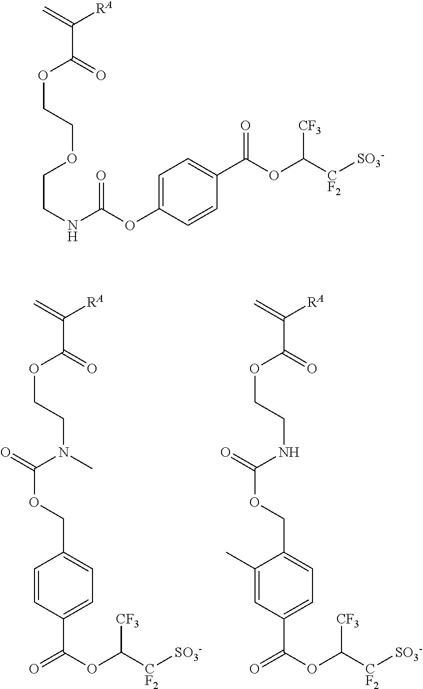

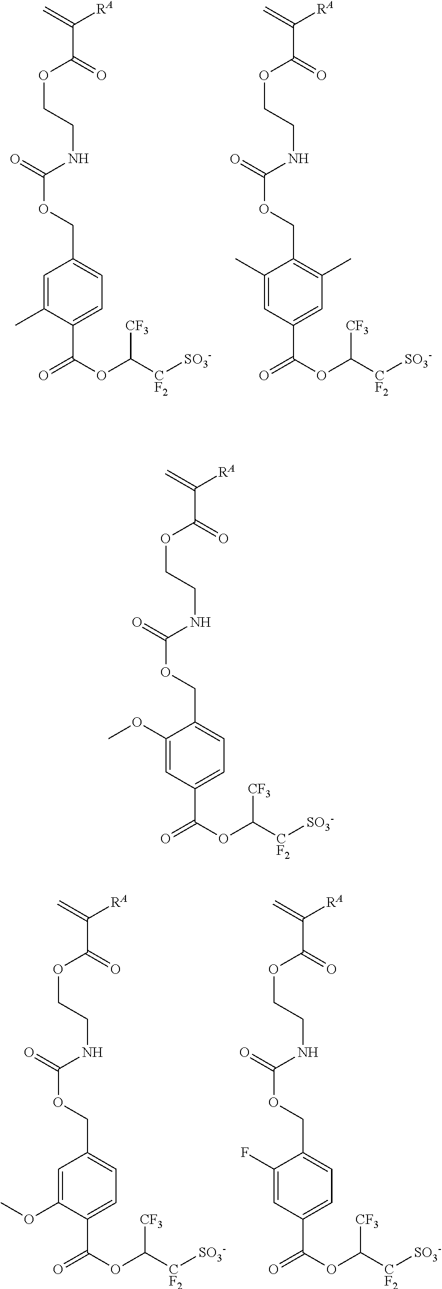

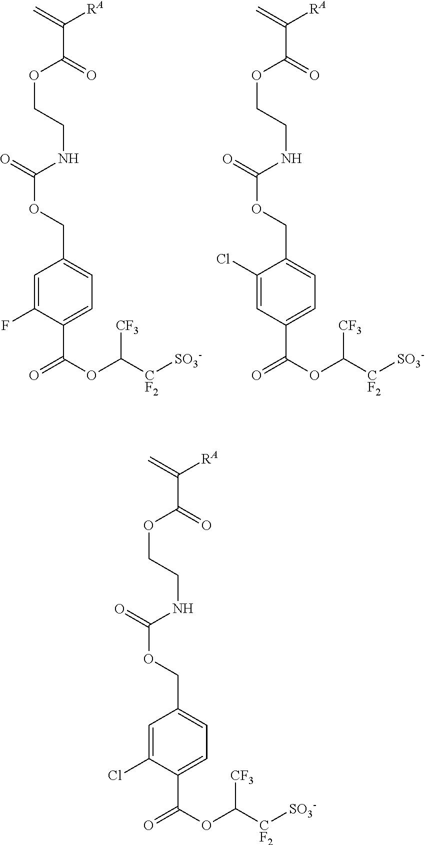

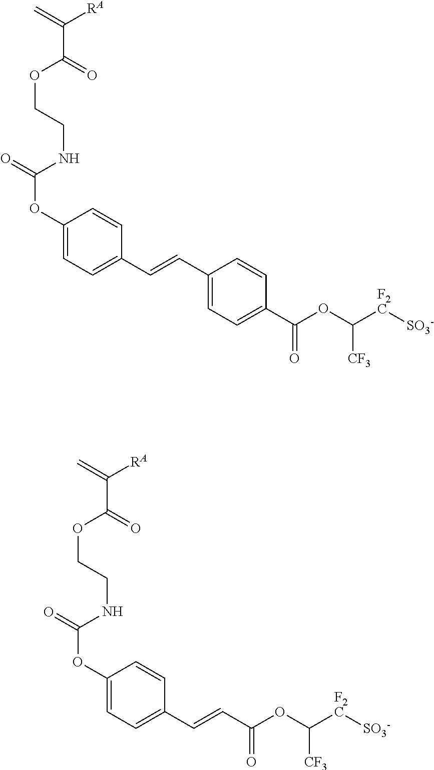

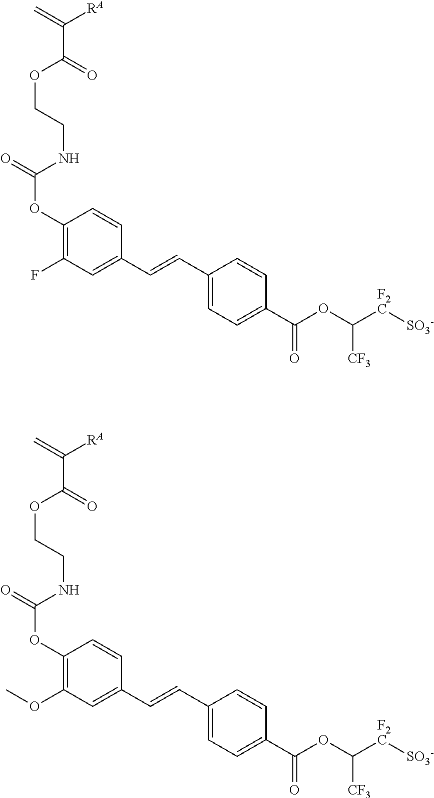

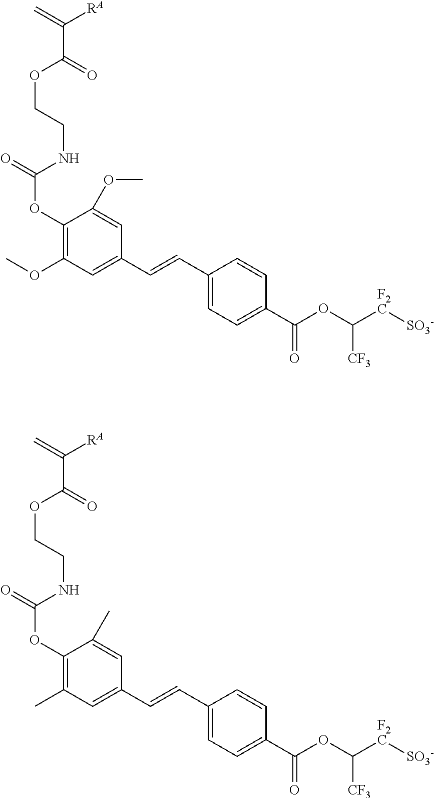

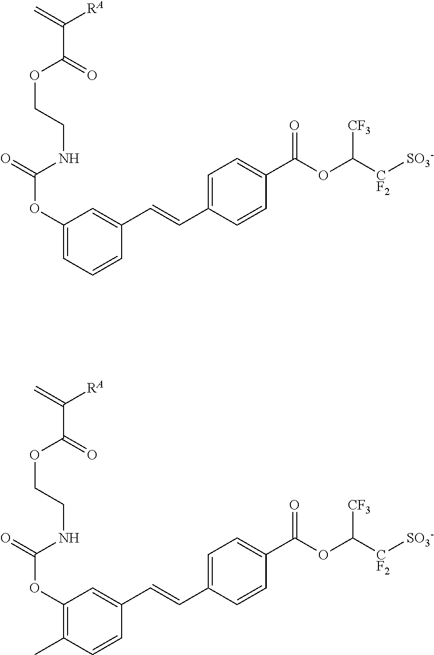

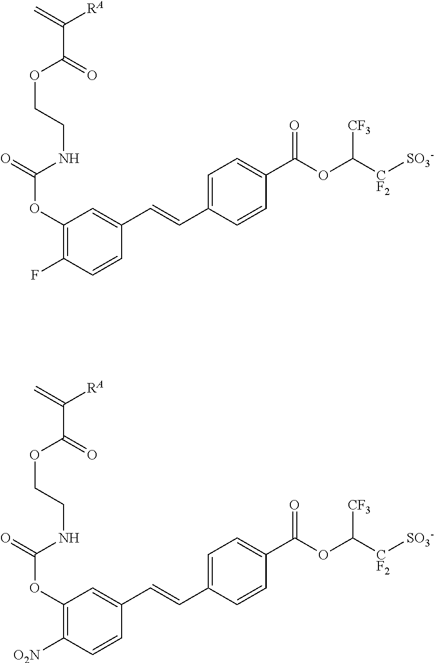

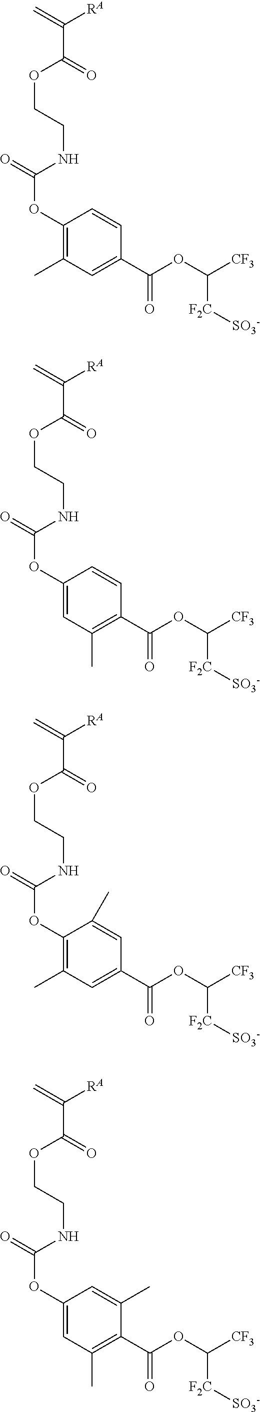

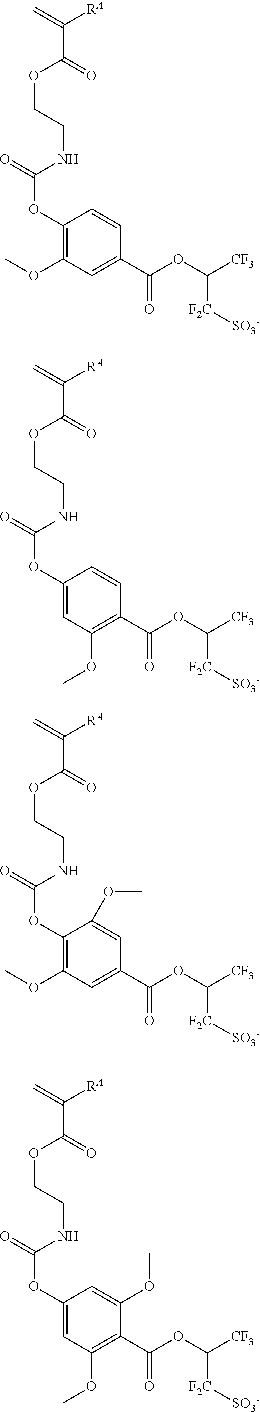

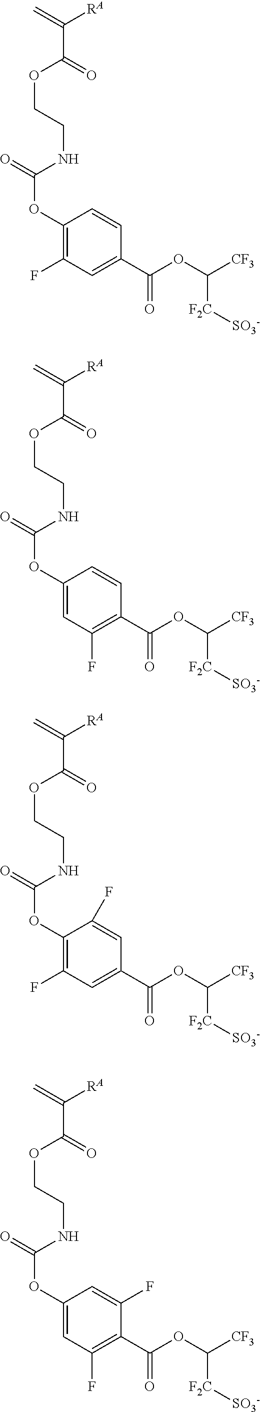

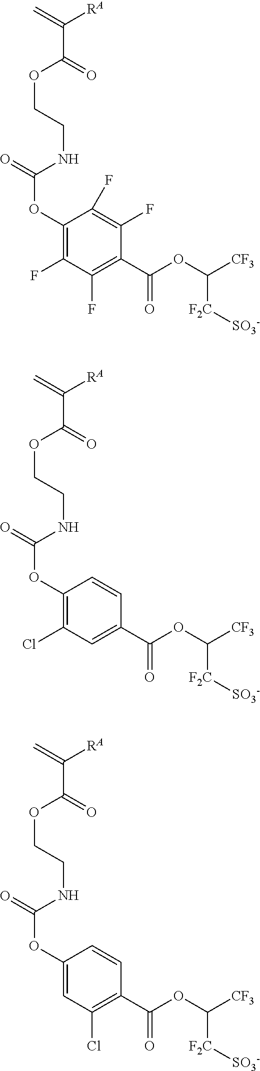

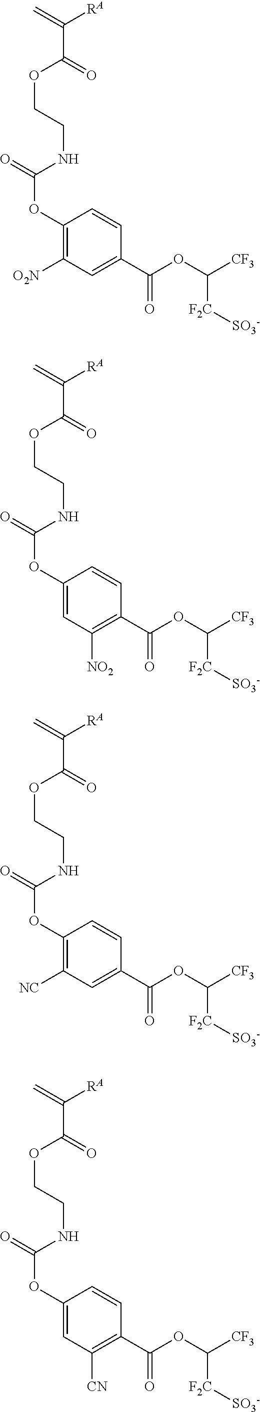

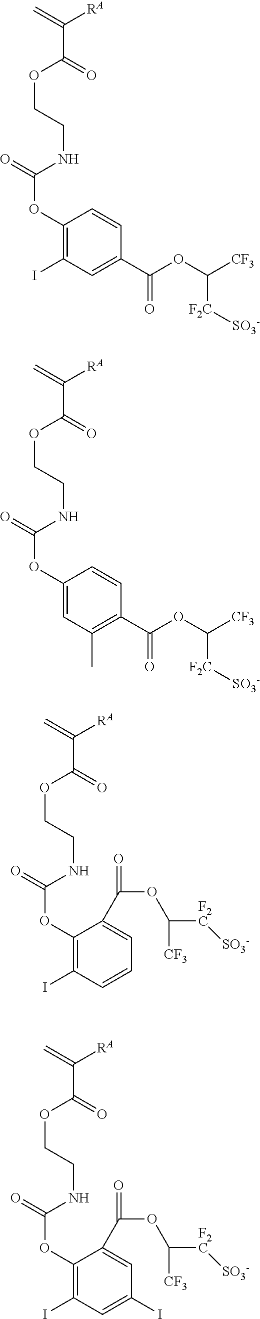

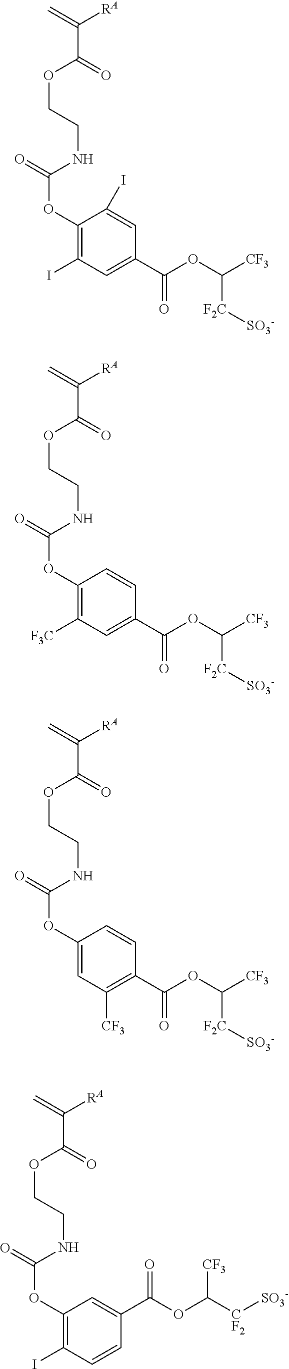

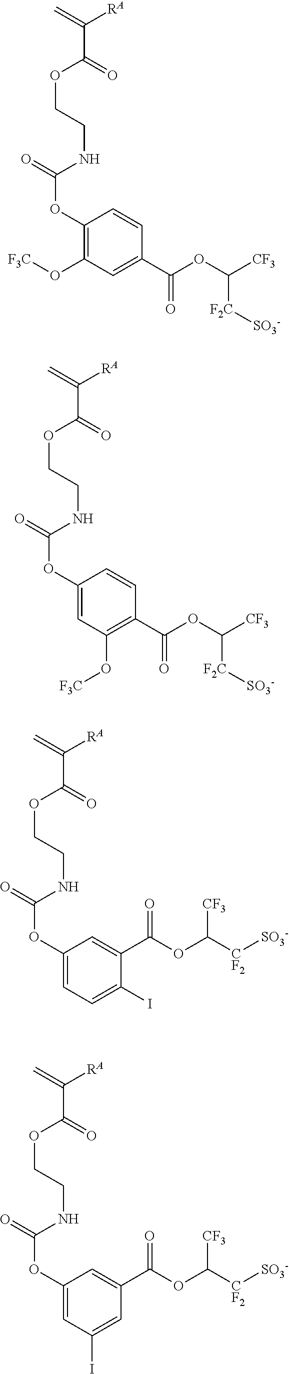

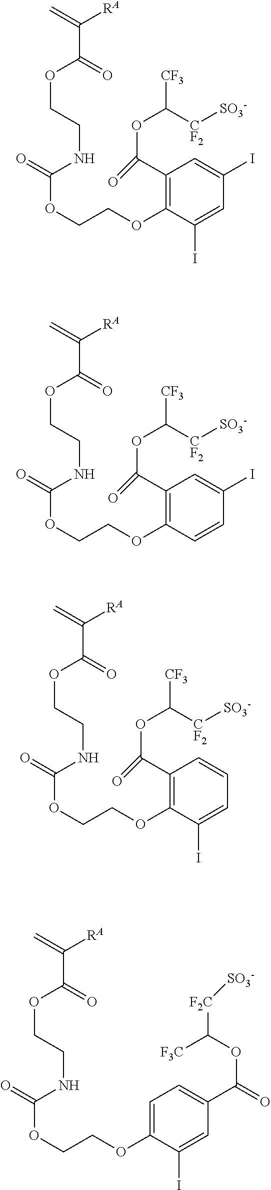

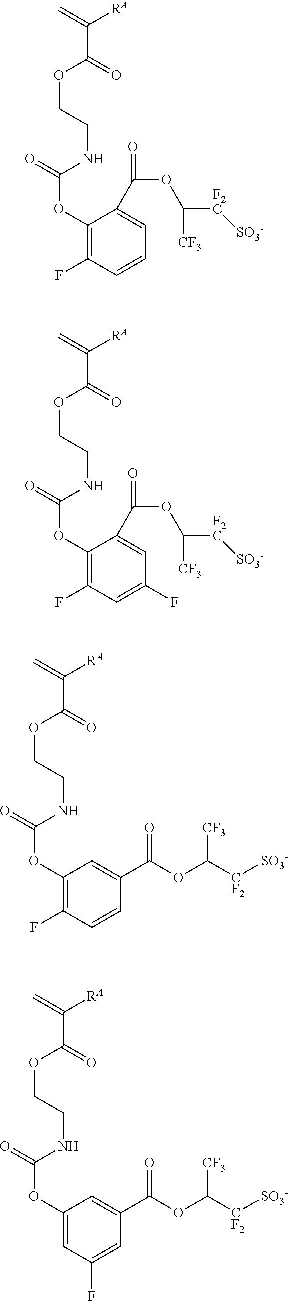

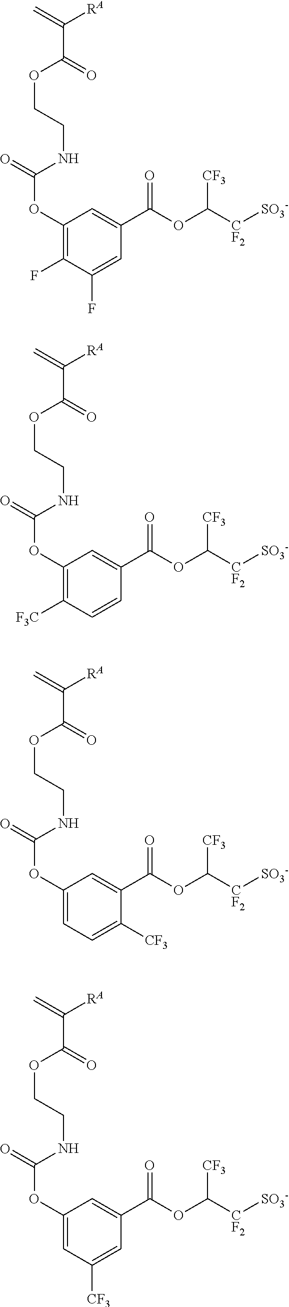

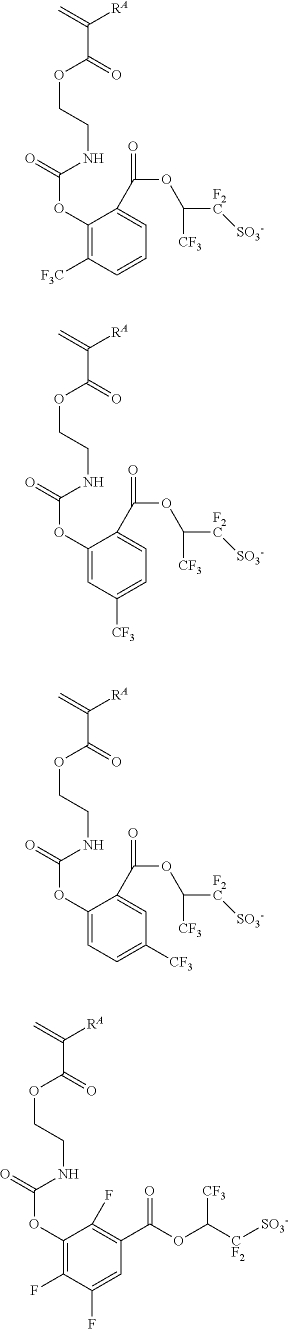

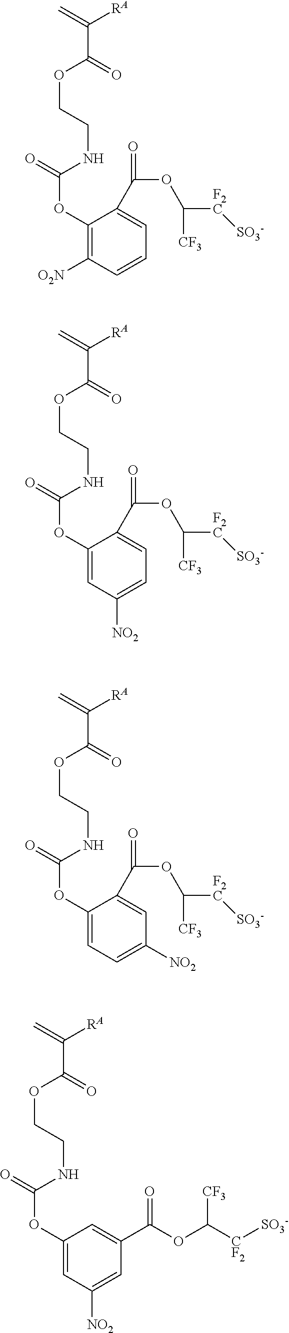

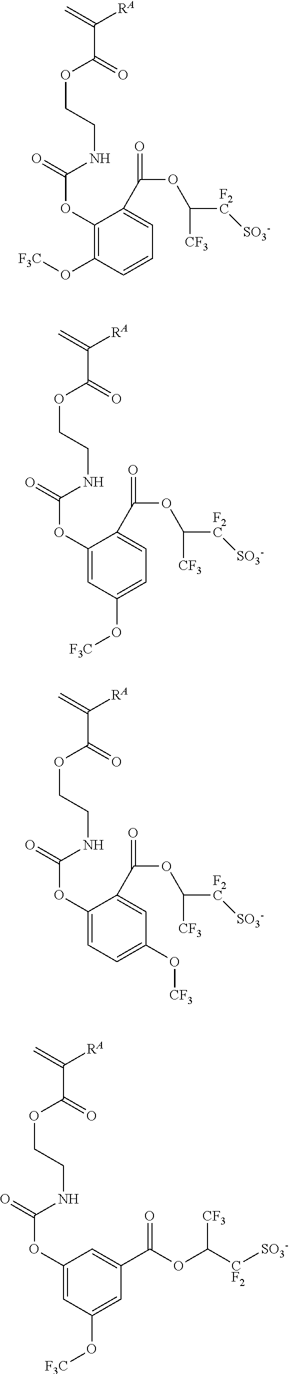

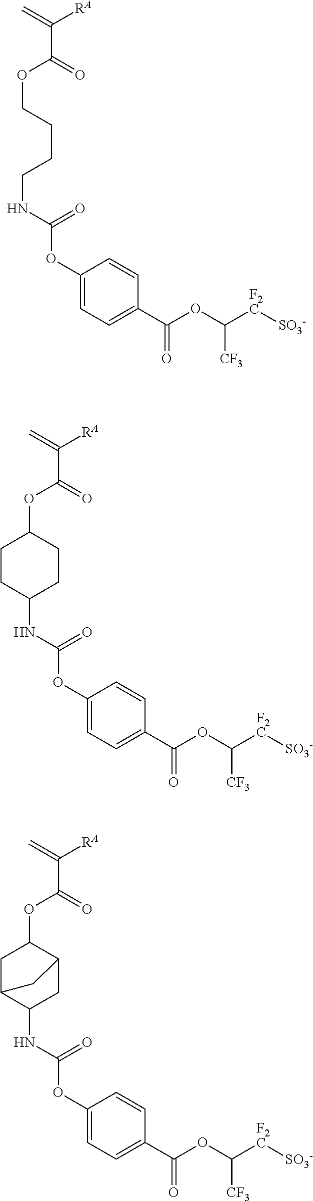

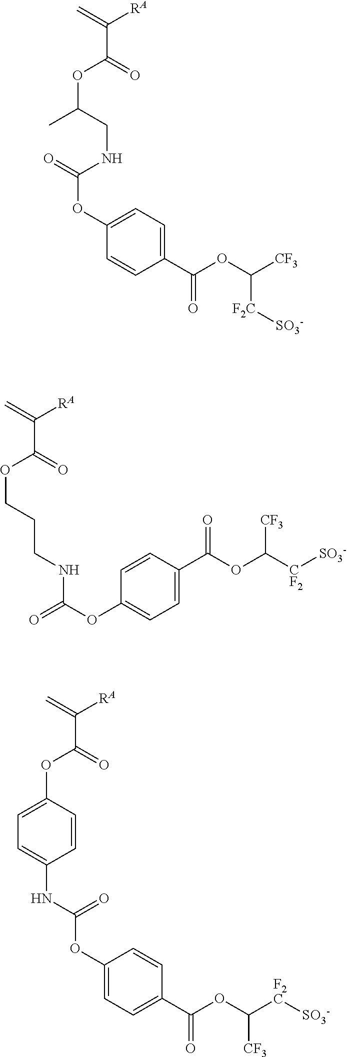

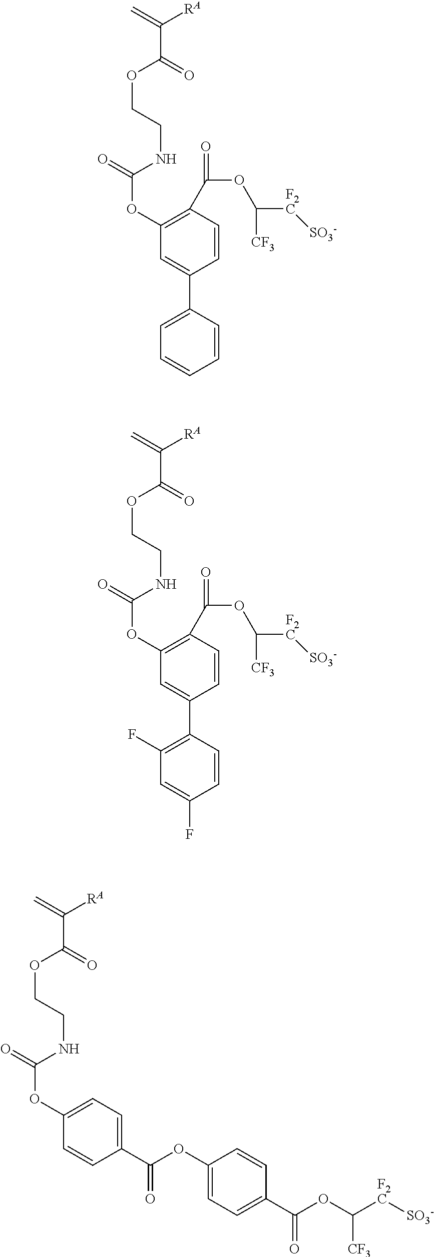

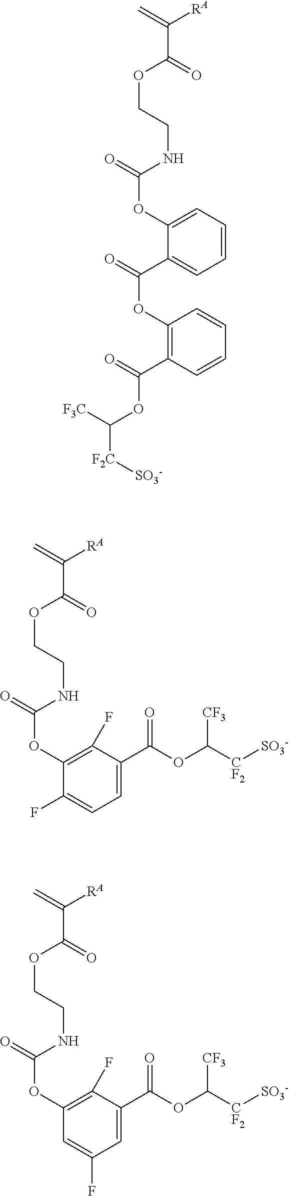

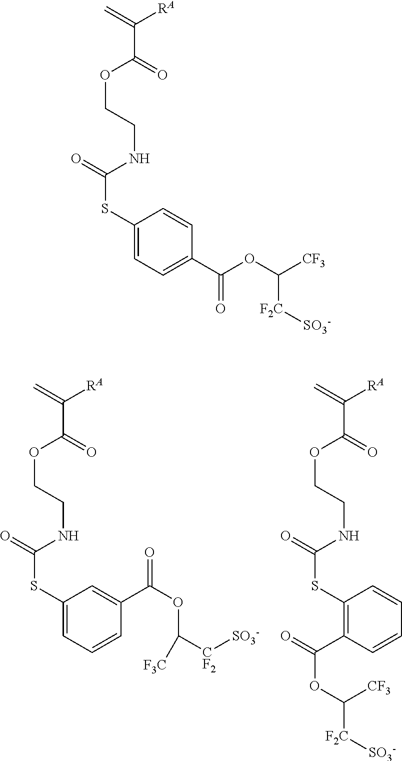

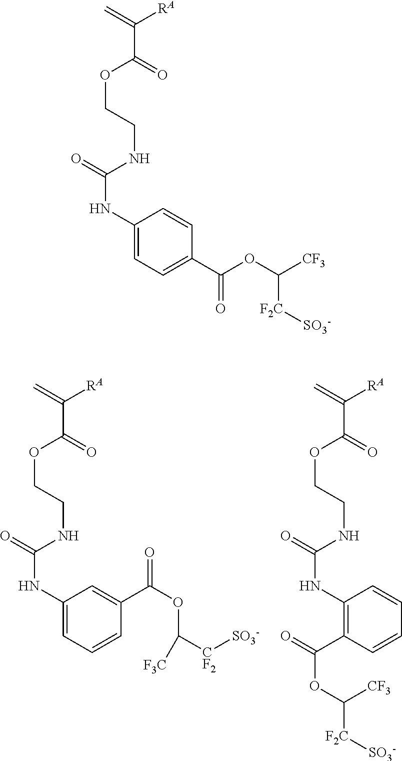

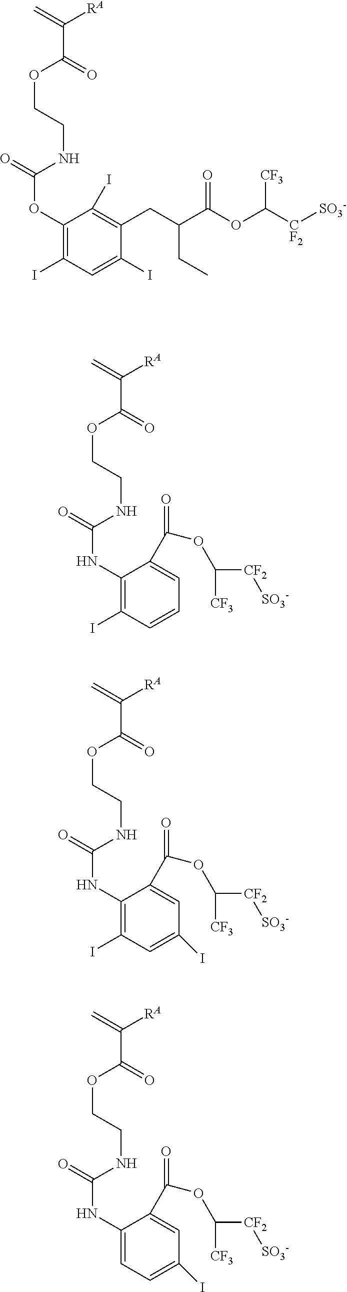

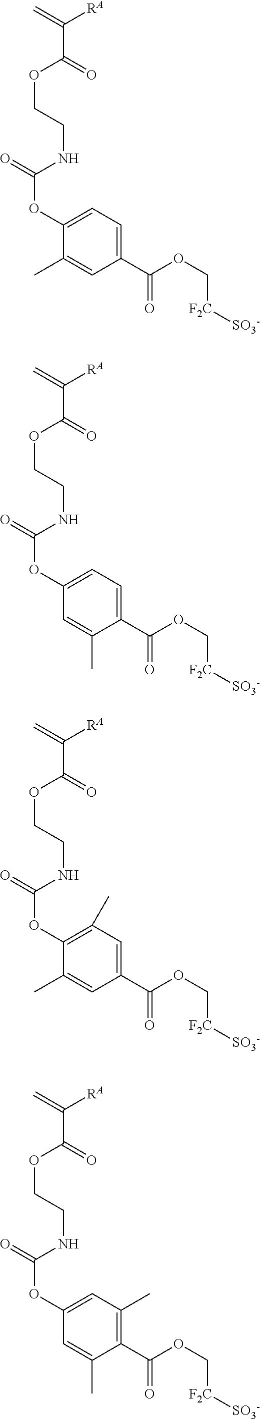

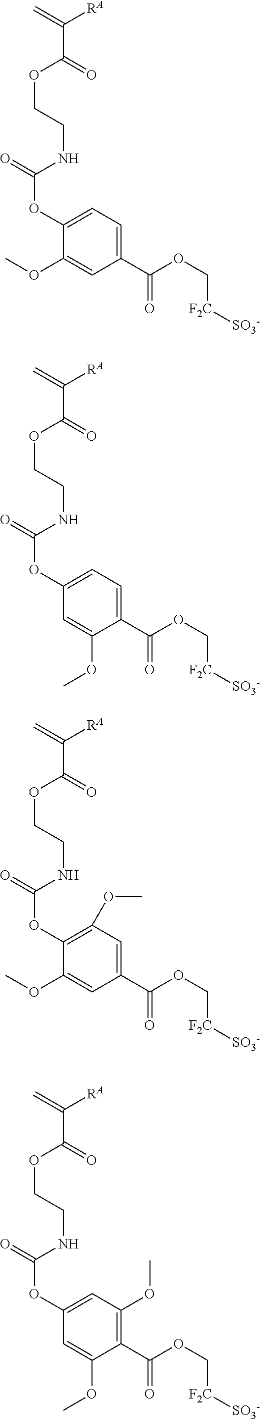

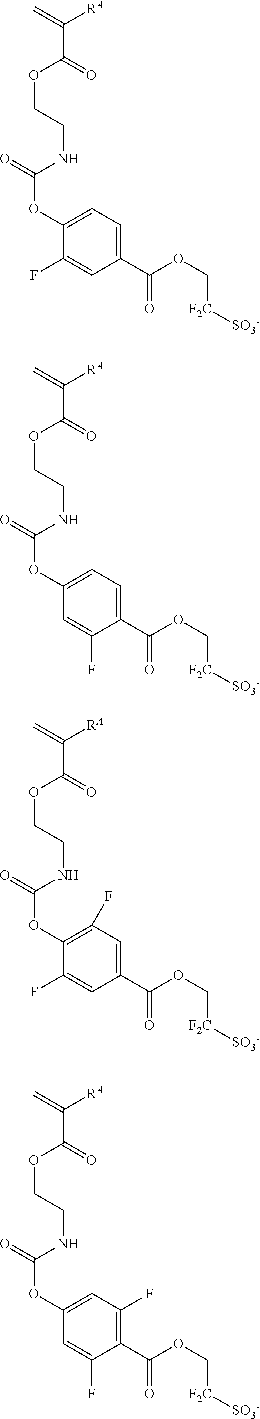

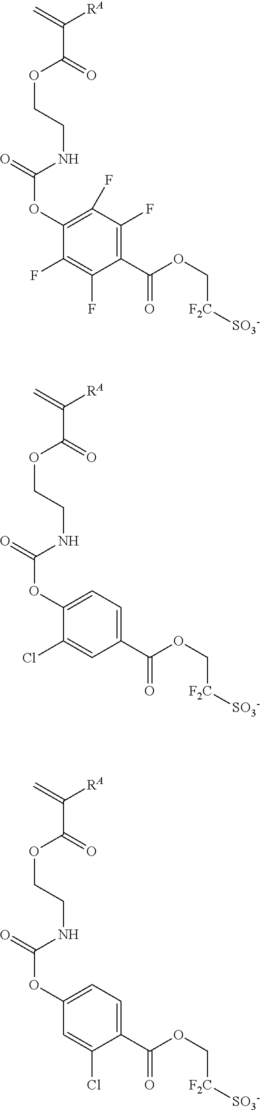

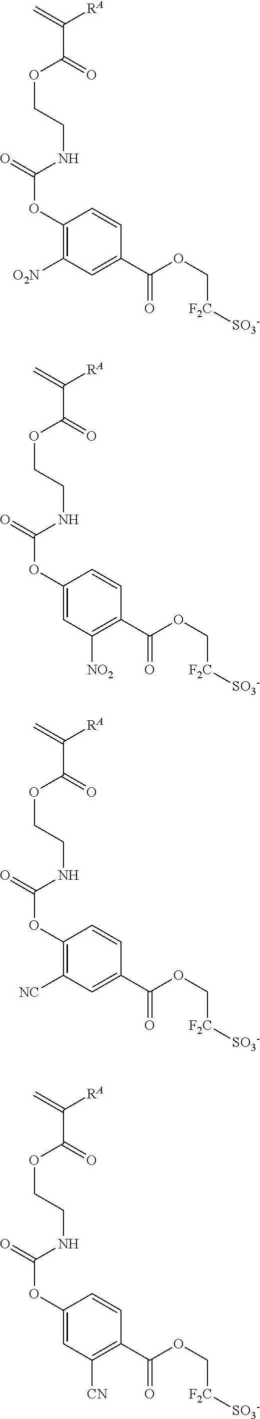

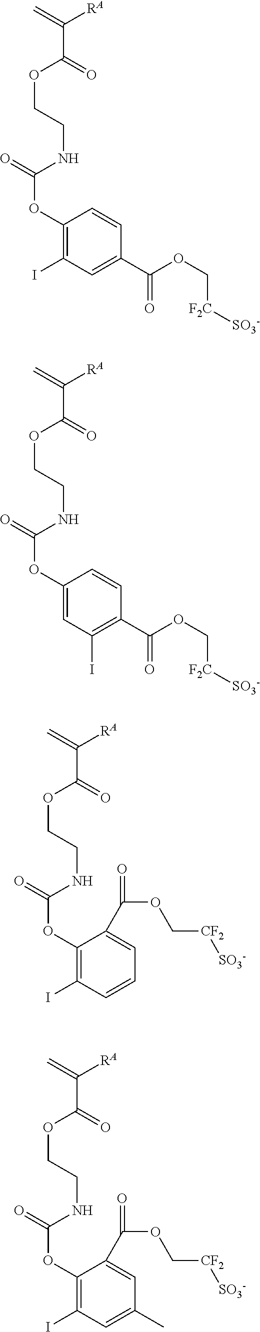

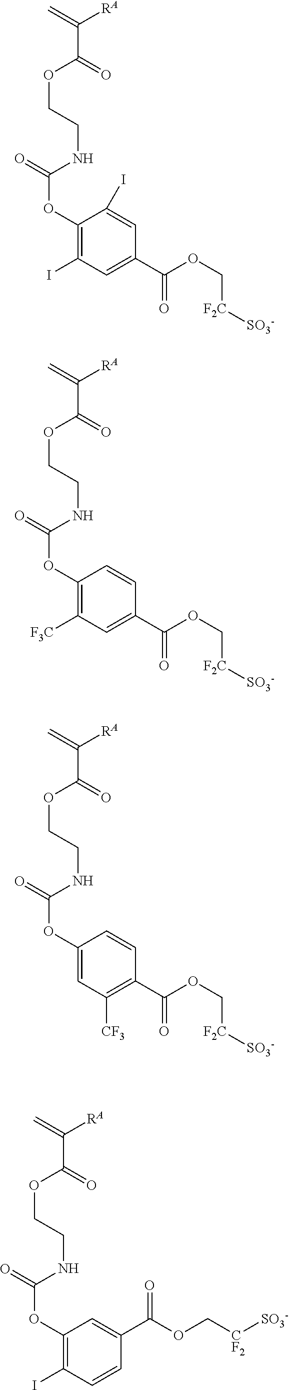

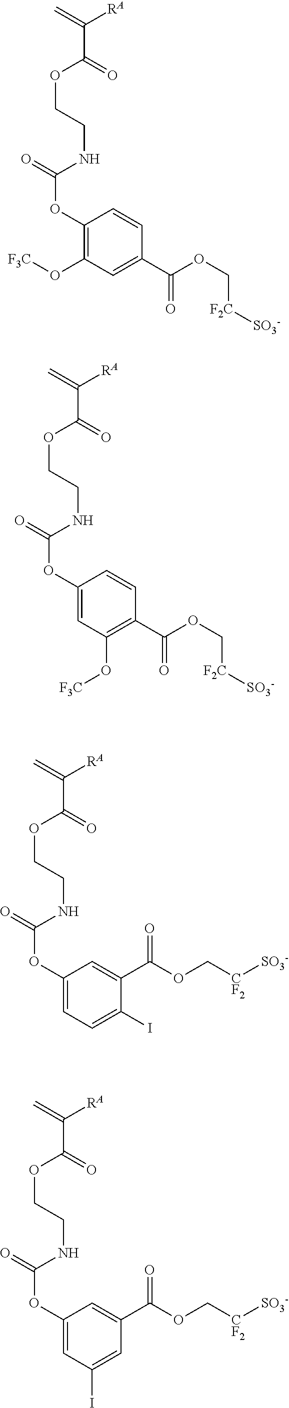

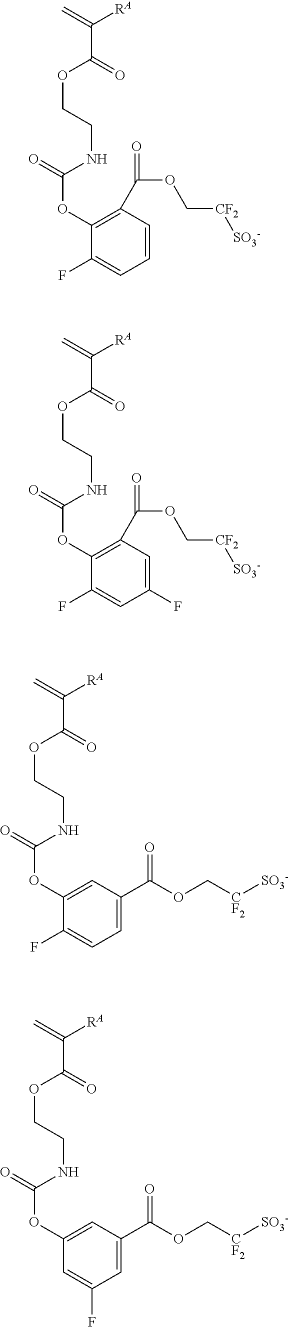

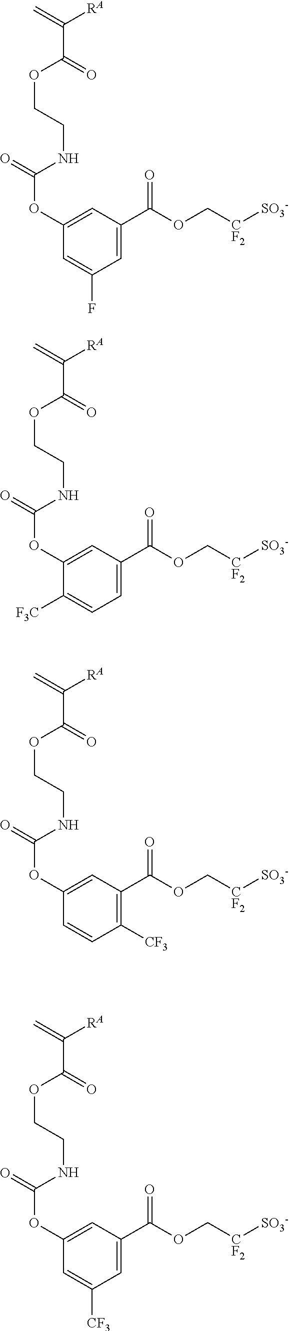

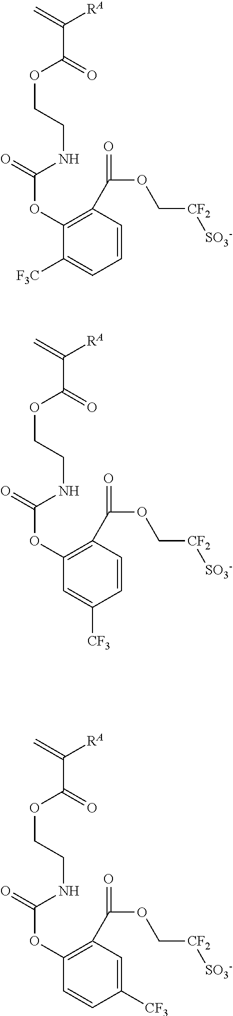

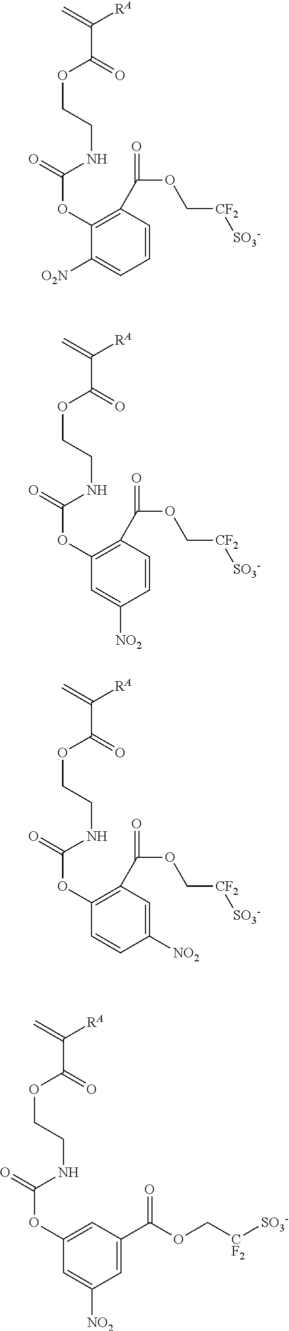

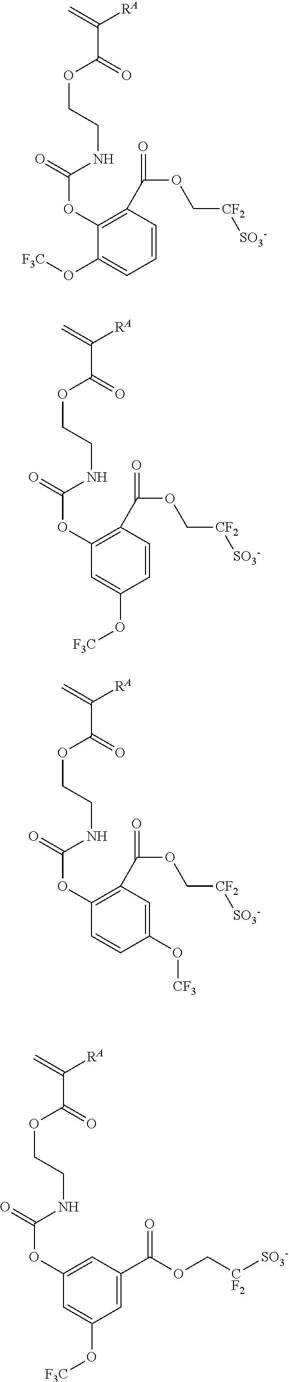

Examples of the anion in the monomer from which repeat unit (f3) is derived are shown below, but not limited thereto. R A is as defined above.

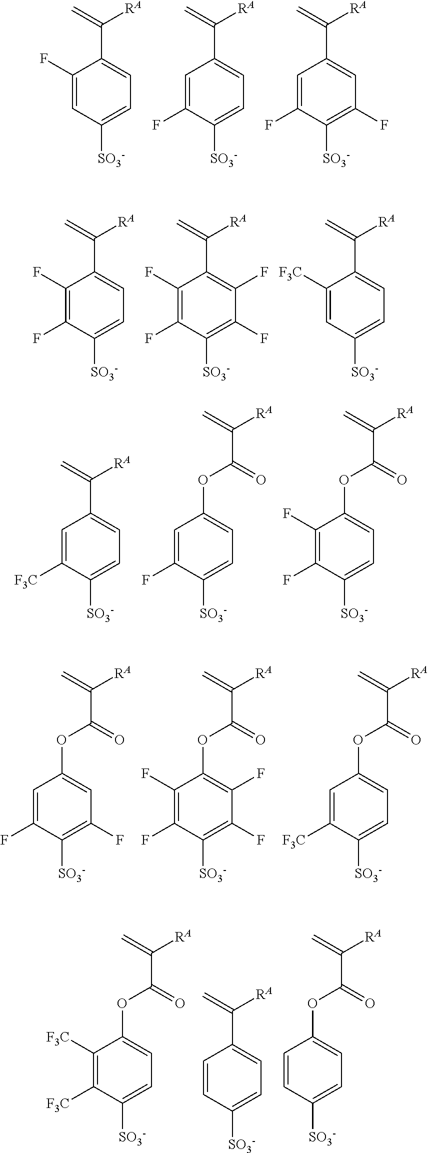

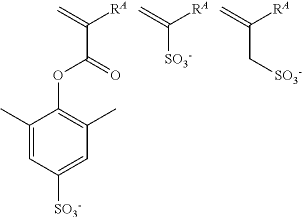

Repeat units (f1) to (f3) have an acid generator function. The attachment of an acid generator to the polymer main chain is effective in restraining acid diffusion, thereby preventing a reduction of resolution due to blur by acid diffusion. Also, LWR or CDU is improved since the acid generator is uniformly distributed. Where a base polymer containing repeat units (f), i.e., polymer-bound acid generator is used, the blending of an acid generator of addition type (to be described later) may be omitted.

In the base polymer, a fraction of units (a1), (a2), (b), (c), (d), (e), (f1), (f2) and (f3) is: preferably 0≤a1≤0.9, 0≤a2≤0.9, 0≤a1+a2≤0.9, 0≤b≤0.9, 0≤c≤0.9, 0≤d≤0.5, 0≤e≤0.5, 0×f1≤0.5, 0≤f2≤0.5, 0≤f3≤0.5, and 0≤f1+f2+f3≤0.5; more preferably 0≤a1≤0.8, 0≤a2≤0.8, 0≤a1+a2≤0.8, 0≤b≤0.8, 0≤c≤0.8, 0≤d≤0.4, 0≤e≤0.4, 0≤f1≤0.4, 0≤f2≤0.4, 0≤f3≤0.4, and 0≤f1+f2+f3≤0.4; and even more preferably 0≤a1≤0.7, 0≤a2≤0.7, 0≤a1+a2≤0.7, 0≤b≤0.7, 0≤c≤0.7, 0≤d≤0.3, 0≤e≤0.3, 0≤f1≤0.3, 0≤f2≤0.3, 0≤f3≤0.3, and 0≤f1+f2+f3≤0.3. Notably, a1+a2+b+c+d+e+f1+f2+f3=1.0.

The base polymer may be synthesized by any desired methods, for example, by dissolving one or more monomers selected from the monomers corresponding to the foregoing repeat units in an organic solvent, adding a radical polymerization initiator thereto, and heating for polymerization. Examples of the organic solvent which can be used for polymerization include toluene, benzene, tetrahydrofuran (THF), diethyl ether, and dioxane. Examples of the polymerization initiator used herein include 2,2′-azobisisobutyronitrile (AIBN), 2,2′-azobis(2,4-dimethylvaleronitrile), dimethyl 2,2-azobis(2-methylpropionate), benzoyl peroxide, and lauroyl peroxide. Preferably, the reaction temperature is 50 to 80° C. and the reaction time is 2 to 100 hours, more preferably 5 to 20 hours.

Where a monomer having a hydroxy group is copolymerized, the hydroxy group may be replaced by an acetal group susceptible to deprotection with acid, typically ethoxyethoxy, prior to polymerization, and the polymerization be followed by deprotection with weak acid and water. Alternatively, the hydroxy group may be replaced by an acetyl, formyl, pivaloyl or similar group prior to polymerization, and the polymerization be followed by alkaline hydrolysis.

When hydroxystyrene or hydroxyvinylnaphthalene is copolymerized, an alternative method is possible. Specifically, acetoxystyrene or acetoxyvinylnaphthalene is used instead of hydroxystyrene or hydroxyvinylnaphthalene, and after polymerization, the acetoxy group is deprotected by alkaline hydrolysis, for thereby converting the polymer product to hydroxystyrene or hydroxyvinylnaphthalene. For alkaline hydrolysis, a base such as aqueous ammonia or triethylamine may be used. Preferably the reaction temperature is −20° C. to 100° C., more preferably 0° C. to 60° C., and the reaction time is 0.2 to 100 hours, more preferably 0.5 to 20 hours.

The base polymer should preferably have a weight average molecular weight (Mw) in the range of 1,000 to 500,000, and more preferably 2,000 to 30,000, as measured by GPC versus polystyrene standards using tetrahydrofuran (THF) solvent. A Mw in the range ensures that the resist film is fully heat resistant and dissolvable in alkaline developer.

If a base polymer has a wide molecular weight distribution or dispersity (Mw/Mn), which indicates the presence of lower and higher molecular weight polymer fractions, there is a possibility that foreign matter is left on the pattern or the pattern profile is degraded. The influences of Mw and Mw/Mn become stronger as the pattern rule becomes finer. Therefore, the base polymer should preferably have a narrow dispersity (Mw/Mn) of 1.0 to 2.0, especially 1.0 to 1.5, in order to provide a resist composition suitable for micropatterning to a small feature size.

It is understood that a blend of two or more polymers which differ in compositional ratio, Mw or Mw/Mn is acceptable.

Acid Generator

The resist composition may comprise an acid generator capable of generating a strong acid (referred to as acid generator of addition type, hereinafter). As used herein, the term “strong acid” refers to a compound having a sufficient acidity to induce deprotection reaction of an acid labile group on the base polymer in the case of a chemically amplified positive resist composition, or a compound having a sufficient acidity to induce acid-catalyzed polarity switch reaction or crosslinking reaction in the case of a chemically amplified negative resist composition. The inclusion of such an acid generator ensures that Sulfonium Salt A functions as a quencher and the inventive resist composition functions as a chemically amplified positive or negative resist composition.

The acid generator is typically a compound (PAG) capable of generating an acid in response to actinic ray or radiation. Although the PAG used herein may be any compound capable of generating an acid upon exposure to high-energy radiation, those compounds capable of generating sulfonic acid, imide acid (imidic acid) or methide acid are preferred. Suitable PAGs include sulfonium salts, iodonium salts, sulfonyldiazomethane, N-sulfonyloxyimide, and oxime-O-sulfonate acid generators. Exemplary PAGs are described in JP-A 2008-111103, paragraphs [0122]-[0142] (U.S. Pat. No. 7,537,880).

As the PAG used herein, sulfonium salts having the formula (3-1) and iodonium salts having the formula (3-2) are also preferred.

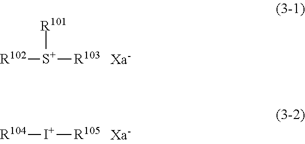

In formulae (3-1) and (3-2), R¹⁰¹ to R¹⁰⁵ are each independently halogen or a C₁-C₂₀ hydrocarbyl group which may contain a heteroatom.

Of the groups represented by R¹⁰¹ to R¹⁰⁵, suitable halogen atoms include fluorine, chlorine, bromine and iodine.

The C₁-C₂₀ hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, propenyl, butenyl, and hexenyl; C₂-C₂₀ alkynyl groups such as ethynyl, propynyl, and butynyl; C₂-C₂₀ cyclic unsaturated aliphatic hydrocarbyl groups such as cyclohexenyl and norbornenyl; C₆-C₂₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, and tert-butylnaphthyl; C₇-C₂₀ aralkyl groups such as benzyl and phenethyl, and combinations thereof. In the foregoing hydrocarbyl groups, some or all hydrogen may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy, fluorine, chlorine, bromine, iodine, cyano, nitro, mercapto, carbonyl, ether bond, ester bond, sulfonic ester bond, carbonate, lactone ring, sultone ring, carboxylic anhydride (—C(═O)—O—C(═O)—), or haloalkyl moiety.

R¹⁰¹ and R¹⁰² may bond together to form a ring with the sulfur atom to which they are attached. Preferred rings are of the structures shown below.

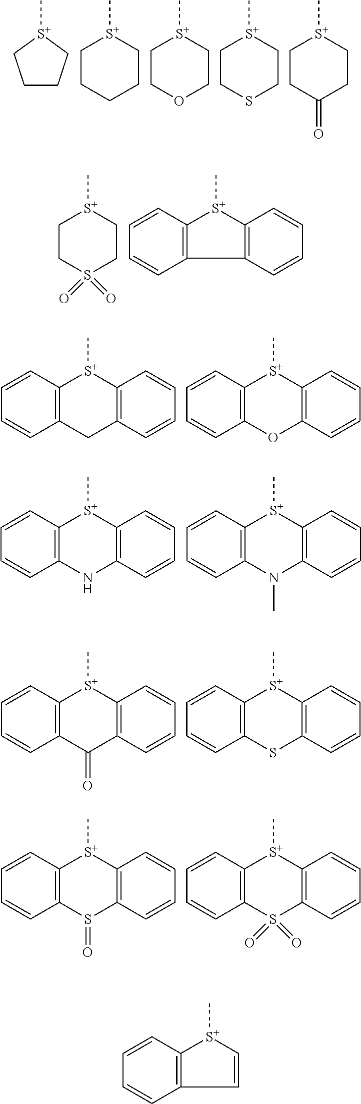

Herein the broken line designates a point of attachment to R¹⁰³.

Examples of the cation in the sulfonium salt having formula (3-1) are shown below, but not limited thereto.

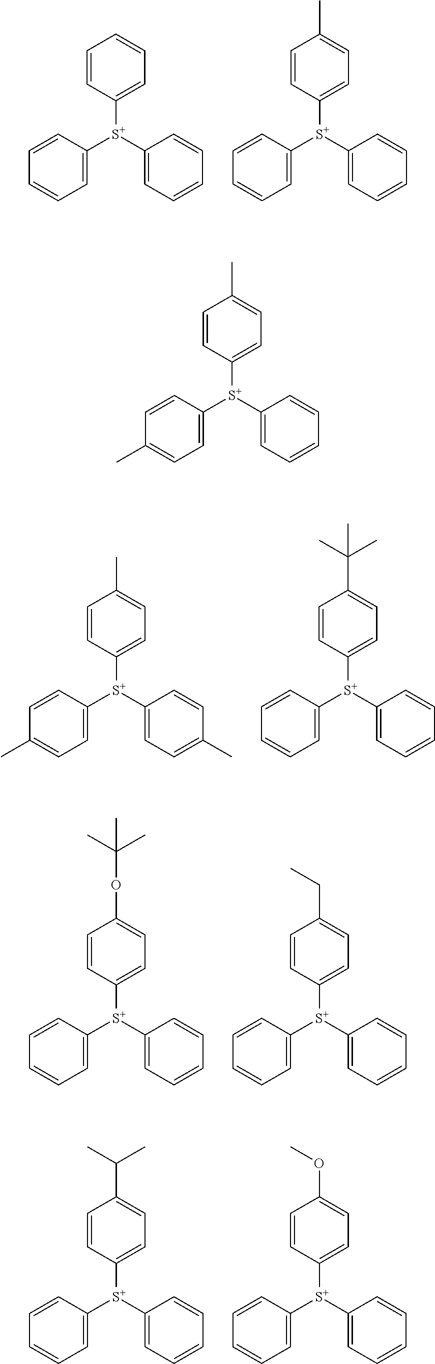

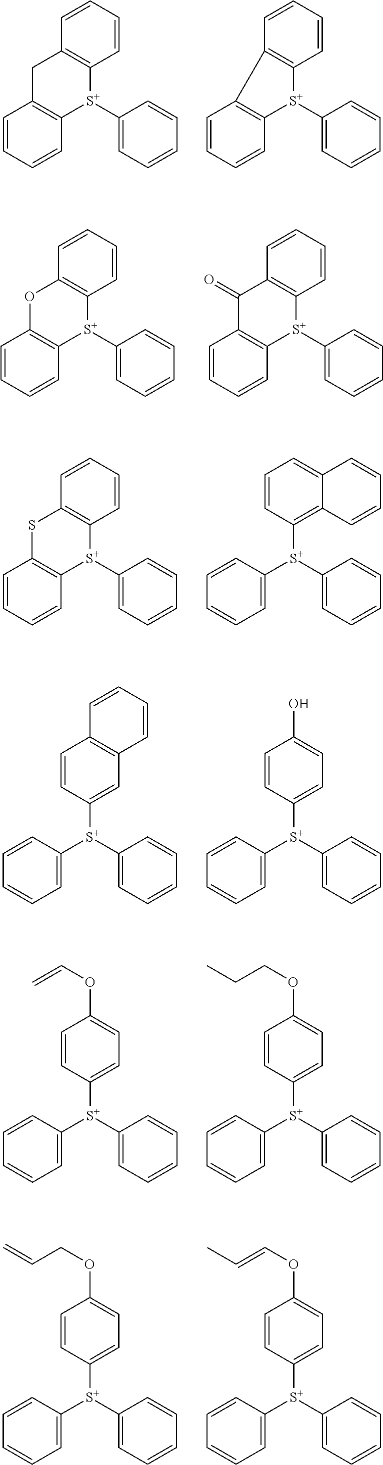

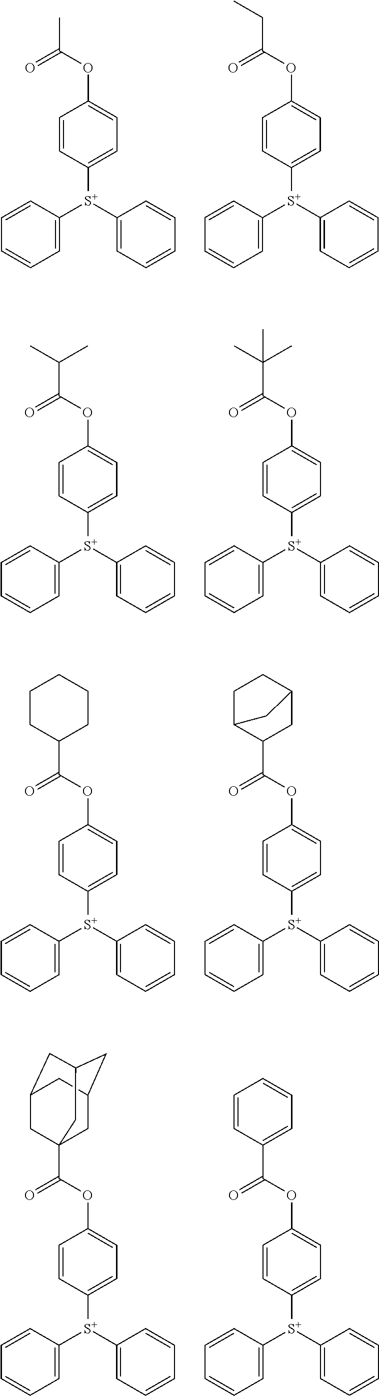

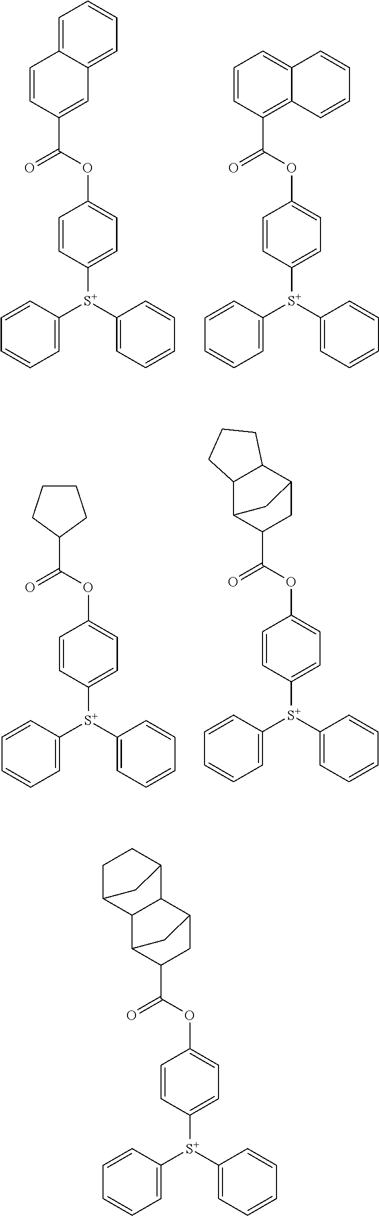 [SMILES: O=C(Oc1ccc([S+](c2ccccc2)c2ccccc2)cc1)c1ccc2ccccc2c1 ; O=C(Oc1ccc([S+](c2ccccc2)c2ccccc2)cc1)c1cccc2ccccc12 ; O=C(Oc1ccc([S+](c2ccccc2)c2ccccc2)cc1)C1CCCC1 ; O=C(Oc1ccc([S+](c2ccccc2)c2ccccc2)cc1)C1CC2CC1C1CCCC21 ; O=C(Oc1ccc([S+](c2ccccc2)c2ccccc2)cc1)C1CC2CC1C1C3CCC(C3)C21]

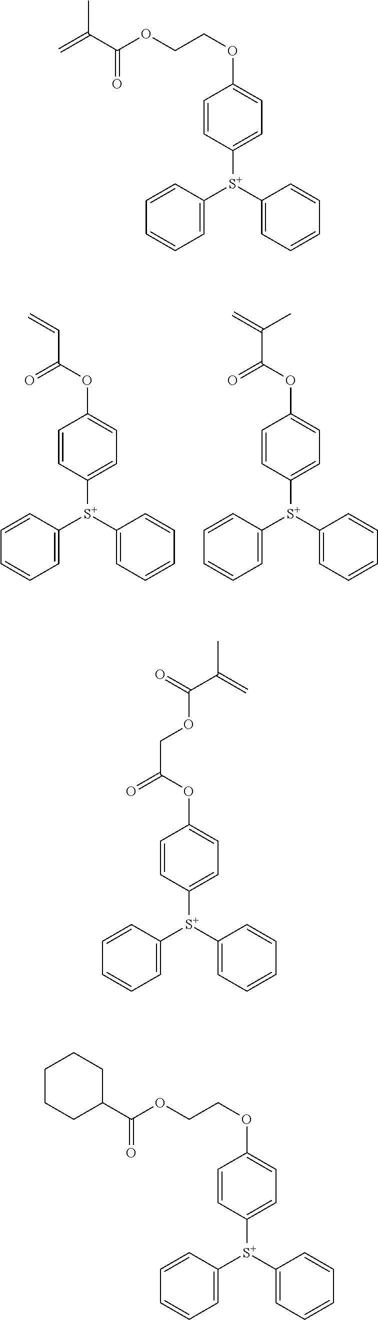 [SMILES: C=C(C)C(=O)OCCOc1ccc([S+](c2ccccc2)c2ccccc2)cc1 ; C=CC(=O)Oc1ccc([S+](c2ccccc2)c2ccccc2)cc1 ; C=C(C)C(=O)Oc1ccc([S+](c2ccccc2)c2ccccc2)cc1 ; C=C(C)C(=O)OCC(=O)Oc1ccc([S+](c2ccccc2)c2ccccc2)cc1 ; O=C(OCCOc1ccc([S+](c2ccccc2)c2ccccc2)cc1)C1CCCCC1]

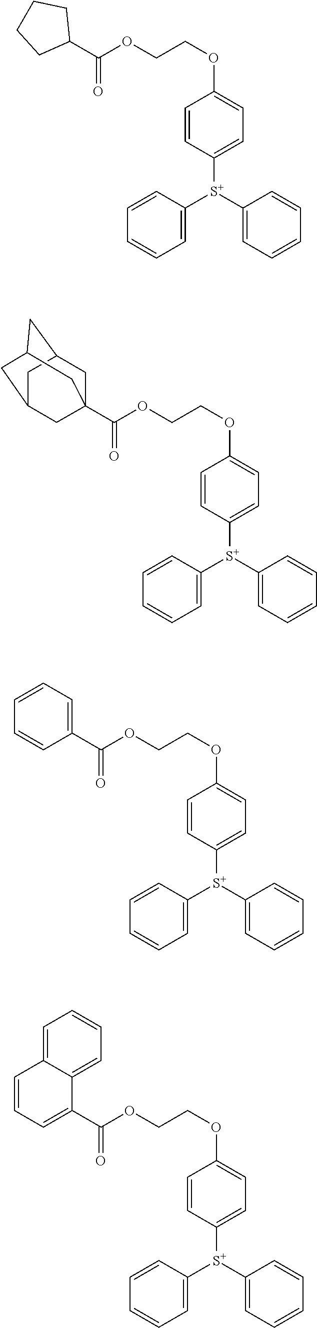 [SMILES: O=C(OCCOc1ccc([S+](c2ccccc2)c2ccccc2)cc1)C1CCCC1 ; O=C(OCCOc1ccc([S+](c2ccccc2)c2ccccc2)cc1)C12CC3CC(CC(C3)C1)C2 ; O=C(OCCOc1ccc([S+](c2ccccc2)c2ccccc2)cc1)c1ccccc1 ; O=C(OCCOc1ccc([S+](c2ccccc2)c2ccccc2)cc1)c1cccc2ccccc12]

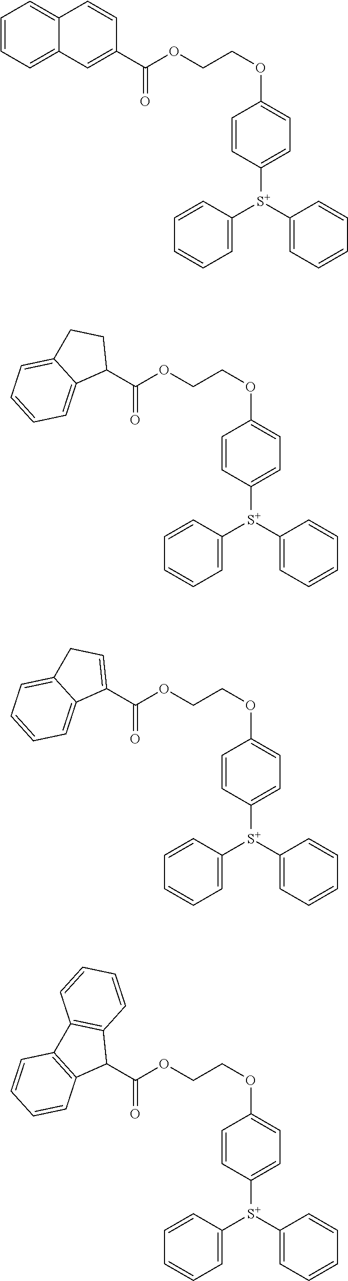 [SMILES: O=C(OCCOc1ccc([S+](c2ccccc2)c2ccccc2)cc1)c1ccc2ccccc2c1 ; O=C(OCCOc1ccc([S+](c2ccccc2)c2ccccc2)cc1)C1CCc2ccccc21 ; O=C(OCCOc1ccc([S+](c2ccccc2)c2ccccc2)cc1)C1=CCc2ccccc21 ; O=C(OCCOc1ccc([S+](c2ccccc2)c2ccccc2)cc1)C1c2ccccc2-c2ccccc21]

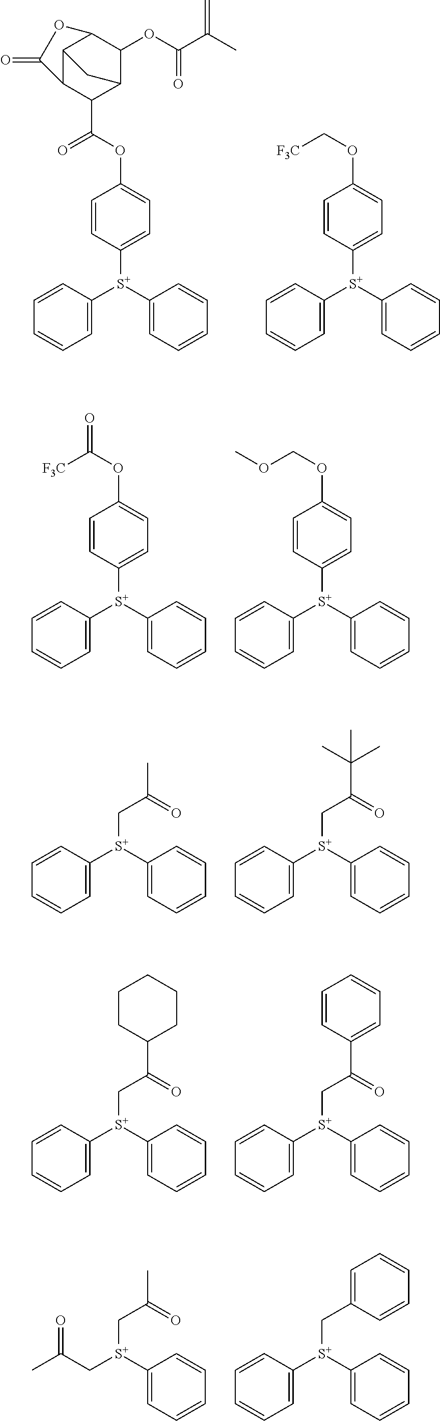

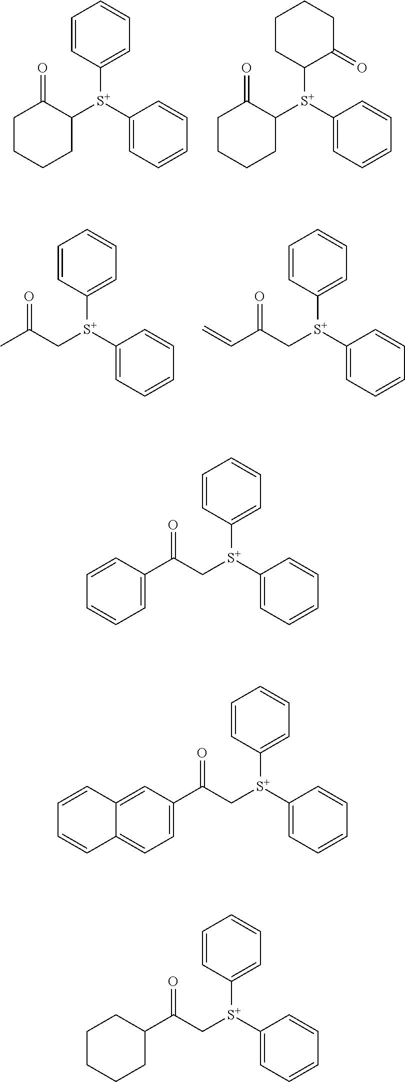

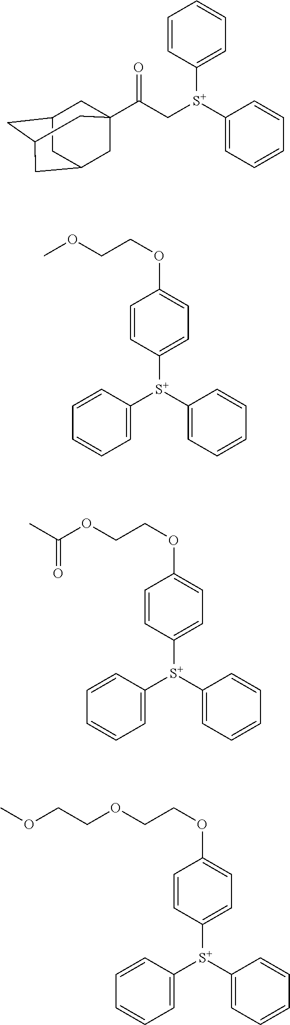 [SMILES: O=C(C[S+](c1ccccc1)c1ccccc1)C12CC3CC(CC(C3)C1)C2 ; COCCOc1ccc([S+](c2ccccc2)c2ccccc2)cc1 ; CC(=O)OCCOc1ccc([S+](c2ccccc2)c2ccccc2)cc1 ; COCCOCCOc1ccc([S+](c2ccccc2)c2ccccc2)cc1]

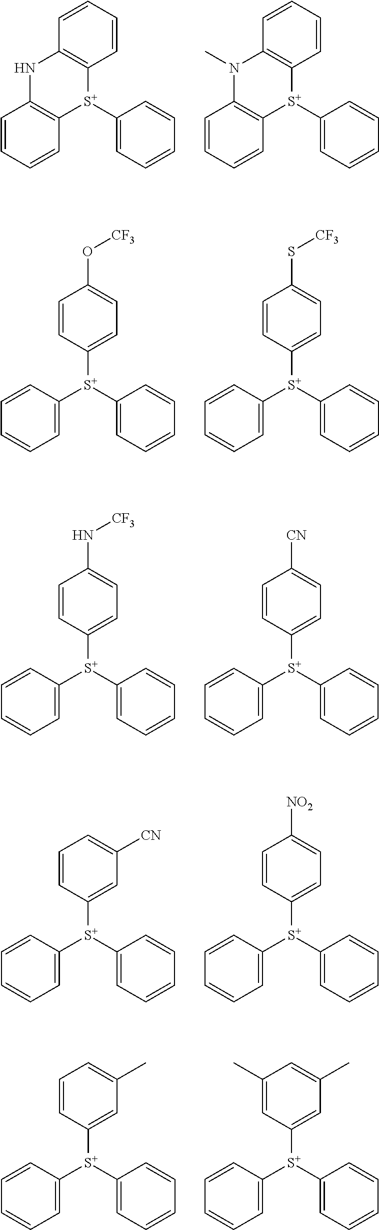

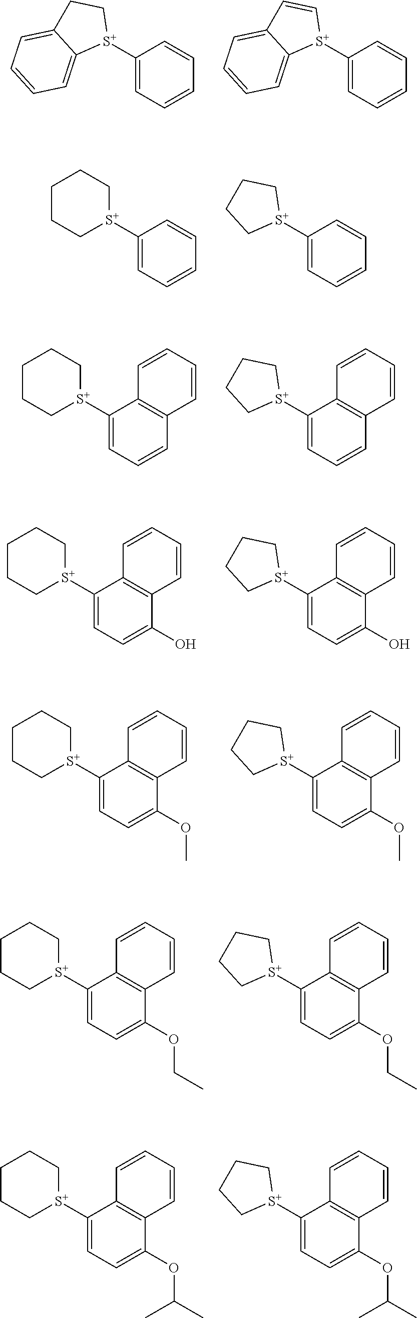

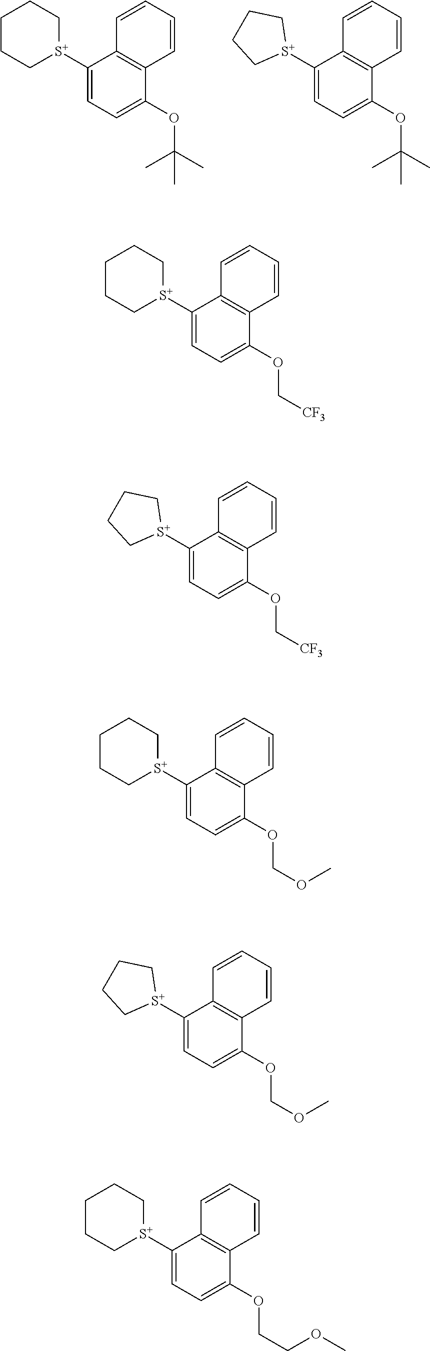

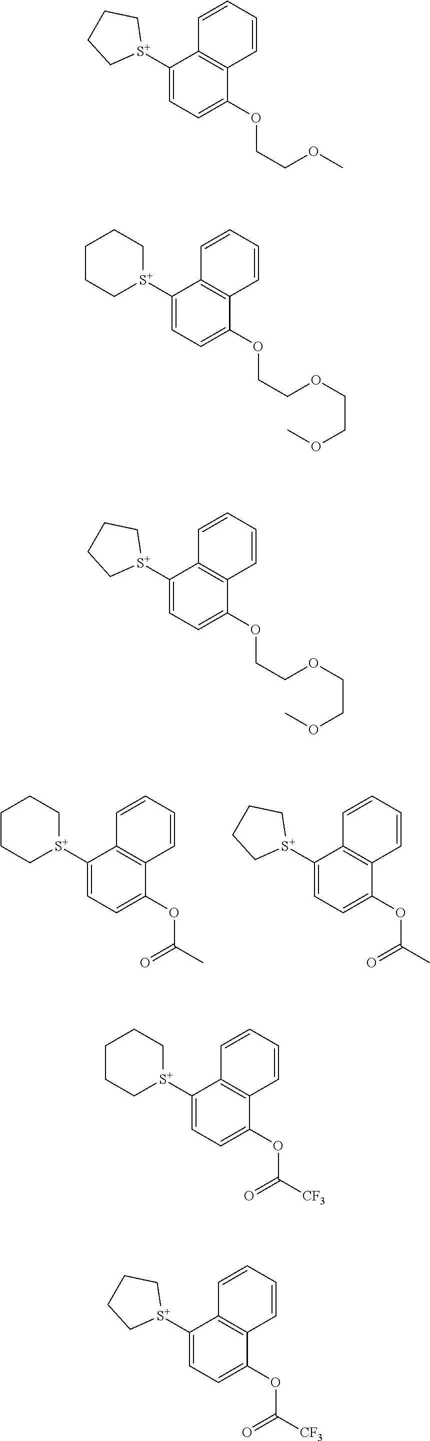

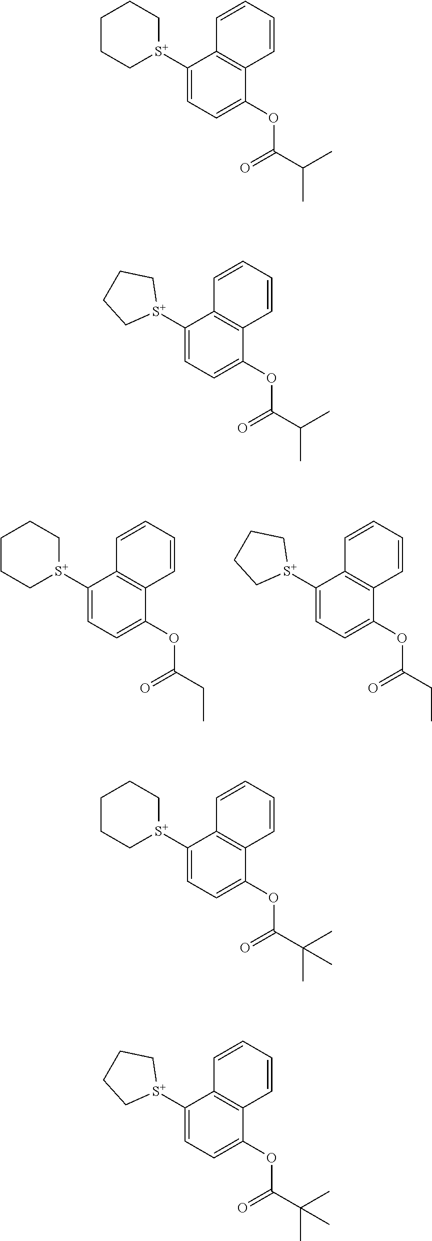 [SMILES: CC(C)C(=O)Oc1ccc([S+]2CCCCC2)c2ccccc12 ; CC(C)C(=O)Oc1ccc([S+]2CCCC2)c2ccccc12 ; CCC(=O)Oc1ccc([S+]2CCCCC2)c2ccccc12 ; CCC(=O)Oc1ccc([S+]2CCCC2)c2ccccc12 ; CC(C)(C)C(=O)Oc1ccc([S+]2CCCCC2)c2ccccc12 ; CC(C)(C)C(=O)Oc1ccc([S+]2CCCC2)c2ccccc12]

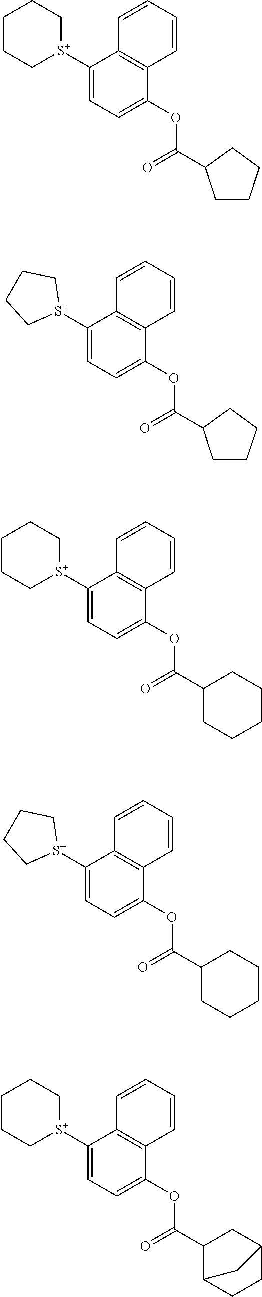 [SMILES: O=C(Oc1ccc([S+]2CCCCC2)c2ccccc12)C1CCCC1 ; O=C(Oc1ccc([S+]2CCCC2)c2ccccc12)C1CCCC1 ; O=C(Oc1ccc([S+]2CCCCC2)c2ccccc12)C1CCCCC1 ; O=C(Oc1ccc([S+]2CCCC2)c2ccccc12)C1CCCCC1 ; O=C(Oc1ccc([S+]2CCCCC2)c2ccccc12)C1CC2CCC1C2]

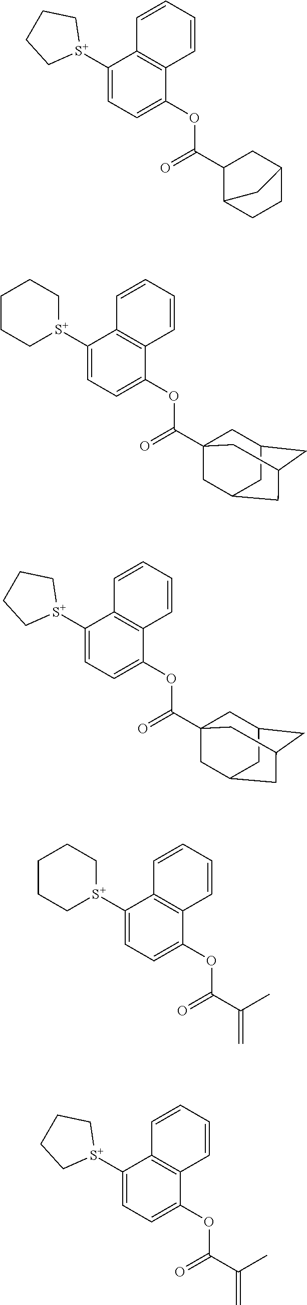 [SMILES: O=C(Oc1ccc([S+]2CCCC2)c2ccccc12)C1CC2CCC1C2 ; O=C(Oc1ccc([S+]2CCCCC2)c2ccccc12)C12CC3CC(CC(C3)C1)C2 ; O=C(Oc1ccc([S+]2CCCC2)c2ccccc12)C12CC3CC(CC(C3)C1)C2 ; C=C(C)C(=O)Oc1ccc([S+]2CCCCC2)c2ccccc12 ; C=C(C)C(=O)Oc1ccc([S+]2CCCC2)c2ccccc12]

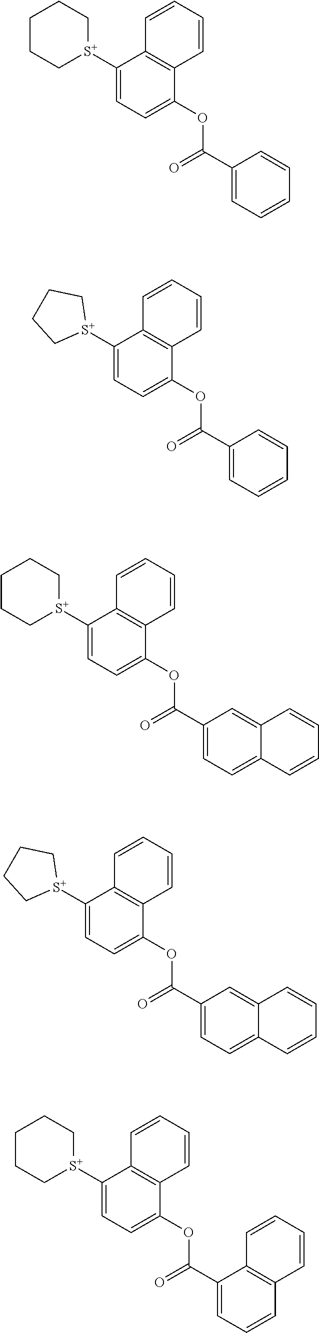 [SMILES: O=C(Oc1ccc([S+]2CCCCC2)c2ccccc12)c1ccccc1 ; O=C(Oc1ccc([S+]2CCCC2)c2ccccc12)c1ccccc1 ; O=C(Oc1ccc([S+]2CCCCC2)c2ccccc12)c1ccc2ccccc2c1 ; O=C(Oc1ccc([S+]2CCCC2)c2ccccc12)c1ccc2ccccc2c1 ; O=C(Oc1ccc([S+]2CCCCC2)c2ccccc12)c1cccc2ccccc12]

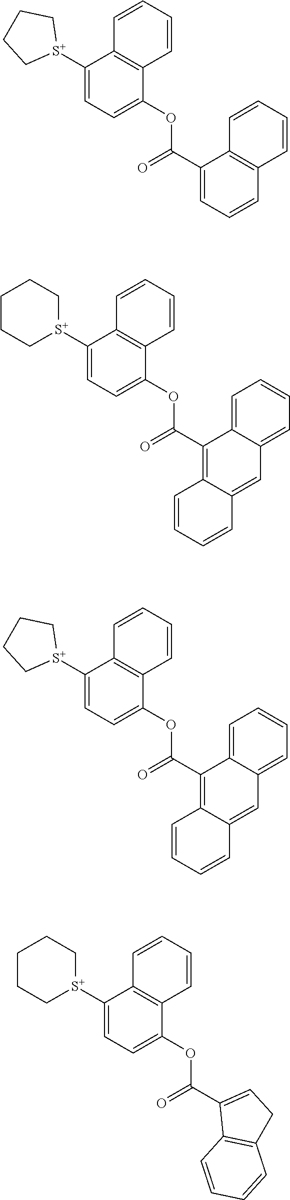 [SMILES: O=C(Oc1ccc([S+]2CCCC2)c2ccccc12)c1cccc2ccccc12 ; O=C(Oc1ccc([S+]2CCCCC2)c2ccccc12)c1c2ccccc2cc2ccccc12 ; O=C(Oc1ccc([S+]2CCCC2)c2ccccc12)c1c2ccccc2cc2ccccc12 ; O=C(Oc1ccc([S+]2CCCCC2)c2ccccc12)C1=CCc2ccccc21]

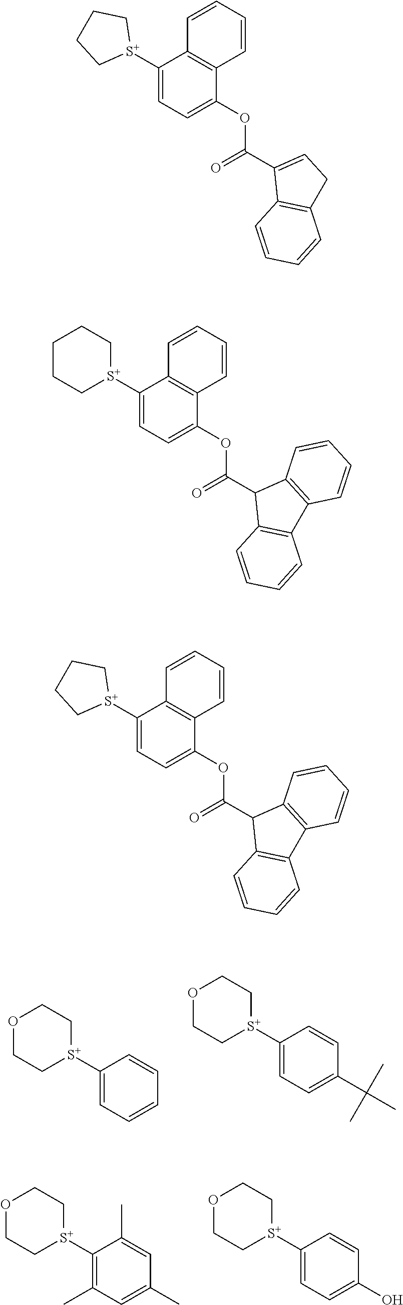

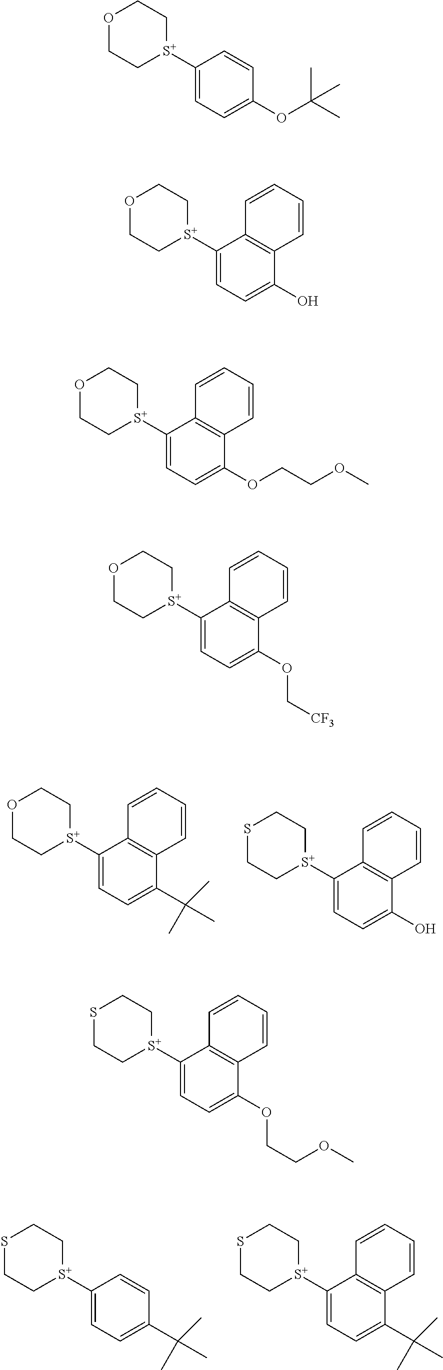

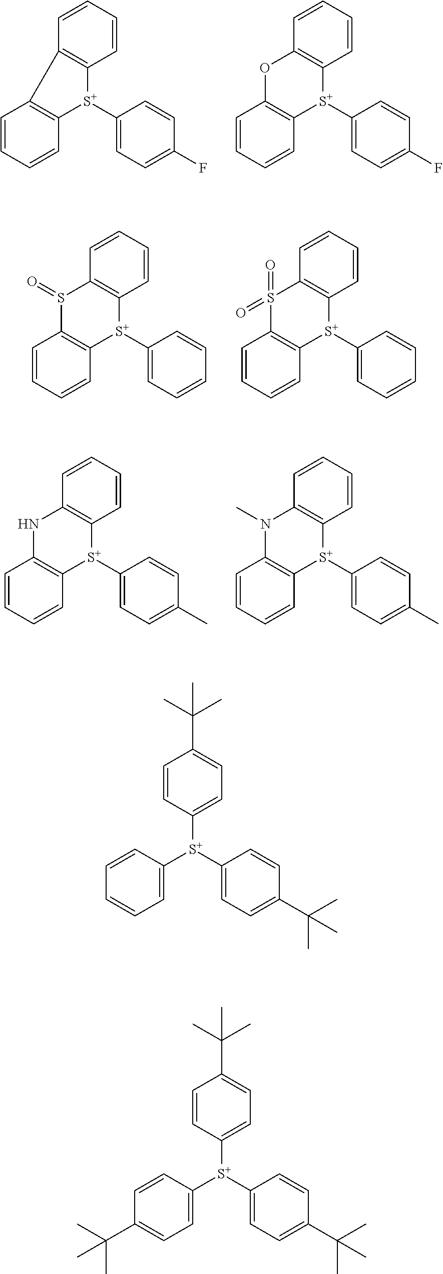

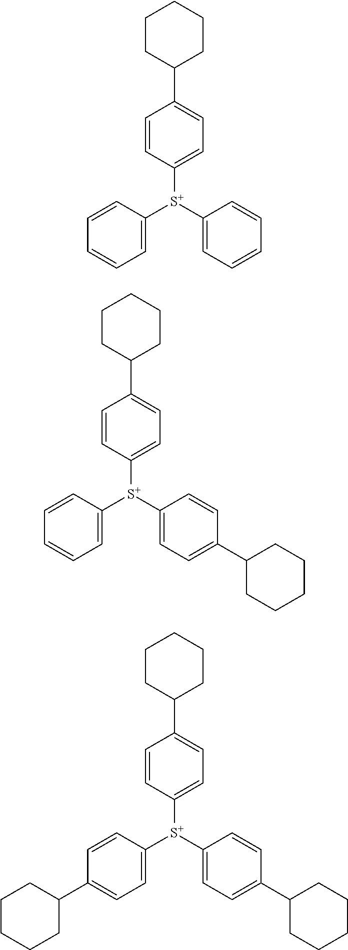 [SMILES: c1ccc([S+](c2ccccc2)c2ccc(C3CCCCC3)cc2)cc1 ; c1ccc([S+](c2ccc(C3CCCCC3)cc2)c2ccc(C3CCCCC3)cc2)cc1 ; c1cc([S+](c2ccc(C3CCCCC3)cc2)c2ccc(C3CCCCC3)cc2)ccc1C1CCCCC1]

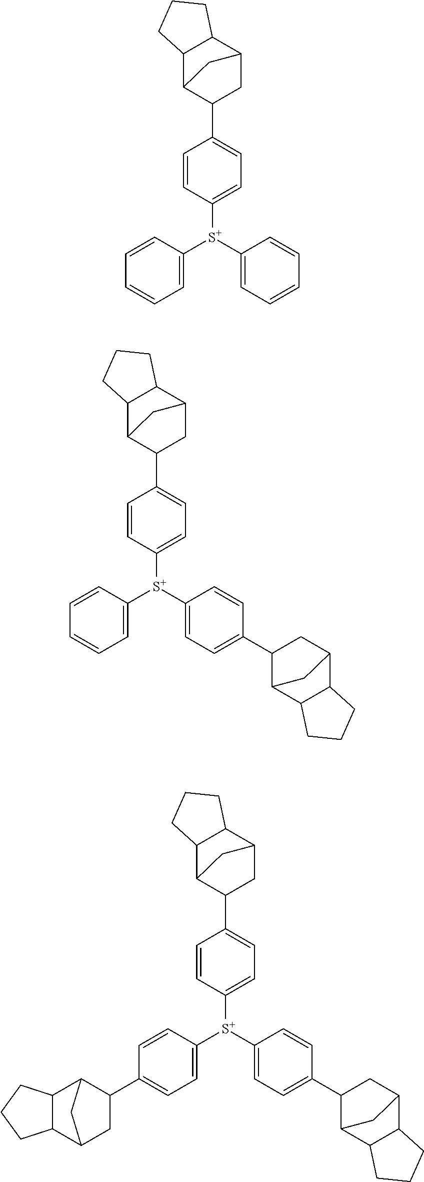 [SMILES: c1ccc([S+](c2ccccc2)c2ccc(C3CC4CC3C3CCCC43)cc2)cc1 ; c1ccc([S+](c2ccc(C3CC4CC3C3CCCC43)cc2)c2ccc(C3CC4CC3C3CCCC43)cc2)cc1 ; c1cc([S+](c2ccc(C3CC4CC3C3CCCC43)cc2)c2ccc(C3CC4CC3C3CCCC43)cc2)ccc1C1CC2CC1C1CCCC21]

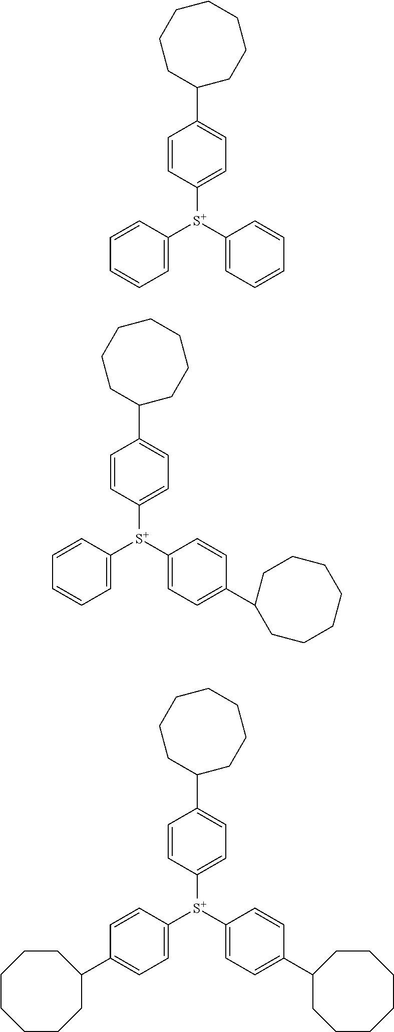 [SMILES: c1ccc([S+](c2ccccc2)c2ccc(C3CCCCCCC3)cc2)cc1 ; c1ccc([S+](c2ccc(C3CCCCCCC3)cc2)c2ccc(C3CCCCCCC3)cc2)cc1 ; c1cc([S+](c2ccc(C3CCCCCCC3)cc2)c2ccc(C3CCCCCCC3)cc2)ccc1C1CCCCCCC1]

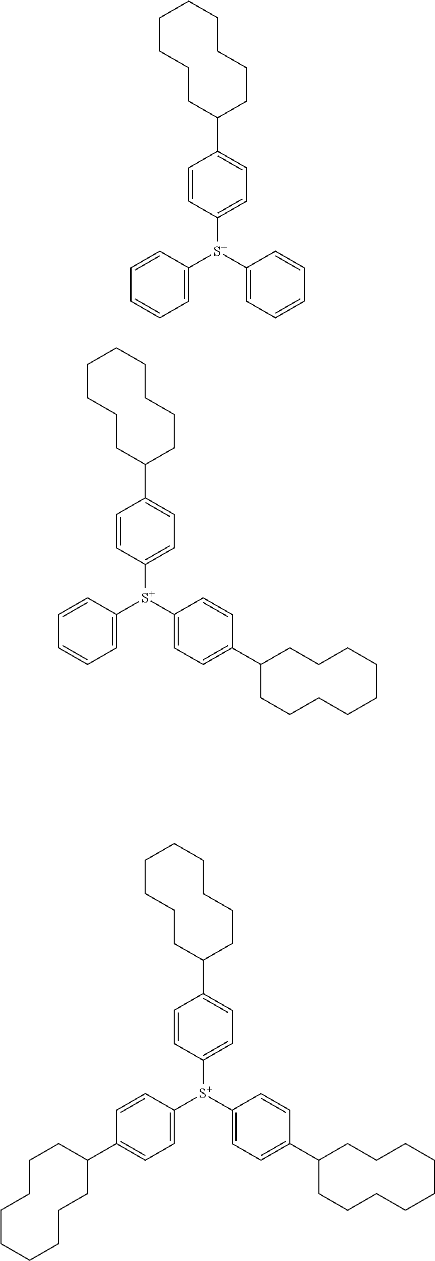 [SMILES: c1ccc([S+](c2ccccc2)c2ccc(C3CCCCCCCCC3)cc2)cc1 ; c1ccc([S+](c2ccc(C3CCCCCCCCC3)cc2)c2ccc(C3CCCCCCCCC3)cc2)cc1 ; c1cc([S+](c2ccc(C3CCCCCCCCC3)cc2)c2ccc(C3CCCCCCCCC3)cc2)ccc1C1CCCCCCCCC1]

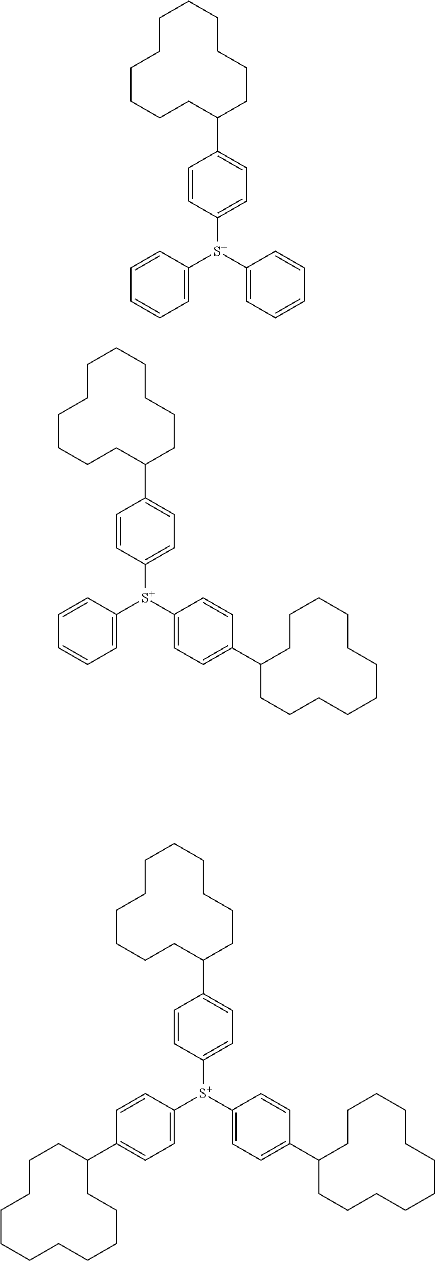 [SMILES: c1ccc([S+](c2ccccc2)c2ccc(C3CCCCCCCCCCC3)cc2)cc1 ; c1ccc([S+](c2ccc(C3CCCCCCCCCCC3)cc2)c2ccc(C3CCCCCCCCCCC3)cc2)cc1 ; c1cc([S+](c2ccc(C3CCCCCCCCCCC3)cc2)c2ccc(C3CCCCCCCCCCC3)cc2)ccc1C1CCCCCCCCCCC1]

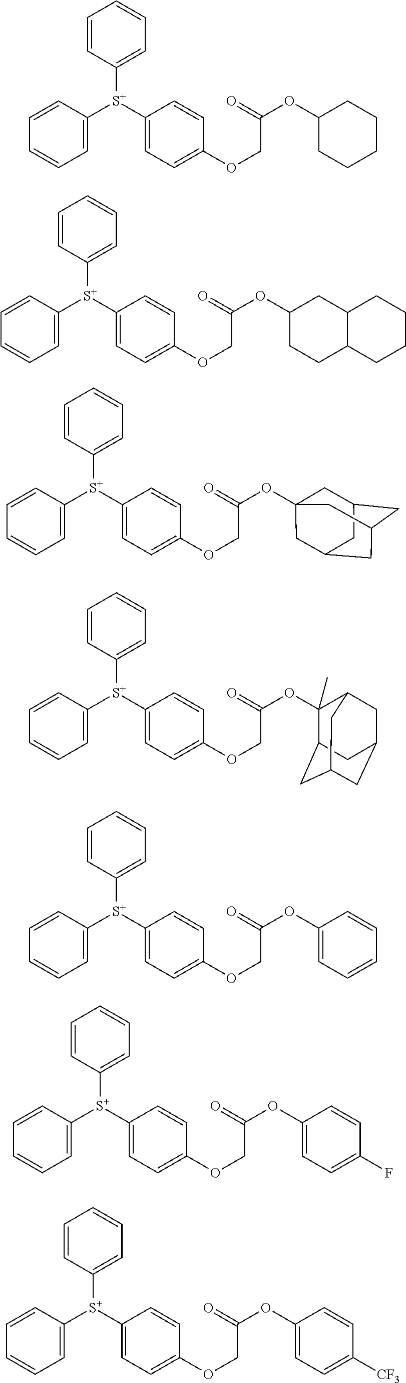

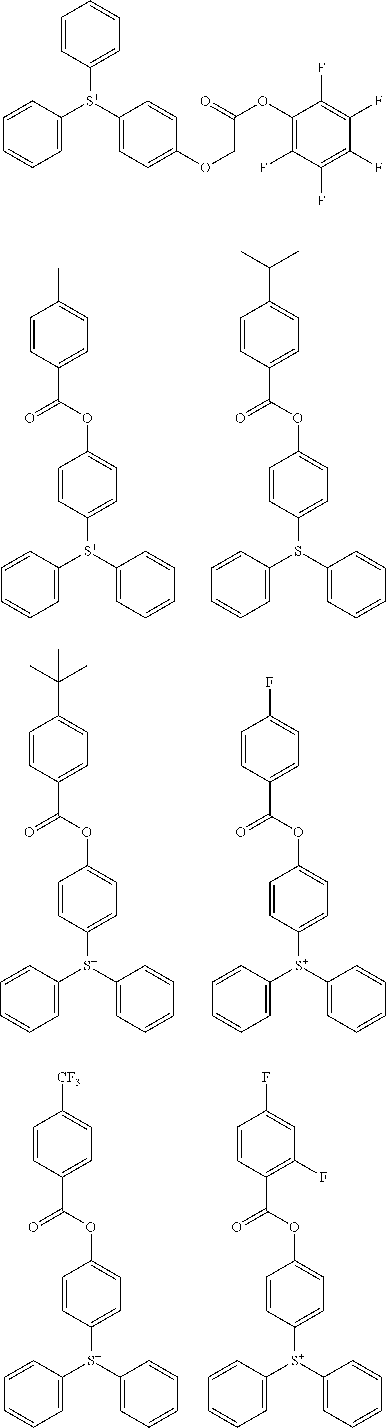

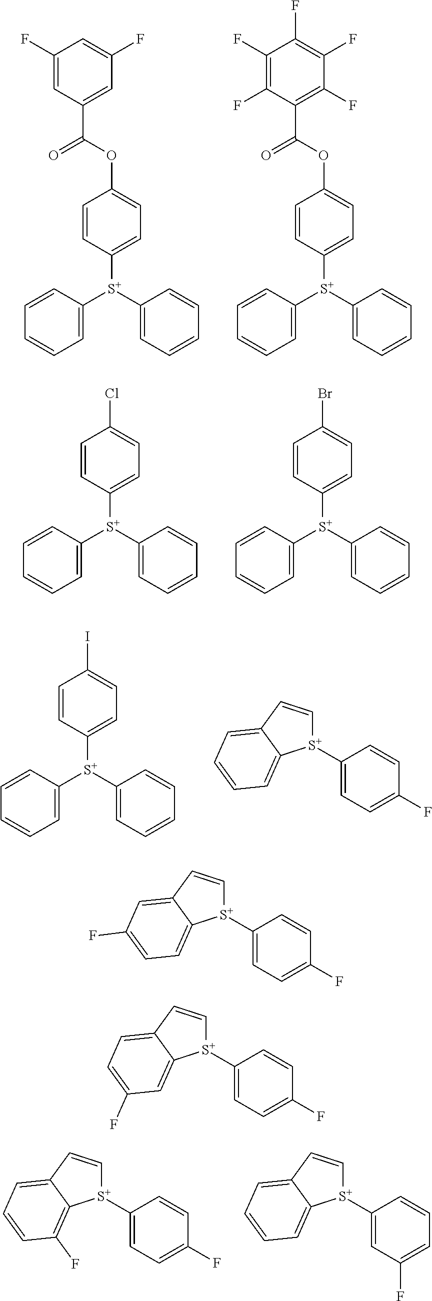

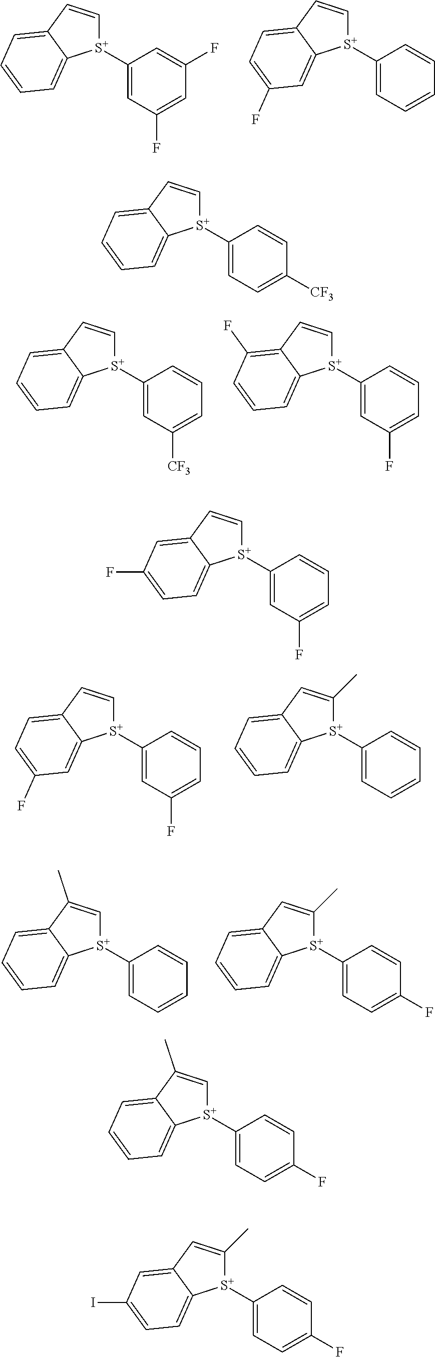

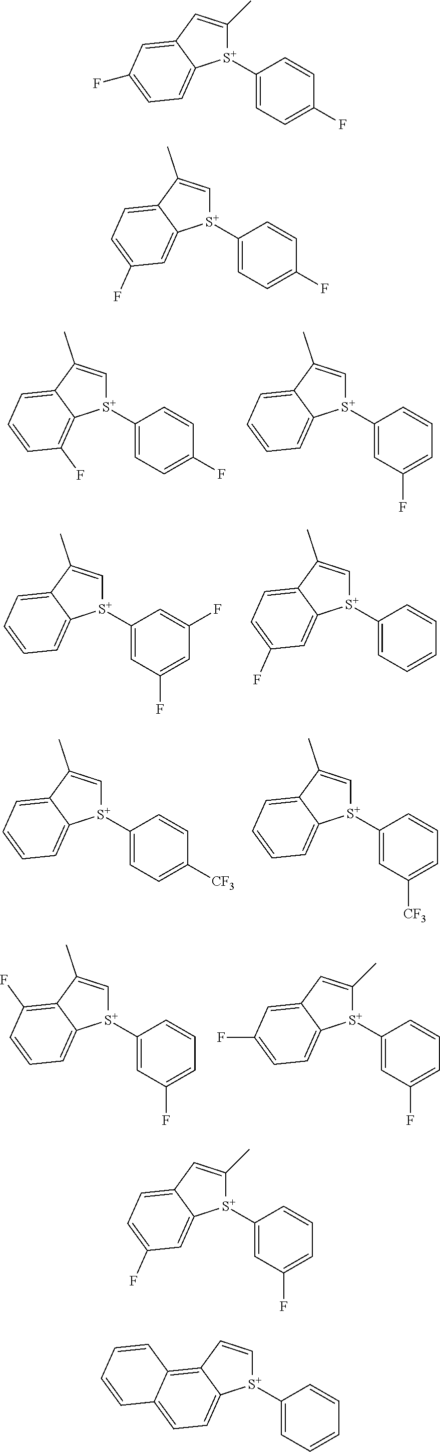

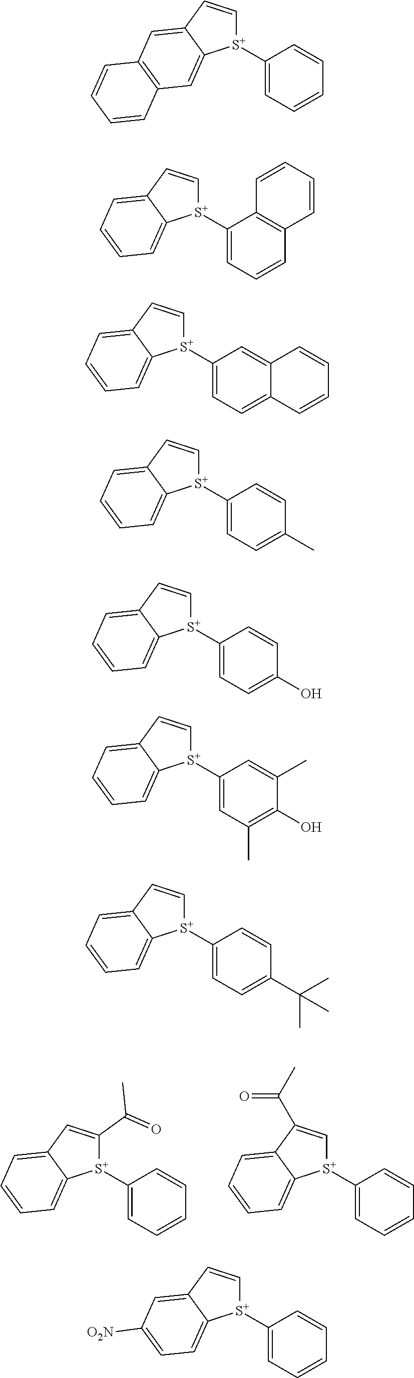

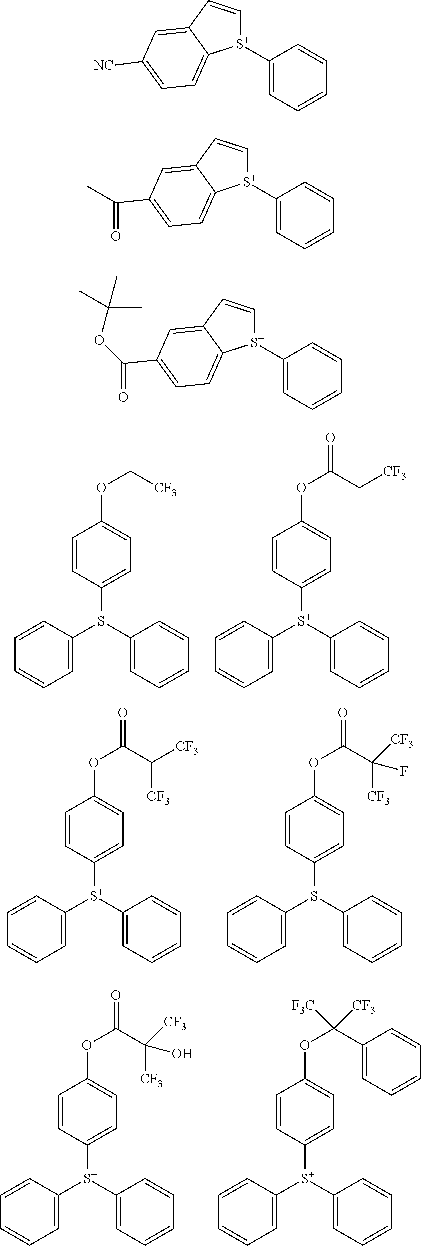

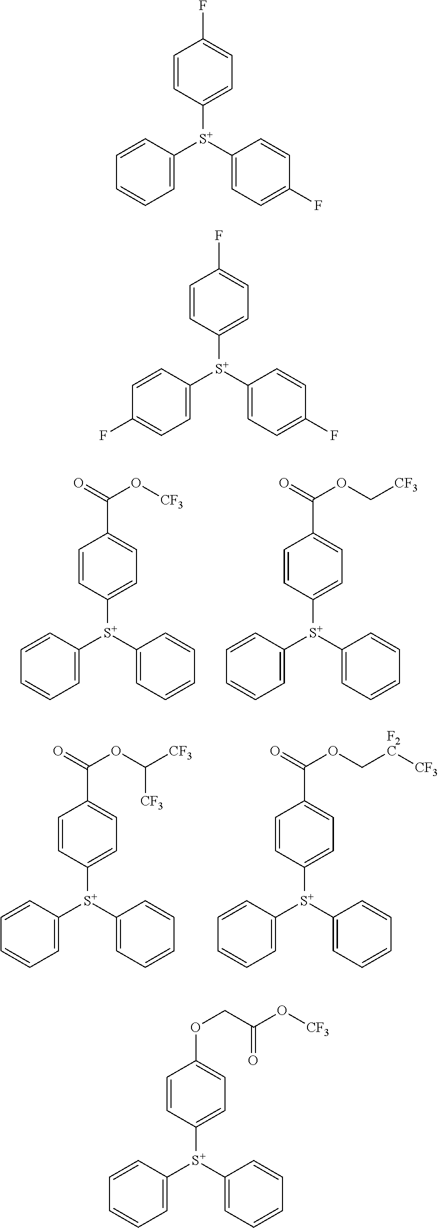

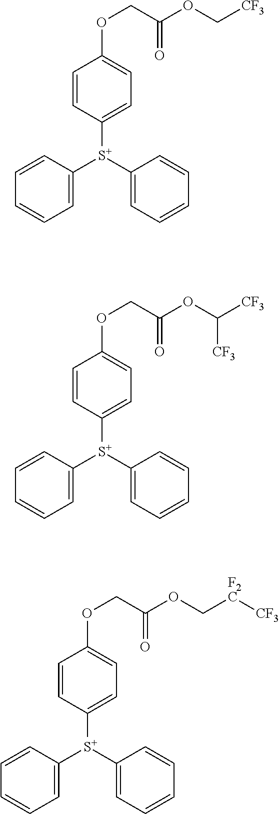 [SMILES: O=C(COc1ccc([S+](c2ccccc2)c2ccccc2)cc1)OCC(F)(F)F ; O=C(COc1ccc([S+](c2ccccc2)c2ccccc2)cc1)OC(C(F)(F)F)C(F)(F)F ; O=C(COc1ccc([S+](c2ccccc2)c2ccccc2)cc1)OCC(F)(F)C(F)(F)F]

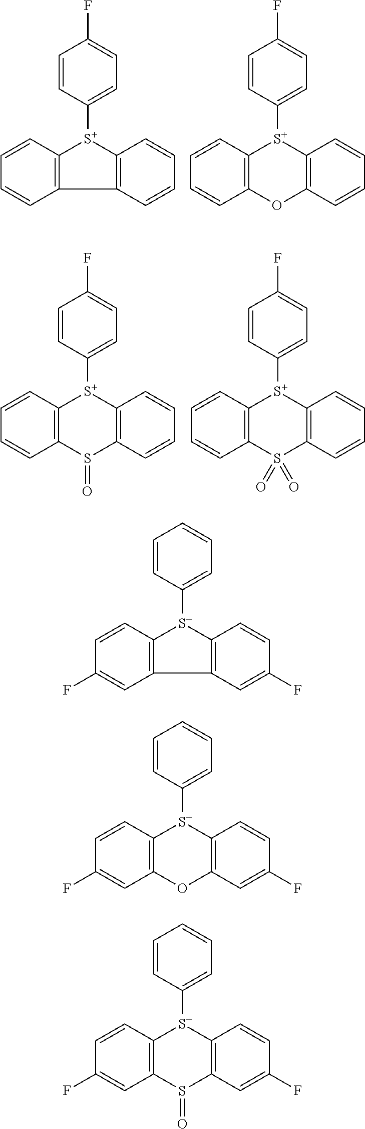

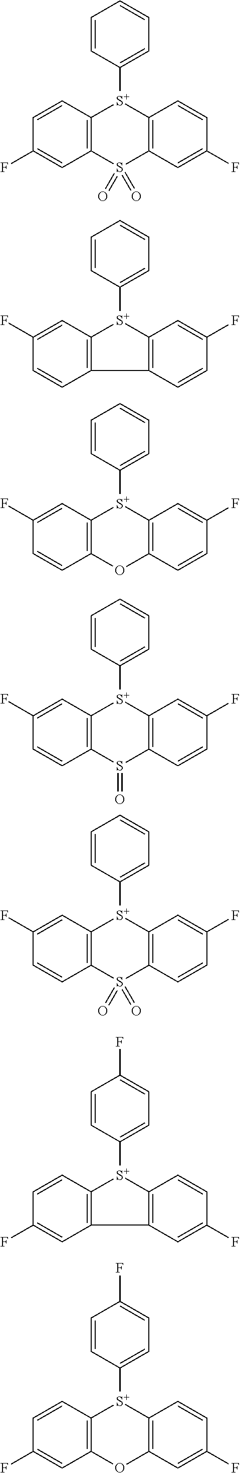

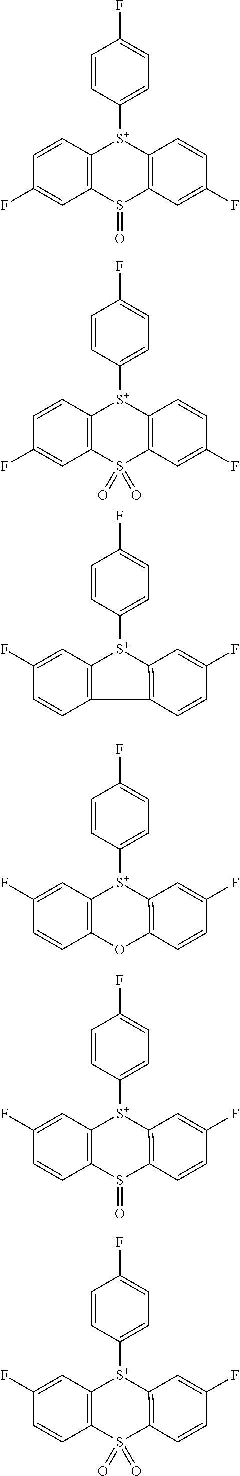 [SMILES: O=S1c2cc(F)ccc2[S+](c2ccc(F)cc2)c2ccc(F)cc21 ; O=S1(=O)c2cc(F)ccc2[S+](c2ccc(F)cc2)c2ccc(F)cc21 ; Fc1ccc(-[s+]2c3cc(F)ccc3c3ccc(F)cc32)cc1 ; Fc1ccc([S+]2c3cc(F)ccc3Oc3ccc(F)cc32)cc1 ; O=S1c2ccc(F)cc2[S+](c2ccc(F)cc2)c2cc(F)ccc21 ; O=S1(=O)c2ccc(F)cc2[S+](c2ccc(F)cc2)c2cc(F)ccc21]

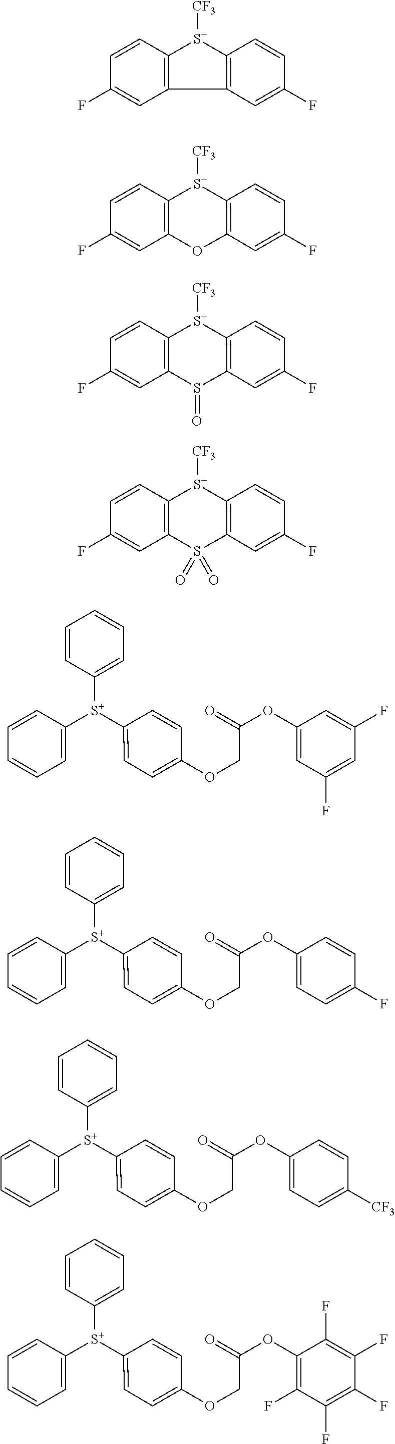

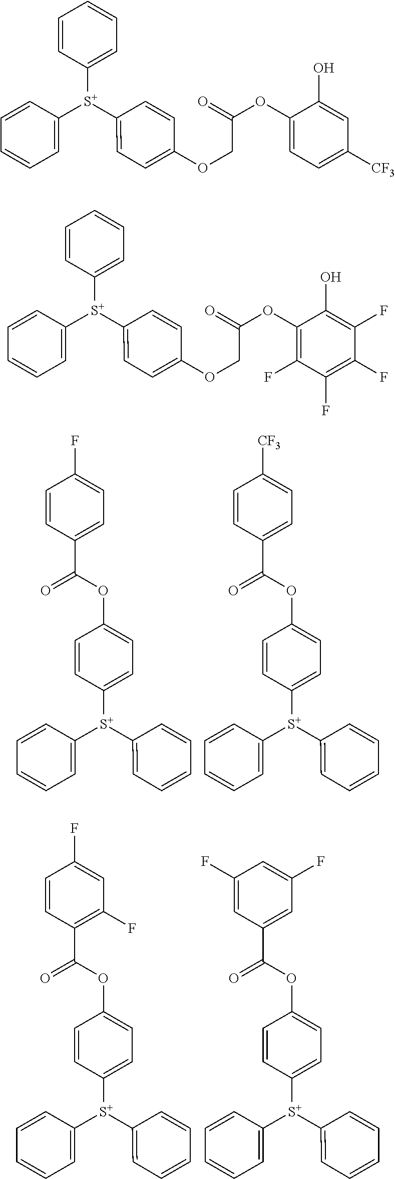 [SMILES: O=C(COc1ccc([S+](c2ccccc2)c2ccccc2)cc1)Oc1ccc(C(F)(F)F)cc1O ; O=C(COc1ccc([S+](c2ccccc2)c2ccccc2)cc1)Oc1c(O)c(F)c(F)c(F)c1F ; O=C(Oc1ccc([S+](c2ccccc2)c2ccccc2)cc1)c1ccc(F)cc1 ; O=C(Oc1ccc([S+](c2ccccc2)c2ccccc2)cc1)c1ccc(C(F)(F)F)cc1 ; O=C(Oc1ccc([S+](c2ccccc2)c2ccccc2)cc1)c1ccc(F)cc1F ; O=C(Oc1ccc([S+](c2ccccc2)c2ccccc2)cc1)c1cc(F)cc(F)c1]

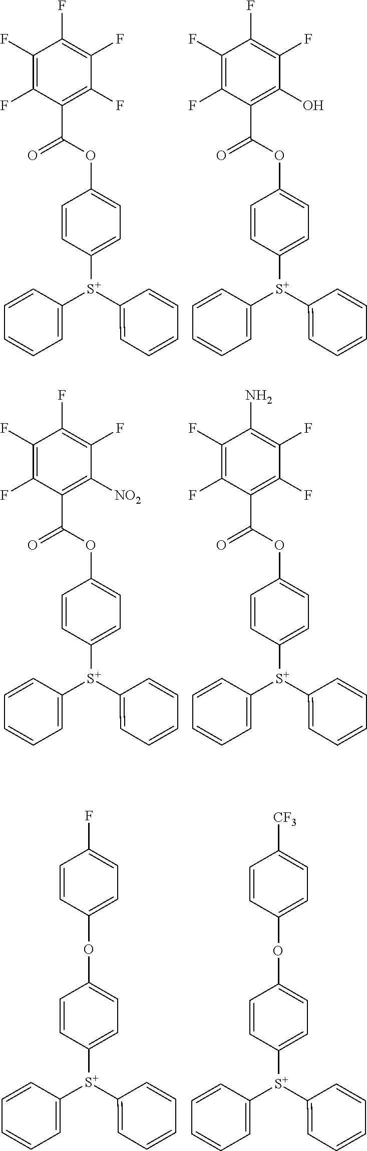 [SMILES: O=C(Oc1ccc([S+](c2ccccc2)c2ccccc2)cc1)c1c(F)c(F)c(F)c(F)c1F ; O=C(Oc1ccc([S+](c2ccccc2)c2ccccc2)cc1)c1c(O)c(F)c(F)c(F)c1F ; O=C(Oc1ccc([S+](c2ccccc2)c2ccccc2)cc1)c1c(F)c(F)c(F)c(F)c1[N+](=O)[O-] ; Nc1c(F)c(F)c(C(=O)Oc2ccc([S+](c3ccccc3)c3ccccc3)cc2)c(F)c1F ; Fc1ccc(Oc2ccc([S+](c3ccccc3)c3ccccc3)cc2)cc1 ; FC(F)(F)c1ccc(Oc2ccc([S+](c3ccccc3)c3ccccc3)cc2)cc1]

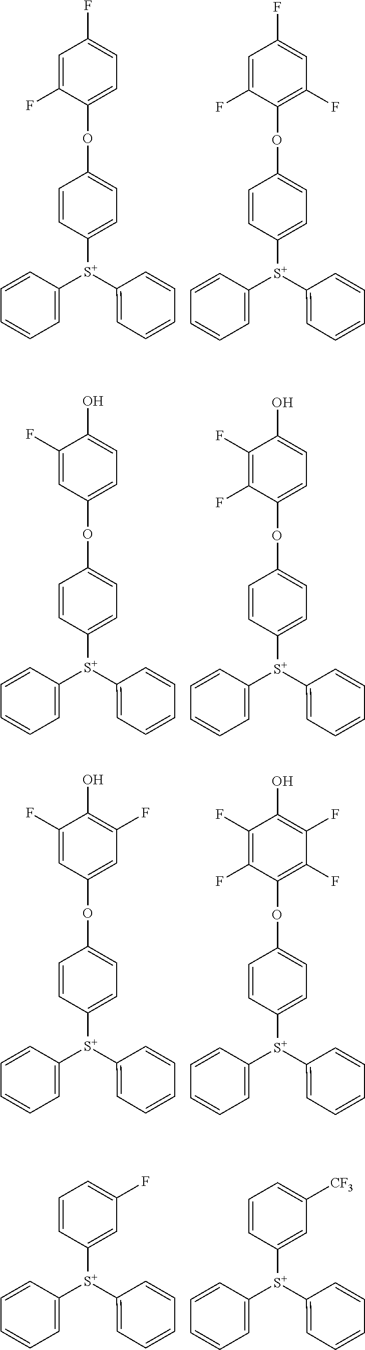

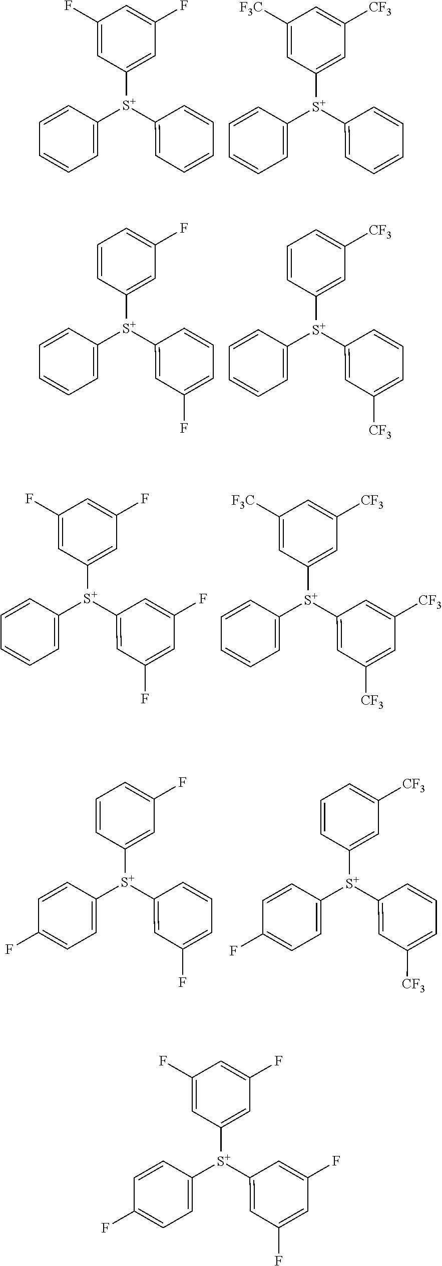

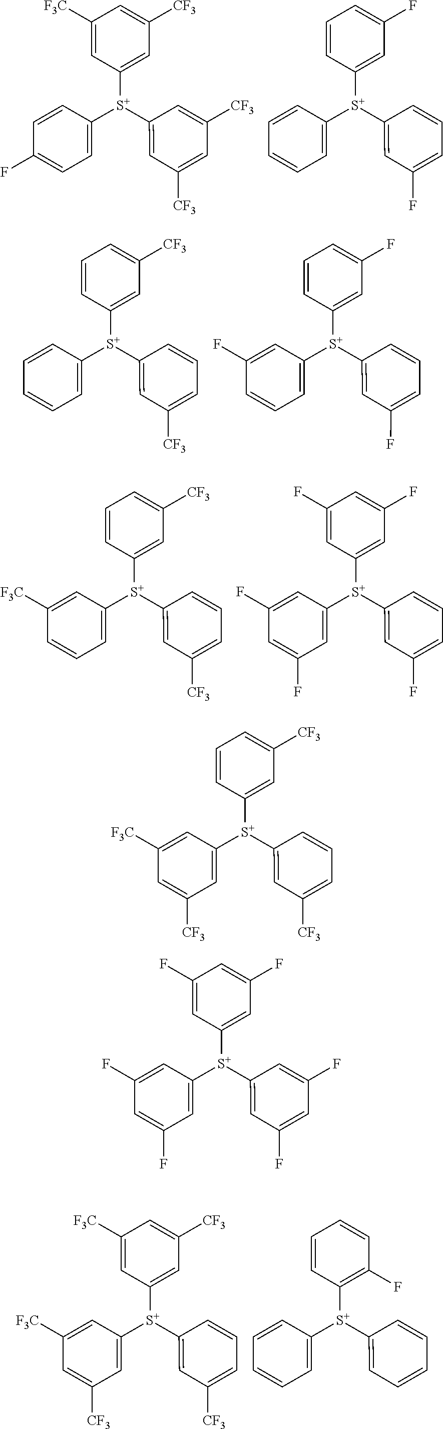

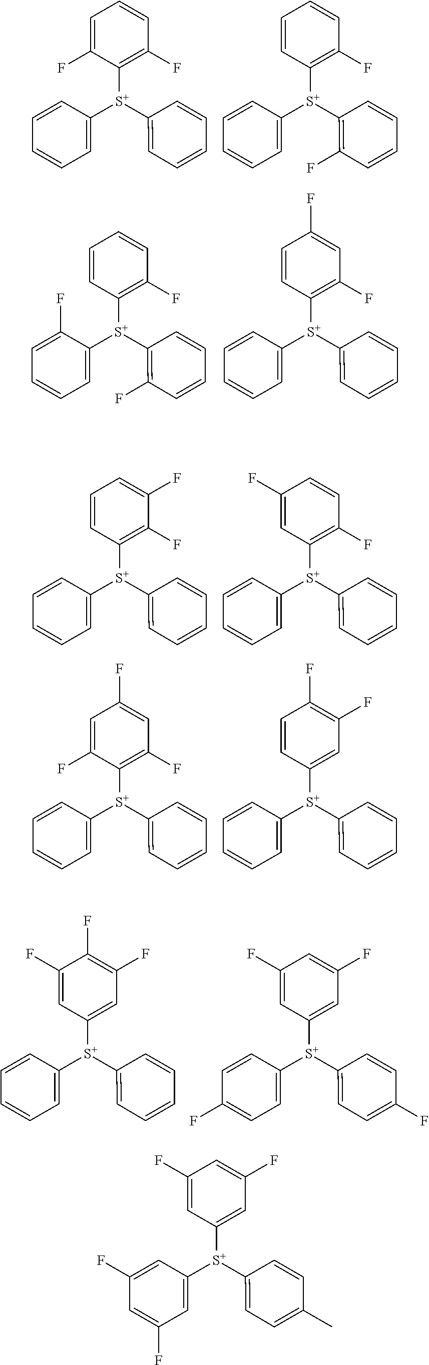

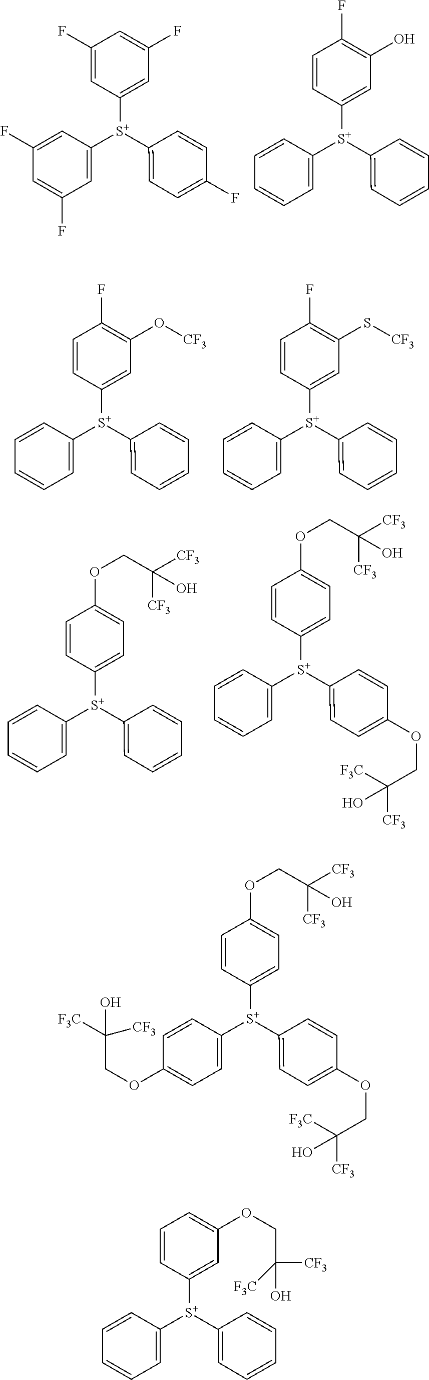

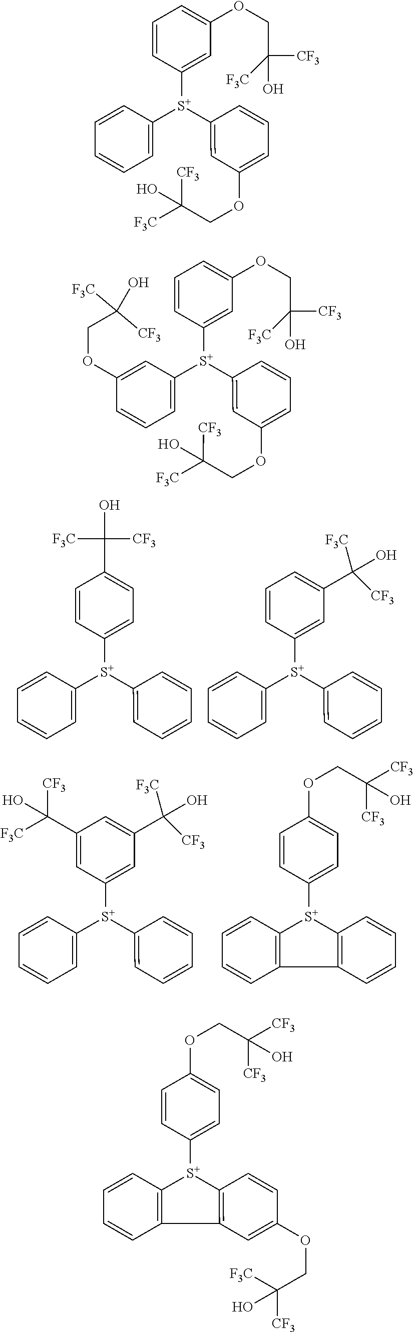

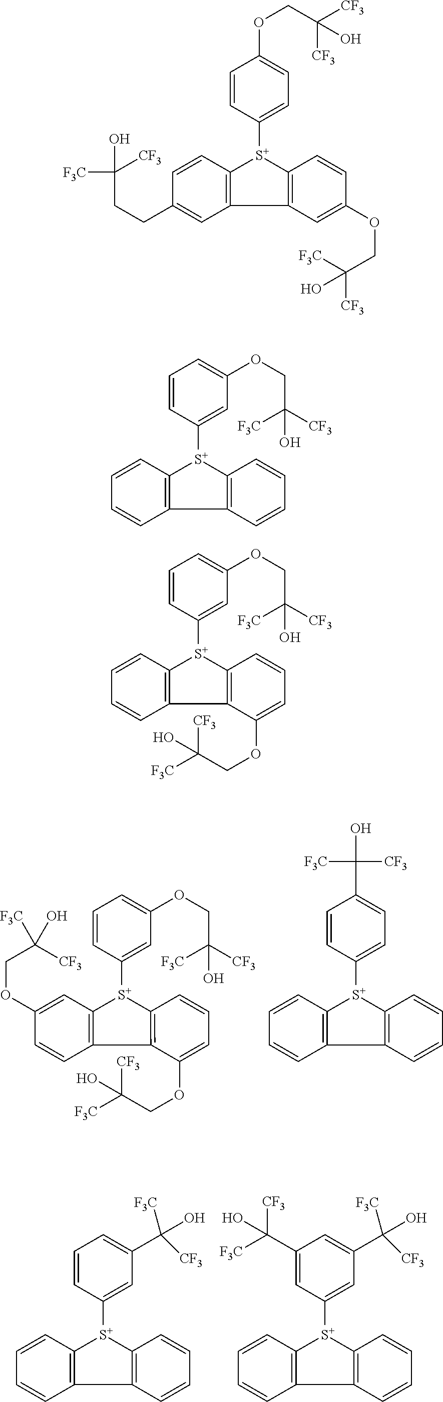

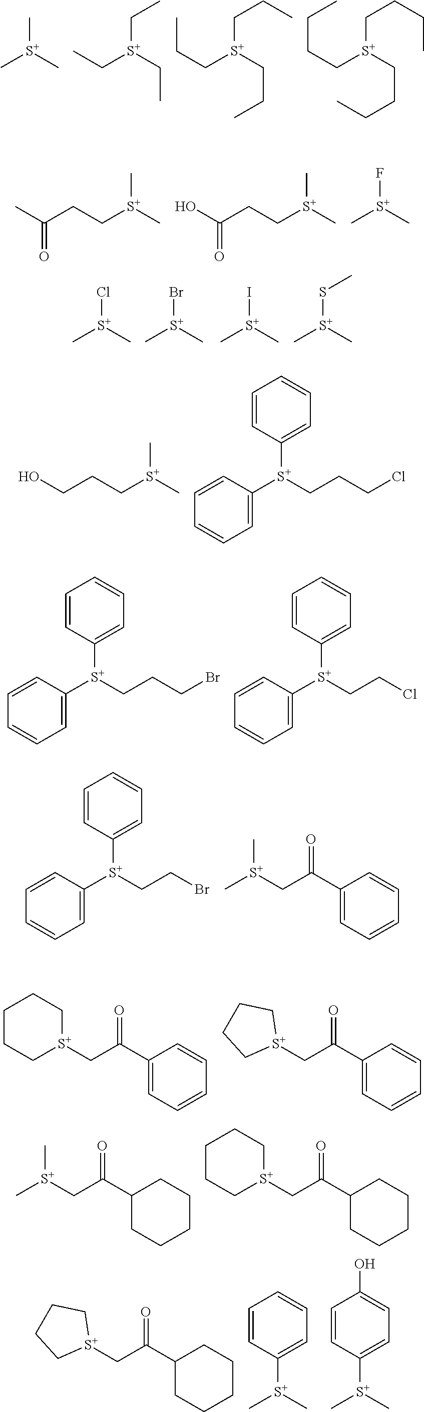

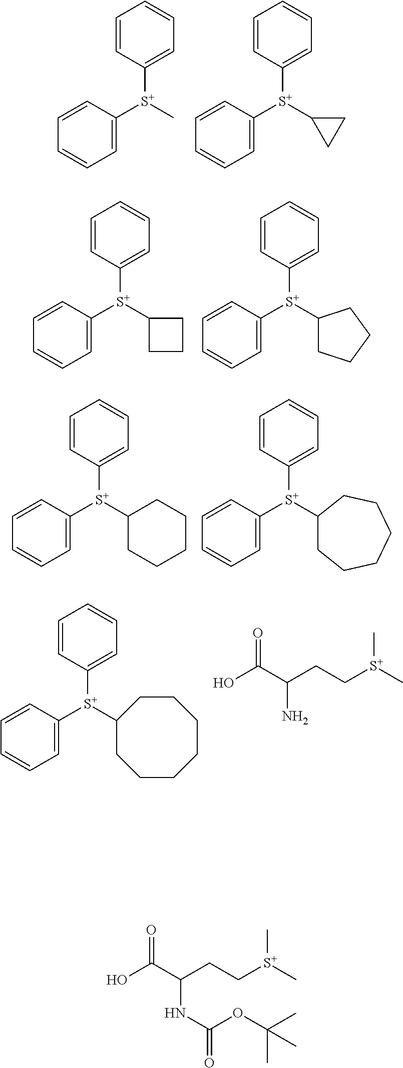

The cations having formula (1) are also useful as the cation in the sulfonium salt having formula (3-1).

Examples of the cation in the iodonium salt having formula (3-2) are shown below, but not limited thereto.

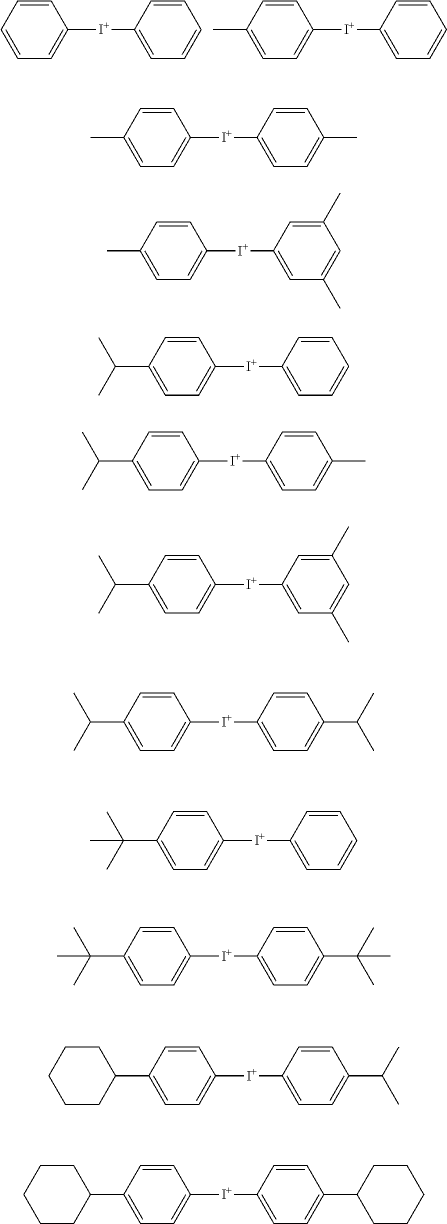

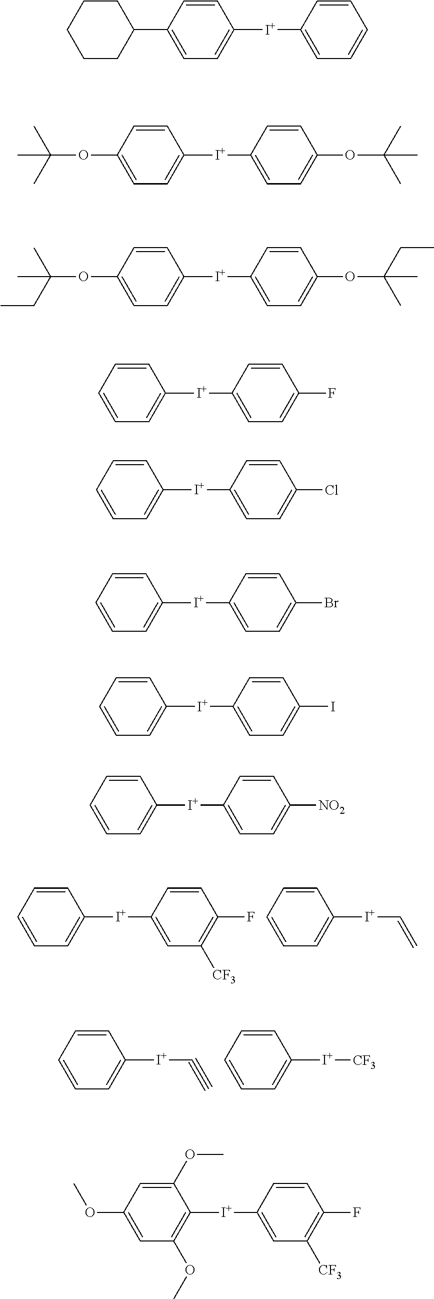

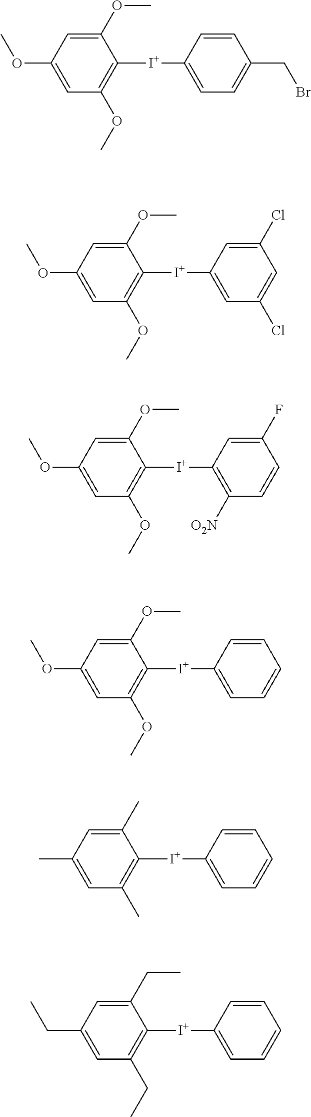 [SMILES: COc1cc(OC)c([I+]c2ccc(CBr)cc2)c(OC)c1 ; COc1cc(OC)c([I+]c2cc(Cl)cc(Cl)c2)c(OC)c1 ; COc1cc(OC)c([I+]c2cc(F)ccc2[N+](=O)[O-])c(OC)c1 ; COc1cc(OC)c([I+]c2ccccc2)c(OC)c1 ; Cc1cc(C)c([I+]c2ccccc2)c(C)c1 ; CCc1cc(CC)c([I+]c2ccccc2)c(CC)c1]

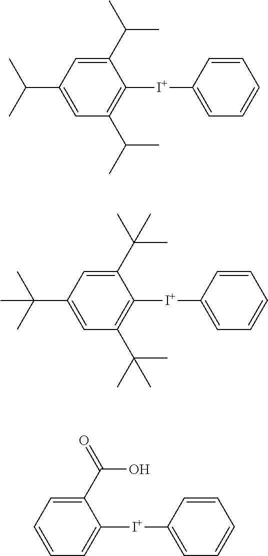 [SMILES: CC(C)c1cc(C(C)C)c([I+]c2ccccc2)c(C(C)C)c1 ; CC(C)(C)c1cc(C(C)(C)C)c([I+]c2ccccc2)c(C(C)(C)C)c1 ; O=C(O)c1ccccc1[I+]c1ccccc1]

In formulae (3-1) and (3-2), Xa⁻ is an anion of the following formula (3A), (3B), (3C) or (3D).

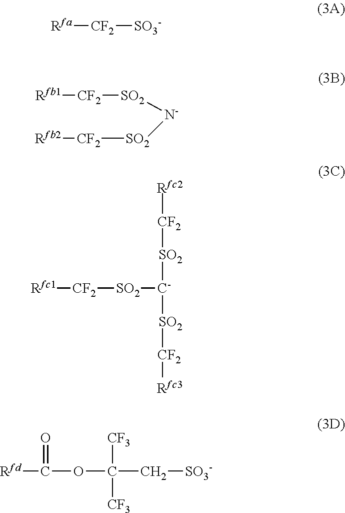

In formula (3A), R f a is fluorine or a C₁-C₄₀ hydrocarbyl group which may contain a heteroatom. The hydrocarbyl group may be saturated or unsaturated and straight, branched or cyclic. Examples thereof are as will be exemplified later for hydrocarbyl group R^{fa1} in formula (3A′).

Of the anions of formula (3A), a structure having formula (3A′) is preferred.

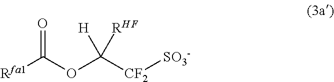

In formula (3A′), R^{HF} is hydrogen or trifluoromethyl, preferably trifluoromethyl.

R^{fa1} is a C₁-C₃₈ hydrocarbyl group which may contain a heteroatom. Suitable heteroatoms include oxygen, nitrogen, sulfur and halogen, with oxygen being preferred. Of the hydrocarbyl groups, those of 6 to 30 carbon atoms are preferred because a high resolution is available in fine pattern formation. The hydrocarbyl group R^{fa1} may be saturated or unsaturated and straight, branched or cyclic. Suitable hydrocarbyl groups include C₁-C₃₈ alkyl groups such as methyl, ethyl, n-propyl, isopropyl, n-butyl, isobutyl, sec-butyl, tert-butyl, pentyl, neopentyl, hexyl, heptyl, 2-ethylhexyl, nonyl, undecyl, tridecyl, pentadecyl, heptadecyl, icosyl; C₃-C₃₈ cyclic saturated hydrocarbyl groups such as cyclopentyl, cyclohexyl, 1-adamantyl, 2-adamantyl, 1-adamantylmethyl, norbornyl, norbornylmethyl, tricyclodecyl, tetracyclododecyl, tetracyclododecylmethyl, and dicyclohexylmethyl; C₂-C₃₈ unsaturated aliphatic hydrocarbyl groups such as allyl and 3-cyclohexenyl; C₆-C₃₈ aryl groups such as phenyl, 1-naphthyl, 2-naphthyl; C₇-C₃₈ aralkyl groups such as benzyl and diphenylmethyl; and combinations thereof.

In the hydrocarbyl group, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonic ester bond, carbonate bond, lactone ring, sultone ring, carboxylic anhydride (—C(═O)—O—C(═O)—) or haloalkyl moiety. Examples of the heteroatom-containing hydrocarbyl group include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl, and 3-oxocyclohexyl.

With respect to the synthesis of the sulfonium salt having an anion of formula (3A′), reference is made to JP-A 2007-145797, JP-A 2008-106045, JP-A 2009-007327, and JP-A 2009-258695. Also useful are the sulfonium salts described in JP-A 2010-215608, JP-A 2012-041320, JP-A 2012-106986, and JP-A 2012-153644.

Examples of the anion having formula (3A) are as exemplified for the anion having formula (1A) in US 20180335696 (JP-A 2018-197853).

In formula (3B), R^{fb1} and R^{fb2} are each independently fluorine or a C₁-C₄₀ hydrocarbyl group which may contain a heteroatom. The hydrocarbyl group may be saturated or unsaturated and straight, branched or cyclic. Suitable hydrocarbyl groups are as exemplified above for R^{fa1} in formula (3A′). Preferably R^{fb1} and R^{fb2} each are fluorine or a straight C₁-C₄ fluorinated alkyl group. A pair of R^{fb1} and R^{fb2} may bond together to form a ring with the linkage (—CF₂—SO₂—N⁻—SO₂—CF₂—) to which they are attached, and the ring-forming pair R^{fb1}-R^{fb2} is preferably a fluorinated ethylene or fluorinated propylene group.

In formula (3C), R^{fc1}, R^{fc2} and R^{fc3} are each independently fluorine or a C₁-C₄₀ hydrocarbyl group which may contain a heteroatom. The hydrocarbyl group may be saturated or unsaturated and straight, branched or cyclic. Suitable hydrocarbyl groups are as exemplified above for R^{fa1} in formula (3A′). Preferably R^{fc1}, R^{fc2} and R^{fc3} each are fluorine or a straight C₁-C₄ fluorinated alkyl group. A pair of R^{fc1} and R^{fc2} may bond together to form a ring with the linkage (—CF₂—SO₂—C⁻—SO₂—CF₂—) to which they are attached, and the ring-forming pair R^{fc1}-R^{fc2} is preferably a fluorinated ethylene or fluorinated propylene group.

In formula (3D), R^{fd} is a C₁-C₄₀ hydrocarbyl group which may contain a heteroatom. The hydrocarbyl group may be saturated or unsaturated and straight, branched or cyclic. Suitable hydrocarbyl groups are as exemplified above for R^{fa1} in formula (3A′).

With respect to the synthesis of the sulfonium salt having an anion of formula (3D), reference is made to JP-A 2010-215608 and JP-A 2014-133723.

Examples of the anion having formula (3D) are as exemplified for the anion having formula (1D) in US 20180335696 (JP-A 2018-197853).

The compound having the anion of formula (3D) has a sufficient acid strength to cleave acid labile groups in the base polymer because it is free of fluorine at α-position of sulfo group, but has two trifluoromethyl groups at β-position. Thus the compound is a useful PAG.

Also compounds having the formula (4) are useful as the PAG.

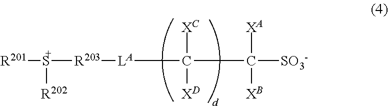

In formula (4), R²⁰¹ and R²⁰² are each independently halogen or a C₁-C₃₀ hydrocarbyl group which may contain a heteroatom. R²⁰³ is a C₁-C³⁰ hydrocarbylene group which may contain a heteroatom. Any two of R²⁰¹, R²⁰² and R²⁰³ may bond together to form a ring with the sulfur atom to which they are attached. Exemplary rings are as described above for the ring that R¹⁰¹ and R¹⁰² in formula (3-1), taken together, form with the sulfur atom to which they are attached.

The hydrocarbyl groups R²⁰¹ and R²⁰² may be saturated or unsaturated and straight, branched or cyclic. Examples thereof include C₁-C₃₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl, and n-decyl; C₃-C₃₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, oxanorbornyl, tricyclo[5.2.1.0^{2,6}]decyl, and adamantyl; C₆-C₃₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl, and anthracenyl; and combinations thereof. In the hydrocarbyl group, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonic ester bond, carbonate bond, lactone ring, sultone ring, carboxylic anhydride (—C(═O)—O—C(═O)—) or haloalkyl moiety.

The hydrocarbylene group R²⁰³ may be saturated or unsaturated and straight, branched or cyclic. Examples thereof include C₁-C₃₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, and heptadecane-1,17-diyl; C₃-C₃₀ cyclic saturated hydrocarbylene groups such as cyclopentanediyl, cyclohexanediyl, norbornanediyl and adamantanediyl; C₆-C₃₀ arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene and tert-butylnaphthylene; and combinations thereof. In the hydrocarbylene group, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonic ester bond, carbonate bond, lactone ring, sultone ring, carboxylic anhydride (—C(═O)—O—C(═O)—) or haloalkyl moiety. Of the heteroatoms, oxygen is preferred.

In formula (4), L A is a single bond, ether bond or a C₁-C₂₀ hydrocarbylene group which may contain a heteroatom. The hydrocarbylene group may be saturated or unsaturated and straight, branched or cyclic. Examples thereof are as exemplified above for R²⁰³.

In formula (4), X^{A}, X^{B}, X^{C} and X^{D} are each independently hydrogen, fluorine or trifluoromethyl, with the proviso that at least one of X^{A}, X^{B}, X^{C} and X^{D} is fluorine or trifluoromethyl.

In formula (4), d is an integer of 0 to 3.

Of the PAGs having formula (4), those having the formula (4′) are preferred.

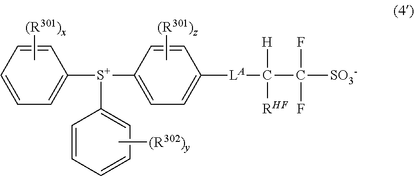

In formula (4′), L^{A} is as defined above. R^{HF} is hydrogen or trifluoromethyl, preferably trifluoromethyl. R³⁰¹, R³⁰² and R³⁰³ are each independently hydrogen or a C₁-C₂₀ hydrocarbyl group which may contain a heteroatom. The hydrocarbyl group may be saturated or unsaturated and straight, branched or cyclic. Examples thereof are as exemplified above for R^{fa1} in formula (3A′). The subscripts x and y are each independently an integer of 0 to 5, and z is an integer of 0 to 4.

Examples of the PAG having formula (4) are as exemplified for the PAG having formula (2) in JP-A 2017-026980.

Of the foregoing PAGs, those having an anion of formula (3A′) or (3D) are especially preferred because of reduced acid diffusion and high solubility in the solvent. Also those having formula (4′) are especially preferred because of extremely reduced acid diffusion.

Also a sulfonium or iodonium salt having an anion containing an iodized or brominated aromatic ring may be used as the PAG. Suitable are sulfonium and iodonium salts having the formulae (5-1) and (5-2).

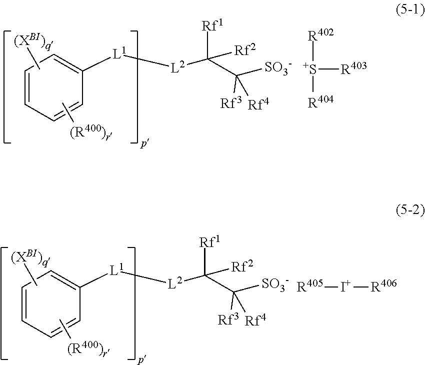

In formulae (5-1) and (5-2), p′ is an integer of 1 to 3, q′ is an integer of 1 to 5, and r′ is an integer of 0 to 3, and 1 q′+r′ 5. Preferably, q′ is 1, 2 or 3, more preferably 2 or 3, and r′ is 0, 1 or 2.

In formulae (5-1) and (5-2), X^{B1} is iodine or bromine, and may be the same or different when p′ and/or q′ is 2 or more.

L¹ is a single bond, ether bond, ester bond, or a C₁-C₆ saturated hydrocarbylene group which may contain an ether bond or ester bond. The saturated hydrocarbylene group may be straight, branched or cyclic.

L² is a single bond or a C₁-C₂₀ divalent linking group when p′ is 1, and a C₁-C₂₀ (p′+1)-valent linking group which may contain oxygen, sulfur or nitrogen when p′ is 2 or 3.

R⁴⁰¹ is a hydroxy group, carboxy group, fluorine, chlorine, bromine, amino group, or a C₁-C₂₀ hydrocarbyl, C₁-C₂₀ hydrocarbyloxy, C₂-Cao hydrocarbylcarbonyl, C₂-C₂₀ hydrocarbyloxycarbonyl, C₂-Cao hydrocarbylcarbonyloxy or C₁-C₂₀ hydrocarbylsulfonyloxy group, which may contain fluorine, chlorine, bromine, hydroxy, amino or ether bond, or —N(R^{401A})(R^{401B}), —N(R^{401C})—C(═O)—R^{401D} or —N(R^{401C}), —C(═O)—O—R^{401D}. R^{401A} and R^{401B} are each independently hydrogen or a C₁-C₆ saturated hydrocarbyl group. R^{401C} is hydrogen or a C₁-C₆ saturated hydrocarbyl group which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety. R^{401D} is a C₁-C₁₆ aliphatic hydrocarbyl group, C₆-C₁₄ aryl group or C₇-C₁₅ aralkyl group, which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety. The aliphatic hydrocarbyl group may be saturated or unsaturated and straight, branched or cyclic. The hydrocarbyl, hydrocarbyloxy, hydrocarbylcarbonyl, hydrocarbyloxycarbonyl, hydrocarbylcarbonyloxy and hydrocarbylsulfonyloxy groups may be straight, branched or cyclic. Groups R⁴⁰¹ may be the same or different when p′ and/or r′ is 2 or more. Of these, R⁴⁰¹ is preferably hydroxy, —N(R^{401C})—C(═O)—R^{401D}, —N(R^{401C})—(═O)—O—R^{401D}, fluorine, chlorine, bromine, methyl or methoxy.

In formulae (5-1) and (5-2), Rf¹ to Rf⁴ are each independently hydrogen, fluorine or trifluoromethyl, at least one of Rf¹ to Rf⁴ is fluorine or trifluoromethyl. Rf¹ and Rf², taken together, may form a carbonyl group. Preferably, both Rf³ and Rf⁴ are fluorine.

R⁴⁰² to R⁴⁰⁶ are each independently halogen or a C₁-C₂₀ hydrocarbyl group which may contain a heteroatom. The hydrocarbyl group may be saturated or unsaturated and straight, branched or cyclic. Examples thereof are as exemplified above for the hydrocarbyl groups R¹⁰¹ to R¹⁰⁵ in formulae (3-1) and (3-2). In the hydrocarbyl group, some or all of the hydrogen atoms may be substituted by hydroxy, carboxy, halogen, cyano, nitro, mercapto, sultone ring, sulfo, or sulfonium salt-containing moiety, and some constituent —CH₂— may be replaced by an ether bond, ester bond, carbonyl moiety, amide bond, carbonate bond or sulfonic ester bond. R⁴⁰² and R⁴⁰³ may bond together to form a ring with the sulfur atom to which they are attached. Exemplary rings are as described above for the ring that R¹⁰¹ and R¹⁰² in formula (3-1), taken together, form with the sulfur atom to which they are attached.

Examples of the cation in the sulfonium salt having formula (5-1) include those exemplified above as the cation in the sulfonium salt having formula (3-1). Examples of the cation in the iodonium salt having formula (5-2) include those exemplified above as the cation in the iodonium salt having formula (3-2).

Examples of the anion in the onium salts having formulae (5-1) and (5-2) are shown below, but not limited thereto. Herein X^{B1} is as defined above.

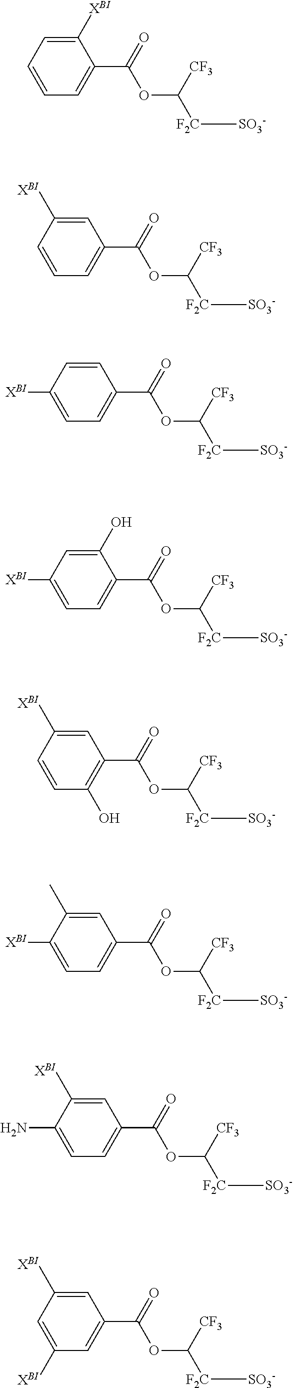

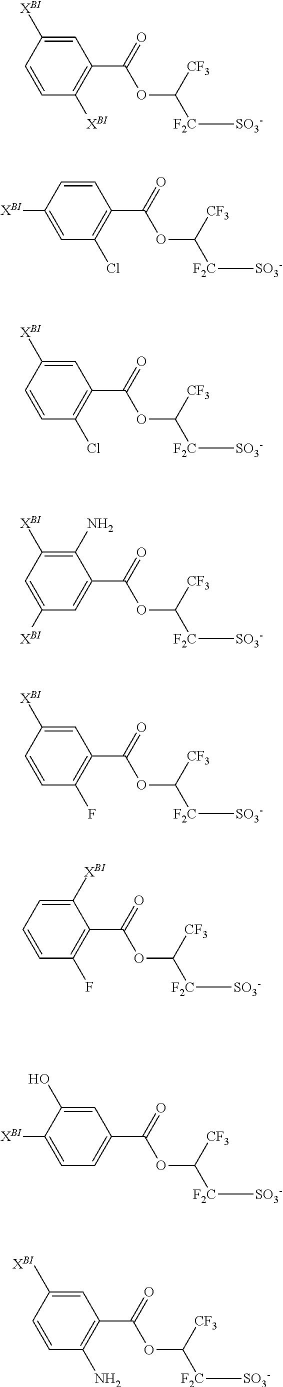

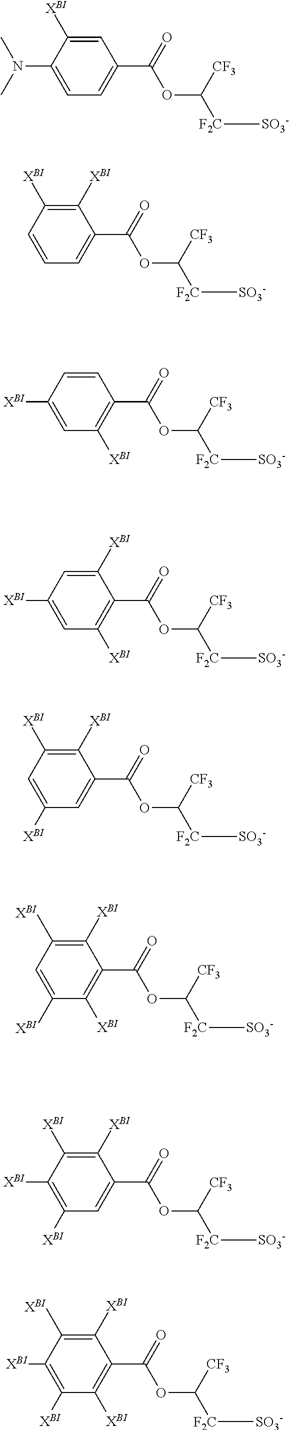

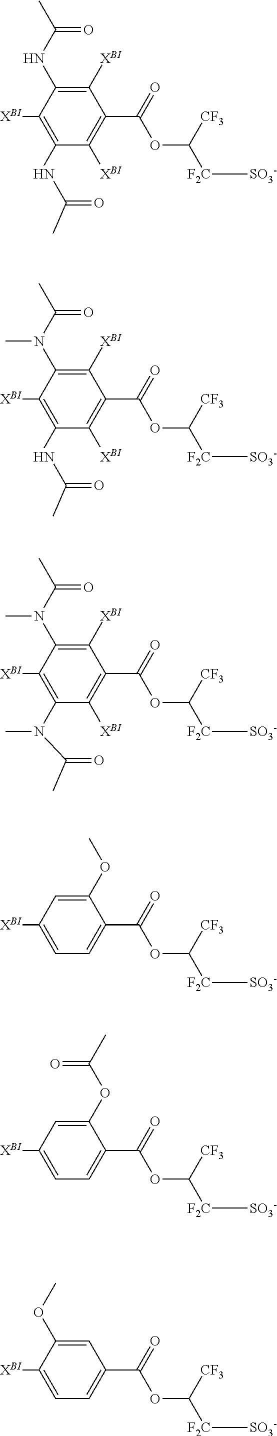

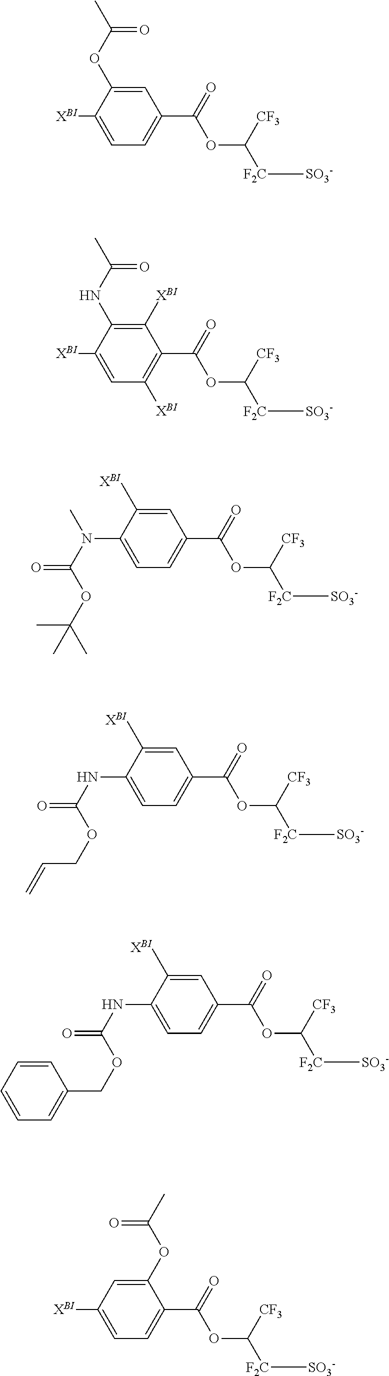

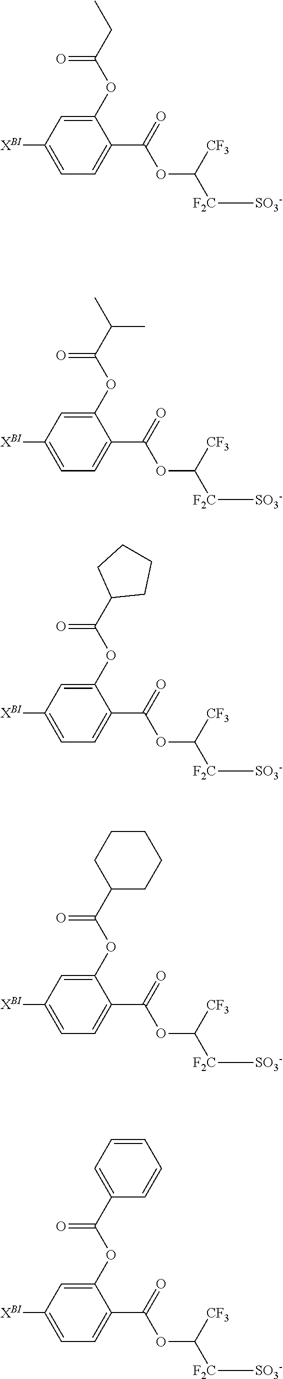

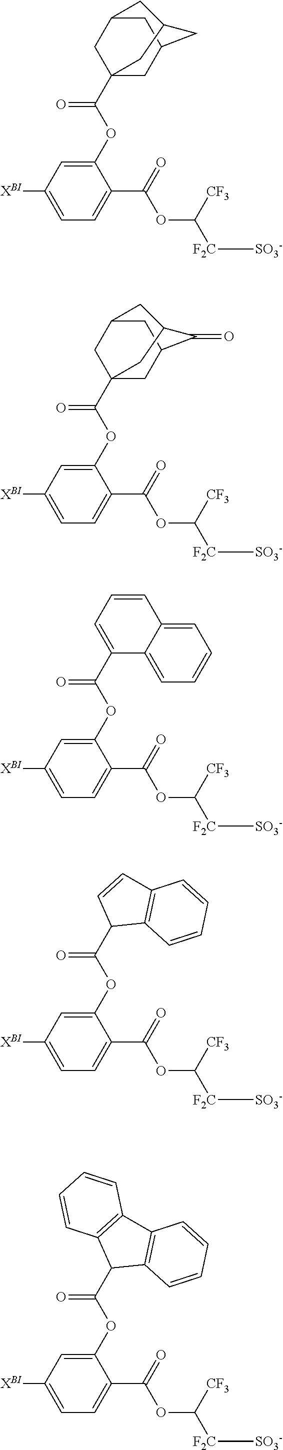

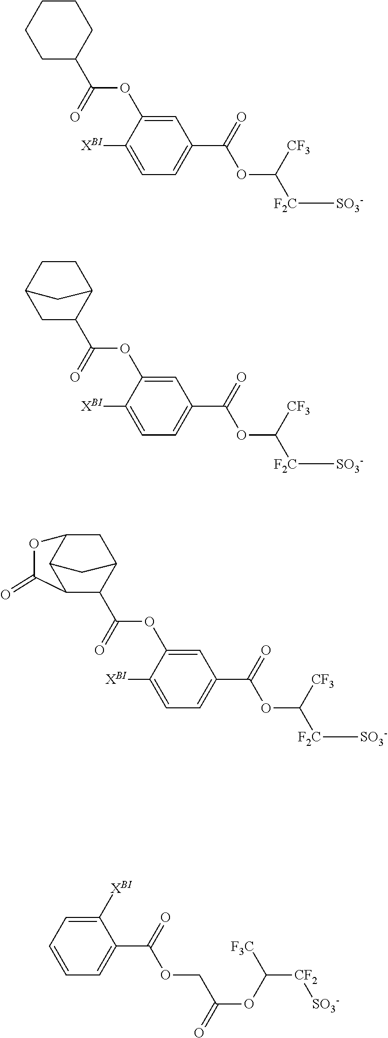

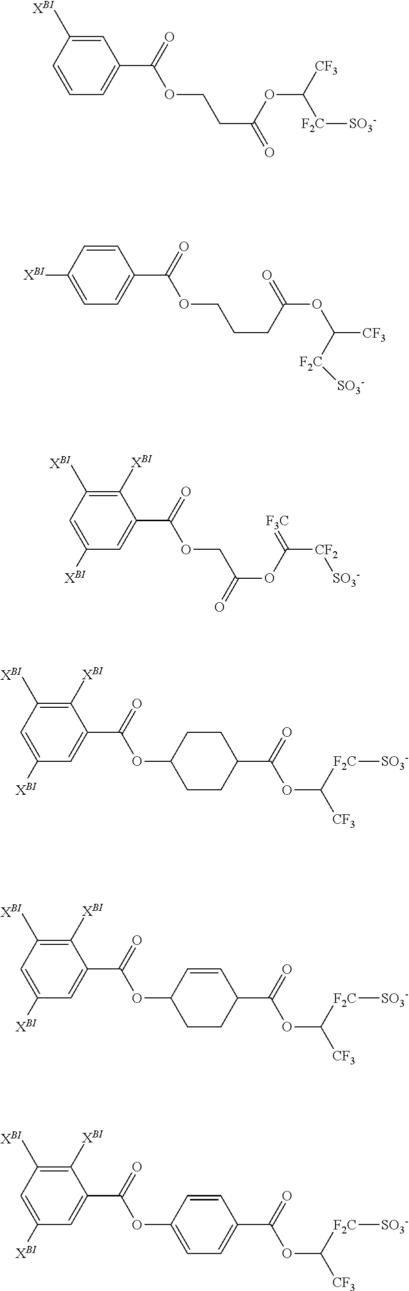

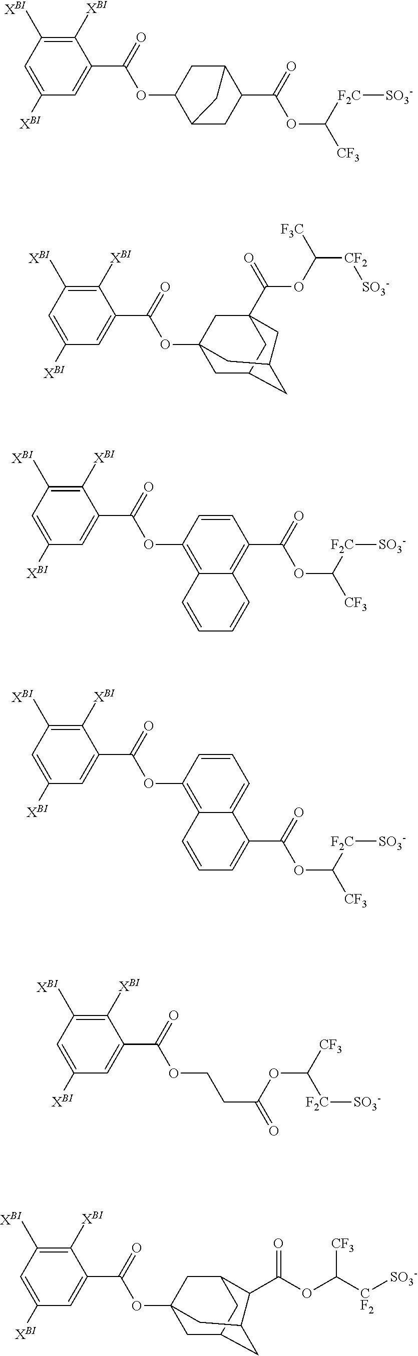

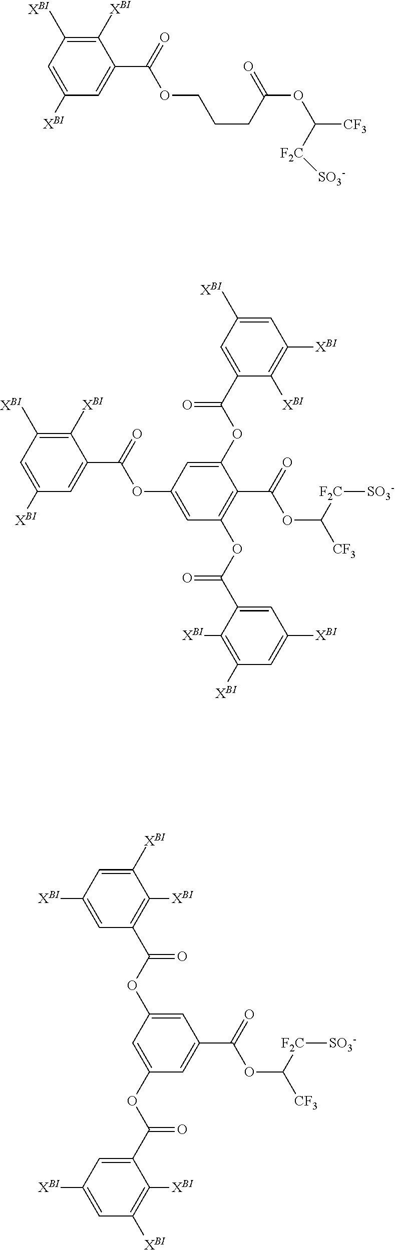

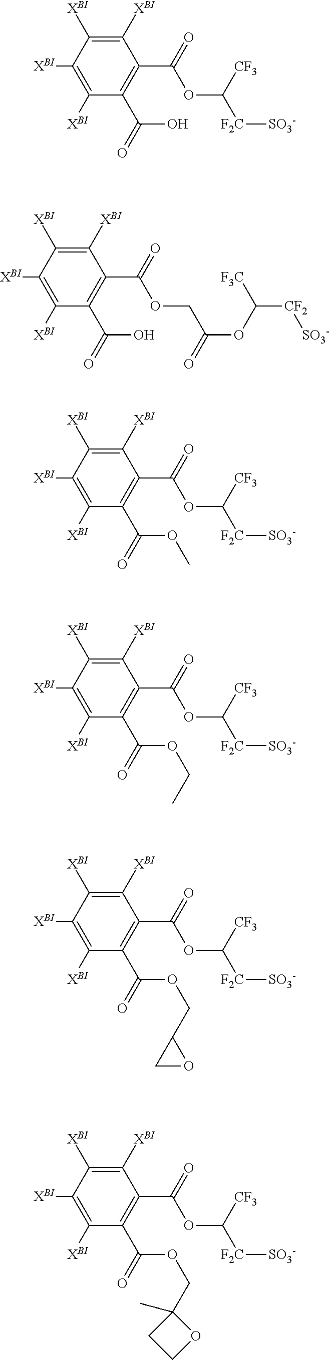

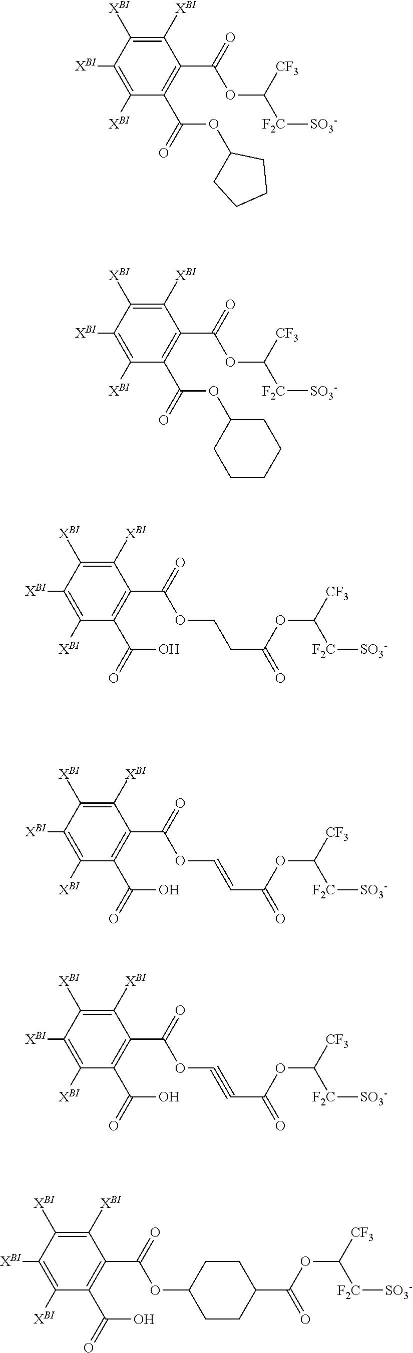

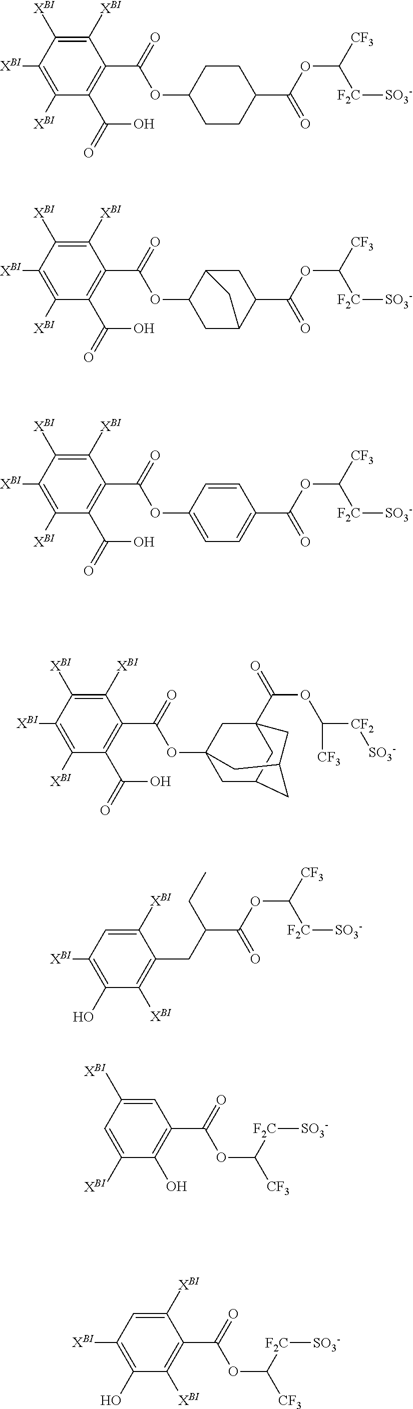

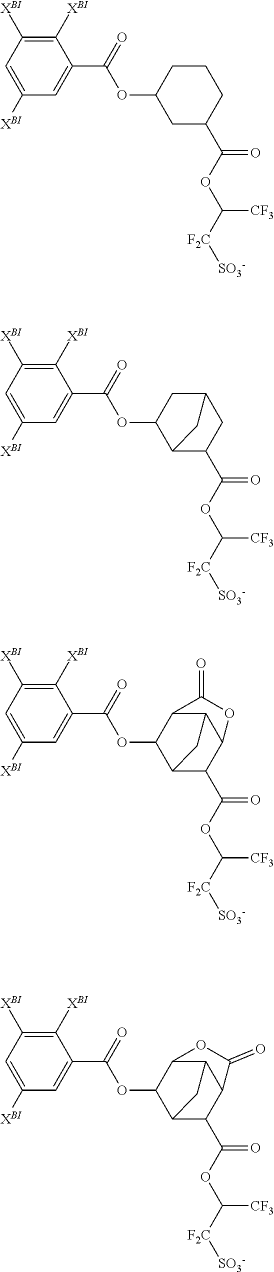

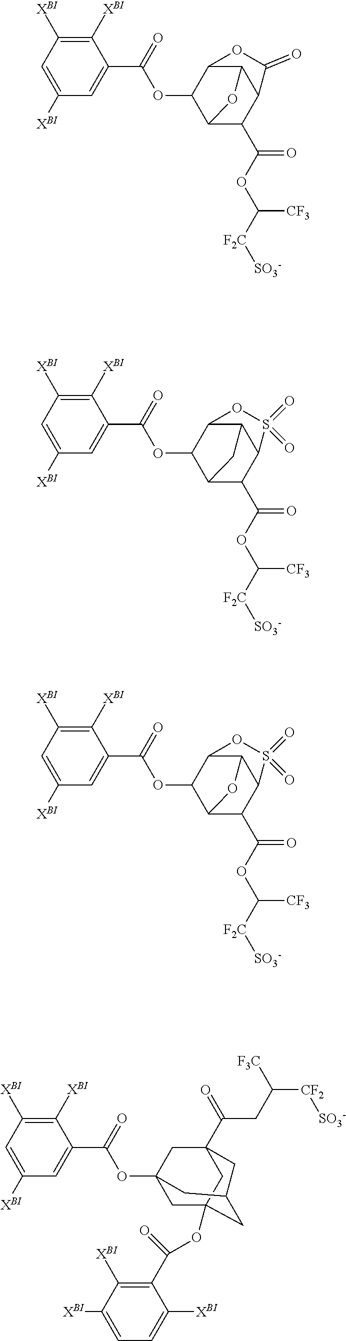

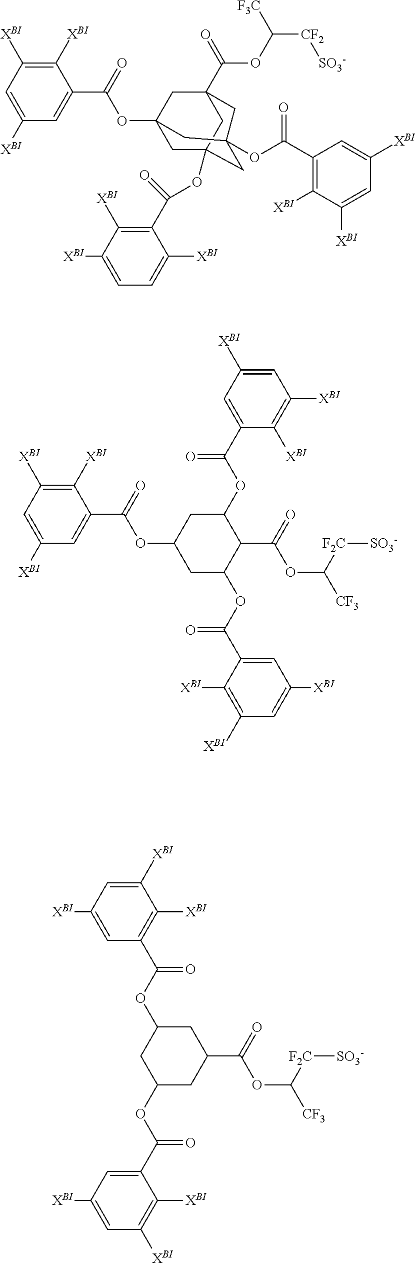

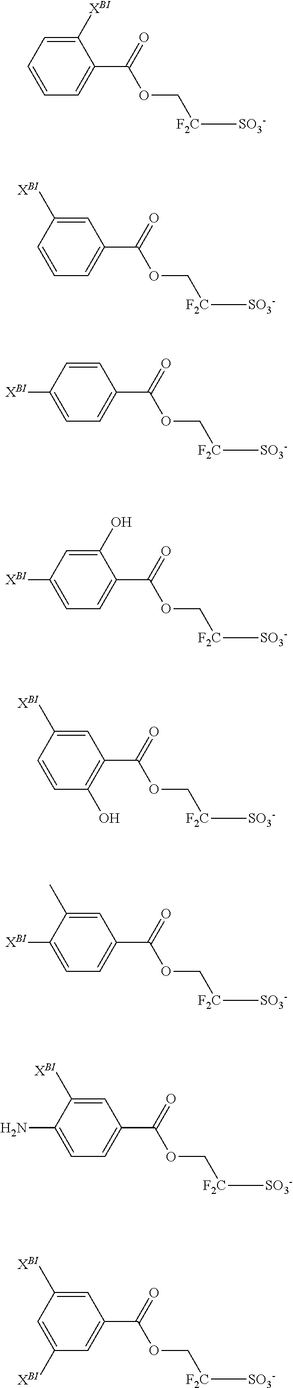

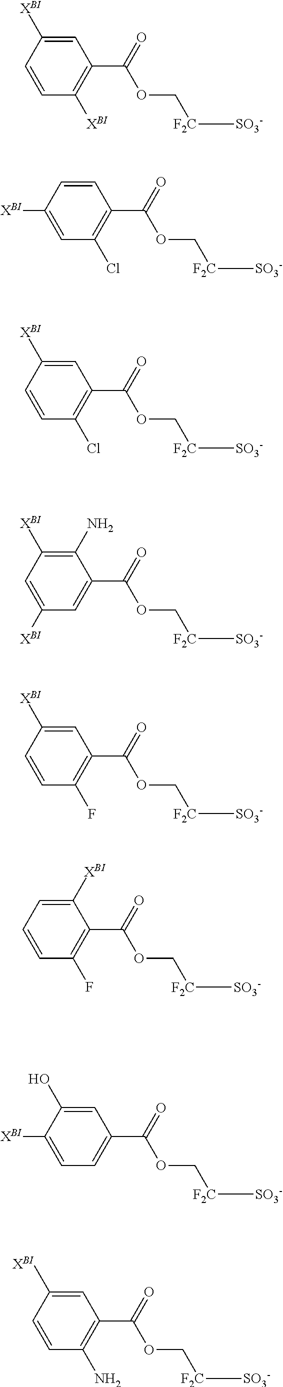

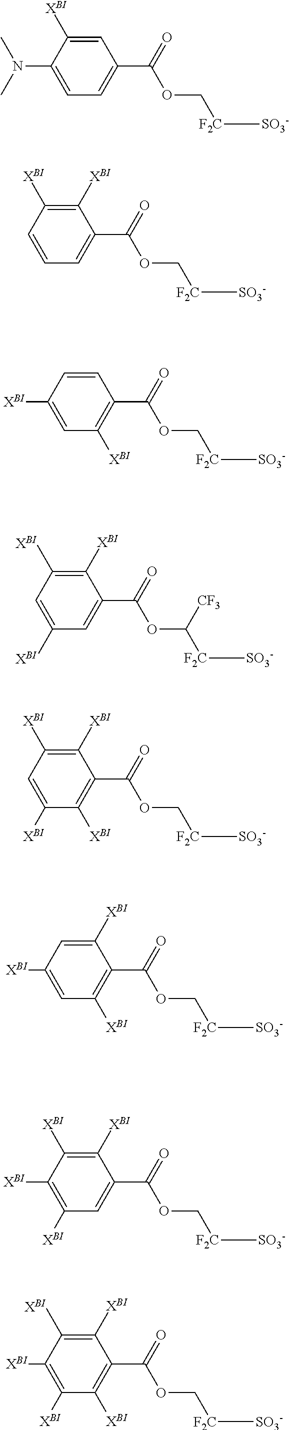

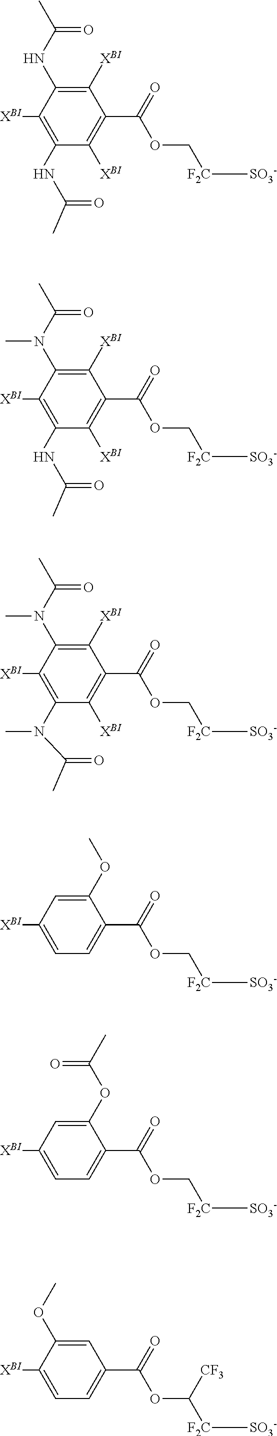

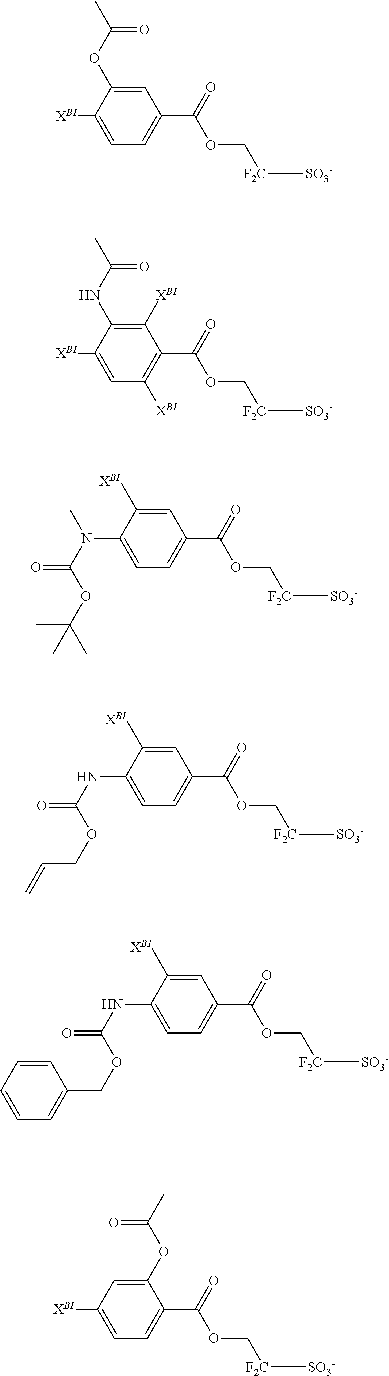

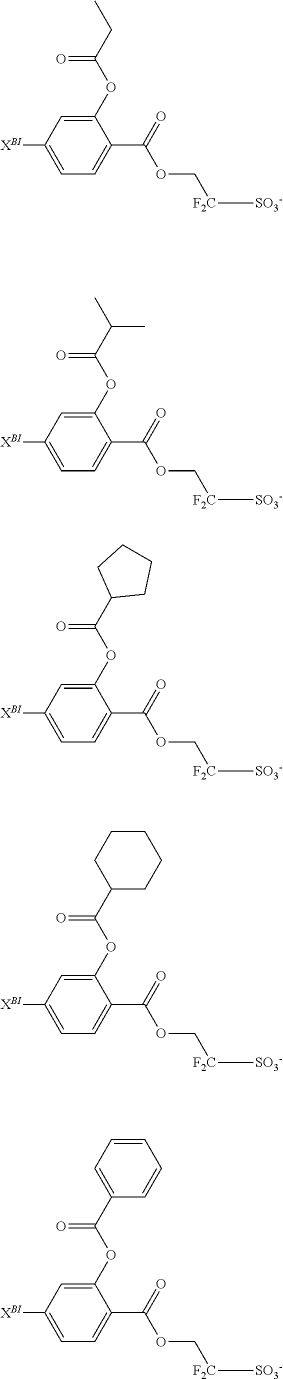

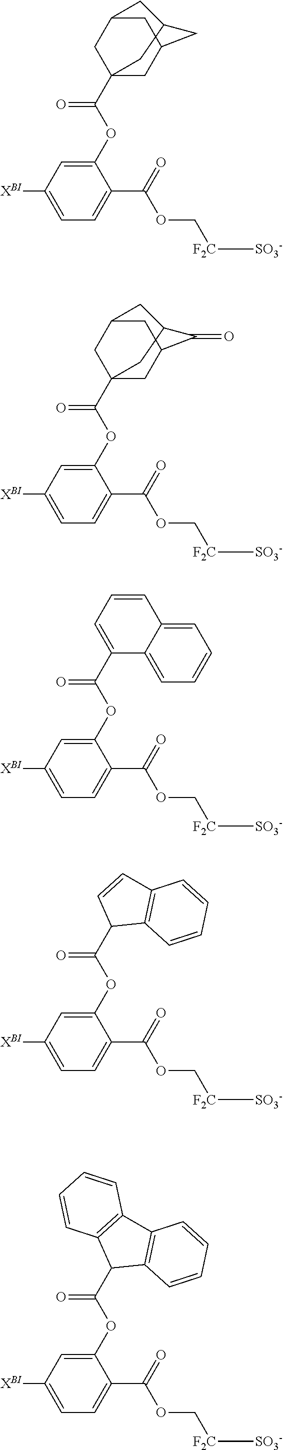

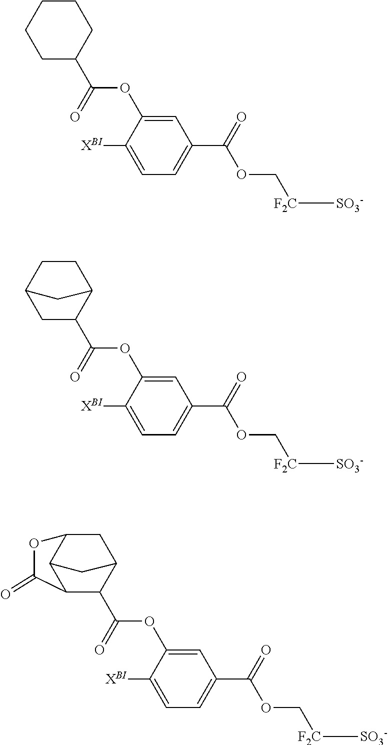

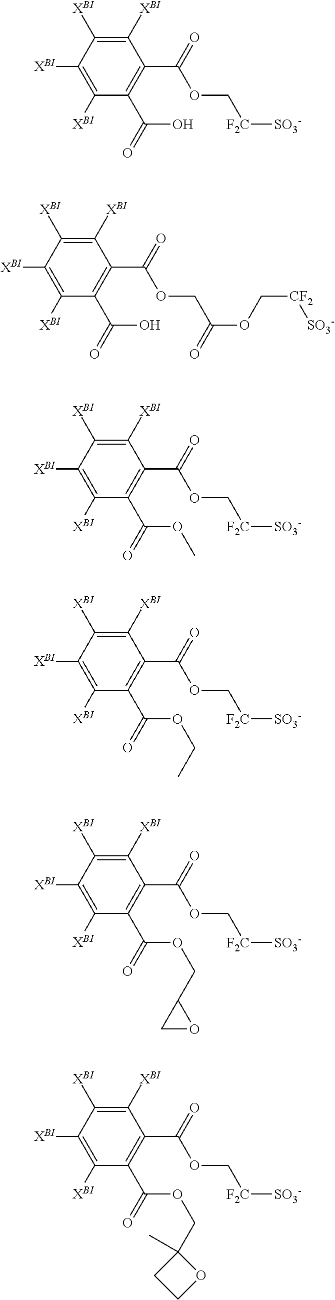

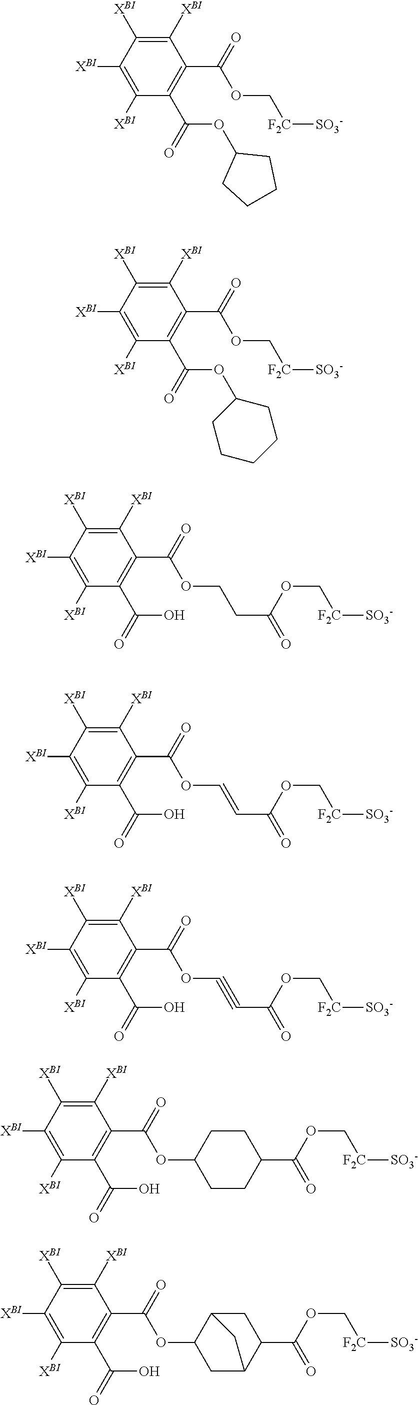

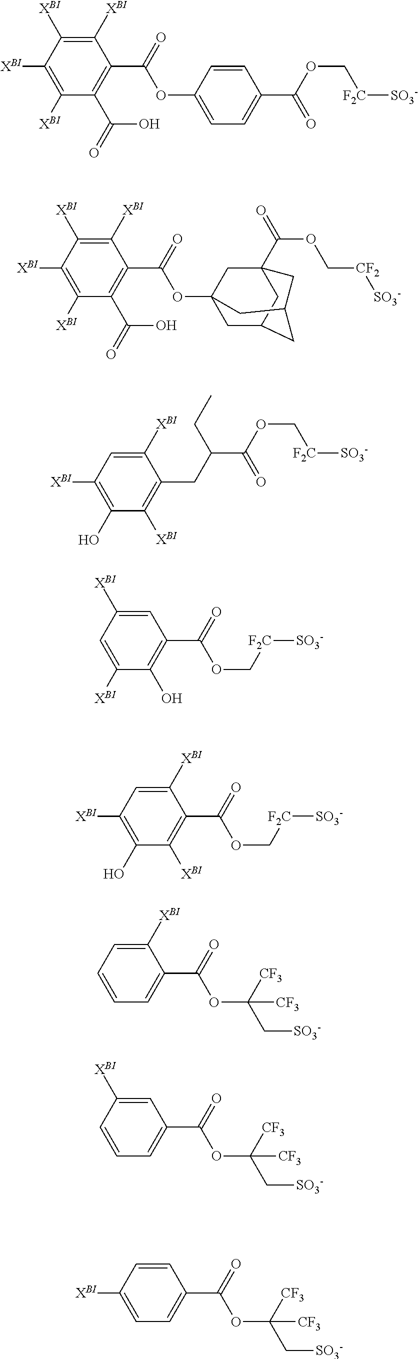

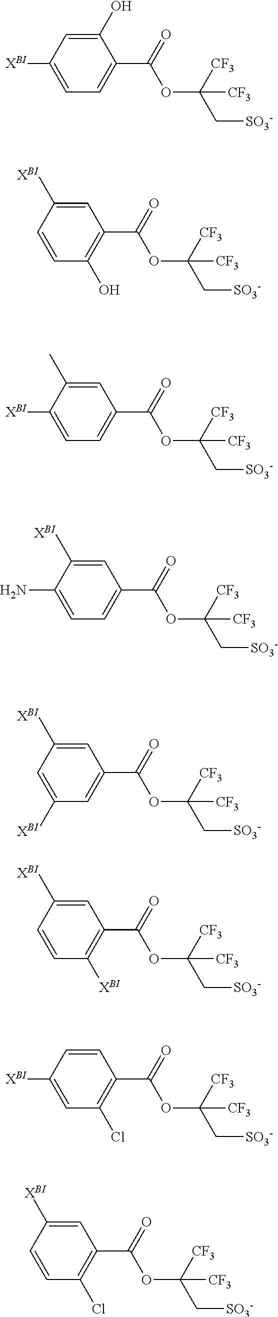

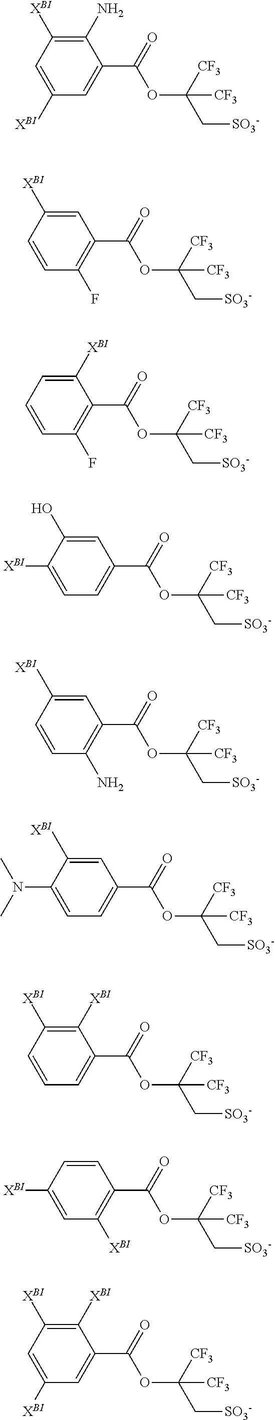

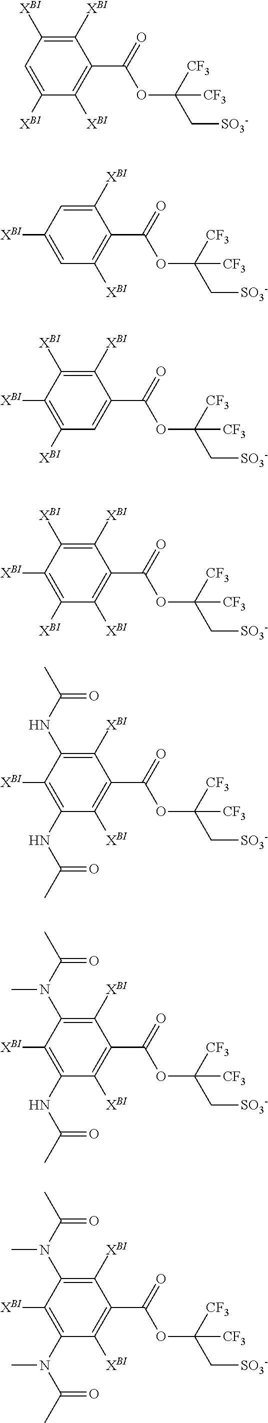

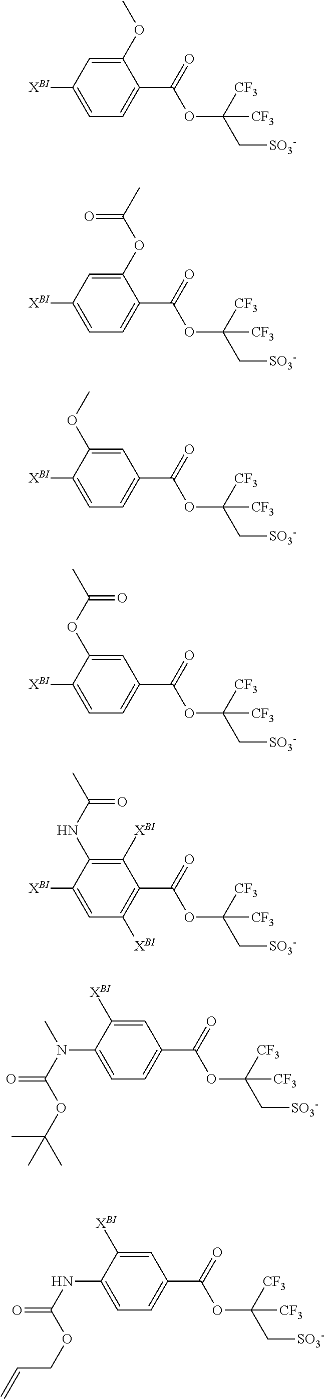

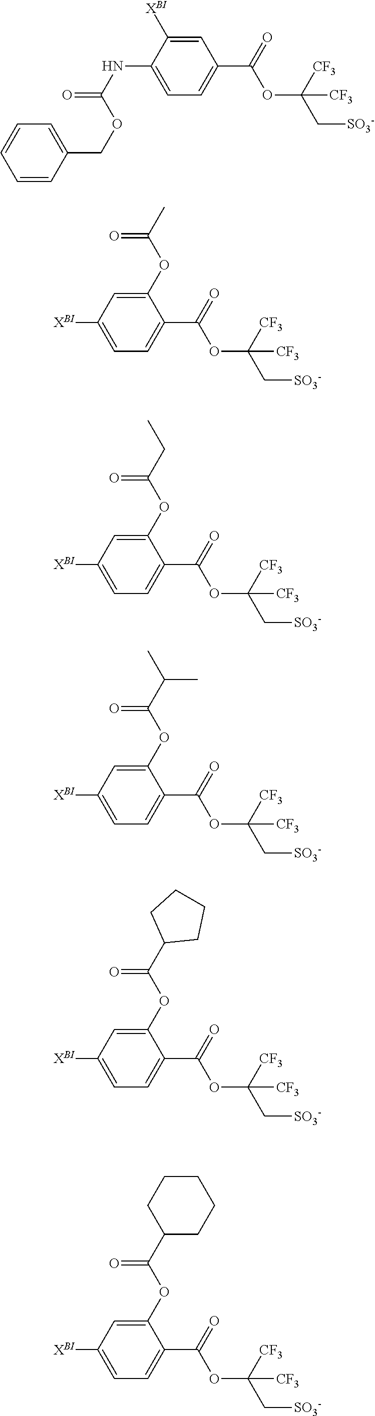

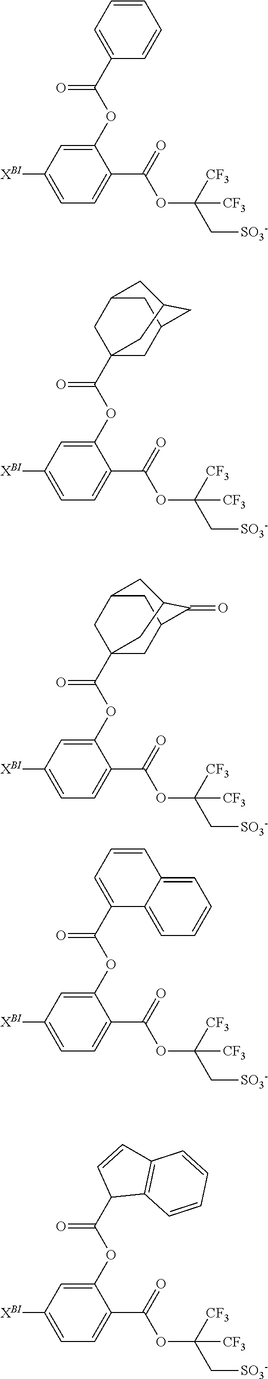

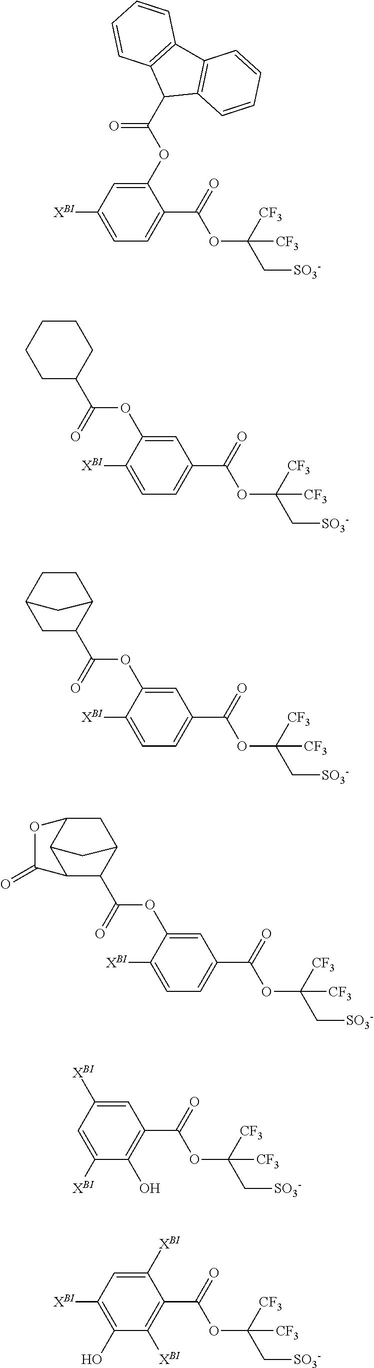

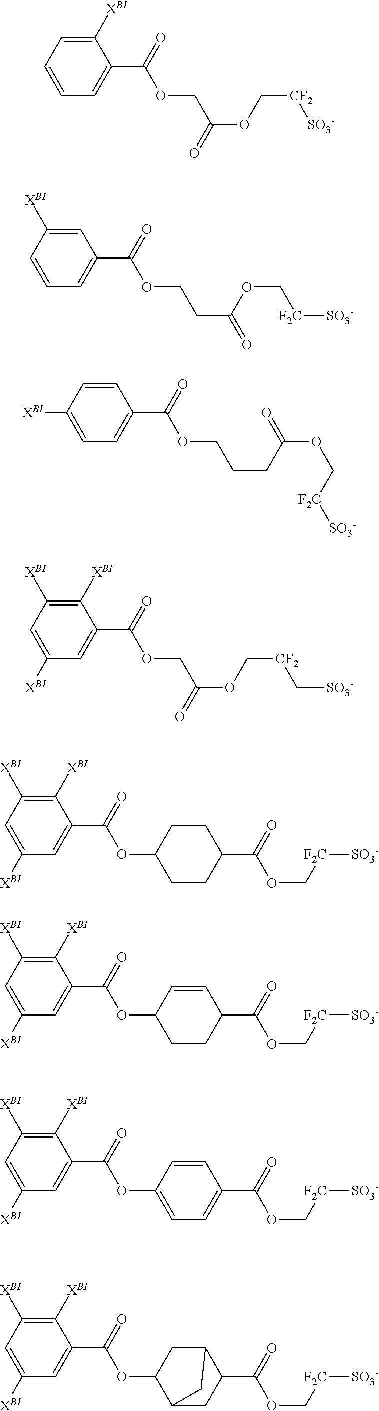

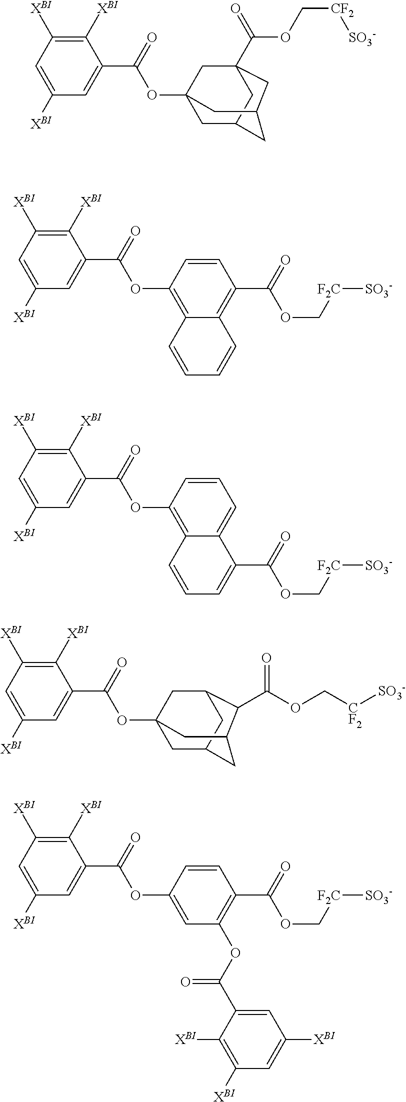

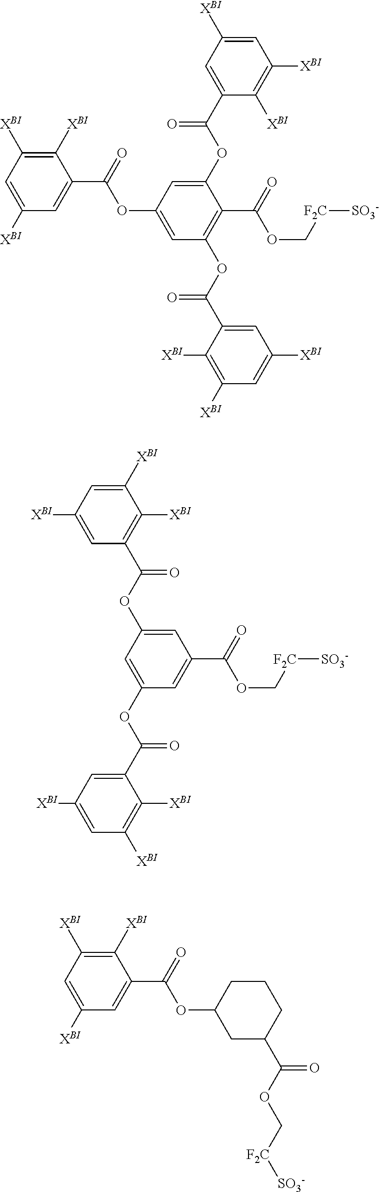

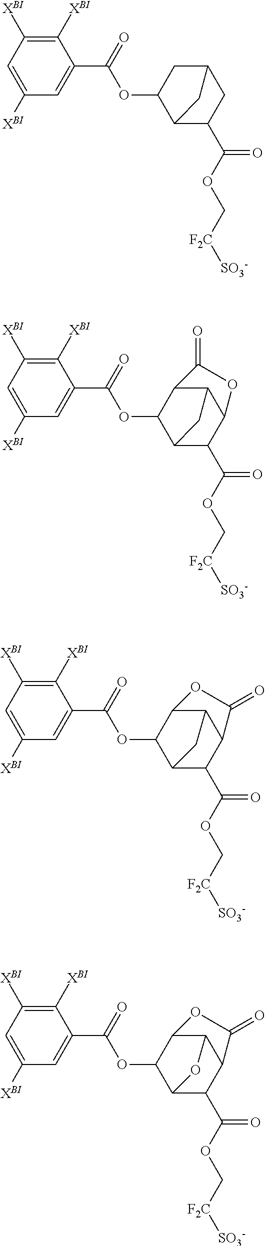

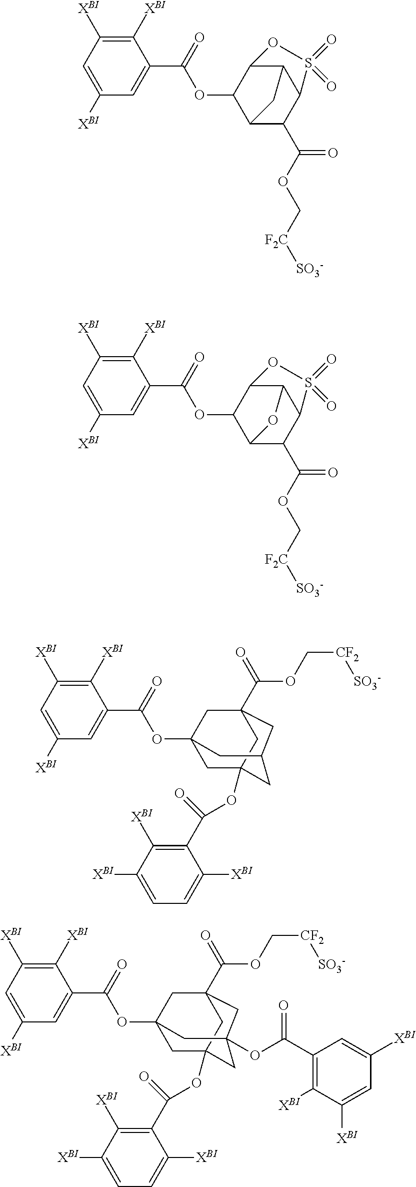

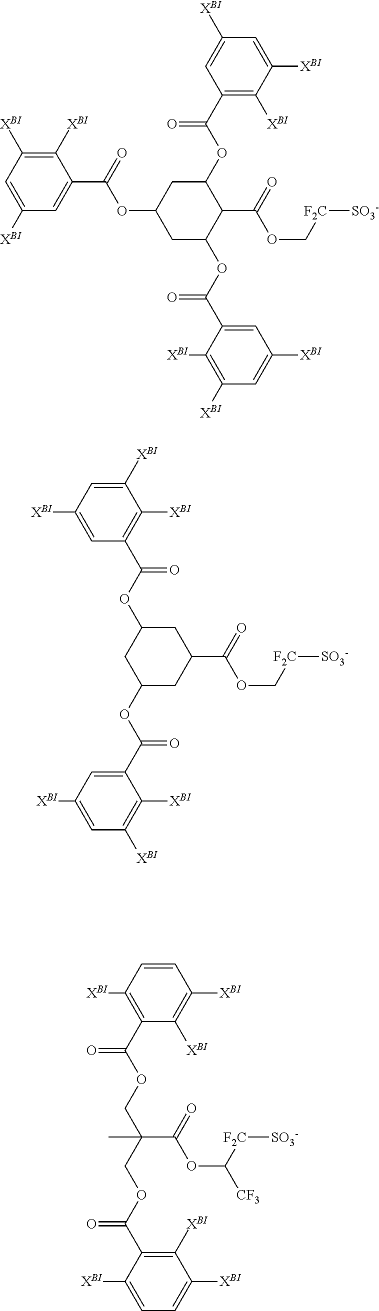

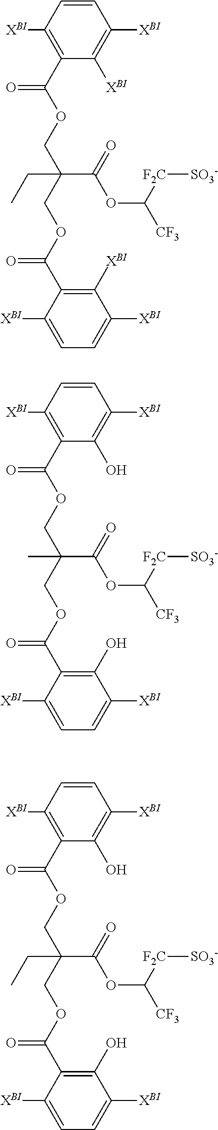

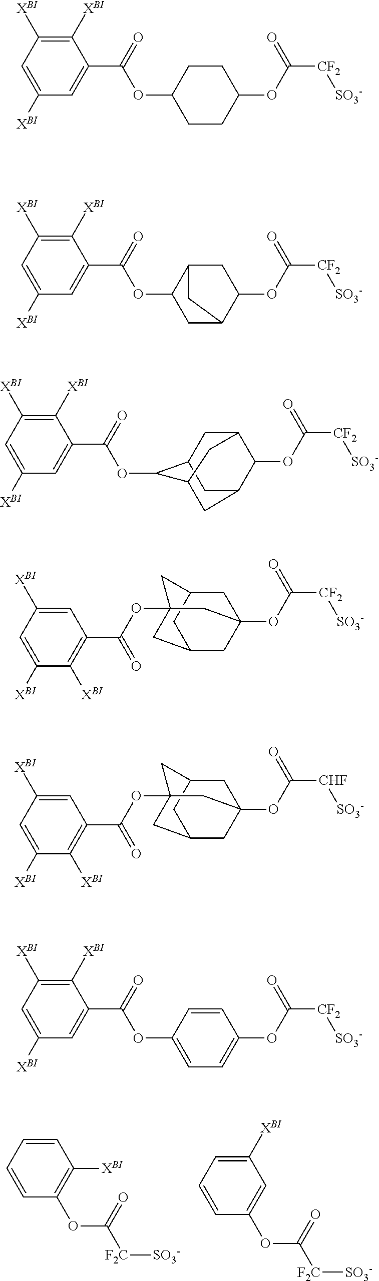

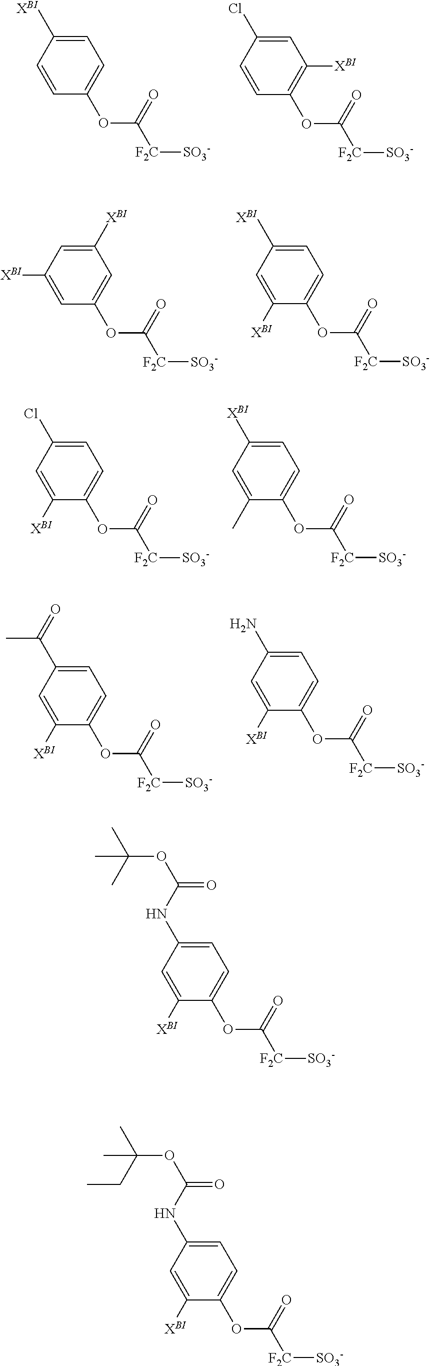

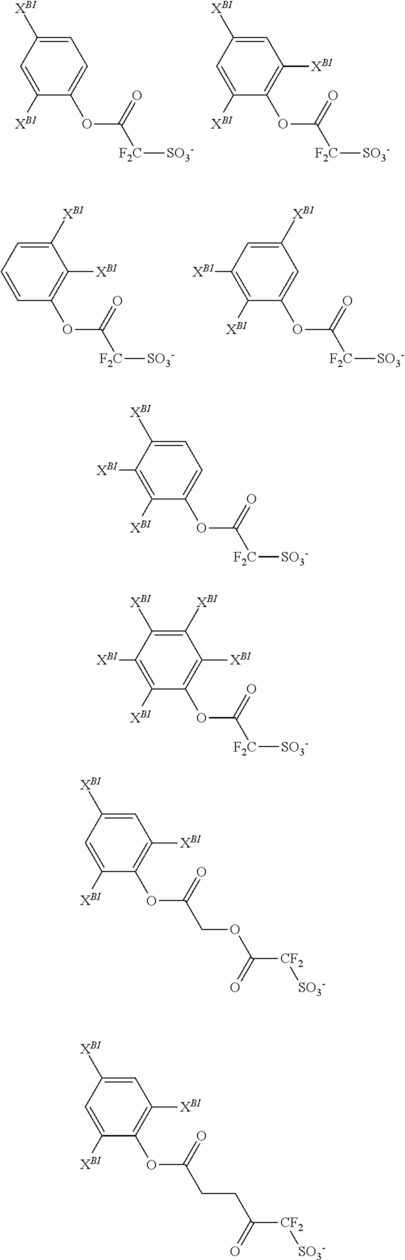

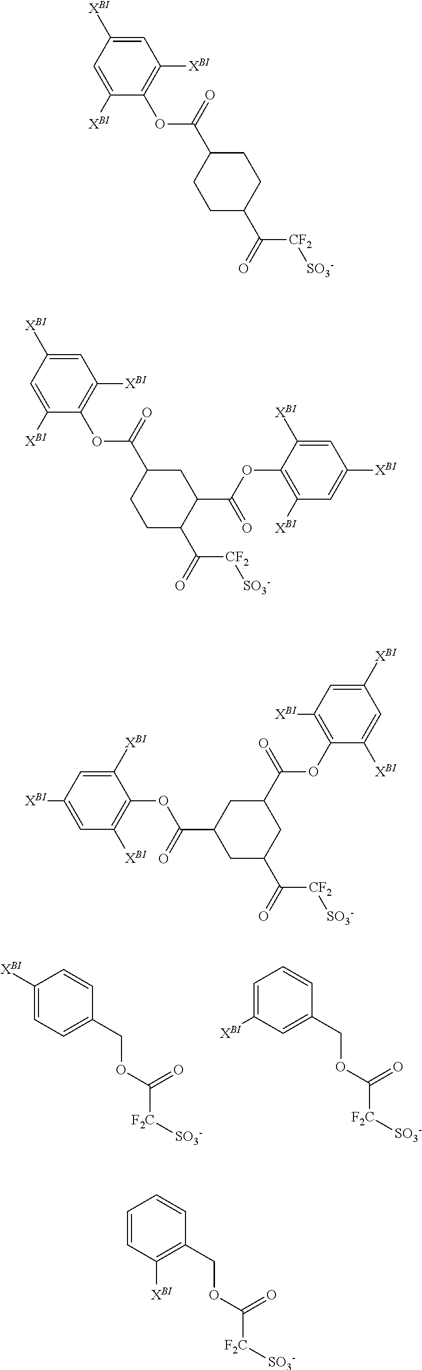

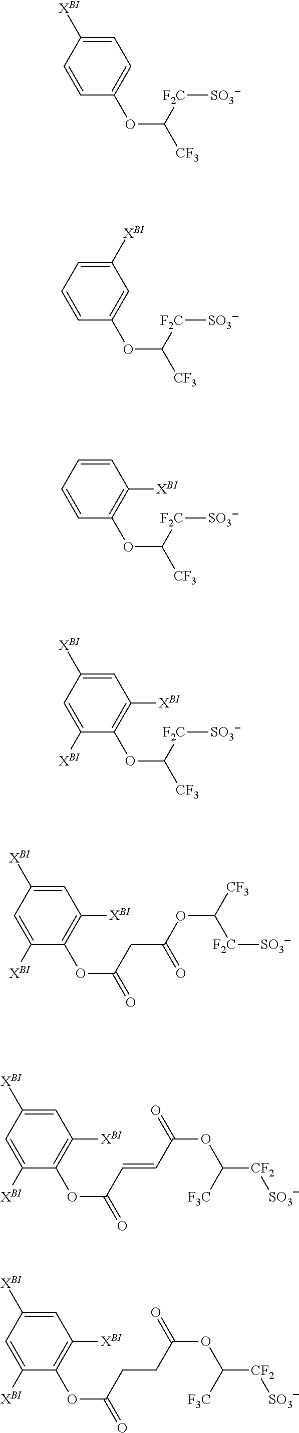

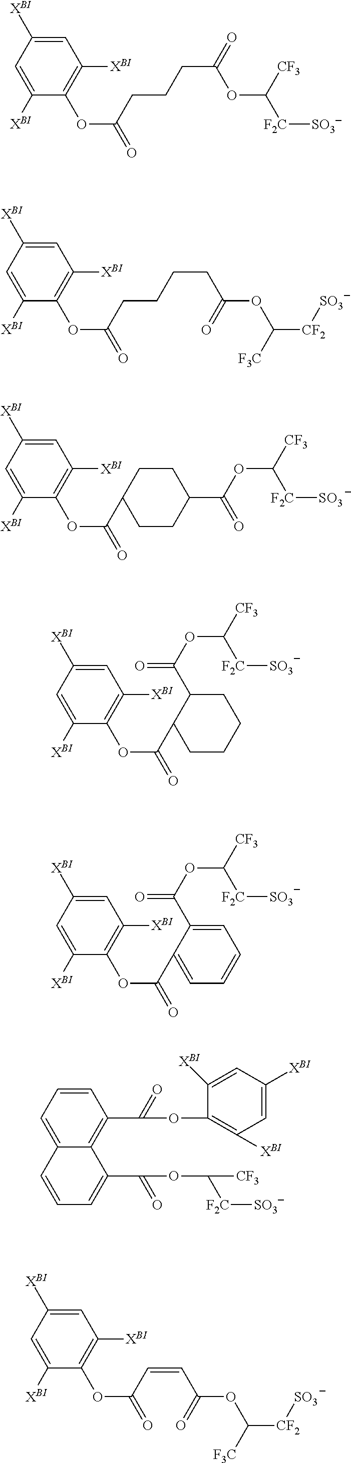

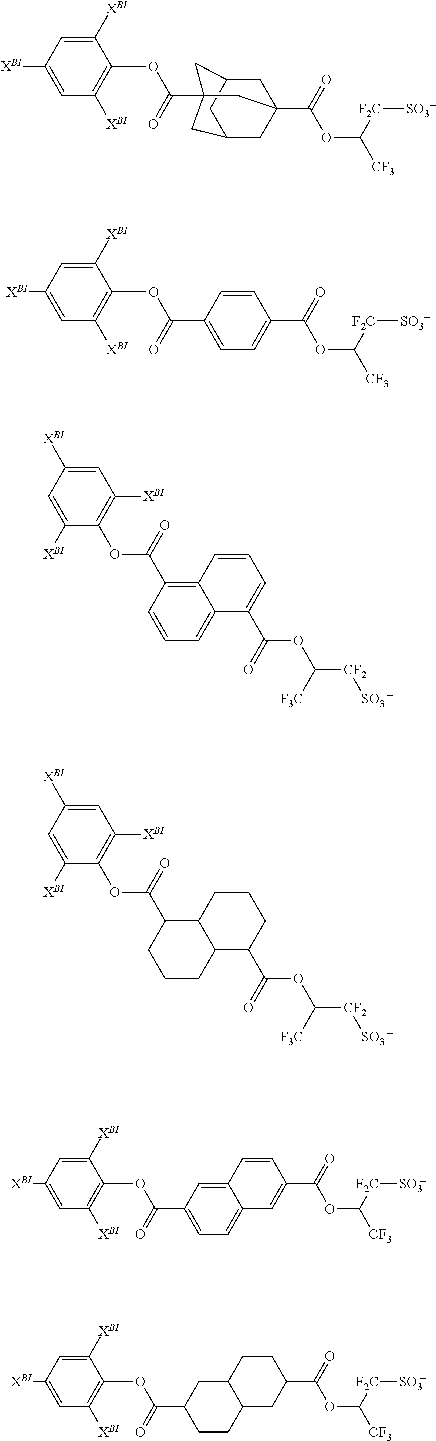

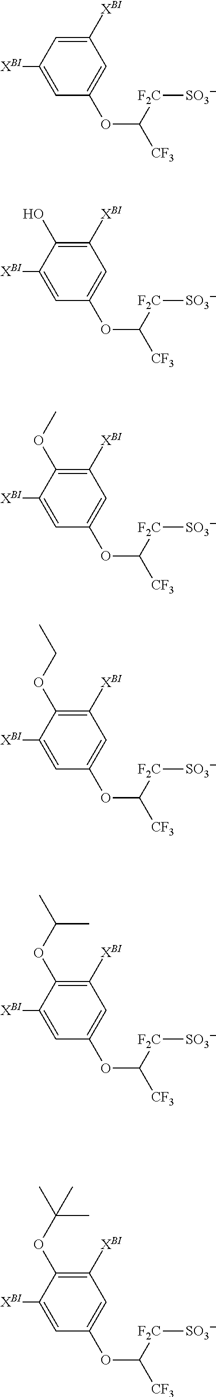

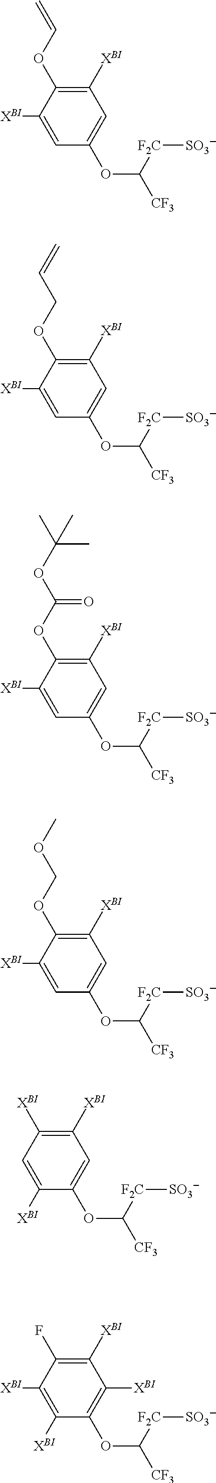

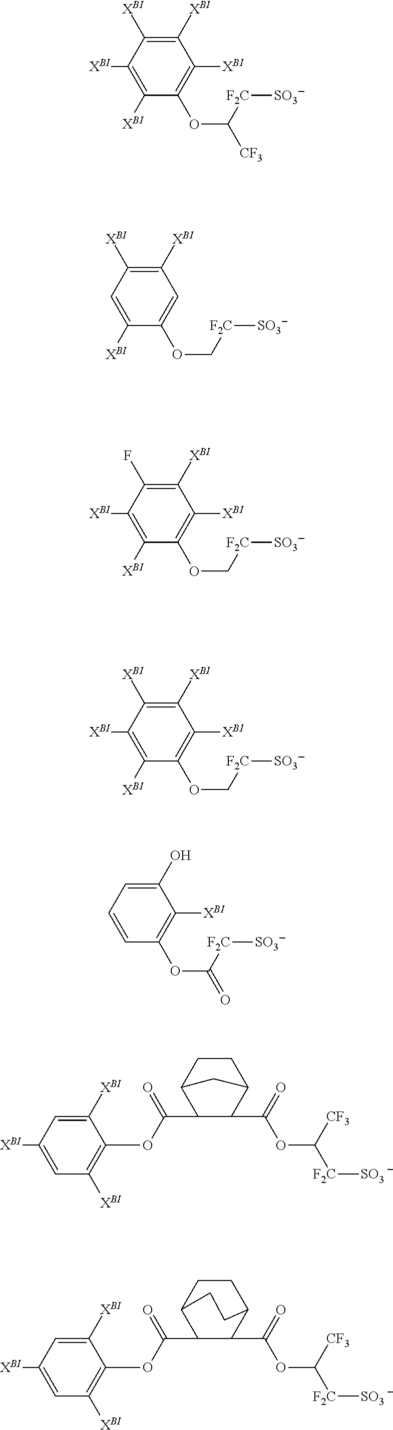

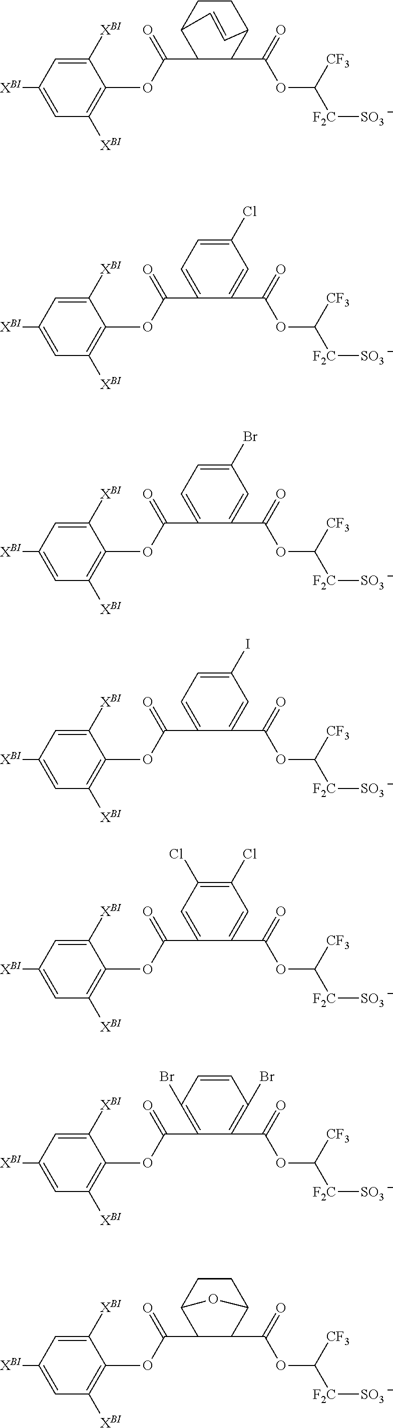

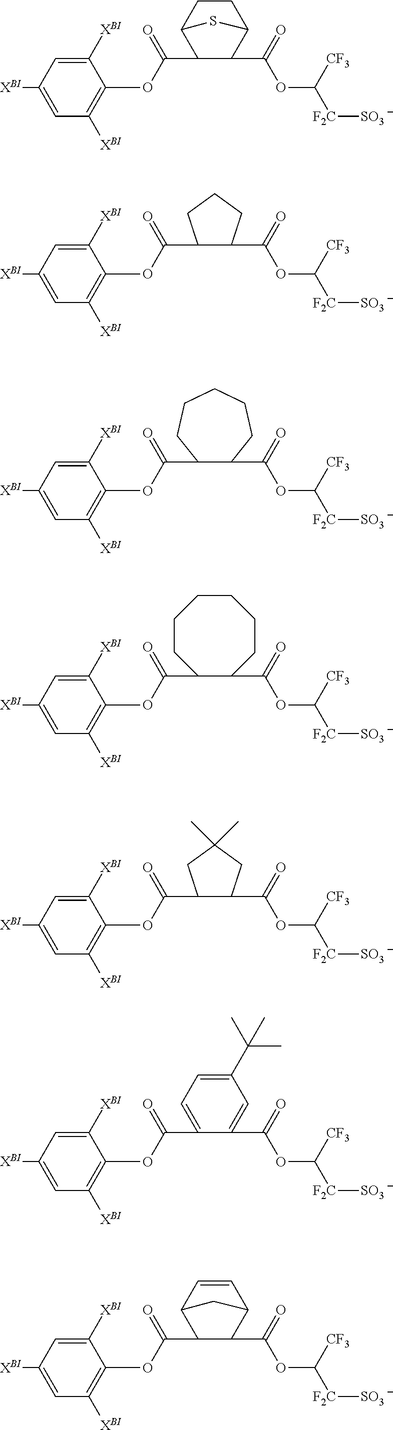

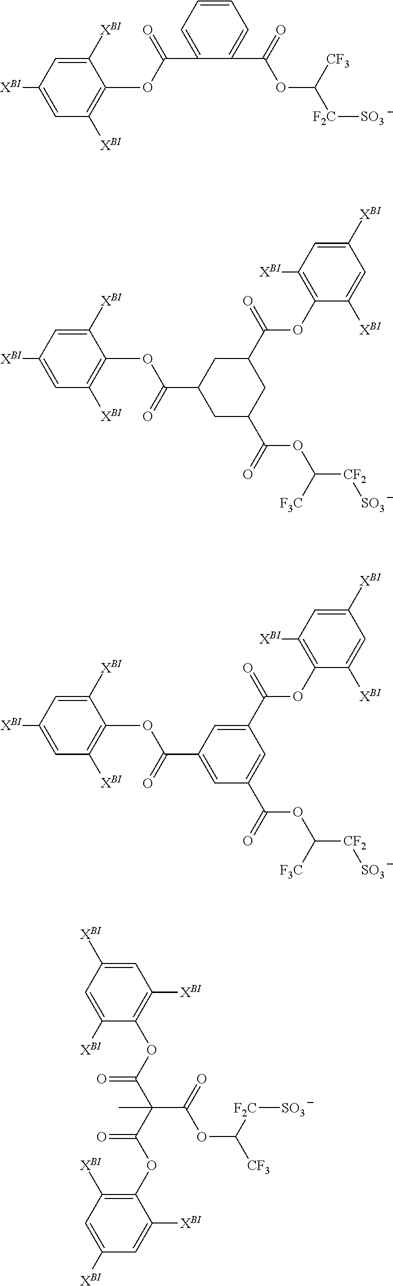

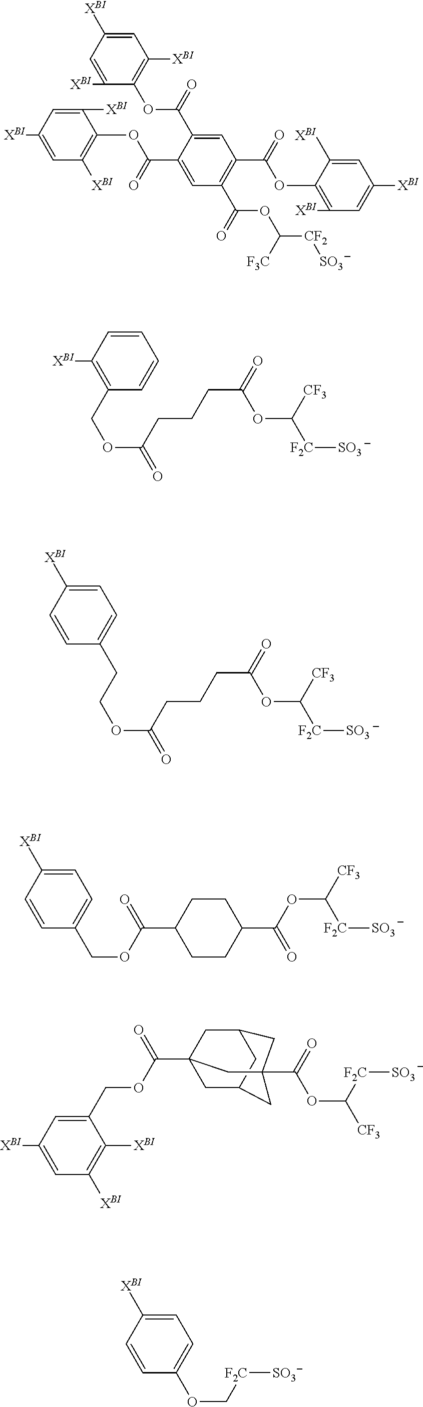

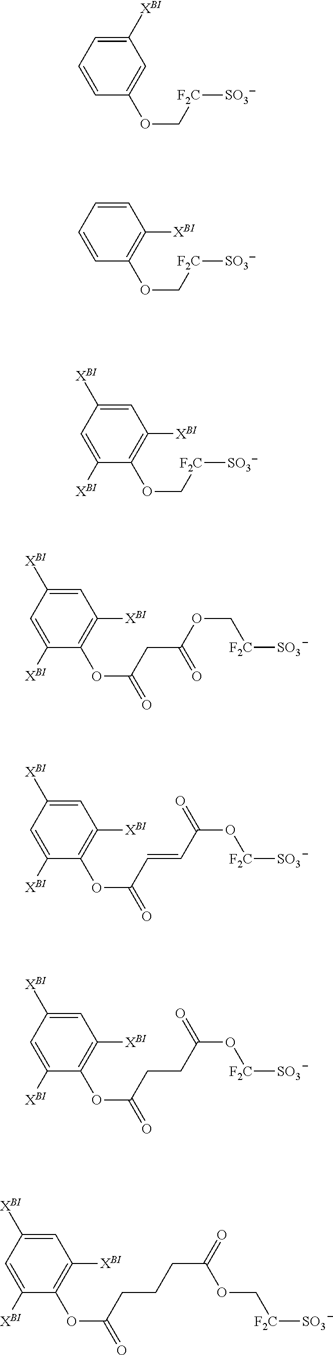

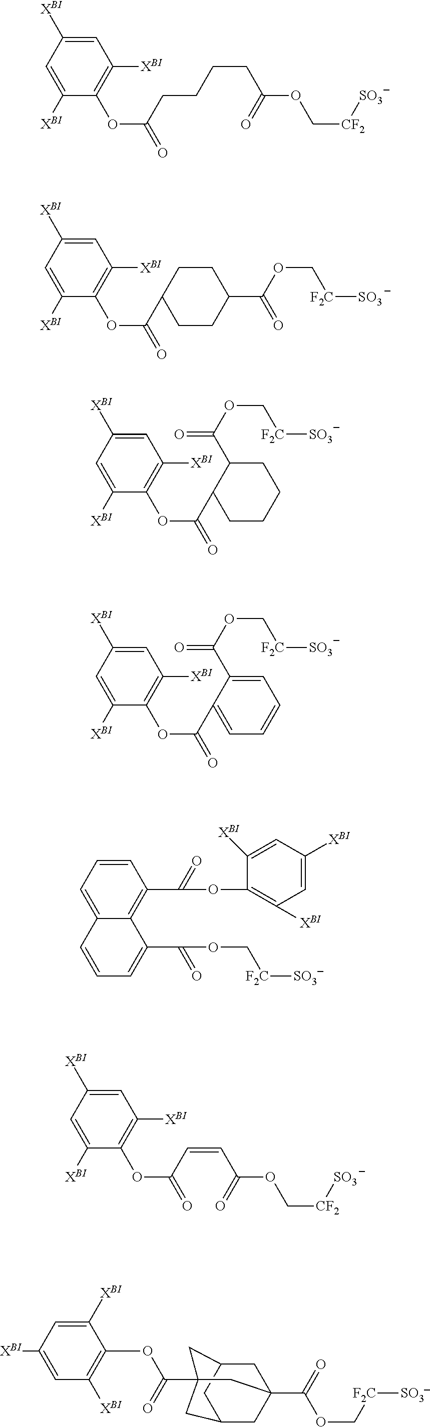

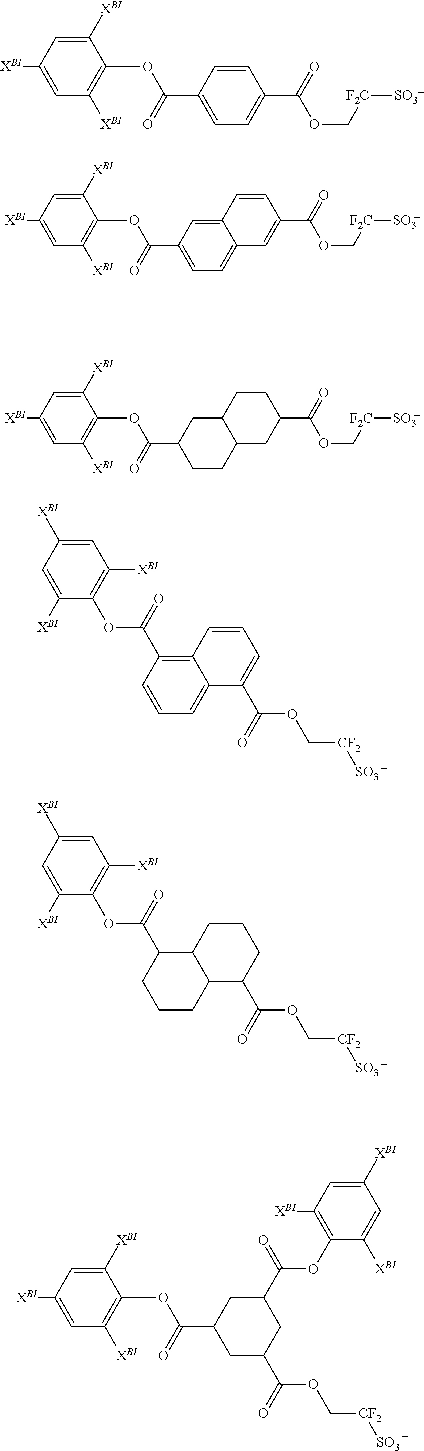

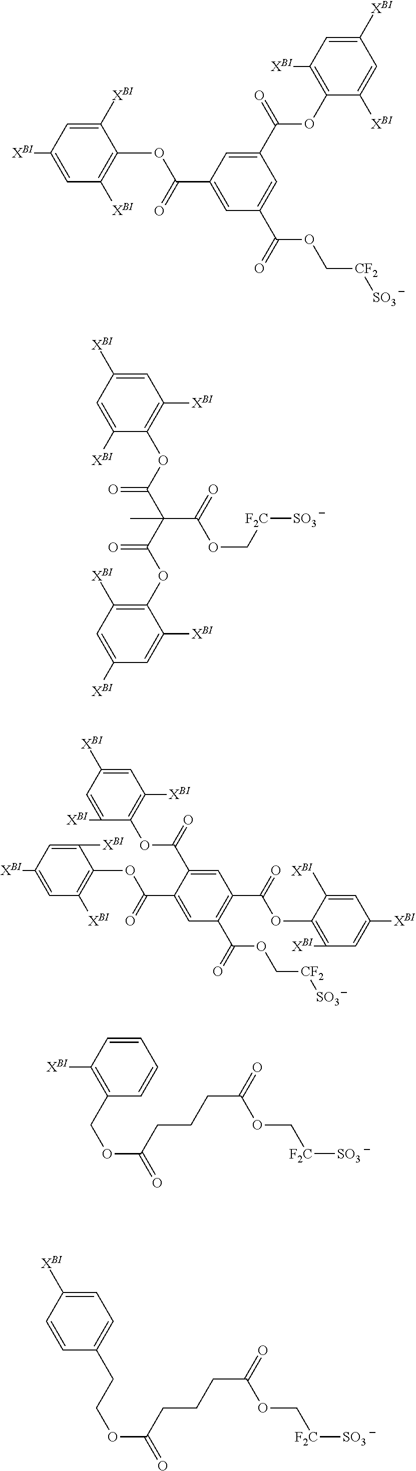

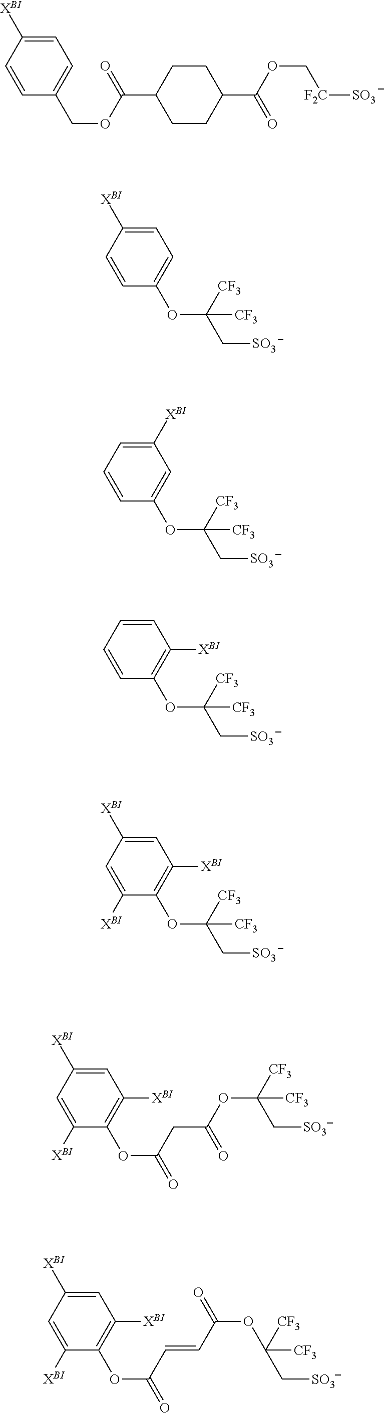

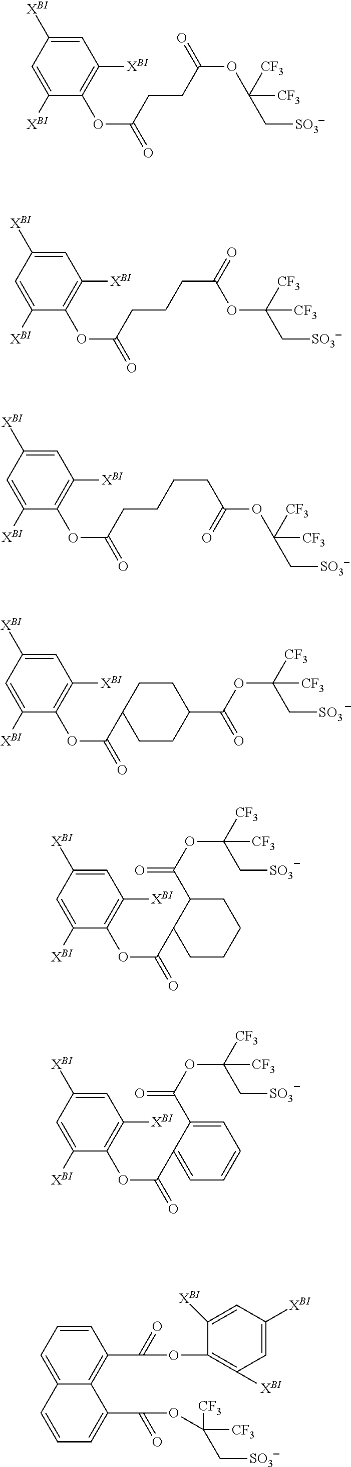

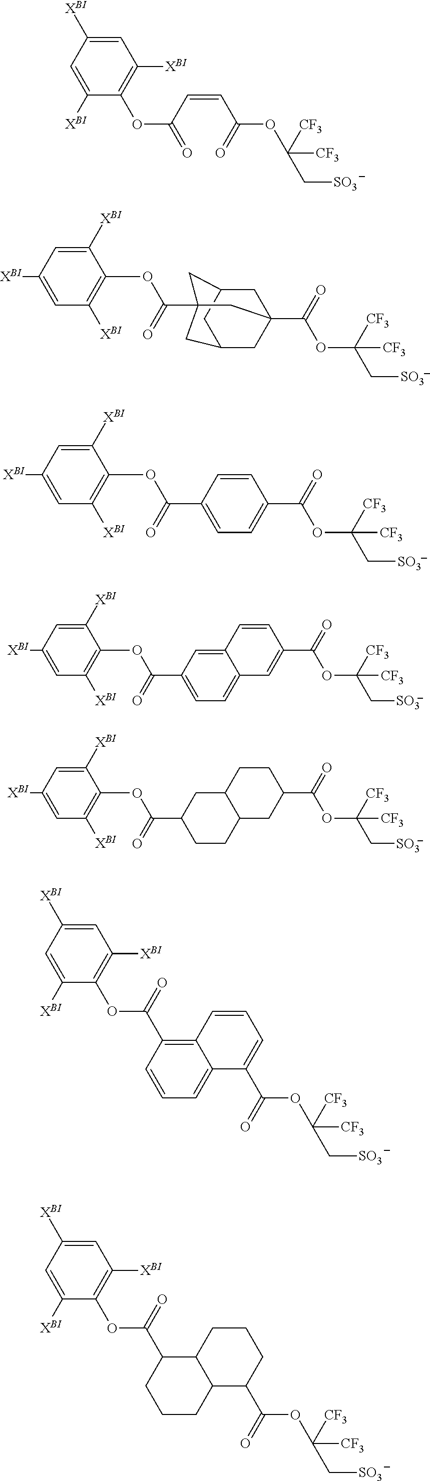

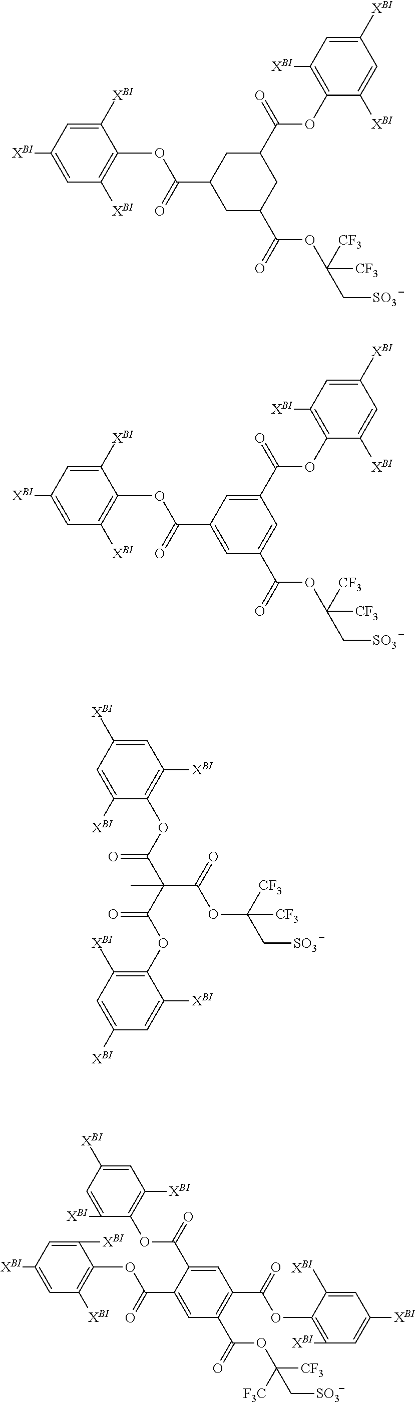

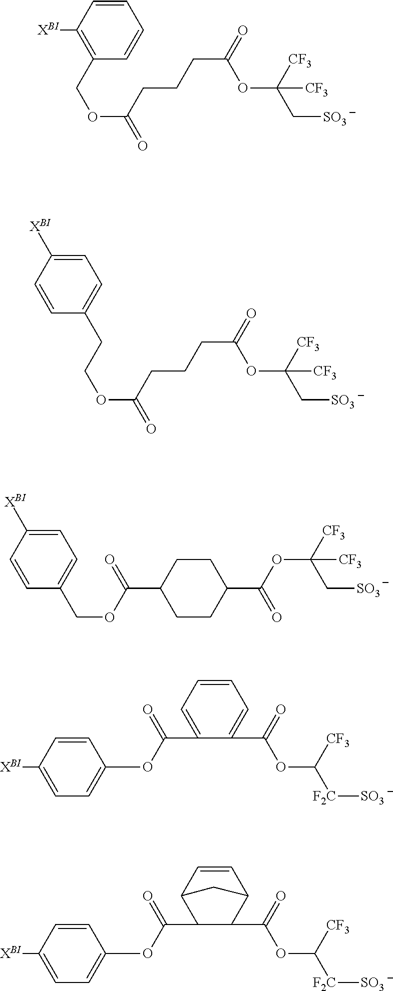

When used, the acid generator of addition type is preferably added in an amount of 0.1 to 50 parts, and more preferably 1 to 40 parts by weight per 100 parts by weight of the base polymer. The resist composition functions as a chemically amplified resist composition when the base polymer includes repeat units (f) and/or the acid generator of addition type is contained.

Organic Solvent

An organic solvent may be added to the resist composition. The organic solvent used herein is not particularly limited as long as the foregoing and other components are soluble therein. Examples of the organic solvent are described in JP-A 2008-111103, paragraphs [0144]-[0145] (U.S. Pat. No. 7,537,880). Exemplary solvents include ketones such as cyclohexanone, cyclopentanone, methyl-2-n-pentyl ketone and 2-heptanone; alcohols such as 3-methoxybutanol, 3-methyl-3-methoxybutanol, 1-methoxy-2-propanol, 1-ethoxy-2-propanol, and diacetone alcohol (DAA); ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as γ-butyrolactone.

The organic solvent is preferably added in an amount of 100 to 10,000 parts, and more preferably 200 to 8,000 parts by weight per 100 parts by weight of the base polymer. The organic solvent may be used alone or in admixture.

Other Components

In addition to the foregoing components, the resist composition may further comprise other components such as a surfactant, dissolution inhibitor, crosslinker, quencher other than Sulfonium Salt A, water repellency improver, and acetylene alcohol. Each of additional components may be used alone or in admixture of two or more.

Exemplary surfactants are described in JP-A 2008-111103, paragraphs [0165]-[0166]. Inclusion of a surfactant may improve or control the coating characteristics of the resist composition. When used, the surfactant is preferably added in an amount of 0.0001 to 10 parts by weight per 100 parts by weight of the base polymer.

When the resist composition is of positive tone, the inclusion of a dissolution inhibitor may lead to an increased difference in dissolution rate between exposed and unexposed areas and a further improvement in resolution. The dissolution inhibitor which can be used herein is a compound having at least two phenolic hydroxy groups on the molecule, in which an average of from 0 to 100 mol % of all the hydrogen atoms on the phenolic hydroxy groups are replaced by acid labile groups or a compound having at least one carboxy group on the molecule, in which an average of 50 to 100 mol % of all the hydrogen atoms on the carboxy groups are replaced by acid labile groups, both the compounds having a molecular weight of 100 to 1,000, and preferably 150 to 800. Typical are bisphenol A, trisphenol, phenolphthalein, cresol novolac, naphthalenecarboxylic acid, adamantanecarboxylic acid, and cholic acid derivatives in which the hydrogen atom on the hydroxy or carboxy group is replaced by an acid labile group, as described in U.S. Pat. No. 7,771,914 (JP-A 2008-122932, paragraphs [0155]-[0178]).

When the resist composition is of positive tone and contains a dissolution inhibitor, the dissolution inhibitor is preferably added in an amount of 0 to 50 parts, more preferably 5 to 40 parts by weight per 100 parts by weight of the base polymer.

When the resist composition is of negative tone, a negative pattern may be formed by adding a crosslinker to reduce the dissolution rate of a resist film in exposed area. Suitable crosslinkers include epoxy compounds, melamine compounds, guanamine compounds, glycoluril compounds and urea compounds having substituted thereon at least one group selected from among methylol, alkoxymethyl and acyloxymethyl groups, isocyanate compounds, azide compounds, and compounds having a double bond such as an alkenyloxy group. These compounds may be used as an additive or introduced into a polymer side chain as a pendant. Hydroxy-containing compounds may also be used as the crosslinker.

Examples of the epoxy compound include tris(2,3-epoxypropyl) isocyanurate, trimethylolmethane triglycidyl ether, trimethylolpropane triglycidyl ether, and triethylolethane triglycidyl ether. Examples of the melamine compound include hexamethylol melamine, hexamethoxymethyl melamine, hexamethylol melamine compounds having 1 to 6 methylol groups methoxymethylated and mixtures thereof, hexamethoxyethyl melamine, hexaacyloxymethyl melamine, hexamethylol melamine compounds having 1 to 6 methylol groups acyloxymethylated and mixtures thereof. Examples of the guanamine compound include tetramethylol guanamine, tetramethoxymethyl guanamine, tetramethylol guanamine compounds having 1 to 4 methylol groups methoxymethylated and mixtures thereof, tetramethoxyethyl guanamine, tetraacyloxyguanamine, tetramethylol guanamine compounds having 1 to 4 methylol groups acyloxymethylated and mixtures thereof. Examples of the glycoluril compound include tetramethylol glycoluril, tetramethoxyglycoluril, tetramethoxymethyl glycoluril, tetramethylol glycoluril compounds having 1 to 4 methylol groups methoxymethylated and mixtures thereof, tetramethylol glycoluril compounds having 1 to 4 methylol groups acyloxymethylated and mixtures thereof. Examples of the urea compound include tetramethylol urea, tetramethoxymethyl urea, tetramethylol urea compounds having 1 to 4 methylol groups methoxymethylated and mixtures thereof, and tetramethoxyethyl urea.

Suitable isocyanate compounds include tolylene diisocyanate, diphenylmethane diisocyanate, hexamethylene diisocyanate and cyclohexane diisocyanate. Suitable azide compounds include 1,1′-biphenyl-4,4′-bisazide, 4,4′-methylidenebisazide, and 4,4′-oxybisazide. Examples of the alkenyloxy group-containing compound include ethylene glycol divinyl ether, triethylene glycol divinyl ether, 1,2-propanediol divinyl ether, 1,4-butanediol divinyl ether, tetramethylene glycol divinyl ether, neopentyl glycol divinyl ether, trimethylol propane trivinyl ether, hexanediol divinyl ether, 1,4-cyclohexanediol divinyl ether, pentaerythritol trivinyl ether, pentaerythritol tetravinyl ether, sorbitol tetravinyl ether, sorbitol pentavinyl ether, and trimethylol propane trivinyl ether.

When the resist composition is of negative tone and contains a crosslinker, the crosslinker is preferably added in an amount of 0.1 to 50 parts, more preferably 1 to 40 parts by weight per 100 parts by weight of the base polymer.

The other quencher is typically selected from conventional basic compounds. Conventional basic compounds include primary, secondary, and tertiary aliphatic amines, mixed amines, aromatic amines, heterocyclic amines, nitrogen-containing compounds with carboxy group, nitrogen-containing compounds with sulfonyl group, nitrogen-containing compounds with hydroxy group, nitrogen-containing compounds with hydroxyphenyl group, alcoholic nitrogen-containing compounds, amide derivatives, imide derivatives, and carbamate derivatives. Also included are primary, secondary, and tertiary amine compounds, specifically amine compounds having a hydroxy group, ether bond, ester bond, lactone ring, cyano group, or sulfonic ester bond as described in JP-A 2008-111103, paragraphs [0146]-[0164], and compounds having a carbamate group as described in JP 3790649. Addition of a basic compound may be effective for further suppressing the diffusion rate of acid in the resist film or correcting the pattern profile.

Onium salts such as sulfonium, iodonium and ammonium salts of sulfonic acids which are not fluorinated at α-position as described in U.S. Pat. No. 8,795,942 (JP-A 2008-158339) and similar onium salts of carboxylic acid may also be used as the quencher. While an α-fluorinated sulfonic acid, imide acid, and methide acid are necessary to deprotect the acid labile group of carboxylic acid ester, an α-non-fluorinated sulfonic acid and a carboxylic acid are released by salt exchange with an α-non-fluorinated onium salt. An α-non-fluorinated sulfonic acid and a carboxylic acid function as a quencher because they do not induce deprotection reaction.

Also useful are quenchers of polymer type as described in U.S. Pat. No. 7,598,016 (JP-A 2008-239918). The polymeric quencher segregates at the resist surface and thus enhances the rectangularity of resist pattern. When a protective film is applied as is often the case in the immersion lithography, the polymeric quencher is also effective for preventing a film thickness loss of resist pattern or rounding of pattern top.

When used, the other quencher is preferably added in an amount of 0 to 5 parts, more preferably 0 to 4 parts by weight per 100 parts by weight of the base polymer.

To the resist composition, a water repellency improver may also be added for improving the water repellency on surface of a resist film. The water repellency improver may be used in the topcoatless immersion lithography. Suitable water repellency improvers include polymers having a fluoroalkyl group and polymers of specific structure having a 1,1,1,3,3,3-hexafluoro-2-propanol residue and are described in JP-A 2007-297590 and JP-A 2008-111103, for example. The water repellency improver should be soluble in the alkaline developer and organic solvent developer. The water repellency improver of specific structure having a 1,1,1,3,3,3-hexafluoro-2-propanol residue is well soluble in the developer. A polymer comprising repeat units having an amino group or amine salt may serve as the water repellent additive and is effective for preventing evaporation of acid during PEB, thus preventing any hole pattern opening failure after development. An appropriate amount of the water repellency improver is 0 to 20 parts, more preferably 0.5 to 10 parts by weight per 100 parts by weight of the base polymer.

Also, an acetylene alcohol may be blended in the resist composition. Suitable acetylene alcohols are described in JP-A 2008-122932, paragraphs [0179]-[0182]. An appropriate amount of the acetylene alcohol blended is 0 to 5 parts by weight per 100 parts by weight of the base polymer.

Process

The resist composition is used in the fabrication of various integrated circuits. Pattern formation using the resist composition may be performed by well-known lithography processes. The process generally involves the steps of applying the resist composition onto a substrate to form a resist film thereon, exposing the resist film to high-energy radiation, and developing the exposed resist film in a developer. If necessary, any additional steps may be added.

Specifically, the resist composition is first applied onto a substrate on which an integrated circuit is to be formed (e.g., Si, SiO₂, SiN, SiON, TiN, WSi, BPSG, SOG, or organic antireflective coating) or a substrate on which a mask circuit is to be formed (e.g., Cr, CrO, CrON, MoSi₂, or SiO₂) by a suitable coating technique such as spin coating, roll coating, flow coating, dipping, spraying or doctor coating. The coating is prebaked on a hotplate preferably at a temperature of 60 to 150° C. for 10 seconds to 30 minutes, more preferably at 80 to 120° C. for 30 seconds to 20 minutes. The resulting resist film is generally 0.01 to 2 μm thick.

The resist film is then exposed to a desired pattern of high-energy radiation such as UV, deep-UV, EB, EUV of wavelength 3 to 15 nm, x-ray, soft x-ray, excimer laser light, γ-ray or synchrotron radiation. When UV, deep-UV, EUV, x-ray, soft x-ray, excimer laser light, γ-ray or synchrotron radiation is used as the high-energy radiation, the resist film is exposed thereto directly or through a mask having a desired pattern in a dose of preferably about 1 to 200 mJ/cm², more preferably about 10 to 100 mJ/cm². When EB is used as the high-energy radiation, the resist film is exposed thereto directly or through a mask having a desired pattern in a dose of preferably about 0.1 to 300 μC/cm², more preferably about 0.5 to 200 μC/cm². It is appreciated that the inventive resist composition is suited in micropatterning using KrF excimer laser, ArF excimer laser, EB, EUV, x-ray, soft x-ray, γ-ray or synchrotron radiation, especially in micropatterning using EB or EUV.

After the exposure, the resist film may be baked (PEB) on a hotplate or in an oven preferably at 30 to 150° C. for 10 seconds to 30 minutes, more preferably at 50 to 120° C. for 30 seconds to 20 minutes.

After the exposure or PEB, the resist film is developed in a developer in the form of an aqueous base solution for 3 seconds to 3 minutes, preferably 5 seconds to 2 minutes by conventional techniques such as dip, puddle and spray techniques. A typical developer is a 0.1 to 10 wt %, preferably 2 to 5 wt % aqueous solution of tetramethylammonium hydroxide (TMAH), tetraethylammonium hydroxide (TEAH), tetrapropylammonium hydroxide (TPAH), or tetrabutylammonium hydroxide (TBAH). In the case of positive tone, the resist film in the exposed area is dissolved in the developer whereas the resist film in the unexposed area is not dissolved. In this way, the desired positive pattern is formed on the substrate. In the case of negative tone, inversely the resist film in the exposed area is insolubilized whereas the resist film in the unexposed area is dissolved away.

In an alternative embodiment, a negative pattern can be obtained from the positive resist composition comprising a base polymer containing acid labile groups by effecting organic solvent development. The developer used herein is preferably selected from among 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate, and mixtures thereof.

At the end of development, the resist film is rinsed. As the rinsing liquid, a solvent which is miscible with the developer and does not dissolve the resist film is preferred. Suitable solvents include alcohols of 3 to 10 carbon atoms, ether compounds of 8 to 12 carbon atoms, alkanes, alkenes, and alkynes of 6 to 12 carbon atoms, and aromatic solvents. Specifically, suitable alcohols of 3 to 10 carbon atoms include n-propyl alcohol, isopropyl alcohol, 1-butyl alcohol, 2-butyl alcohol, isobutyl alcohol, t-butyl alcohol, 1-pentanol, 2-pentanol, 3-pentanol, t-pentyl alcohol, neopentyl alcohol, 2-methyl-1-butanol, 3-methyl-1-butanol, 3-methyl-3-pentanol, cyclopentanol, 1-hexanol, 2-hexanol, 3-hexanol, 2,3-dimethyl-2-butanol, 3,3-dimethyl-1-butanol, 3,3-dimethyl-2-butanol, 2-ethyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 4-methyl-3-pentanol, cyclohexanol, and 1-octanol. Suitable ether compounds of 8 to 12 carbon atoms include di-n-butyl ether, diisobutyl ether, di-s-butyl ether, di-n-pentyl ether, diisopentyl ether, di-s-pentyl ether, di-t-pentyl ether, and di-n-hexyl ether. Suitable alkanes of 6 to 12 carbon atoms include hexane, heptane, octane, nonane, decane, undecane, dodecane, methylcyclopentane, dimethylcyclopentane, cyclohexane, methylcyclohexane, dimethylcyclohexane, cycloheptane, cyclooctane, and cyclononane. Suitable alkenes of 6 to 12 carbon atoms include hexene, heptene, octene, cyclohexene, methylcyclohexene, dimethylcyclohexene, cycloheptene, and cyclooctene. Suitable alkynes of 6 to 12 carbon atoms include hexyne, heptyne, and octyne. Suitable aromatic solvents include toluene, xylene, ethylbenzene, isopropylbenzene, t-butylbenzene and mesitylene.

Rinsing is effective for minimizing the risks of resist pattern collapse and defect formation. However, rinsing is not essential. If rinsing is omitted, the amount of solvent used may be reduced.

A hole or trench pattern after development may be shrunk by the thermal flow, RELACS® or DSA process. A hole pattern is shrunk by coating a shrink agent thereto, and baking such that the shrink agent may undergo crosslinking at the resist surface as a result of the acid catalyst diffusing from the resist layer during bake, and the shrink agent may attach to the sidewall of the hole pattern. The bake is preferably at a temperature of 70 to 180° C., more preferably 80 to 170° C., for a time of 10 to 300 seconds. The extra shrink agent is stripped and the hole pattern is shrunk.

### EXAMPLES

Examples of the invention are given below by way of illustration and not by way of limitation. All parts are by weight (pbw).

### Synthesis Example 1-1 Synthesis of Quencher Q-1

### (1) Synthesis of Intermediate In-1

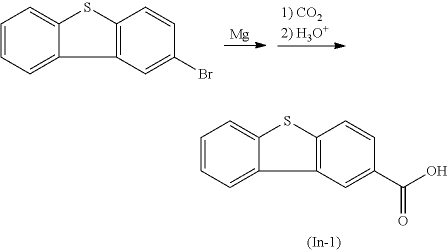

In nitrogen atmosphere, a Grignard reagent was prepared from 100 g of 2-bromodibenzothiophene, 9.7 g of magnesium, and 500 g of THF. The Grignard reagent was added to another vessel which was charged with dry ice and THF. At the end of addition, the reaction solution was aged for 2 hours. Subsequently, dilute hydrochloric acid was added dropwise and 1 L of ethyl acetate was then added. The organic layer was taken out. The organic layer was washed with ultrapure water and concentrated under reduced pressure. Toluene was added to the concentrate for recrystallization. The crystals were filtered and dried in vacuum, obtaining 58 g of the desired intermediate In-1 (yield 67%).

### (2) Synthesis of Intermediate In-2

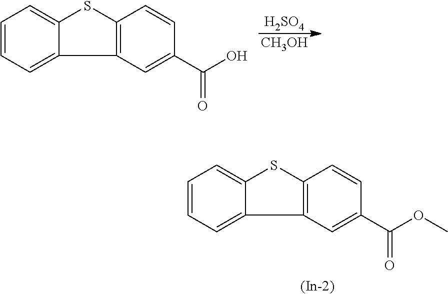

A mixture of 20 g of Intermediate In-1, 70 g of methanol and 0.4 g of conc. sulfuric acid was heated under reflux for 24 hours, after which triethylamine was added thereto to quench the reaction. Toluene was added to the reaction solution, after which the organic layer was taken out, washed with ultrapure water, and concentrated under reduced pressure. To the concentrate, n-hexane was added for recrystallization. The crystals were filtered and dried in vacuum, obtaining 18 g of the desired intermediate In-2 (yield 87%).

(3) Synthesis of Intermediate In-3

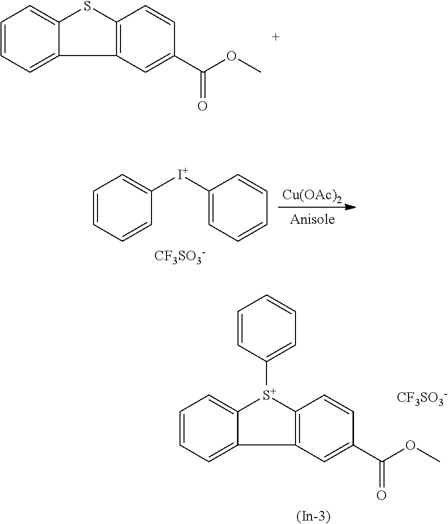

In nitrogen atmosphere, a mixture of 10 g of Intermediate In-2, 20 g of diphenyliodonium triflate, 0.3 g of copper acetate, and 50 g of anisole was heated and stirred at 100° C. for 2 hours. Diisopropyl ether was added to the reaction solution for crystallization. Crude crystals were collected by filtration. This was followed by recrystallization from dichloromethane/methyl isobutyl ketone. Crystals were collected by filtration and vacuum dried, obtaining 15 g of the desired intermediate In-3 (yield 80%).

(4) Synthesis of Intermediate In-4

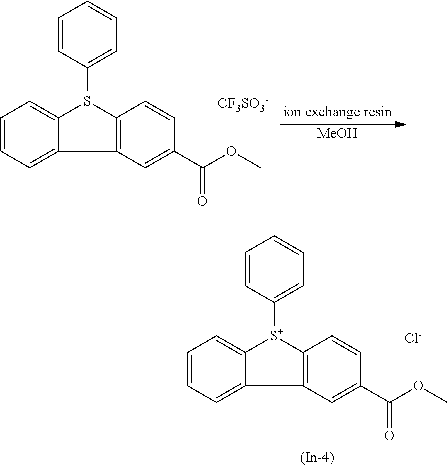

A mixture of 13 g of Intermediate In-3, 40 g of an ion exchange resin (Amberlite® IRA410JC1 by Organo Co., Ltd.) and 100 g of methanol was stirred at room temperature for 2 hours. The reaction solution was filtered and the filtrate was concentrated under reduced pressure. To the concentrate, n-hexane was added for recrystallization. The crystals were collected by filtration and vacuum dried, obtaining 9.4 g of the desired intermediate In-4 (yield 95%).

(5) Synthesis of Quencher Q-1

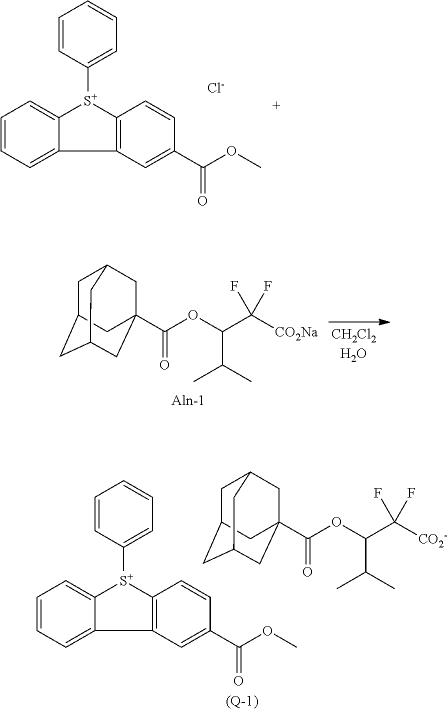

A mixture of 3.5 g of anion intermediate AIn-1 which had been synthesized with reference to JP 5904180, 3.5 g of Intermediate In-4, 20 g of methylene chloride and 10 g of water was stirred at room temperature for 1 hour. The organic layer was taken out, washed with ultrapure water, and concentrated under reduced pressure to remove the organic solvent. Diisopropyl ether was added to the concentrate for recrystallization. The crystals were collected by filtration and vacuum dried, obtaining 5.8 g of the target quencher Q-1 (yield 89%).

### Synthesis Examples 1-2 to 1-21 Synthesis of Quenchers Q-2 to Q-21

Quenchers Q-2 to Q-21 were synthesized by the same procedure as in Synthesis Example 1-1. The structure of Quenchers Q-1 to Q-21 is shown below.

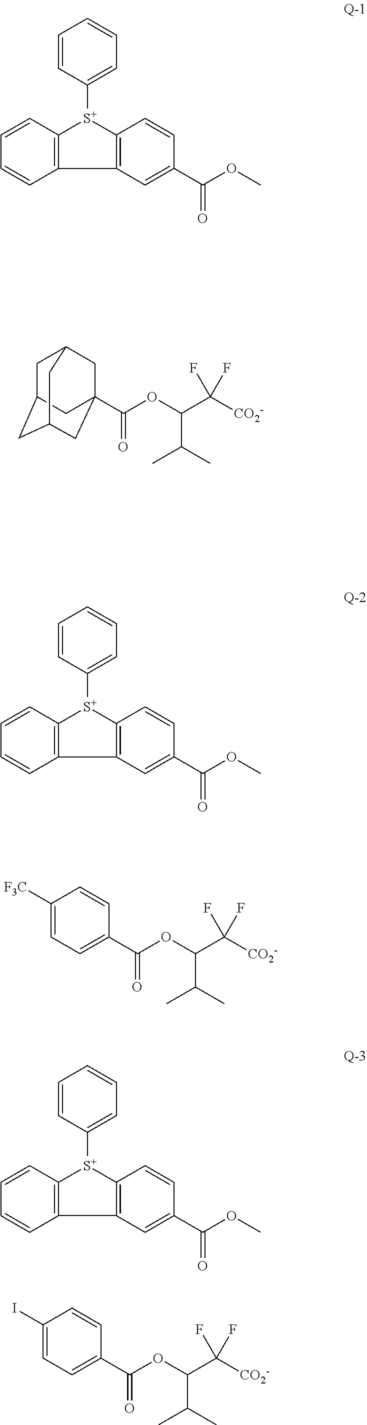

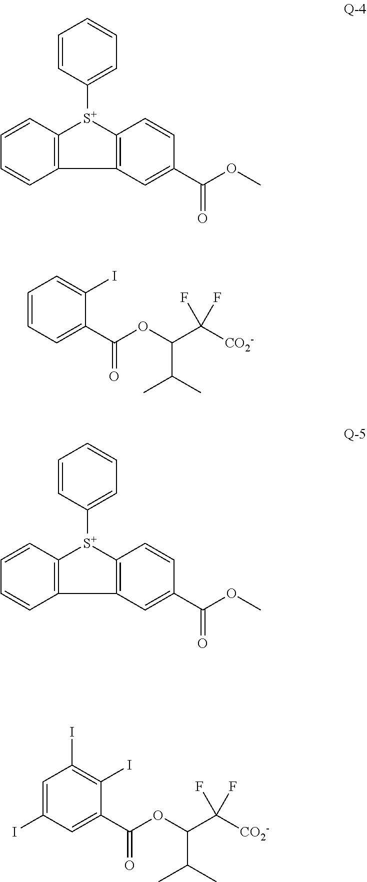

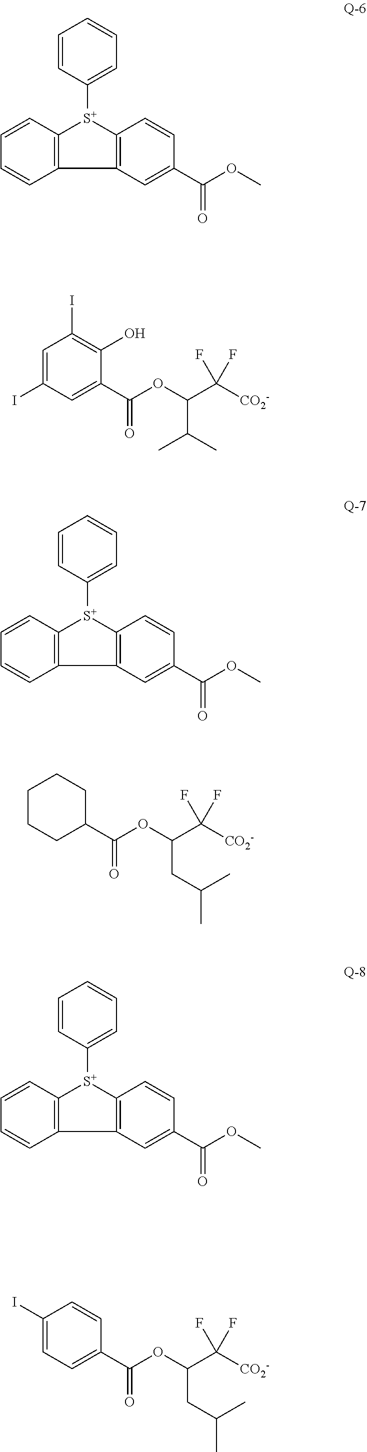

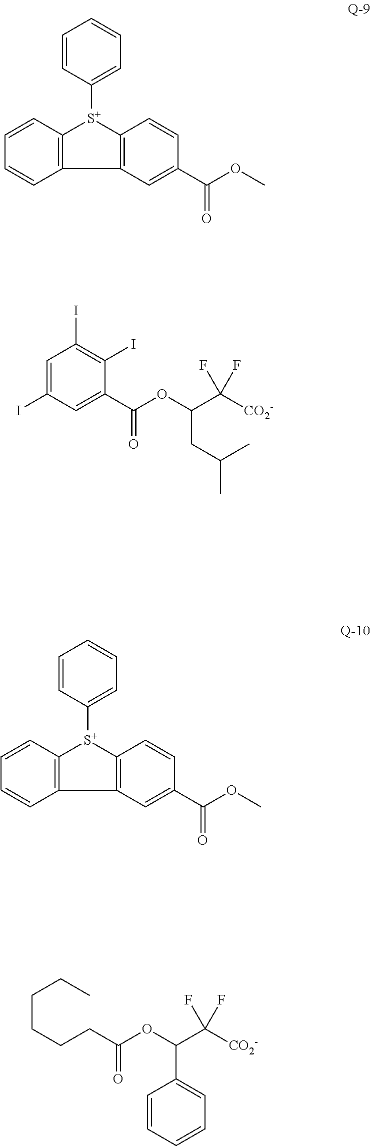

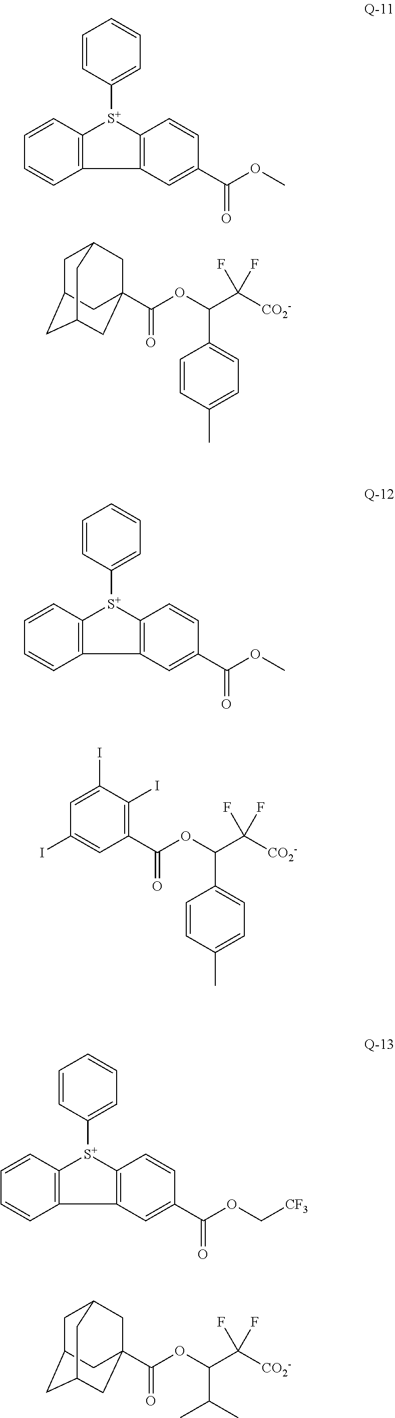

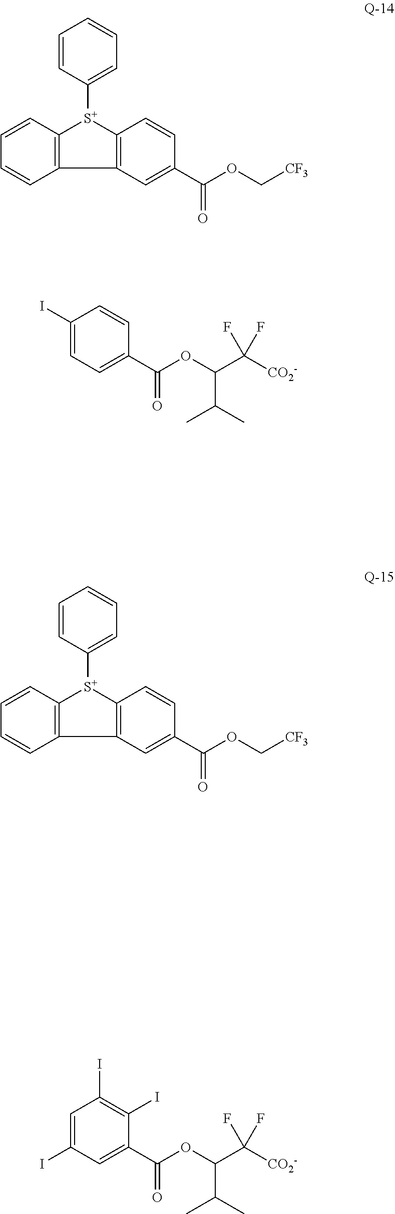

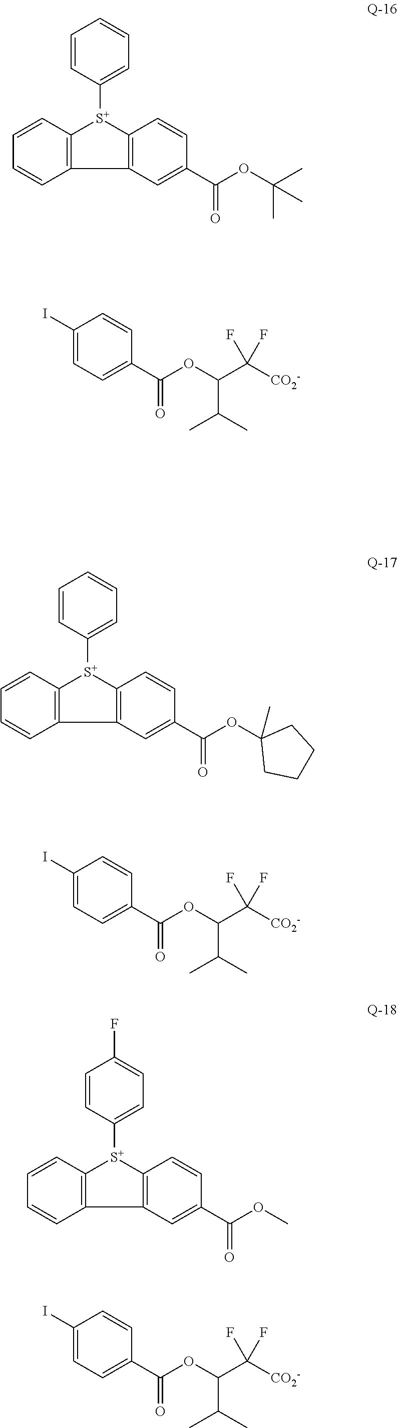

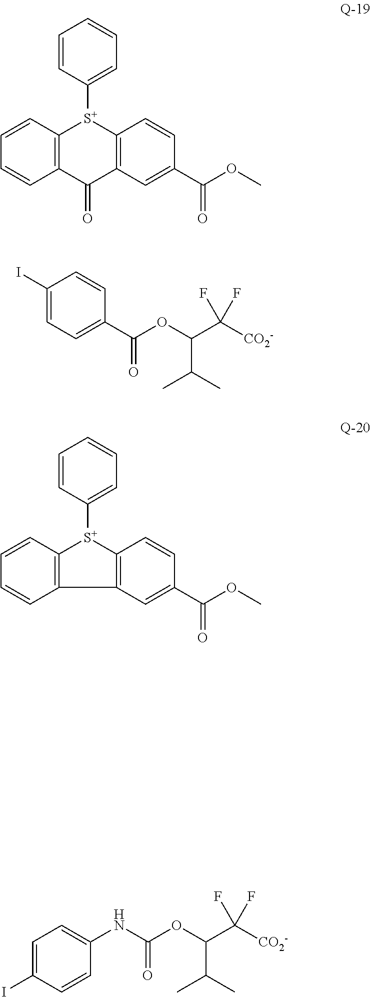

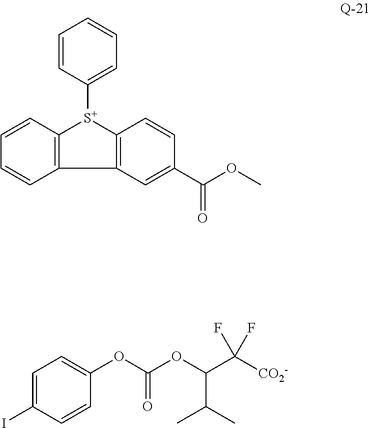

### Synthesis Example 2-2 Synthesis of Base Polymers P-1 to P-5

Base polymers P-1 to P-5 were synthesized by combining monomers, performing copolymerization reaction in THF solvent, pouring the reaction solution to methanol for precipitation, washing the solid precipitate with hexane, isolation and drying. The polymer was analyzed for composition by ¹H-NMR spectroscopy and for Mw and Mw/Mn by GPC versus polystyrene standards using THF solvent.

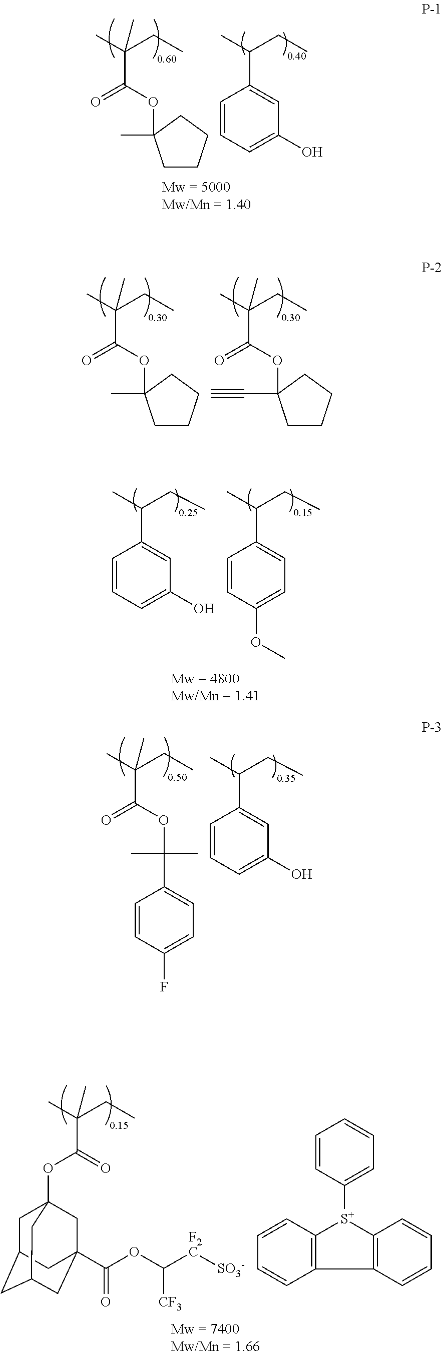

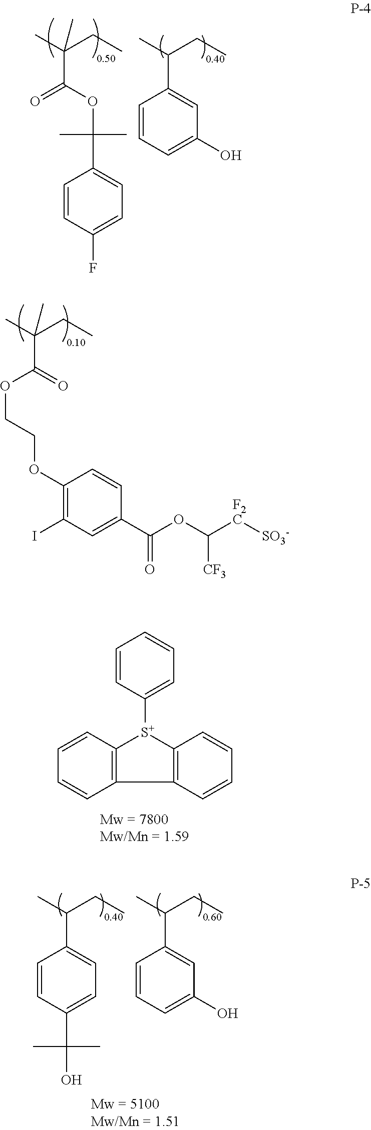

### Examples 1 to 25 and Comparative Examples 1 to 4

(1) Preparation of Resist Compositions

Resist compositions in solution form were prepared by dissolving selected components in a solvent in accordance with the formulation shown in Tables 1 and 2, and filtering through a filter with a pore size of 0.2 μm. The resist compositions of Examples 1 to 24 and Comparative Examples 1 to 3 were of positive tone whereas the resist compositions of Example 25 and Comparative Example 4 were of negative tone.

The components in Tables 1 and 2 are identified below.

Organic Solvents:
PGMEA (propylene glycol monomethyl ether acetate)
DAA (diacetone alcohol)
EL (ethyl acetate)

Acid Generators: PAG-1 to PAG-4

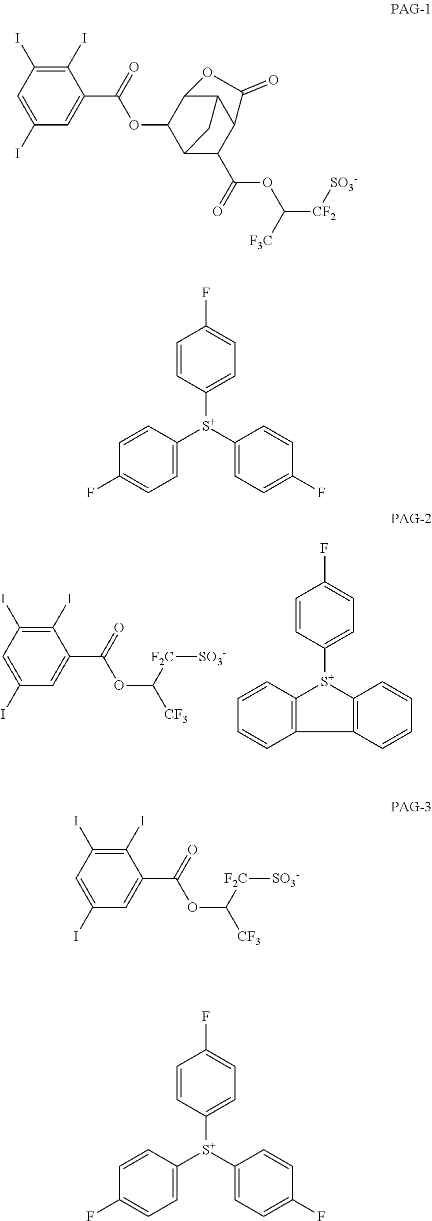

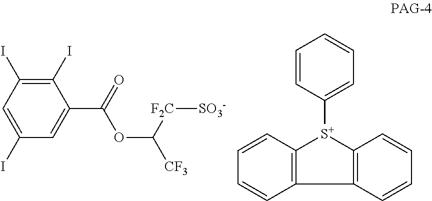

Blend Quenchers: bQ-1 and bQ-2

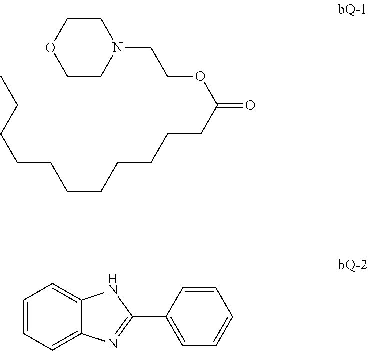 [SMILES: CCCCCCCCCCCC(=O)OCCN1CCOCC1 ; c1ccc(-c2nc3ccccc3[nH]2)cc1]

Comparative Quenchers: cQ-1 to cQ-3

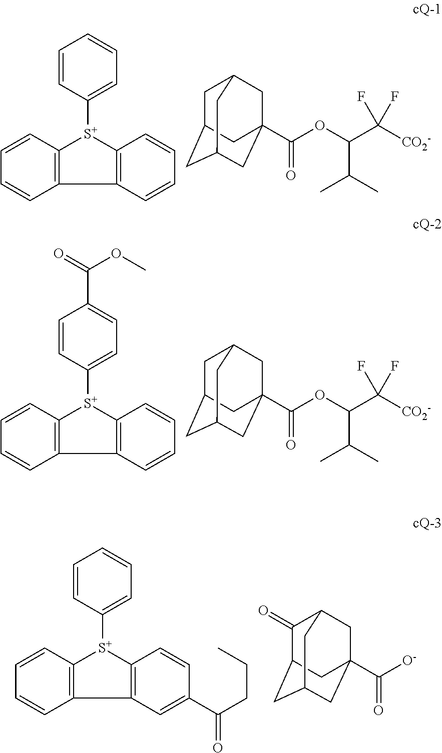

(2) EUV Lithography Test

Each of the resist compositions in Tables 1 and 2 was spin coated on a silicon substrate having a 20-nm coating of silicon-containing spin-on hard mask SHB-A940 (Shin-Etsu Chemical Co., Ltd., silicon content 43 wt %) and prebaked on a hotplate at 100° C. for 60 seconds to form a resist film of 60 nm thick. Using an EUV scanner NXE3400 (ASML, NA 0.33, σ0.9/0.6, quadrupole illumination), the resist film was exposed to EUV through a mask bearing a hole pattern having a pitch of 44 nm+20% bias (on-wafer size). The resist film was baked (PEB) on a hotplate at the temperature shown in Tables 1 and 2 for 60 seconds and developed in a 2.38 wt % TMAH aqueous solution for 30 seconds. Hole patterns with a size of 22 nm were obtained in Examples 1 to 24 and Comparative Examples 1 to 3 whereas dot patterns with a size of 22 nm were obtained in Example 25 and Comparative Example 4.

The hole or dot pattern was observed under CD-SEM (CG6300, Hitachi High-Technologies Corp.). The exposure dose that provides a hole or dot pattern having a size of 22 nm was determined and reported as sensitivity. The size of 50 holes or dots at that dose was measured, from which a 3-fold value (36) of the standard deviation (a) was computed and reported as CDU. The results are also shown in Tables 1 and 2.

TABLE 1

| | | Base | Acid | | Organic | PEB | | |
|---|---|---|---|---|---|---|---|---|
| | | polymer | generator | Quencher | solvent | temp. | Sensitivity | CDU |
| | | (pbw) | (pbw) | (pbw) | (pbw) | (° C.) | (mJ/cm²) | (nm) |
| Example | 1 | P-1 | PAG-1 | Q-1 | PGMEA (3000) | 90 | 28 | 2.6 |
| | | (100) | (30.2) | (6.5) | DAA (500) | | | |
| | 2 | P-1 | PAG-2 | Q-2 | PGMEA (3000) | 90 | 26 | 2.5 |
| | | (100) | (24.9) | (6.6) | DAA (500) | | | |
| | 3 | P-1 | PAG-2 | Q-3 | PGMEA (3000) | 90 | 26 | 2.5 |
| | | (100) | (24.9) | (7.2) | DAA (500) | | | |
| | 4 | P-1 | PAG-2 | Q-4 (3.6) | PGMEA (3000) | 90 | 27 | 2.6 |
| | | (100) | (24.9) | bQ-1 (1.6) | DAA (500) | | | |
| | 5 | P-1 | PAG-2 | Q-5 | PGMEA (3000) | 90 | 23 | 2.3 |
| | | (100) | (24.9) | (9.7) | DAA (500) | | | |
| | 6 | P-1 | PAG-2 | Q-6 | PGMEA (3000) | 90 | 24 | 2.4 |
| | | (100) | (24.9) | (8.6) | DAA (500) | | | |
| | 7 | P-1 | PAG-2 | Q-7 | PGMEA (3000) | 90 | 27 | 2.7 |
| | | (100) | (24.9) | (6.1) | DAA (500) | | | |
| | 8 | P-1 | PAG-2 | Q-8 | PGMEA (3000) | 90 | 26 | 2.6 |
| | | (100) | (24.9) | (7.3) | DAA (500) | | | |
| | 9 | P-1 | PAG-3 | Q-9 (4.9) | PGMEA (3000) | 90 | 25 | 2.7 |
| | | (100) | (25.9) | bQ-2 (1.0) | DAA (500) | | | |
| | 10 | P-1 | PAG-3 | Q-10 | PGMEA (3000) | 90 | 27 | 2.7 |
| | | (100) | (25.9) | (6.3) | DAA (500) | | | |
| | 11 | P-1 | PAG-3 | Q-11 | PGMEA (3000) | 90 | 26 | 2.6 |
| | | (100) | (25.9) | (8.1) | DAA (500) | | | |
| | 12 | P-1 | PAG-3 | Q-12 | EL (3000) | 90 | 24 | 2.5 |
| | | (100) | (25.9) | (11.3) | DAA (500) | | | |
| | 13 | P-1 | PAG-3 | Q-13 | EL (3500) | 90 | 27 | 2.5 |
| | | (100) | (25.9) | (7.2) | | | | |
| | 14 | P-1 | PAG-3 | Q-14 | PGMEA (3000) | 90 | 25 | 2.4 |
| | | (100) | (25.9) | (7.9) | DAA (500) | | | |
| | 15 | P-1 | PAG-3 | Q-15 | PGMEA (3000) | 90 | 22 | 2.3 |
| | | (100) | (25.9) | (10.4) | DAA (500) | | | |
| | 16 | P-1 | PAG-3 | Q-16 | PGMEA (3000) | 90 | 23 | 2.5 |
| | | (100) | (25.9) | (7.6) | DAA (500) | | | |
| | 17 | P-1 | PAG-3 | Q-17 | PGMEA (3000) | 90 | 24 | 2.5 |
| | | (100) | (25.9) | (7.9) | DAA (500) | | | |
| | 18 | P-1 | PAG-3 | Q-18 | PGMEA (3000) | 90 | 24 | 2.4 |
| | | (100) | (25.9) | (7.4) | DAA (500) | | | |
| | 19 | P-1 | PAG-3 | Q-19 | PGMEA (3000) | 90 | 28 | 2.6 |
| | | (100) | (25.9) | (7.5) | DAA (500) | | | |
| | 20 | P-1 | PAG-3 | Q-20 | PGMEA (3000) | 90 | 24 | 2.4 |
| | | (100) | (25.9) | (7.3) | DAA (500) | | | |
| | 21 | P-1 | PAG-4 | Q-21 | PGMEA (3000) | 90 | 24 | 2.3 |
| | | (100) | (24.5) | (7.3) | DAA (500) | | | |
| | 22 | P-2 | PAG-2 | Q-5 | PGMEA (3000) | 90 | 23 | 2.4 |
| | | (100) | (24.9) | (9.7) | DAA (500) | | | |
| | 23 | P-3 | — | Q-5 | PGMEA (3000) | 90 | 24 | 2.3 |
| | | (100) | | (9.7) | DAA (500) | | | |
| | 24 | P-4 | — | Q-5 | PGMEA (3000) | 90 | 23 | 2.3 |
| | | (100) | | (9.7) | DAA (500) | | | |
| | 25 | P-5 | PAG-1 | Q-5 | PGMEA (3000) | 110 | 30 | 3.1 |
| | | (100) | (30.2) | (9.7) | DAA (500) | | | |

TABLE 2

| | | Base | Acid | | Organic | PEB | | |
|---|---|---|---|---|---|---|---|---|
| | | polymer | generator | Quencher | solvent | temp. | Sensitivity | CDU |
| | | (pbw) | (pbw) | (pbw) | (pbw) | (° C.) | (mJ/cm²) | (nm) |
| Comparative | 1 | P-1 | PAG-2 | cQ-1 | PGMEA (3000) | 90 | 33 | 3.1 |
| Example | | (100) | (24.9) | (5.9) | DAA (500) | | | |
| | 2 | P-1 | PAG-2 | cQ-2 | PGMEA (3000) | 90 | 32 | 3.2 |
| | | (100) | (24.9) | (6.5) | DAA (500) | | | |
| | 3 | P-1 | PAG-2 | cQ-3 | PGMEA (3000) | 90 | 34 | 3.3 |
| | | (100) | (24.9) | (5.3) | DAA (500) | | | |
| | 4 | P-5 | PAG-2 | cQ-1 | PGMEA (3000) | 110 | 38 | 4.1 |
| | | (100) | (24.9) | (5.9) | DAA (500) | | | |

It is demonstrated in Tables 1 and 2 that resist compositions comprising Sulfonium Salt A offer a high sensitivity and improved CDU.

Japanese Patent Application No. 2022-176346 is incorporated herein by reference. Although some preferred embodiments have been described, many modifications and variations may be made thereto in light of the above teachings. It is therefore to be understood that the invention may be practiced otherwise than as specifically described without departing from the scope of the appended claims.

1. A sulfonium salt having the formula (1): 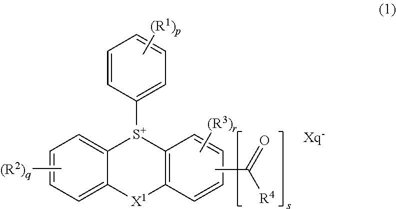
wherein p, q and r are each independently an integer of 0 to 3, s is 1 or 2, and r+s is from 1 to 3,
R¹ and R² are each independently halogen, trifluoromethyl, trifluoromethoxy, trifluoromethylthio, nitro, cyano, —C(═O)—R⁴, —O—C(═O)—R⁵ or —O—R⁵,
R³ is halogen, trifluoromethyl, trifluoromethoxy, trifluoromethylthio, nitro, cyano, —O—C(═O)—R⁵ or —O—R⁵,
R⁴ is a C₁-C₁₀ hydrocarbyl group, C₁-C₁₀ hydrocarbyloxy group, or —O—R^{4A}, the hydrocarbyl group and hydrocarbyloxy group may be substituted with fluorine or hydroxy, R^{4A} is an acid labile group,
R⁵ is a C₁-C₁₀ hydrocarbyl group,
X¹ is a single bond, ether bond, carbonyl group, —N(R)—, sulfide bond or sulfonyl group, R is hydrogen or a C₁-C₆ saturated hydrocarbyl group,
Xq⁻ is an anion having the formula (2): 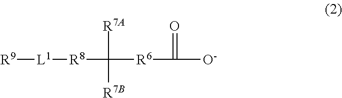
wherein R⁶ is a single bond or C₁-C₄ alkanediyl group,
R^{7A} and R^{7B} are each independently hydrogen, fluorine, or trifluoromethyl, at least one thereof being fluorine or trifluoromethyl,
R⁸ is a single bond or C₁-C₂₀ hydrocarbylene group which may contain a heteroatom,
R⁹ is a C₆-C₂₀ straight or branched hydrocarbyl group which may contain a heteroatom or C₃-C₂₀ cyclic hydrocarbyl group which may contain a heteroatom, and
L¹ is an ether bond, ester bond, carbonate bond or urethane bond.

2. The sulfonium salt of claim 1 wherein R⁶ is a single bond, R^{7A} and R^{7B} are fluorine.

3. A resist composition comprising a quencher containing the sulfonium salt of claim 1.

4. The resist composition of claim 3, further comprising a base polymer.

5. The resist composition of claim 4 wherein the base polymer comprises repeat units having the formula (a1) or repeat units having the formula (a2): 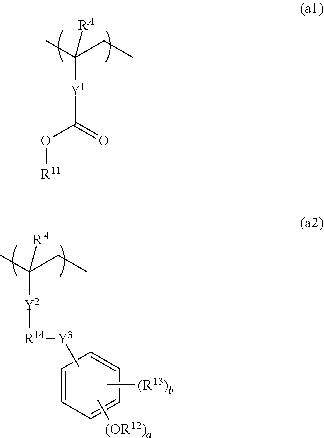
wherein R^{A} is each independently hydrogen or methyl,
Y¹ is a single bond, phenylene, naphthylene, or a C₁-C₁₂ linking group containing an ester bond and/or lactone ring,
Y² is a single bond or ester bond,
Y³ is a single bond, ether bond or ester bond,
R¹¹ and R¹² are each independently an acid labile group,
R¹³ is fluorine, trifluoromethyl, cyano or a C₁-C₆ saturated hydrocarbyl group,
R¹⁴ is a single bond or a C₁-C₆ alkanediyl group in which some carbon may be replaced by an ether bond or ester bond,
a is 1 or 2, b is an integer of 0 to 4, and a+b is from 1 to 5.

6. The resist composition of claim 5 which is a chemically amplified positive resist composition.

7. The resist composition of claim 4 wherein the base polymer is free of an acid labile group.

8. The resist composition of claim 7 which is a chemically amplified negative resist composition.

9. The resist composition of claim 4 wherein the base polymer comprises repeat units of at least one type selected from repeat units having the formulae (f1) to (f3): 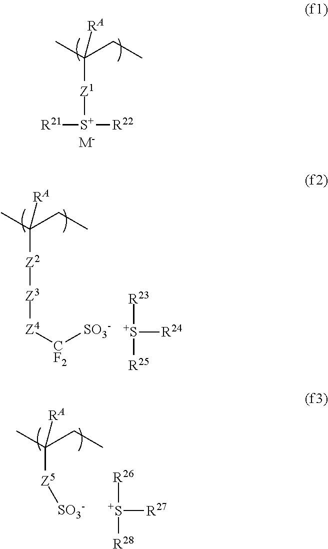
wherein R^{A} is each independently hydrogen or methyl,
Z¹ is a single bond, a C₁-C₆ aliphatic hydrocarbylene group, phenylene group, naphthylene group, or C₇-Cis group obtained by combining the foregoing, or —O—Z¹¹—, —C(═O)—O—Z¹¹— or —C(═O)—NH—Z¹¹—, Z¹¹ is a C₁-C₆ aliphatic hydrocarbylene group, phenylene group, naphthylene group, or C₇-C₁₈ group obtained by combining the foregoing, which may contain a carbonyl moiety, ester bond, ether bond or hydroxy moiety,
Z² is a single bond or ester bond,
Z³ is a single bond, —Z³¹—C(═O)—O—, —Z³¹—O— or —Z³¹—O—C(═O)—, Z³¹ is a C₁-C₁₂ aliphatic hydrocarbylene group, phenylene group, or C₇-C₁₈ group obtained by combining the foregoing, which may contain a carbonyl moiety, ester bond, ether bond, urethane bond, nitro, cyano, fluorine, iodine or bromine,
Z⁴ is methylene, 2,2,2-trifluoro-1,1-ethanediyl or carbonyl,
Z⁵ is a single bond, methylene, ethylene, phenylene, fluorinated phenylene, trifluoromethyl-substituted phenylene group, —O—Z⁵¹—, —C(═O)—O—Z⁵¹—, or —C(═O)—NH—Z⁵¹—, Z⁵¹ is a C₁-C₆ aliphatic hydrocarbylene group, phenylene group, fluorinated phenylene group, or trifluoromethyl-substituted phenylene group, which may contain a carbonyl moiety, ester bond, ether bond, halogen or hydroxy moiety,
R²¹ to R²⁸ are each independently halogen or a C₁-C₂₀ hydrocarbyl group which may contain a heteroatom, a pair of R²³ and R²⁴ or R²⁶ and R²⁷ may bond together to form a ring with the sulfur atom to which they are attached, and
M⁻ is a non-nucleophilic counter ion.

10. The resist composition of claim 3, further comprising an acid generator capable of generating a strong acid.

11. The resist composition of claim 10 wherein the acid generator generates a sulfonic acid, imide acid or methide acid.

12. The resist composition of claim 3, further comprising an organic solvent.

13. The resist composition of claim 3, further comprising a surfactant.

14. A pattern forming process comprising the steps of applying the resist composition of claim 3 onto a substrate to form a resist film thereon, exposing the resist film to high-energy radiation, and developing the exposed resist film in a developer.

15. The process of claim 14 wherein the high-energy radiation is KrF excimer laser, ArF excimer laser, EB or EUV of wavelength 3 to 15 nm.
